(19) United States
(12) Patent Application Publication
(10) Pub. No.: US 2023/0063017 A1
(43) Pub. Date: Mar. 2, 2023

(54) BIOMARKER SIGNATURES OF SYSTEMIC LUPUS ERYTHEMATOSUS AND USES THEREOF

(71) Applicant: IMMUNOVIA AB, Lund (SE)

(72) Inventors: Carl BORREBAECK, Lund (SE); Payam DELFANI, Malmö (SE); Linda Dexlin MELLBY, Dalby (SE); Christer WINGREN, Södra Sandby (SE)

(21) Appl. No.: 17/848,365

(22) Filed: Jun. 23, 2022

Related U.S. Application Data
(63) Continuation of application No. 16/308,275, filed on Dec. 7, 2018, now abandoned.

(30) Foreign Application Priority Data
Jun. 7, 2016 (GB) 1609951.7

(51) Int. Cl. G01N 33/564

(52) U.S. Cl. CPC G01N 33/564; G01N 2800/104; G01N 2800/56

(57) ABSTRACT

The invention provides a method for determining a systemic lupus erythematosus-associated disease state in a subject comprising the steps of (a) providing a sample to be tested; and (b) measuring the presence and/or amount in the test sample of one or more biomarker(s) selected from the group defined in Table A, wherein the presence and/or amount in the test sample of the one or more biomarker(s) selected from the group defined in Table A is indicative of a systemic Lupus-associated disease state. The invention also provides an array and a kit suitable for use in the methods of the invention.

This application is a continuation of U.S. patent application Ser. No. 16/308,275, filed Dec. 7, 2018, which is a national stage application under 35 U.S.C. § 371 of PCT Application No. PCT/EP2017/063855, filed Jun. 7, 2017, which claims the benefit of Great Britain Patent Application No. 1609951.7, filed Jun. 7, 2016.

### FIELD OF INVENTION

The present invention relates to biomarkers for determining a systemic lupus erythematosus-associated disease state, as well as signatures and arrays thereof and methods for use of the same.

### BACKGROUND OF THE INVENTION

Systemic lupus erythematosus (SLE) is a severe, chronic systemic autoimmune disease with heterogeneous presentation (1, 2). The disease is characterized by alternating periods of flares and remission in a yet unpredictable manner. The treatment of SLE is so far restricted to dealing with the symptoms, essentially trying to reduce and minimize the effects of the flares (3). Accumulation of damage is a function of disease activity over time, side-effects of treatment and/or comorbid conditions, and is linked to morbidity and mortality. Hence, novel means to predict, detect, and monitor the onset, severity, and response of flares, especially to the level of renal activity, would thus be essential to therapeutic regime choice and treatment modifications, as well as prognosis (2-4). Currently, disease activity is assessed using indices, such as SLEDAI-2K, which is based on 24 descriptors in 9 organs (studied over 10 or 30 days) (5). Monitoring of the renal involvement in SLE is often dependent on microscopic evaluation of urine, which has been shown to be associated with large methodological shortcomings (6). So far, renal biopsy has been the gold standard for assessing disease activity and renal involvement, but the need for rapid, minimally invasive methods, such as blood-based tests, is evident.

Historically, mainly single serological biomarkers, such as C1q, C3, C4, IL-6, TNF-α, and various autoantibodies, have been explored for detecting and monitoring flares, but the performance has varied and is generally low (4, 7-11). In more recent years, attempts have therefore been made to decipher panels of biomarkers, better reflecting SLE and SLE disease activity (12-17). As for example, Li and colleagues used 30-plex antigen arrays to delineate candidate serum autoantibody clusters that attempted to distinguish lupus patients with more severe disease activity (18). Further, Bauer and colleagues pre-validated a 3-plex panel of serum chemokines (IP-10, MCP-1, and MIP3B) for disease activity using conventional antibody microarrays (16, 17).

More recently, high-performing recombinant antibody micro- and nano-array set-ups have been used for deciphering multiplexed serum and urine biomarkers associated with SLE (19, 20) (Nordström et al, submitted; Delfani et al, 2016, Lupus 26(4):373-387). In those projects, the authors explored the use of the immune system as a specific and sensitive sensor for SLE in an approach denoted clinical immunoproteomics (21) by targeting mainly immunoregulatory proteins. The results showed that multiplexed candidate serum (and urine) biomarkers reflecting SLE, SLE phenotype and SLE disease activity could be deciphered.

However, there remains are continuing need to identify biomarkers and biomarker signatures for disease activity in order to rapidly identify active (flaring) and passive (remissive/non-flaring) disease states to allow quick treatment of flares and withdrawal of therapy during non-flares to minimise the damage caused by non-treatment of the active disease and overtreatment (treatment administration during non-active SLE).

### SUMMARY OF THE INVENTION

The present invention stems from a study of serum protein expression profiling performed using SLE samples collected during flare and remission. A re-optimized recombinant antibody microarray platform, displaying improved performances and targeting a larger set of immunoregulatory proteins (19) (Delfani et al, 2016, supra) was applied. The results showed that condensed, high-performing serum biomarker signatures reflecting disease activity could be deciphered from crude serum samples.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-E show serum biomarker panel discriminating Active SLE vs. Normal. FIG. 1A shows backward elimination analysis of the training set, resulting in a condensed set of 25 antibodies (marked with an arrow) providing the best classification. FIG. 1B shows AUC ROC curve for the test set, based on the frozen SVM model and 25-plex antibody signature. FIG. 1C shows principle component analysis (PCA) plot of the training set onto which the test set was then mapped. FIG. 1D shows PCA plot of the test set only where the training set is removed from the plot for a clearer view of the separation of samples in the test set. FIG. 1E shows heat map for the test set, based on the 25-plex antibody signature (red represents up-regulated, green represents down-regulated, and black represents unchanged).

FIGS. 2A-H show serum biomarker panels classifying NonActive SLE vs. Normal (FIGS. 2A-D), and Active SLE vs. NonActive SLE (FIGS. 2E-H). FIGS. 2A and 2E show AUC ROC curves for the test sets, based on the frozen SVM models and 25-plex antibody signatures for each corresponding comparison. FIGS. 2B and 2F show principle component analysis (PCA) plots of the training sets onto which the test sets were then mapped with respect to corresponding comparison. FIGS. 2C and 2G show PCA plot of the test sets only where the training sets are removed from the plots for a clearer view of the separation of samples in the test sets. FIGS. 2D and 2H show heat maps for the test sets, based on the 25-plex antibody signatures (red represents up-regulated, green represents down-regulated, and black represents unchanged).

FIGS. 3A-F show robustness of the data set on the classification of Active SLE vs. Normal (FIGS. 3A and 3D), NonActive SLE vs. Normal (FIGS. 3B and 3E), and Active SLE vs. NonActive SLE (FIGS. 3C and 3F). FIGS. 3A-C show boxplots of the AUC ROC values for the test sets, based on the frozen SVM models and 25-plex antibody signatures, iterated ten times, i.e. using ten different pairs of training and test sets for each corresponding comparison. FIGS. 3D-F show frequencies (>50%) at which each biomarker occurred in the ten 25-plex antibody signatures in each corresponding comparison are presented as tables.

FIG. 4A shows heat map for Active SLE vs. Normal, NonActive SLE vs. Normal, and Active SLE vs. NonActive SLE, based on the comparison of 31 non-redundant antigen proteins including the top 25 statistically differentially expressed analytes and the six significantly de-regulated proteins (based on the differences in fold change) where red represents up-regulated, green represents down-regulated, and black represents unchanged. FIG. 4B shows protein expression profiles of three selected key biomarkers are shown as boxplots. The median values are indicated (thick line) and the hinges represent the 25th percentile and the 75th percentile, respectively. The protein expression levels are shown for two complement proteins (C1q and C4) and cystatin C. FIG. 4C shows the protein expression level of complement factor C1q are shown as boxplots, when comparing NonActive SLE vs. Active SLE, with respect to array data and obtained clinic data measured by ELISA.

FIGS. 5A-B show serum biomarker panels discriminating HighActive SLE vs. Normal, and HighActive SLE vs. NonActive SLE. FIG. 5A shows HighActive SLE vs. Normal, illustrated by ROC AUC curve and heat map (20 top differentially expressed biomarkers; where red represents up-regulated, green represents down-regulated, and black represents unchanged). Normal is colored as (blue) and HighActive SLE as (green). FIG. 5B shows HighActive SLE vs. NonActive SLE, illustrated by ROC AUC curve and heat map (20 top differentially expressed biomarkers). SLE subsets are colored as: HighActive SLE (green) and NonActive SLE (brown).

FIG. 6 shows the protein expression levels of complement factors C4 and C1q as boxplots, when comparing HighActive SLE vs. NonActive SLE, with respect to array data and obtained clinic data measured by ELISA.

FIGS. 7A-C show classification of SLE patients, grouped according to disease severity (i.e. SLE1-SLE3), using SVM leave-one-out cross-validation procedure. SLE1 samples were classified when comparing Active SLE vs. Normal (FIG. 7A). SLE2 samples were classified when comparing Active SLE vs. Normal/NonActive SLE, and NonActive SLE vs. Normal (FIG. 7B). SLE3 samples were classified when comparing Active SLE vs. Normal/NonActive SLE, and NonActive SLE vs. N (FIG. 7C).

FIGS. 8A-D show longitudinal analysis of SLE samples (four samples per patient (n=4; denoted A-D)) collected at four different time-points during follow-up. The top (≤20) deregulated expressed serum proteins were identified using multi-group comparisons, and the samples were visualized using supervised hierarchical clustering in combination with heat-maps. The disease activity status of samples were started at time 0 and the time of sample collection were recorded (≤3.3 years). In the heat maps, red represents up-regulated, green represents down-regulated, and black represents unchanged.

### DETAILED DESCRIPTION OF THE INVENTION

The present invention stems from a study of serum protein expression profiling performed using SLE samples collected during flare and remission. A re-optimized recombinant antibody microarray platform, displaying improved performances and targeting a larger set of immunoregulatory proteins (19) (Delfani et al, 2016, supra) was applied. The results showed that condensed, high-performing serum biomarker signatures reflecting disease activity could be deciphered from crude serum samples.

Accordingly, the first aspect the invention provides a method for determining a systemic lupus erythematosus-associated disease state in a subject measuring the presence and/or amount in a test sample of one or more biomarker selected from the group defined in Table A, wherein the presence and/or amount in the one more test sample of the one or more biomarker(s) selected from the group defined in Table A is indicative of a systemic lupus erythematosus-associated disease state.

Alternative or additionally the method comprises or consists of the steps of:
a) providing a sample to be tested; and
b) measuring the presence and/or amount in the test sample of one or more biomarker(s) selected from the group defined in Table A;

wherein the presence and/or amount in the test sample of the one or more biomarker(s) selected from the group defined in Table A is indicative of the systemic lupus erythematosus-associated disease state in the subject.

Thus, the invention provides biomarkers and biomarker signatures for determining a systemic lupus erythematosus-associated disease state in a subject.

The term “Systemic lupus erythematosus-associated disease state” may mean or include (i) the presence or absence of SLE (e.g., discriminating active SLE from non-SLE, non-active SLE from non-SLE and/or highly active SLE from non-SLE), and (ii) the activity of SLE (e.g., discriminating active SLE from non-active SLE, and/or discriminating highly-active SLE from non-active SLE).

Thus, in one embodiment, the method is for diagnosing active SLE (e.g., an SLE flare) in a subject.

Preferably, the individual is a human, but may be any mammal such as a domesticated mammal (preferably of agricultural or commercial significance including a horse, pig, cow, sheep, dog and cat).

For the avoidance of doubt, test samples from more than one disease state may be provided in step (a), for example, ≥2, ≥3, ≥4, ≥5, ≥6 or ≥7 different disease states. Step (a) may provide at least two test samples, for example, ≥3, ≥4, ≥5, ≥6, ≥7, ≥8, ≥9, ≥10, ≥15, ≥20, ≥25, ≥50 or ≥100 test samples. Where multiple test samples are provided, they may be of the same type (e.g., all serum or urine samples) or of different types (e.g., serum and urine samples).

Alternatively or additionally the method further comprises the steps of:
c) providing a control sample from an individual with a different systemic lupus erythematosus-associated disease state to the test subject; and
d) measuring the presence and/or amount in the control sample of the one or more biomarkers measured in step (b);

wherein the systemic lupus erythematosus-associated disease state is identified in the event that the presence and/or amount in the test sample of the one or more biomarkers measured in step (b) is different from the presence and/or amount in the control sample.

For the avoidance of doubt, control samples from more than one disease state may be provided in step (c), for example, ≥2, ≥3, ≥4, ≥5, ≥6 or ≥7 different disease states. Step (c) may provide at least two control samples, for example, ≥3, ≥4, ≥5, ≥6, ≥7, ≥8, ≥9, ≥10, ≥15, ≥20, ≥25, ≥50 or ≥100 control samples. Where multiple control samples are provided, they may be of the same type (e.g., all serum or urine samples) or of different types (e.g., serum and urine samples). Preferably the test samples types and control samples types are matched/corresponding.

By “is different to the presence and/or amount in a control sample” we mean or include the presence and/or amount of the one or more biomarker in the test sample differs from that of the one or more control sample (or to predefined reference values representing the same). Preferably the presence and/or amount in the test sample differs from the presence or amount in the one or more control sample (or mean of the control samples) by at least ±5%, for example, at least ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, ±24%, ±25%, ±26%, ±27%, ±28%, ±29%, ±30%, ±31%, ±32%, ±33%, ±34%, ±35%, ±36%, ±37%, ±38%, ±39%, ±40%, ±41%, ±42%, ±43%, ±44%, ±45%, ±41%, ±42%, ±43%, ±44%, ±55%, ±60%, ±65%, ±66%, ±67%, ±68%, ±69%, ±70%, ±71%, ±72%, ±73%, ±74%, ±75%, ±76%, ±77%, ±78%, ±79%, ±80%, ±81%, ±82%, ±83%, ±84%, ±85%, ±86%, ±87%, ±88%, ±89%, ±90%, ±91%, ±92%, ±93%, ±94%, ±95%, ±96%, ±97%, ±98%, ±99%, ±100%, ±125%, ±150%, ±175%, ±200%, ±225%, ±250%, ±275%, ±300%, ±350%, ±400%, ±500% or at least ±1000% of the one or more control sample (e.g., the negative control sample).

Alternatively or additionally, the presence or amount in the test sample differs from the mean presence or amount in the control samples by at least >1 standard deviation from the mean presence or amount in the control samples, for example, ≥1.5, ≥2, ≥3, ≥4, ≥5, ≥6, ≥7, ≥8, ≥9, ≥10, ≥11, ≥12, ≥13, ≥14 or ≥15 standard deviations from the from the mean presence or amount in the control samples. Any suitable means may be used for determining standard deviation (e.g., direct, sum of square, Welford's), however, in one embodiment, standard deviation is determined using the direct method (i.e., the square root of [the sum the squares of the samples minus the mean, divided by the number of samples]).

Alternatively or additionally, by “is different to the presence and/or amount in a control sample” we mean or include that the presence or amount in the test sample does not correlate with the amount in the control sample in a statistically significant manner. By “does not correlate with the amount in the control sample in a statistically significant manner” we mean or include that the presence or amount in the test sample correlates with that of the control sample with a p-value of >0.001, for example, >0.002, >0.003, >0.004, >0.005, >0.01, >0.02, >0.03, >0.04 >0.05, >0.06, >0.07, >0.08, >0.09 or >0.1. Any suitable means for determining p-value known to the skilled person can be used, including z-test, t-test, Student's t-test, f-test, Mann-Whitney U test, Wilcoxon signed-rank test and Pearson's chi-squared test.

Alternatively or additionally the method further comprises the steps of:
e) providing a control sample from an individual with the same systemic lupus erythematosus-associated disease state to the test subject; and
f) measuring the presence and/or amount in the control sample of the one or more biomarkers measured in step (b);

wherein the systemic lupus erythematosus-associated disease state is identified in the event that the expression in the test sample of the one or more biomarkers measured in step (b) corresponds to the expression in the control sample of the one or more biomarkers measured in step (f).

For the avoidance of doubt, control samples from more than one disease state may be provided in step (e), for example, ≥2, ≥3, ≥4, ≥5, ≥6 or ≥7 different disease states. Step (e) may provide at least two control samples, for example, ≥3, ≥4, ≥5, ≥6, ≥7, ≥8, ≥9, ≥10, ≥15, ≥20, ≥25, ≥50 or ≥100 control samples. Where multiple control samples are provided, they may be of the same type (e.g., all serum or urine samples) or of different types (e.g., serum and urine samples). Preferably the test samples types and control samples types are matched/corresponding.

By “corresponds to the presence and/or amount in a control sample” we mean or include the presence and/or amount is identical to that of a positive control sample; or closer to that of one or more positive control sample than to one or more negative control sample (or to predefined reference values representing the same). Preferably the presence and/or amount is within ±40% of that of the one or more control sample (or mean of the control samples), for example, within ±39%, ±38%, ±37%, ±36%, ±35%, ±34%, ±33%, ±32%, ±31%, ±30%, ±29%, ±28%, ±27%, ±26%, ±25%, ±24%, ±23%, ±22%, ±21%, ±20%, ±19%, ±18%, ±17%, ±16%, ±15%, ±14%, ±13%, ±12%, ±11%, ±10%, ±9%, ±8%, ±7%, ±6%, ±5%, ±4%, ±3%, ±2%, ±1%, ±0.05% or within 0% of the one or more control sample (e.g., the positive control sample).

Alternatively or additionally, the difference in the presence or amount in the test sample is ≤55 standard deviation from the mean presence or amount in the control samples, for example, ≤4.5, ≤4, ≤3.5, ≤3, ≤2.5, ≤2, ≤1.5, ≤1.4, ≤1.3, ≤1.2, ≤1.1, ≤1, ≤0.9, ≤0.8, ≤0.7, ≤0.6, ≤0.5, ≤0.4, ≤0.3, ≤0.2, ≤0.1 or 0 standard deviations from the from the mean presence or amount in the control samples, provided that the standard deviation ranges for differing and corresponding biomarker expressions do not overlap (e.g., abut, but no not overlap).

Alternatively or additionally, by “corresponds to the presence and/or amount in a control sample” we mean or include that the presence or amount in the test sample correlates with the amount in the control sample in a statistically significant manner. By “correlates with the amount in the control sample in a statistically significant manner” we mean or include that the presence or amount in the test sample correlates with the that of the control sample with a p-value of ≤0.05, for example, ≤0.04, ≤0.03, ≤0.02, ≤0.01, ≤0.005, ≤0.004, ≤0.003, ≤0.002, ≤0.001, ≤0.0005 or ≤0.0001.

Differential expression (up-regulation or down regulation) of biomarkers, or lack thereof, can be determined by any suitable means known to a skilled person. Differential expression is determined to a p value of a least less than 0.05 (p=<0.05), for example, at least <0.04, <0.03, <0.02, <0.01, <0.009, <0.005, <0.001, <0.0001, <0.00001 or at least <0.000001. Alternatively or additionally, differential expression is determined using a support vector machine (SVM). Alternatively or additionally, the SVM is an SVM as described below.

It will be appreciated by persons skilled in the art that differential expression may relate to a single biomarker or to multiple biomarkers considered in combination (i.e. as a biomarker signature). Thus, a p value may be associated with a single biomarker or with a group of biomarkers. Indeed, proteins having a differential expression p value of greater than 0.05 when considered individually may nevertheless still be useful as biomarkers in accordance with the invention when their expression levels are considered in combination with one or more other biomarkers.

As exemplified in the accompanying examples, the expression of certain biomarkers in a tissue, blood, serum or plasma test sample may be indicative of an SLE-associated disease state in an individual. For example, the relative expression of certain serum proteins in a single test sample may be indicative of the activity of SLE in an individual.

In an alternative or additional embodiment, the presence and/or amount in the test sample of the one or more biomarkers measured in step (b) are compared against predetermined reference values representative of the measurements in steps (d) and/or (f).

Alternatively or additionally, step (b) comprises or consists of measuring the presence and/or amount in the test sample of one or more of the biomarkers defined in Table A, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68 or 69 of the biomarkers defined in Table A.

Alternatively or additionally, step (b) comprises, consists of or excludes measuring the presence and/or amount of CHX10 (3); Alternatively or additionally, step (b) comprises, consists of or excludes measuring the presence and/or amount of LUM; Alternatively or additionally, step (b) comprises, consists of or excludes measuring the presence and/or amount of Cyst. C; Alternatively or additionally, step (b) comprises, consists of or excludes measuring the presence and/or amount of ATP5B (2); Alternatively or additionally, step (b) comprises, consists of or excludes measuring the presence and/or amount of Beta-galactosidase; Alternatively or additionally, step (b) comprises, consists of or excludes measuring the presence and/or amount of DUSP9; Alternatively or additionally, step (b) comprises, consists of or excludes measuring the presence and/or amount of IL-1 alpha; Alternatively or additionally, step (b) comprises, consists of or excludes measuring the presence and/or amount of IL-1 beta; Alternatively or additionally, step (b) comprises, consists of or excludes measuring the presence and/or amount of Motif (13); Alternatively or additionally, step (b) comprises, consists of or excludes measuring the presence and/or amount of Motif (14); Alternatively or additionally, step (b) comprises, consists of or excludes measuring the presence and/or amount of Motif (3); Alternatively or additionally, step (b) comprises, consists of or excludes measuring the presence and/or amount of Motif (4); Alternatively or additionally, step (b) comprises, consists of or excludes measuring the presence and/or amount of Motif (5); Alternatively or additionally, step (b) comprises, consists of or excludes measuring the presence and/or amount of Motif (7); Alternatively or additionally, step (b) comprises, consists of or excludes measuring the presence and/or amount of Motif (8); Alternatively or additionally, step (b) comprises, consists of or excludes measuring the presence and/or amount of MYOM2 (1); Alternatively or additionally, step (b) comprises, consists of or excludes measuring the presence and/or amount of PSA; Alternatively or additionally, step (b) comprises, consists of or excludes measuring the presence and/or amount of Sox11a; Alternatively or additionally, step (b) comprises, consists of or excludes measuring the presence and/or amount of Surface Ag X; Alternatively or additionally, step (b) comprises, consists of or excludes measuring the presence and/or amount of TBC1 D9 (2); Alternatively or additionally, step (b) comprises, consists of or excludes measuring the presence and/or amount of Angiomotin (1); Alternatively or additionally, step (b) comprises, consists of or excludes measuring the presence and/or amount of APOA1 (1); Alternatively or additionally, step (b) comprises, consists of or excludes measuring the presence and/or amount of BTK (1); Alternatively or additionally, step (b) comprises, consists of or excludes measuring the presence and/or amount of C1 est. inh. (3); Alternatively or additionally, step (b) comprises, consists of or excludes measuring the presence and/or amount of C1q; Alternatively or additionally, step (b) comprises, consists of or excludes measuring the presence and/or amount of C1s; Alternatively or additionally, step (b) comprises, consists of or excludes measuring the presence and/or amount of C3; Alternatively or additionally, step (b) comprises, consists of or excludes measuring the presence and/or amount of C4; Alternatively or additionally, step (b) comprises, consists of or excludes measuring the presence and/or amount of C5 (1); Alternatively or additionally, step (b) comprises, consists of or excludes measuring the presence and/or amount of CD40; Alternatively or additionally, step (b) comprises, consists of or excludes measuring the presence and/or amount of CD40 ligand; Alternatively or additionally, step (b) comprises, consists of or excludes measuring the presence and/or amount of Eotaxin (3); Alternatively or additionally, step (b) comprises, consists of or excludes measuring the presence and/or amount of Factor B; Alternatively or additionally, step (b) comprises, consists of or excludes measuring the presence and/or amount of GLP-1; Alternatively or additionally, step (b) comprises, consists of or excludes measuring the presence and/or amount of GM-CSF; Alternatively or additionally, step (b) comprises, consists of or excludes measuring the presence and/or amount of HLA-DR/DP; Alternatively or additionally, step (b) comprises, consists of or excludes measuring the presence and/or amount of ICAM-1; Alternatively or additionally, step (b) comprises, consists of or excludes measuring the presence and/or amount of IFN-gamma (1); Alternatively or additionally, step (b) comprises, consists of or excludes measuring the presence and/or amount of IgM; Alternatively or additionally, step (b) comprises, consists of or excludes measuring the presence and/or amount of IL-10 (1); Alternatively or additionally, step (b) comprises, consists of or excludes measuring the presence and/or amount of IL-11 (1); Alternatively or additionally, step (b) comprises, consists of or excludes measuring the presence and/or amount of IL-12 (1); Alternatively or additionally, step (b) comprises, consists of or excludes measuring the presence and/or amount of IL-13 (1); Alternatively or additionally, step (b) comprises, consists of or excludes measuring the presence and/or amount of IL-16 (1); Alternatively or additionally, step (b) comprises, consists of or excludes measuring the presence and/or amount of IL-18; Alternatively or additionally, step (b) comprises, consists of or excludes measuring the presence and/or amount of IL-1ra; Alternatively or additionally, step (b) comprises, consists of or excludes measuring the presence and/or amount of IL-2 (2); Alternatively or additionally, step (b) comprises, consists of or excludes measuring the presence and/or amount of IL-3; Alternatively or additionally, step (b) comprises, consists of or excludes measuring the presence and/or amount of IL-4; Alternatively or additionally, step (b) comprises, consists of or excludes measuring the presence and/or amount of IL-5; Alternatively or additionally, step (b) comprises, consists of or excludes measuring the presence and/or amount of IL-6; Alternatively or additionally, step (b) comprises, consists of or excludes measuring the presence and/or amount of IL-7; Alternatively or additionally, step (b) comprises, consists of or excludes measuring the presence and/or amount of IL-8; Alternatively or additionally, step (b) comprises, consists of or excludes measuring the presence and/or amount of IL-9; Alternatively or additionally, step (b) comprises, consists of or excludes measuring the presence and/or amount of Integrin alpha-10; Alternatively or additionally, step (b) comprises, consists of or excludes measuring the presence and/or amount of JAK3; Alternatively or additionally, step (b) comprises, consists of or excludes measuring the presence and/or amount of LDL (1); Alternatively or additionally, step (b) comprises, consists of or excludes measuring the presence and/or amount of Leptin; Alternatively or additionally, step (b) comprises, consists of or excludes measuring the presence and/or amount of Lewis x (1); Alternatively or additionally, step (b) comprises, consists of or excludes measuring the presence and/or amount of MCP-1; Alternatively or additionally, step (b) comprises, consists of or excludes measuring the presence and/or amount of MCP-3 (1); Alternatively or additionally, step (b) comprises, consists of or excludes measuring the presence and/or amount of MCP-4 (2); Alternatively or additionally, step (b) comprises, consists of or excludes measuring the presence and/or amount of Procathepsin W; Alternatively or additionally, step (b) comprises, consists of or excludes measuring the presence and/or amount of RANTES; Alternatively or additionally, step (b) comprises, consists of or excludes measuring the presence and/or amount of Sialle x; Alternatively or additionally, step (b) comprises, consists of or excludes measuring the presence and/or amount of TGF-beta1; Alternatively or additionally, step (b) comprises, consists of or excludes measuring the presence and/or amount of TNF-alpha (1); Alternatively or additionally, step (b) comprises, consists of or excludes measuring the presence and/or amount of TNF-beta; Alternatively or additionally, step (b) comprises, consists of or excludes measuring the presence and/or amount of VEGF (1).

In one embodiment, the biomarker mRNA and/or amino acid sequences correspond to those available on the GenBank database (http://www.ncbi.nlm.nih.gov/genbank/) and natural variants thereof. In a further embodiment, the biomarker mRNA and/or amino acid sequences correspond to those available on the GenBank database on 7 Jun. 2016.

Alternatively or additionally, the method excludes the use of biomarkers that are not listed in Table A and/or the present Examples section.

Alternatively or additionally step (b) comprises or consists of measuring the presence and/or amount in the test sample of one or more of the biomarkers defined in Table A(I) and/or (II), for example, 2, 3, 4, 5, 6, 7, 8, 9, 10, 11, 12, 13, 14, 15, 16, 17, 18, 19 or 20 of the biomarkers defined in Table A(I) and/or (II).

Alternatively or additionally step (b) comprises or consists of measuring the presence and/or amount in the test sample of one or more of the biomarkers defined in Table A(III), for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 21, 32, 33, 34, 35, 36, 37, 38, 39, 40, 41, 42, 43, 44, 45, 46, 47, 48 or 49 of the biomarkers defined in Table A(III).

Alternatively or additionally step (b) comprises or consists of:
a) measuring the presence and/or amount in the test sample of one or more of the biomarkers defined in Table B(I), for example, 2 or 3 of the biomarkers defined in Table B(I);
b) measuring the presence and/or amount in the test sample of one or more of the biomarkers defined in Table B(II), for example, 2 or 3 of the biomarkers defined in Table B(II);
c) measuring the presence and/or amount in the test sample of one or more of the biomarkers defined in Table B(III), for example, 2 of the biomarkers defined in Table B(III);
d) measuring the presence and/or amount in the test sample of one or more of the biomarkers defined in Table B(IV), for example, 2 of the biomarkers defined in Table B(IV);
e) measuring the presence and/or amount in the test sample of one or more of the biomarkers defined in Table B(V), for example, 2, 3 or 4 of the biomarkers defined in Table B(V);
f) measuring the presence and/or amount in the test sample of one or more of the biomarkers defined in Table B(VI), for example, 2 of the biomarkers defined in Table BV(VI);
g) measuring the presence and/or amount in the test sample the biomarker defined in Table B(VII);
h) measuring the presence and/or amount in the test sample the biomarker defined in Table B(VIII);
i) measuring the presence and/or amount in the test sample the biomarker defined in Table B(IX);
j) measuring the presence and/or amount in the test sample of one or more of the biomarkers defined in Table B(X), for example, 2, 3, 4, 5, 6 or 7 of the biomarkers defined in Table B(X);
k) measuring the presence and/or amount in the test sample of one or more of the biomarkers defined in Table B(XI), for example, 2, 3, 4, 5, 6, 7 or 8 of the biomarkers defined in Table B(XI);
l) measuring the presence and/or amount in the test sample of one or more of the biomarkers defined in Table B(XII), for example, 2 or 3 of the biomarkers defined in Table B(XII);
m) measuring the presence and/or amount in the test sample of one or more of the biomarkers defined in Table B(XIII), for example, 2 of the biomarkers defined in Table B(XIII);
n) measuring the presence and/or amount in the test sample the biomarker defined in Table B(XIV);
o) measuring the presence and/or amount in the test sample the biomarker defined in Table B(XV);
p) measuring the presence and/or amount in the test sample of one or more of the biomarkers defined in Table B(XVI), for example, 2, 3, 4, 5, 6, 7 or 8 of the biomarkers defined in Table B(XVI);
q) measuring the presence and/or amount in the test sample of one or more of the biomarkers defined in Table B(XVII), for example, 2, 3, 4, 5, 6, 7, 8, 9, 10, 11, 12, 13, 14, 15, 16 or 17 of the biomarkers defined in Table B(XVII);
r) measuring the presence and/or amount in the test sample the biomarker defined in Table B(XVIII); and/or
s) measuring the presence and/or amount in the test sample the biomarker defined in Table B(XIX).

Alternatively or additionally the method comprises, consists of, or is for determining whether the SLE-associated-disease state is active SLE or non SLE. Alternatively or additionally step (b) comprises or consists of measuring the presence and/or amount in the test sample of one or more of the biomarkers defined in Table B(I), (II), (III), (IV), (V), (VI), (VIII), (IX), (X), (XI), (XIV) and/or (XVI).

Alternatively or additionally the method comprises, consists of, or is for determining whether the SLE-associated-disease state is non-active SLE or non SLE. Alternatively or additionally step (b) comprises or consists of measuring the presence and/or amount in the test sample of one or more of the biomarkers defined in Table B(I), (II), (III), (V), (VII), (IX), (X), (XII) and/or (XV).

Alternatively or additionally the method comprises, consists of, or is for determining whether the SLE-associated-disease state is highly active SLE or non SLE. Alternatively or additionally step (b) comprises or consists of measuring the presence and/or amount in the test sample of one or more of the biomarkers defined in Table B(I), (II), (IV), (VI), (XII), (XIII), (XIV) and/or (XVIII).

Alternatively or additionally the method comprises, consists of, or is for determining whether the SLE-associated-disease state is active SLE or non-active SLE. Alternatively or additionally step (b) comprises or consists of measuring the presence and/or amount in the test sample of one or more of the biomarkers defined in Table B(I), (II), (Ill), (IV), (V), (VII), (VIII), (XI), (XV) and/or (XVII).

Alternatively or additionally the method comprises, consists of, or is for determining whether the SLE-associated-disease state is highly active SLE or non-active SLE. Alternatively or additionally step (b) comprises or consists of measuring the presence and/or amount in the test sample of one or more of the biomarkers defined in Table B(I), (II), (IV), (VI), (XII), (XIII), (XIV) and/or (XVIII).

Alternatively or additionally the method comprises or consists of measuring all the biomarkers listed in Table A and Table B.

Alternatively or additionally the control sample of step (c) or step (e) is provided from:

a) a healthy individual (non-SLE);

b) an individual with non-active SLE (non-flaring SLE);

c) an individual with active SLE (flaring SLE); or

d) an individual with highly-active SLE (strongly flaring SLE).

The healthy individual may be free from SLE, autoimmune disease and/or renal disease. The healthy individual may be free from any form of disease.

By “non-active” we mean or include SLE with a SLEDAI 2000 of less than five. By “active” we mean or include SLE with a SLEDAI 2000 of five to fifteen (i.e., between five and fifteen). By “high active” or “highly active” SLE we mean or include SLE with a SLEDAI 2000 of sixteen or greater.

SLE disease severity and progression are conventionally determined through a clinical assessment and scoring using the following (SLEDAI-2000) criteria (see Gladman et al., 2002; J. Rheumatol., 29(2):288-91):

| wt | Descriptor | Definition |
|---|---|---|
| 8 | Seizure | Recent onset. Exclude metabolic, infectious or drug cause. |
| 8 | Psychosis | Altered ability to function in normal activity due to severe |
| | | disturbance in the perception of reality. Include hallucinations, |
| | | incoherence, marked loose associations, impoverished thought |
| | | content, marked illogical thinking, bizarre, disorganized, or |
| | | catatonic behaviour. Excluded uraemia and drug causes. |
| 8 | Organic Brain | Altered mental function with impaired orientation, memory or |
| | Syndrome | other intelligent function, with rapid onset fluctuating clinical |
| | | features. Include clouding of consciousness with reduced |
| | | capacity to focus, and inability to sustain attention to environment, |
| | | plus at least two of the following: perceptual disturbance, |
| | | incoherent speech, insomnia or daytime drowsiness, or increased |
| | | or decreased psychomotor activity. Exclude metabolic, infectious |
| | | or drug causes. |
| 8 | Visual Disturbance | Retinal changes of SLE. Include cytoid bodies, retinal |
| | | hemorrhages, serious exodate or hemorrhages in the choroids, |
| | | or optic neuritis. Exclude hypertension, infection, or drug causes. |
| 8 | Cranial Nerve | New onset of sensory or motor neuropathy involving cranial |
| | Disorder | nerves. |
| 8 | Lupus Headache | Severe persistent headache: may be migrainous, but must be |
| | | non-responsive to narcotic analgesia. |
| 8 | CVA | New onset of cerebrovascular accident(s). Exclude |
| | | arteriosclerosis. |
| 8 | Vasculitis | Ulceration, gangrene, tender finger nodules, periungual, |
| | | infarction, splinter hemorrhages, or biopsy or angiogram proof of |
| | | vasculitis. |
| 4 | Arthritis | More than 2 joints with pain and signs of inflammation (i.e. |
| | | tenderness, swelling, or effusion). |
| 4 | Myositis | Proximal muscle aching/weakness, associated with elevated |
| | | creatine phosphokinase/adolase or electromyogram changes or |
| | | a biopsy showing myositis. |
| 4 | Urinary Casts | Heme-granular or red blood cell casts. |
| 4 | Hematuria | >5 red blood cells/high power field. Exclude stone, infection or |
| | | other cause. |
| 4 | Proteinuria | >0.5 gm/24 hours. New onset or recent increase of more than 0.5 |
| | | gm/24 hours. |
| 4 | Pyuria | >5 white blood cells/high power field. Exclude infection. |
| 2 | Rash | Inflammatory type rash. |
| 2 | Alopecia | Abnormal, patchy or diffuse loss of hair. |
| 2 | Mucosal Ulcers | Oral or nasal ulcerations. |
| 2 | Pleurisy | Pleuritic chest pain with pleural rub or effusion, or pleural |
| | | thickening. |
| 2 | Pericarditis | Pericardial pain with at least one of the following: rub, effusion, or |
| | | electrocardiogram confirmation. |
| 2 | Low Complement | Decrease in CH50, C3, or C4 below the lower limit of normal for |
| | | testing laboratory. |
| 2 | Increased DNA | >25% binding by Fan assay or above normal range for testing |
| | binding | laboratory. |
| 1 | Fever | >38° C. Exclude infectious cause |
| 1 | Thrombocytopenia | <100,000 platelets/×10⁹/L. Exclude drug causes. |
| 1 | Leukopenia | <3,000 White blood cell/×10⁹/L. Exclude drug causes. |

The corresponding score/weight is applied if a descriptor is present at the time of visit or in the proceeding 10 to 30 days. The score is then totalled. A skilled person will appreciate that the SLEDAI boundaries of passive (remissive) SLE and active (flaring) SLE may vary according to the patient group being assessed.

Alternatively or additionally the lower range for passive (remissive) SLE may be any one of 0, 1, 2, 3, 4, 5, 6, 7, 8, 9, 10, 11, 12, 13, 14, 15, 16, 17, 18, 19 or 20; the upper range for passive (remissive) SLE may be any one of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45; the lower range for active or high active (flaring) SLE may be any one of 5, 6, 7, 8, 9, 10, 11, 12, 13, 14, 15, 16, 17, 18, 19 or 20; the upper range for mid severity SLE may be any one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85; the upper range for active or high active (flaring) SLE may be any one of 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102,103, 105 or 105; with the provisos that the lower range of a particular severity level must be of a lower score than its higher range and the ranges of each severity level may not overlap.

Alternatively or additionally, an increase in SLEDAI score of >3 from the previous assessment indicates mild or moderate flare. An increase in SLEDAI score of >12 from the previous assessment indicates severe flare. A decrease in SLEDAI score of >3 from the previous assessment indicates mild or moderate remission. A decrease in SLEDAI score of >12 from the previous assessment indicates advanced remission. An increase or decrease in SLEDAI score of 53 indicates stable (neither flaring nor non-flaring) SLE.

Alternatively or additionally the test sample of step (a) and/or the control sample of step (c) or step (e) is/are individually provided from:

a) an individual with SLE subtype 1 (SLE1);

b) an individual with SLE subtype 2 (SLE2); or

c) an individual with SLE subtype 3 (SLE3).

SLE1 comprises skin and musculoskeletal involvement but lacks serositis, systemic vasculitis and kidney involvement. SLE2 comprises skin and musculoskeletal involvement, serositis and systemic vasculitis but lacks kidney involvement. SLE3 comprises skin and musculoskeletal involvement, serositis, systemic vasculitis and SLE glomerulonephritis. SLE1, SLE2 and SLE3 represent mild/absent, moderate and severe SLE disease states, respectively (e.g., see Sturfelt G, Sjöholm A G. Complement components, complement activation, and acute phase response in systemic lupus erythematosus. Int Arch Allergy Appl Immunol 1984; 75:75-83 which is incorporated herein by reference).

Alternatively or additionally the physical symptoms of the SLE-associated disease state are present, for example, for differentiating between active and highly active SLE, the descriptors used to categorise an individual as ‘active’ or ‘highly active’ according to SLEDAI 2000 are present. In other words, the method of the invention may be diagnostic of an/the SLE-associated disease state.

By “diagnosing” we mean determining whether a subject is suffering from SLE. Conventional methods of diagnosing SLE are well known in the art.

The American College of Rheumatology established eleven criteria in 1982 (see Tan et al., 1982, The 1982 revised criteria for the classification of systemic lupus erythematosus, Arthritis. Rheum., 25:1271-7), which were revised in 1997 as a classificatory instrument to operationalise the definition of SLE in clinical trials (see Hochberg, 1997, Updating the American College of Rheumatology revised criteria for the classification of systemic lupus erythematosus, Arthritis. Rheum., 40:1725). For the purpose of identifying patients for clinical studies, a person is taken to have SLE if any 4 out of 11 symptoms are present simultaneously or serially on two separate occasions.

| Criterion | Definition |
|---|---|
| 1. Malar Rash | Fixed erythema, flat or raised, over the malar |
| | eminences, tending to spare the nasolabial folds |
| 2. Discoid | Erythematous raised patches with adherent keratotic |
| rash | scaling and follicular plugging; atrophic scarring may |
| | occur in older lesions |
| 3. Photo- | Skin rash as a result of unusual reaction to sunlight, by |
| sensitivity | patient history or physician observation |
| 4. Oral ulcers | Oral or nasopharyngeal ulceration, usually painless, |
| | observed by physician |
| 5. Nonerosive | Involving 2 or more peripheral joints, characterized by |
| Arthritis | tenderness, swelling, or effusion |
| 6. Pleuritis or | Pleuritis-convincing history of pleuritic pain or rubbing |
| Pericarditis | heard by a physician or evidence of pleural effusion |
| | OR |
| | Pericarditis-documented by electrocardigram or rub or |
| | evidence of pericardial effusion |
| 7. Renal | Persistent proteinuria > 0.5 grams per day or > than 3+ |
| Disorder | if quantitation not performed |
| | OR |
| | Cellular casts-may be red cell, hemoglobin, granular, |
| | tubular, or mixed |
| 8. Neurologic | Seizures in the absence of offending drugs or known |
| Disorder | metabolic derangements; e.g., uremia, ketoacidosis, or |
| | electrolyte imbalance |
| | OR |
| | Psychosis in the absence of offending drugs or known |
| | metabolic derangements, e.g., uremia, ketoacidosis, or |
| | electrolyte imbalance |
| 9. Hemato- | Hemolytic anemia-with reticulocytosis |
| logic | OR |
| Disorder | Leukopenia-<4,000/mm3 on ≥ 2 occasions |
| | OR |
| | Lyphopenia-<1,500/mm3 on ≥ 2 occasions |
| | OR |
| | Thrombocytopenia-<100,000/mm3 in the absence of |
| | offending drugs |
| 10. Immuno- | Anti-DNA: antibody to native DNA in abnormal titer |
| logic | OR |
| Disorder | Anti-Sm: presence of antibody to Sm nuclear antigen |
| | OR |
| | Positive finding of antiphospholipid antibodies on: |
| | (a) an abnormal serum level of IgG or IgM |
| | anticardiolipin antibodies, |
| | (b) a positive test result for lupus anticoagulant using a |
| | standard method, or |
| | (c) a false-positive test result for at least 6 months |
| | confirmed by Treponema pallidum immobilization or |
| | fluorescent treponemal antibody absorption test |
| 11. Anti- | An abnormal titer of antinuclear antibody by |
| nuclear | immunofluorescence or an equivalent assay at any |
| Antibody | point in time and in the absence of drugs |

Some people, especially those with antiphospholipid syndrome, may have SLE without four of the above criteria, and also SLE may present with features other than those listed in the criteria (see Asherson et al., 2003, Catastrophic antiphospholipid syndrome: international consensus statement on classification criteria and treatment guidelines, Lupus, 12(7):530-4; Sangle et al., 2005, Livedo reticularis and pregnancy morbidity in patients negative for antiphospholipid antibodies, Ann. Rheum. Dis., 64(1):147-8; and Hughes and Khamashta, 2003, Seronegative antiphospholipid syndrome, Ann. Rheum. Dis., 62(12):1127).

Recursive partitioning has been used to identify more parsimonious criteria (see Edworthy et al., 1988, Analysis of the 1982 ARA lupus criteria data set by recursive partitioning methodology: new insights into the relative merit of individual criteria, J. Rheumatol., 15(10):1493-8). This analysis presented two diagnostic classification trees:
Simplest classification tree: SLE is diagnosed if a person has an immunologic disorder (anti-DNA antibody, anti-Smith antibody, false positive syphilis test, or LE cells) or malar rash.
Full classification tree: Uses 6 criteria.

Alternatively or additionally, the diagnosis of SLE in is made according to the principles outlined by Fries and Holman, in: Smith LH Jr, ed. In: Smith LH Jr, ed. major Problems in Internal Medicine. Vol VI., 1976, which is incorporated herein by reference.

Other alternative set of criteria has been suggested, the St. Thomas' Hospital “alternative” criteria in 1998 (see Hughes, 1998, Is it lupus? The St. Thomas' Hospital “alternative” criteria, Clin. Exp. Rheumatol., 16(3):250-2).

However, these criteria were not intended to be used to diagnose individuals. They are time-consuming, subjective, require a high degree of experience to use effectively and have a high frequency of excluding actual SLE sufferers (i.e., diagnosing SLE patients as non-SLE patients). The present invention addresses these problems, providing objective SLE diagnosis.

Alternatively or additionally the SLE-associated disease state is determined before the appearance of the physical symptoms of the SLE-associated disease state, for example, for differentiating between active and highly active SLE, the descriptors used to categorise an individual as ‘active’ or ‘highly active’ according to SLEDAI 2000 are not yet present. Hence, the individual may be categorised as belonging to a first disease state by the method of the present invention but categorised as a second disease state according to SLEDAI 2000. In other words, the method of the invention may be prognostic of an/the SLE-associated disease state.

Alternatively or additionally, the SLE-associated disease state may be determined at least 1 day before the appearance of the physical symptoms of the SLE associated disease state, for example, at least 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 4 months, five f months or, six 6 months, 7 months, 8 months, 9 months, 10 months, 11 months, 12 months, 13 months, 14, months, 15, months, 16 months, 17 months, 18 months, 19 months, 20 months, 21 months, 22 months, 23 months or 24 months before the appearance of the physical symptoms of the SLE-associated disease state.

By “expression” we include the level or amount of a gene product such as mRNA or protein.

By ‘Motif #’ (wherein ‘#’ represents a number) we include a protein comprising the selection motif shown in Table B. Alternatively or additionally we include a protein specifically bound by an antibody having the CDRs defined in Table B in respect of the motif in question. Alternatively or additionally the antibody has a framework region as defined in Olsson et al., 2012, ‘Epitope-specificity of recombinant antibodies reveals promiscuous peptide-binding properties.’ Protein Sci., 21(12):1897-910.

Generally, the systemic lupus erythematosus-associated disease state in a subject is determined with an ROC AUC of at least 0.55, for example with an ROC AUC of at least, 0.60, 0.65, 0.70, 0.75, 0.80, 0.85, 0.90, 0.95, 0.96, 0.97, 0.98 or with an ROC AUC of at least 0.99. Preferably, the systemic lupus erythematosus-associated disease state in an individual is determined with an ROC AUC of at least 0.85.

Typically, the systemic lupus erythematosus-associated disease state in a subject is determined using a support vector machine (SVM), such as those available from http://cran.r-project.org/web/packages/e1071/index.html (e.g. e1071 1.5-24). However, any other suitable means may also be used.

Support vector machines (SVMs) are a set of related supervised learning methods used for classification and regression. Given a set of training examples, each marked as belonging to one of two categories, an SVM training algorithm builds a model that predicts whether a new example falls into one category or the other. Intuitively, an SVM model is a representation of the examples as points in space, mapped so that the examples of the separate categories are divided by a clear gap that is as wide as possible. New examples are then mapped into that same space and predicted to belong to a category based on which side of the gap they fall on.

More formally, a support vector machine constructs a hyperplane or set of hyperplanes in a high or infinite dimensional space, which can be used for classification, regression or other tasks. Intuitively, a good separation is achieved by the hyperplane that has the largest distance to the nearest training datapoints of any class (so-called functional margin), since in general the larger the margin the lower the generalization error of the classifier. For more information on SVMs, see for example, Burges, 1998, Data Mining and Knowledge Discovery, 2:121-167.

In one embodiment of the invention, the SVM is ‘trained’ prior to performing the methods of the invention using proteome samples from subjects assigned to known patient groups (namely, those patients in which the systemic lupus erythematosus-associated disease state is present versus those patients in which it is absent). By running such training samples, the SVM is able to learn what biomarker profiles are associated with the systemic lupus erythematosus-associated disease state. Once the training process is complete, the SVM is then able whether or not the proteome sample tested is from a subject a systemic lupus erythematosus-associated disease state.

However, this training procedure can be by-passed by pre-programming the SVM with the necessary training parameters. For example, a systemic lupus erythematosus-associated disease state in a subject can be determined using the SVM parameters detailed in Table B, based on the measurement of some or all the biomarkers listed in Table A.

It will be appreciated by skilled persons that suitable SVM parameters can be determined for any combination of the biomarkers listed Table A by training an SVM machine with the appropriate selection of data (i.e. biomarker measurements in samples from known patient groups.

Alternatively, the data provided in the present figures and tables may be used to determine a particular SLE-associated disease state according to any other suitable statistical method known in the art, such as Principal Component Analysis (PCA) Orthogonal PCA (OPLS) and other multivariate statistical analyses (e.g., backward stepwise logistic regression model). For a review of multivariate statistical analysis see, for example, Schervish, Mark J. (November 1987). “A Review of Multivariate Analysis”. Statistical Science 2 (4): 396-413 which is incorporated herein by reference.

Preferably, the method of the invention has an accuracy of at least 51%, for example 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% accuracy.

Preferably, the method of the invention has a sensitivity of at least 51%, for example 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sensitivity.

Preferably, the method of the invention has a specificity of at least 51%, for example 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% specificity.

By “accuracy” we mean the proportion of correct outcomes of a method, by “sensitivity” we mean the proportion of all positive chemicals that are correctly classified as positives, and by “specificity” we mean the proportion of all negative chemicals that are correctly classified as negatives.

Alternatively or additionally step (b) and/or step (d) is performed using a binding agent capable of binding to the one or more biomarker(s). Binding agents (also referred to as binding molecules and binding moieties) can be selected from a library, based on their ability to bind a given motif, as discussed below.

By “biomarker” we mean a naturally-occurring biological molecule, or component or fragment thereof, the measurement of which can provide information useful in the prognosis of pancreatic cancer. For example, the biomarker may be a naturally occurring protein, mRNA or carbohydrate moiety, or an antigenic component or fragment thereof.

In an alternative or additional embodiment step (b) comprises measuring the expression of the protein or polypeptide of the one or more biomarker(s).

Methods of detecting and/or measuring the concentration of protein and/or nucleic acid are well known to those skilled in the art, see for example Sambrook and Russell, 2001, Cold Spring Harbor Laboratory Press.

Preferred methods for detection and/or measurement of protein include Western blot, North-Western blot, immunosorbent assays (ELISA), antibody microarray, tissue microarray (TMA), immunoprecipitation, in situ hybridisation and other immunohistochemistry techniques, radioimmunoassay (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference. Antibody staining of cells on slides may be used in methods well known in cytology laboratory diagnostic tests, as well known to those skilled in the art.

Typically, ELISA involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemiluminescent systems based on enzymes such as luciferase can also be used.

Conjugation with the vitamin biotin is frequently used since this can readily be detected by its reaction with enzyme-linked avidin or streptavidin to which it binds with great specificity and affinity.

Alternatively or additionally, the binding agent is an antibody or a fragment thereof.

Thus, a fragment may contain one or more of the variable heavy (V_{H}) or variable light (V_{L}) domains. For example, the term antibody fragment includes Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544).

The term “antibody variant” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecule capable of binding to an antigen in an immunoassay format that is known to those skilled in the art.

A general review of the techniques involved in the synthesis of antibody fragments which retain their specific binding sites is to be found in Winter & Milstein (1991) Nature 349, 293-299.

Additionally or alternatively at least one type, more typically all of the types, of the binding molecules is an aptamer.

Molecular libraries such as antibody libraries (Clackson et al, 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

The molecular libraries may be expressed in vivo in prokaryotic (Clackson et al, 1991, op. cit.; Marks et al, 1991, op. cit.) or eukaryotic cells (Kieke et al, 1999, Proc Natl Acad Sci USA, 96(10):5651-6) or may be expressed in vitro without involvement of cells (Hanes & Pluckthun, 1997, Proc Natl Acad Sci USA 94(10):4937-42; He & Taussig, 1997, Nucleic Acids Res 25(24):5132-4; Nemoto et al, 1997, FEBS Lett, 414(2):405-8).

In cases when protein based libraries are used often the genes encoding the libraries of potential binding molecules are packaged in viruses and the potential binding molecule is displayed at the surface of the virus (Clackson et al, 1991, op. cit.; Marks et al, 1991, op. cit; Smith, 1985, op. cit.).

The most commonly used such system today is filamentous bacteriophage displaying antibody fragments at their surfaces, the antibody fragments being expressed as a fusion to the minor coat protein of the bacteriophage (Clackson et al, 1991, op. cit.; Marks et al, 1991, op. cit). However, also other systems for display using other viruses (EP 39578), bacteria (Gunneriusson et al, 1999, op. cit.; Daugherty et al, 1998, Protein Eng 11(9):825-32; Daugherty et al, 1999, Protein Eng 12(7):613-21), and yeast (Shusta et al, 1999, J Mol Biol 292(5):949-56) have been used.

In addition, recently, display systems utilising linkage of the polypeptide product to its encoding mRNA in so called ribosome display systems (Hanes & Pluckthun, 1997, op. cit.; He & Taussig, 1997, op. cit.; Nemoto et al, 1997, op. cit.), or alternatively linkage of the polypeptide product to the encoding DNA (see U.S. Pat. No. 5,856,090 and WO 98/37186) have been presented.

When potential binding molecules are selected from libraries one or a few selector peptides having defined motifs are usually employed. Amino acid residues that provide structure, decreasing flexibility in the peptide or charged, polar or hydrophobic side chains allowing interaction with the binding molecule may be used in the design of motifs for selector peptides. For example:
(i) Proline may stabilise a peptide structure as its side chain is bound both to the alpha carbon as well as the nitrogen;
(ii) Phenylalanine, tyrosine and tryptophan have aromatic side chains and are highly hydrophobic, whereas leucine and isoleucine have aliphatic side chains and are also hydrophobic;
(iii) Lysine, arginine and histidine have basic side chains and will be positively charged at neutral pH, whereas aspartate and glutamate have acidic side chains and will be negatively charged at neutral pH;
(iv) Asparagine and glutamine are neutral at neutral pH but contain a amide group which may participate in hydrogen bonds;
(v) Serine, threonine and tyrosine side chains contain hydroxyl groups, which may participate in hydrogen bonds.

Typically selection of binding molecules may involve the use of array technologies and systems to analyse binding to spots corresponding to types of binding molecules.

In one embodiment, the antibody or fragment thereof is a recombinant antibody or fragment thereof (such as an scFv).

By “ScFv molecules” we mean molecules wherein the V_{H} and V_{L} partner domains are linked via a flexible oligopeptide.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

The antibodies may be monoclonal or polyclonal. Suitable monoclonal antibodies may be prepared by known techniques, for example those disclosed in “Monoclonal Antibodies: A manual of techniques”, H Zola (CRC Press, 1988) and in “Monoclonal Hybridoma Antibodies: Techniques and applications”, J G R Hurrell (CRC Press, 1982), both of which are incorporated herein by reference.

Alternatively or additionally the antibody or fragment thereof is selected from the group consisting of: scFv; Fab; a binding domain of an immunoglobulin molecule.

Alternatively or additionally, antibody or antigen-binding fragment is capable of competing for binding to a biomarker specified in Table A with an antibody for that biomarker defined in Table C.

By “capable of competing” for binding to a biomarker specified in Table A with an antibody molecule as defined herein (or a variant, fusion or derivative of said antibody or antigen-binding fragment, or a fusion of a said variant or derivative thereof, which retains the binding specificity for the required biomarker) we mean or include that the tested antibody or antigen-binding fragment is capable of inhibiting or otherwise interfering, at least in part, with the binding of an antibody molecule as defined herein.

For example, the antibody or antigen-binding fragment may be capable of inhibiting the binding of an antibody molecule defined herein by at least 10%, for example at least 20%, 30%, 40%, 50%, 60%, 70%, 80%, 90%, 35% or even by 100%.

Competitive binding may be determined by methods well known to those skilled in the art, such as ELISA (as described herein) and/or SPR (as described in the accompanying Examples).

Alternatively or additionally, the antibody or antigen-binding fragment is an antibody defined in Table C or an antigen-binding fragment thereof, or a variant thereof.

Alternatively or additionally, the antibody the antibody or antigen-binding fragment comprises a VH and VL domain specified in Table C, or a variant thereof.

By ‘variants’ of the antibody or antigen-binding fragment of the invention we include insertions, deletions and substitutions, either conservative or non-conservative. In particular we include variants of the sequence of the antibody or antigen-binding fragment where such variations do not substantially alter the activity of the antibody or antigen-binding fragment. In particular, we include variants of the antibody or antigen-binding fragment where such changes do not substantially alter the binding specificity for the respective biomarker specified in Table C.

The polypeptide variant may have an amino acid sequence which has at least 70% identity with one or more of the amino acid sequences of the antibody or antigen-binding fragment of the invention as defined herein—for example, at least 75%, at least 80%, at least 90%, at least 95%, at least 96%, at least 97%, at least 98%, at least 99% or 100% identity with one or more of the amino acid sequences of the antibody or antigen-binding fragment of the invention as defined herein.

The percent sequence identity between two polypeptides may be determined using suitable computer programs, for example the GAP program of the University of Wisconsin Genetic Computing Group and it will be appreciated that percent identity is calculated in relation to polypeptides whose sequences have been aligned optimally.

The alignment may alternatively be carried out using the Clustal W program (as described in Thompson et al., 1994, Nucl. Acid Res. 22:4673-4680, which is incorporated herein by reference).

The parameters used may be as follows:
Fast pair-wise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

The antibodies may share CDRs (e.g., 1, 2, 3, 4, 5 or 6) CDRs with one or more of the antibodies defined in Table C.

CDRs can be defined using any suitable method known in the art. Commonly used methods include Paratome (Kunik, Ashkenazi and Ofran, 2012, ‘Paratome: an online tool for systematic identification of antigen-binding regions in antibodies based on sequence or structure’ Nucl. Acids Res., 40:W521-W524; http://www.ofranlab.org/paratome/), Kabat (Wu and Kabat, 1970, ‘An analysis of the sequences of the variable regions of Bence Jones proteins and myeloma light chains and their implications for antibody complementarity.’ J. Exp. Med., 132:211-250), Chothia (Chothia and Lesk, 1987 ‘Canonical structures for the hypervariable regions of immunoglobulins’ J. Mol. Biol., 196:901-917; Chothia et al., 1989 ‘Conformations of immunoglobulin hypervariable regions’ Nature, 342:877-883) and IMGT (Lefranc et al., 2003 ‘IMGT unique numbering for immunoglobulin and T cell receptor variable domains and Ig superfamily V-like domains’. Dev. Comp. Immunol., 27:55-77; Lefranc et al., 2005 ‘IMGT unique numbering for immunoglobulin and T cell receptor constant domains and Ig superfamily C-like domains’ Dev. Comp. Immunol., 29:185-203; http://www.imgt.org). For example, the method used may be the IMGT method.

Alternatively or additionally, the first binding agent is immobilised on a surface (e.g., on a multiwell plate or array).

Alternatively or additionally the one or more biomarker(s) in the test sample is labelled with a detectable moiety.

Alternatively or additionally the one or more biomarker(s) in the control sample is labelled with a detectable moiety (which may be the same or different from the detectable moiety used to label the test sample).

By a “detectable moiety” we include the meaning that the moiety is one which may be detected and the relative amount and/or location of the moiety (for example, the location on an array) determined.

Detectable moieties are well known in the art.

A detectable moiety may be a fluorescent and/or luminescent and/or chemiluminescent moiety which, when exposed to specific conditions, may be detected. For example, a fluorescent moiety may need to be exposed to radiation (i.e. light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected. Examples of suitable enzymes are discussed in more detail in relation to, for example, ELISA assays.

Alternatively, the detectable moiety may be a radioactive atom which is useful in imaging. Suitable radioactive atoms include ^{99m}Tc and ¹²³I for scintigraphic studies. Other readily detectable moieties include, for example, spin labels for magnetic resonance imaging (MRI) such as ¹²³I again, ¹³¹I, ¹¹¹In, ¹⁹F, ¹³C, ¹⁵N, ¹⁷O, gadolinium, manganese or iron. Clearly, the agent to be detected (such as, for example, the one or more proteins in the test sample and/or control sample described herein and/or an antibody molecule for use in detecting a selected protein) must have sufficient of the appropriate atomic isotopes in order for the detectable moiety to be readily detectable.

The radio- or other labels may be incorporated into the agents of the invention (i.e. the proteins present in the samples of the methods of the invention and/or the binding agents of the invention) in known ways. For example, if the binding moiety is a polypeptide it may be biosynthesised or may be synthesised by chemical amino acid synthesis using suitable amino acid precursors involving, for example, fluorine-19 in place of hydrogen. Labels such as ^{99m}Tc, ¹²³I, ¹⁸⁶Rh, ¹⁸⁸Rh and ¹¹¹In can, for example, be attached via cysteine residues in the binding moiety. Yttrium-90 can be attached via a lysine residue. The IODOGEN method (Fraker et al (1978) Biochem. Biophys. Res. Comm. 80, 49-57) can be used to incorporate ¹²³I. Reference (“Monoclonal Antibodies in Immunoscintigraphy”, J-F Chatal, CRC Press, 1989) describes other methods in detail. Methods for conjugating other detectable moieties (such as enzymatic, fluorescent, luminescent, chemiluminescent or radioactive moieties) to proteins are well known in the art.

Preferably, the detectable moiety is selected from the group consisting of: a fluorescent moiety, a luminescent moiety, a chemiluminescent moiety, a radioactive moiety, and an enzymatic moiety.

In an alternative or additional embodiment step (b), (d) and/or (f) comprises measuring the expression of a nucleic acid molecule encoding the one or more biomarkers.

The nucleic acid molecule may be a cDNA molecule or an mRNA molecule. Preferably the nucleic acid molecule is an mRNA molecule. Also preferably the nucleic acid molecule is a cDNA molecule.

Hence, measuring the expression of the one or more biomarker(s) in step (b) may be performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation. Preferably measuring the expression of the one or more biomarker(s) in step (b) is determined using a DNA microarray. Hence, the method may comprise or consist of measuring the expression of the one or more biomarker(s) in step (b) using one or more binding moiety, each capable of binding selectively to a nucleic acid molecule encoding one of the biomarkers identified in Table A.

In an alternative or additional embodiment step the one or more binding moieties each comprise or consist of a nucleic acid molecule such as DNA, RNA, PNA, LNA, GNA, TNA or PMO (preferably DNA). Preferably the one or more binding moieties are 5 to 100 nucleotides in length. More preferably, the one or more nucleic acid molecules are 15 to 35 nucleotides in length. The binding moiety may comprise a detectable moiety.

Suitable binding agents (also referred to as binding molecules) may be selected or screened from a library based on their ability to bind a given nucleic acid, protein or amino acid motif.

In an alternative or additional embodiment measuring the expression of the one or more biomarker(s) in step (b), (d) and/or (f) is performed using one or more binding moieties, each individually capable of binding selectively to a nucleic acid molecule encoding one of the biomarkers identified in Table A.

In an alternative or additional embodiment, the nucleic acid binding moiety comprises a detectable moiety as defined above.

Alternatively or additionally step (b), (d) and/or (f), where present, is performed using an array. The array may be a bead-based array or a surface-based array. The array may be selected from the group consisting of macroarray, microarray and nanoarray.

Arrays per se are well known in the art. Typically, they are formed of a linear or two-dimensional structure having spaced apart (i.e. discrete) regions (“spots”), each having a finite area, formed on the surface of a solid support. An array can also be a bead structure where each bead can be identified by a molecular code or colour code or identified in a continuous flow. Analysis can also be performed sequentially where the sample is passed over a series of spots each adsorbing the class of molecules from the solution. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g. plastic, polymer, perspex, silicon, amongst others), a plurality of polymeric pins, or a plurality of microtitre wells, or any other surface suitable for immobilising proteins, polynucleotides and other suitable molecules and/or conducting an immunoassay. The binding processes are well known in the art and generally consist of cross-linking covalently binding or physically adsorbing a protein molecule, polynucleotide or the like to the solid support. By using well-known techniques, such as contact or non-contact printing, masking or photolithography, the location of each spot can be defined. For reviews see Jenkins, R. E., Pennington, S. R. (2001, Proteomics, 2, 13-29) and Lal et al (2002, Drug Discov Today 15; 7(18 Suppl):S143-9).

Typically, the array is a microarray. By “microarray” we include the meaning of an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance. The array may also be a macroarray or a nanoarray.

Once suitable binding molecules (discussed above) have been identified and isolated, the skilled person can manufacture an array using methods well known in the art of molecular biology.

Alternatively or additionally step (b), step (d) and/or step (f), where present, is performed using an assay comprising a second binding agent capable of binding to the one or more proteins, the second binding agent having a detectable moiety.

Alternatively or additionally step (b), step (d) and/or step (f), where present, are performed using ELISA (Enzyme Linked Immunosorbent Assay).

Typically, the assay is an ELISA (Enzyme Linked Immunosorbent Assay) which typically involve the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system.

Pyrophosphatase from Escherichia coli provides a good conjugate because the enzyme is not present in tissues, is stable and gives a good reaction colour. Chemi-luminescent systems based on enzymes such as luciferase can also be used.

Conjugation with the vitamin biotin is also employed used since this can readily be detected by its reaction with enzyme-linked avidin or streptavidin to which it binds with great specificity and affinity.

It will be appreciated by persons skilled in the art that there is a degree of fluidity in the biomarker composition of the signatures of the invention. Thus, different combinations of the biomarkers may be equally useful in the diagnosis, prognosis and/or characterisation of SLE. In this way, each biomarker (either alone or in combination with one or more other biomarkers) makes a contribution to the signature.

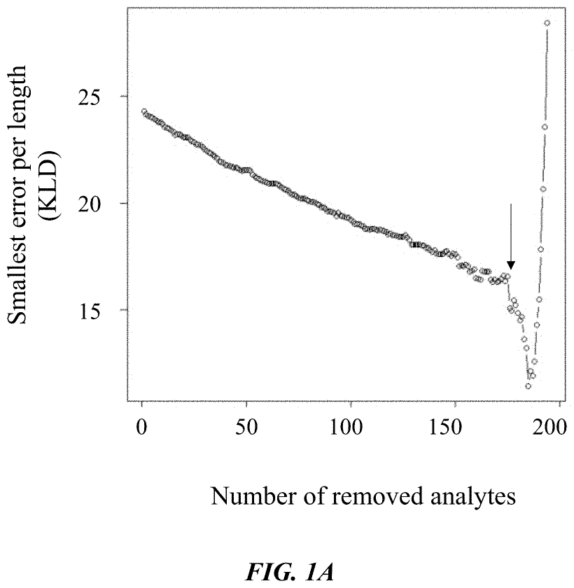

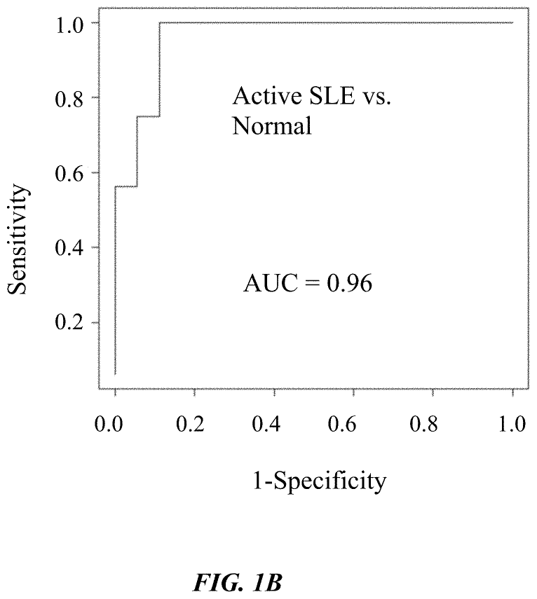

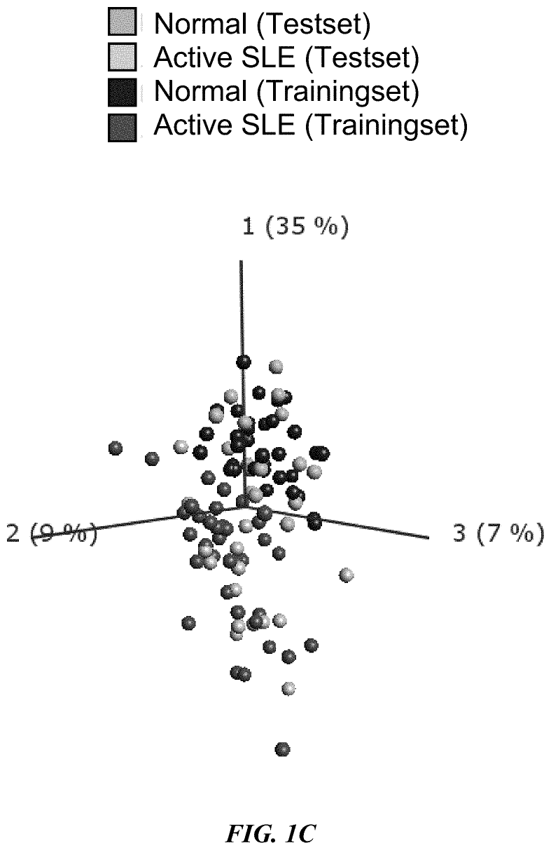

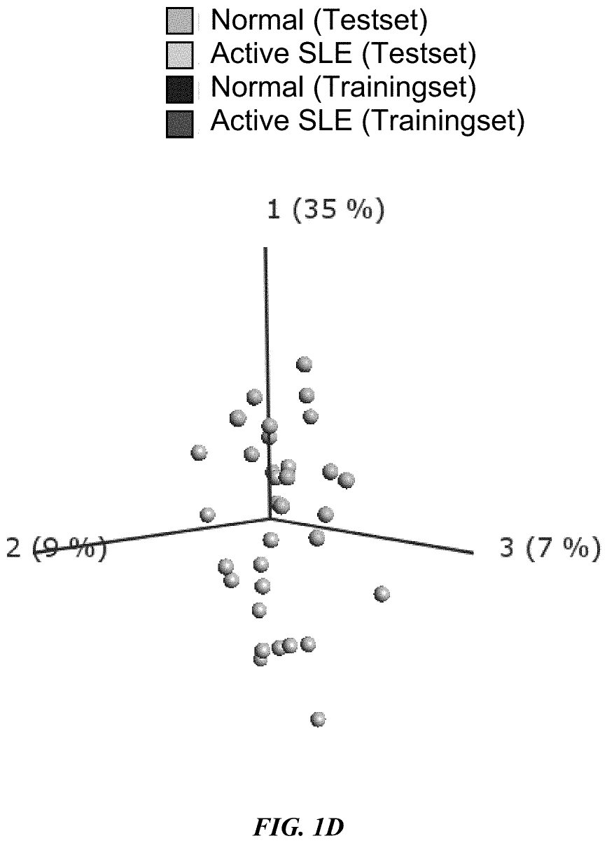

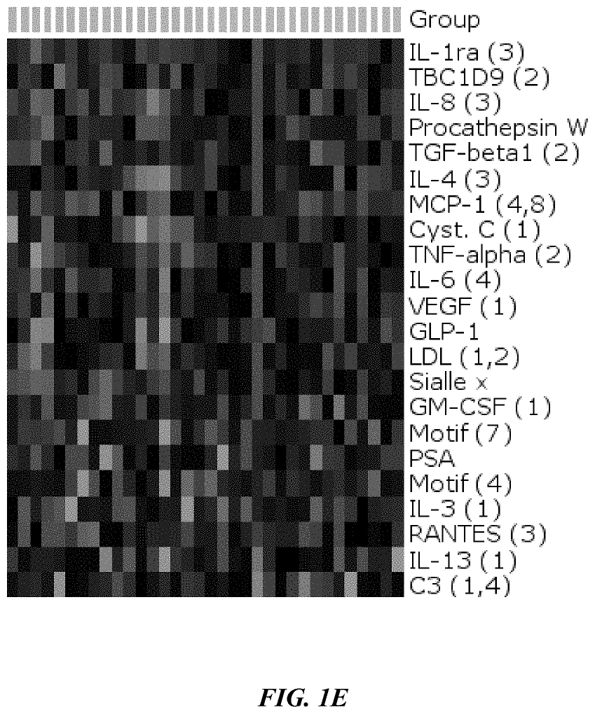

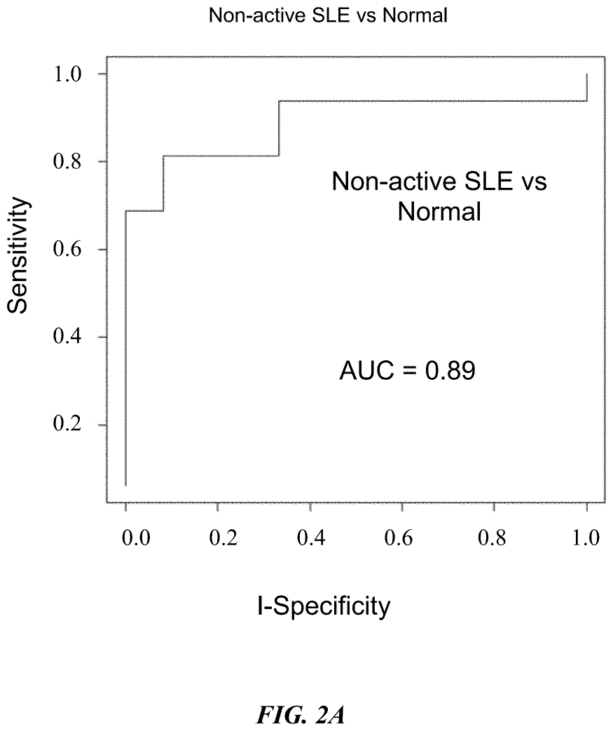

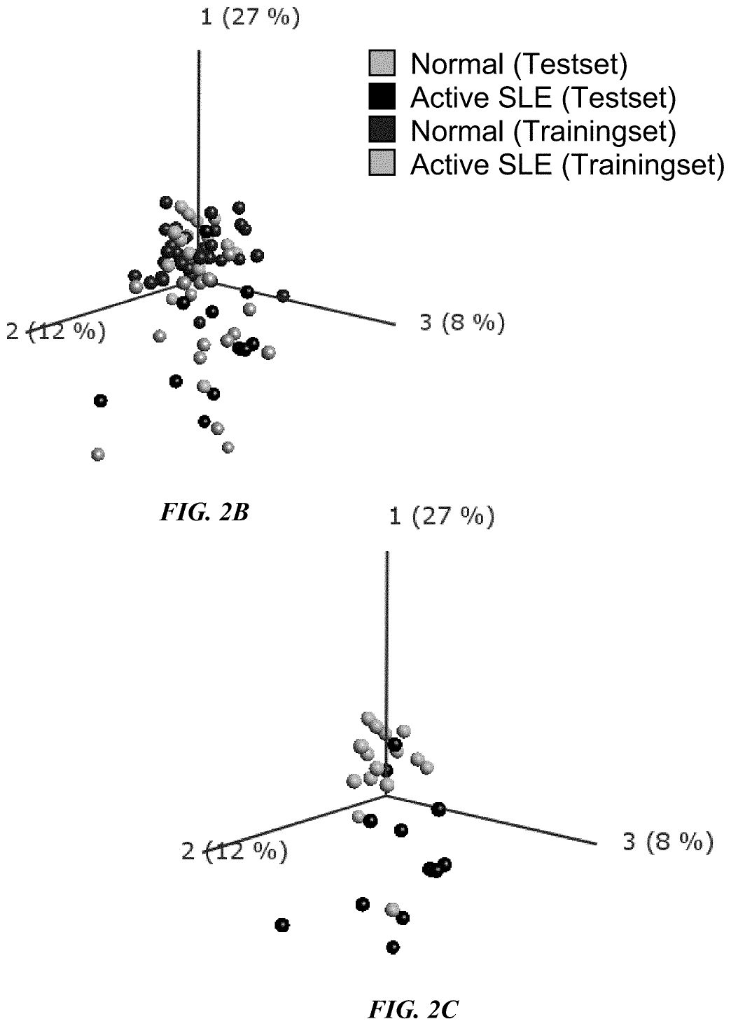

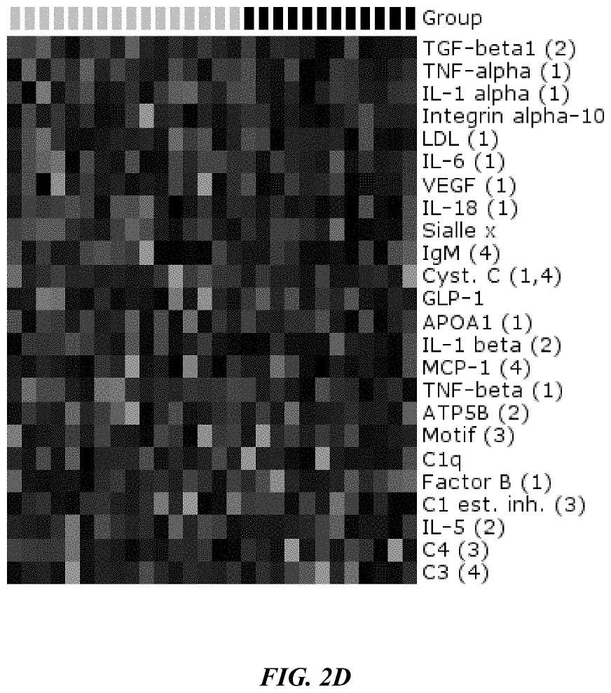

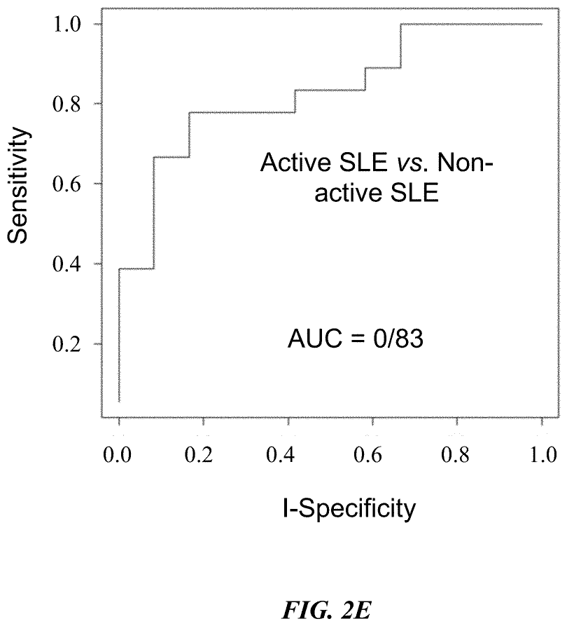

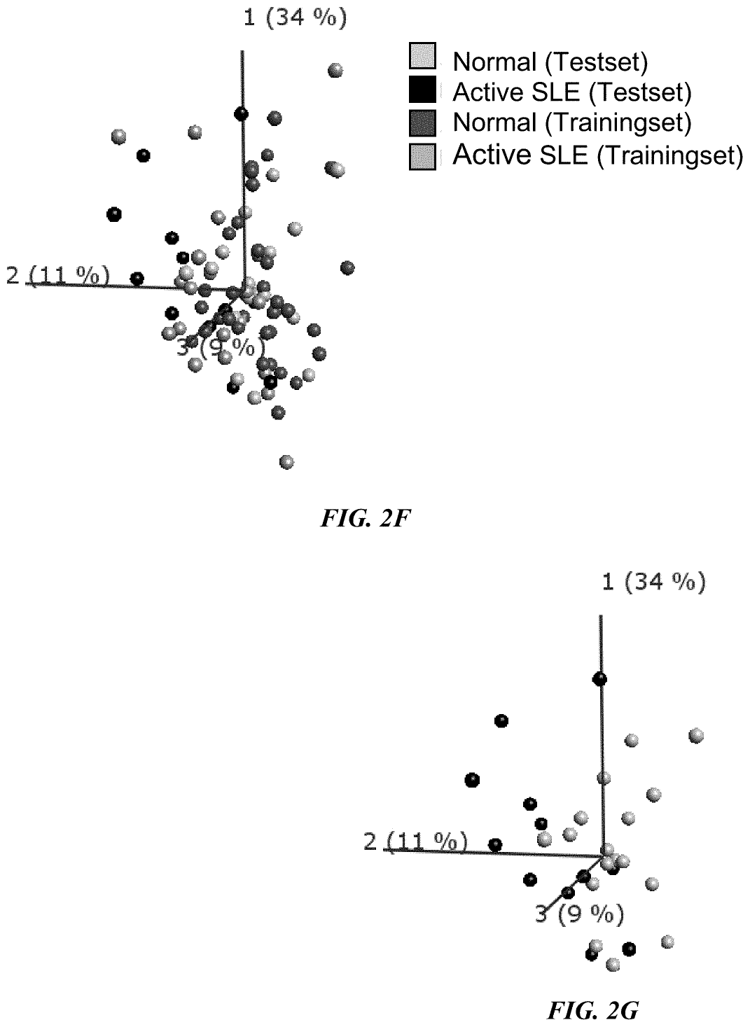

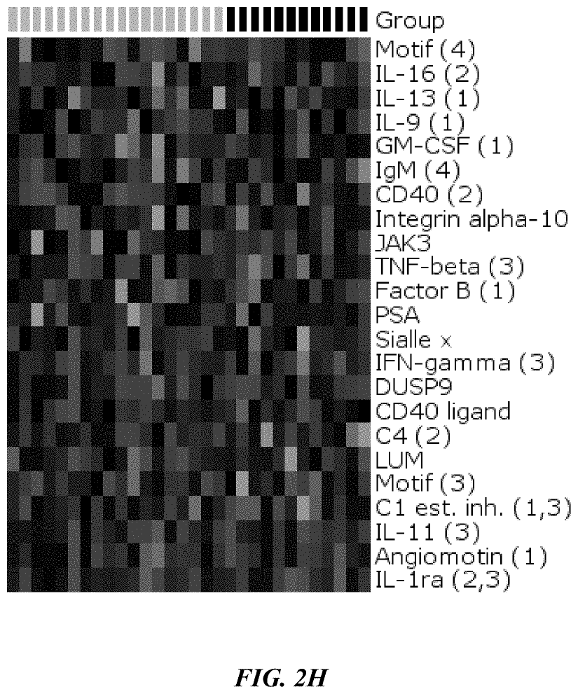

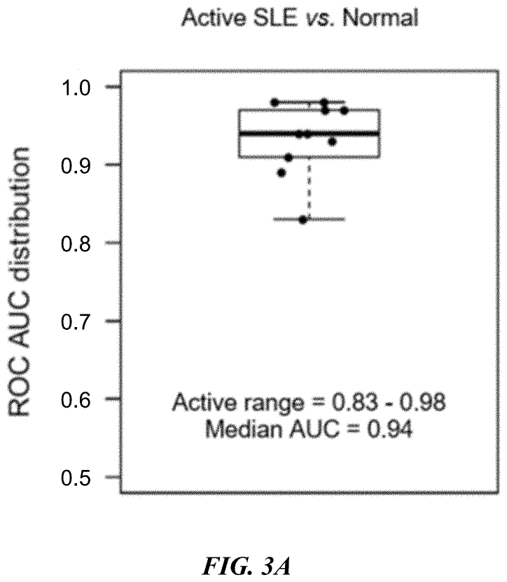

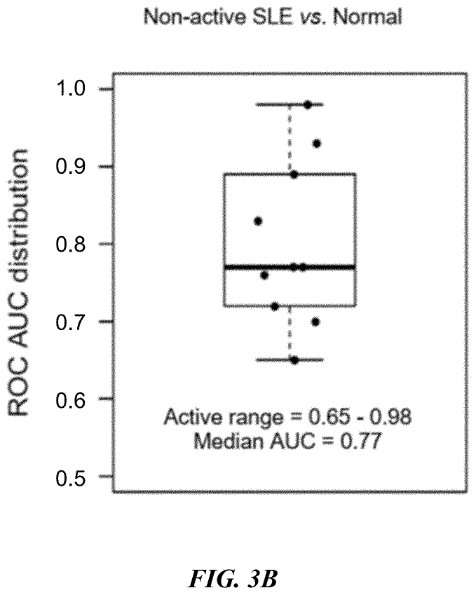

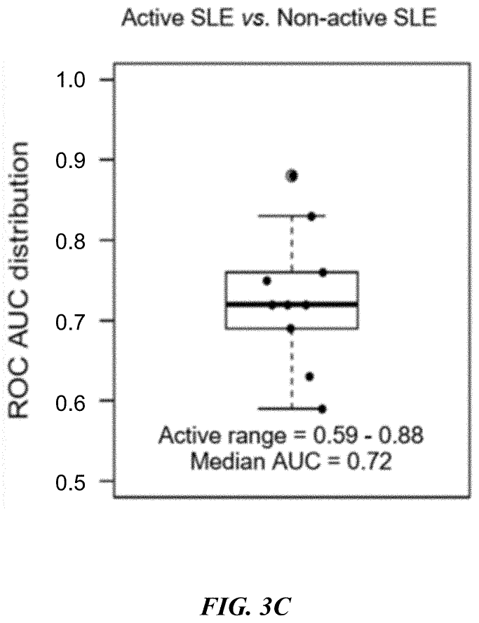

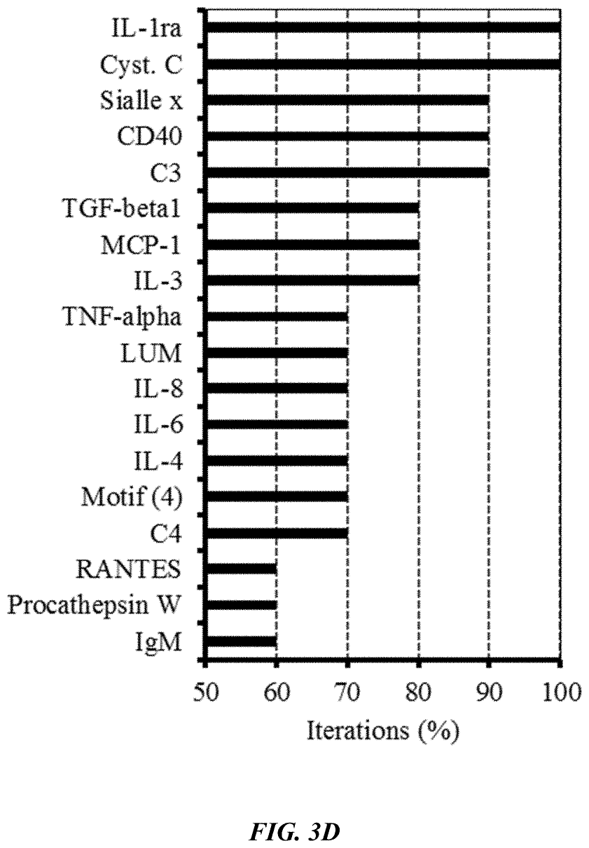

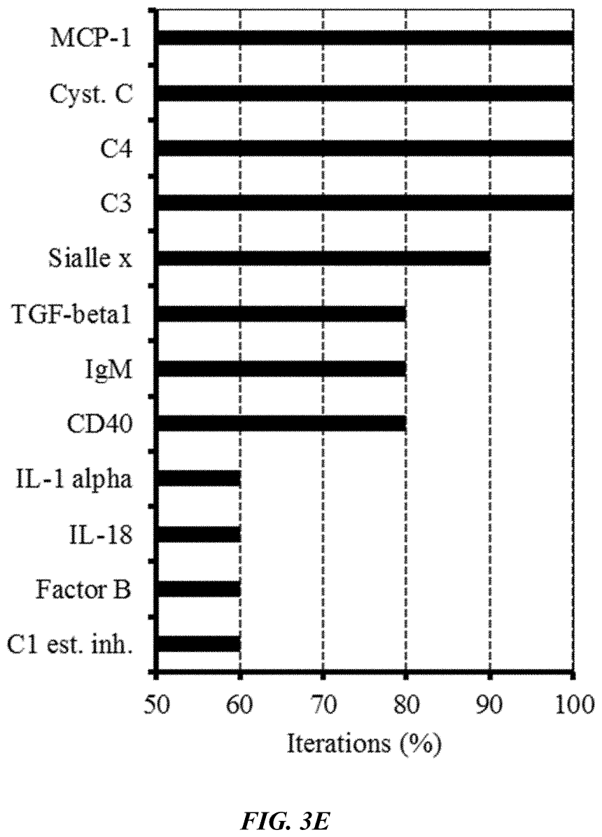

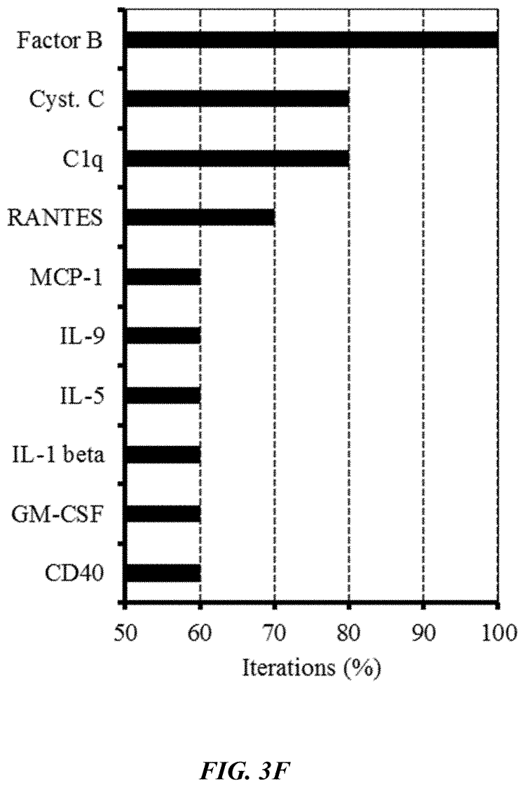

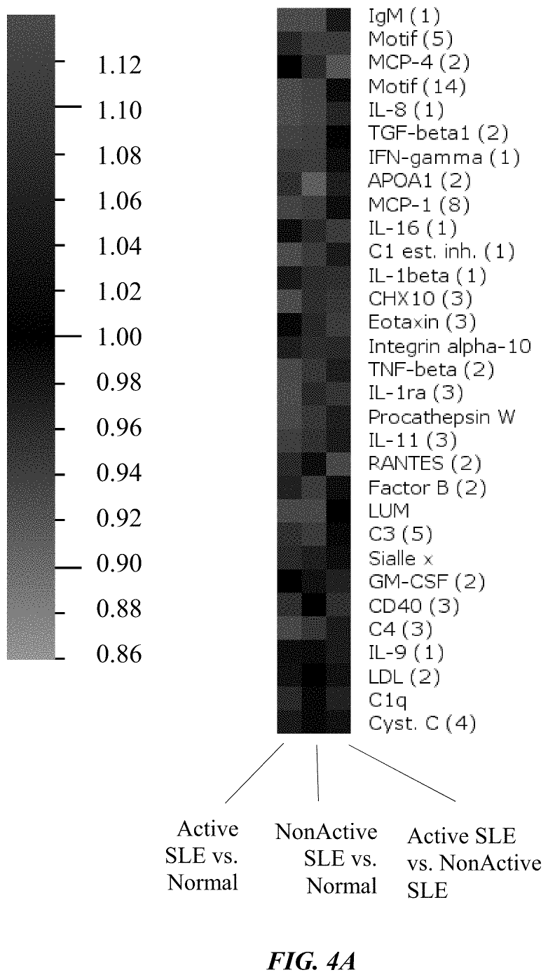

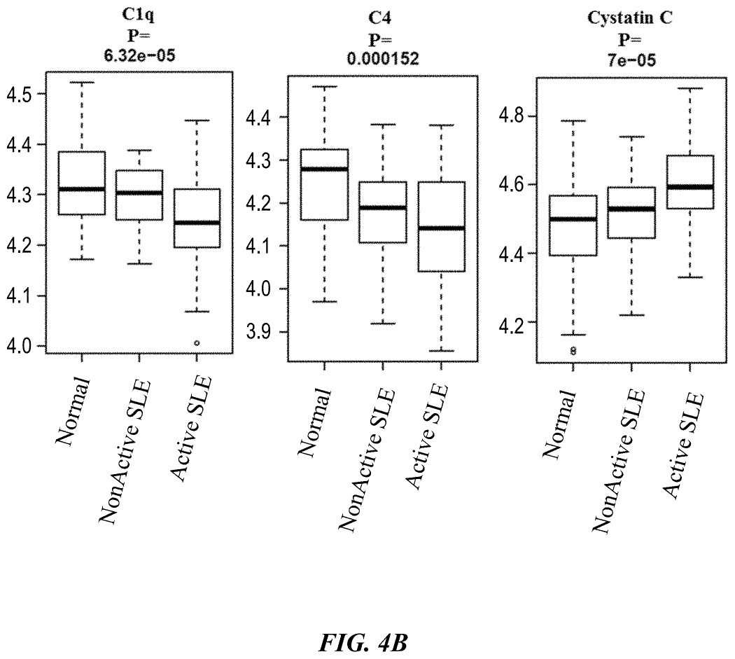

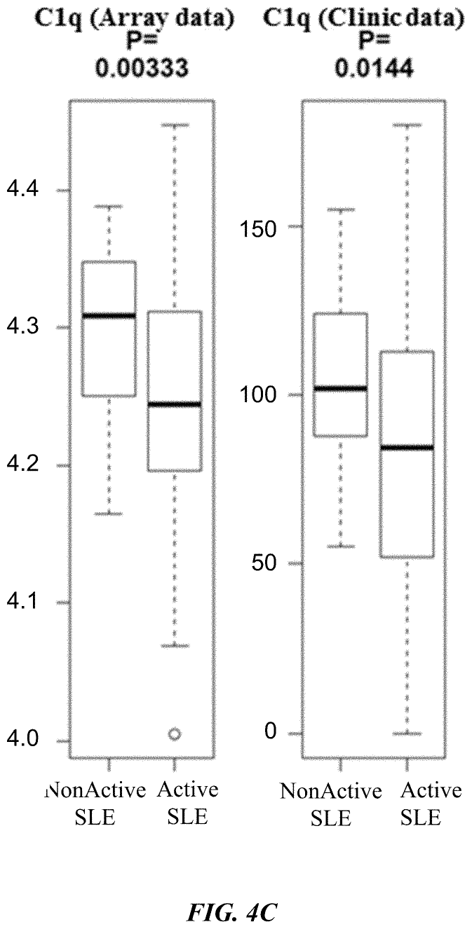

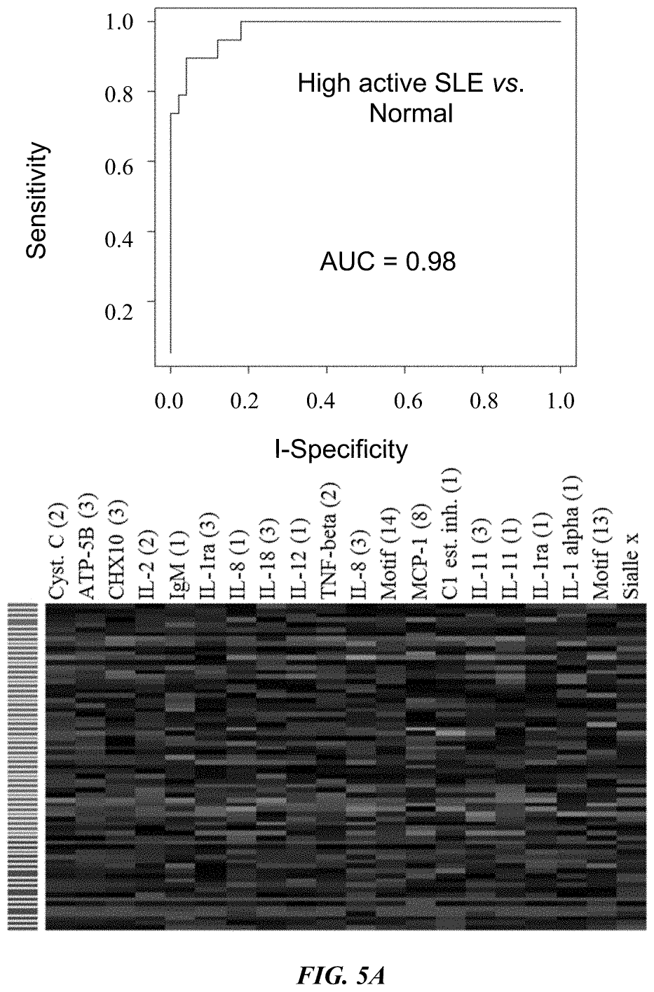

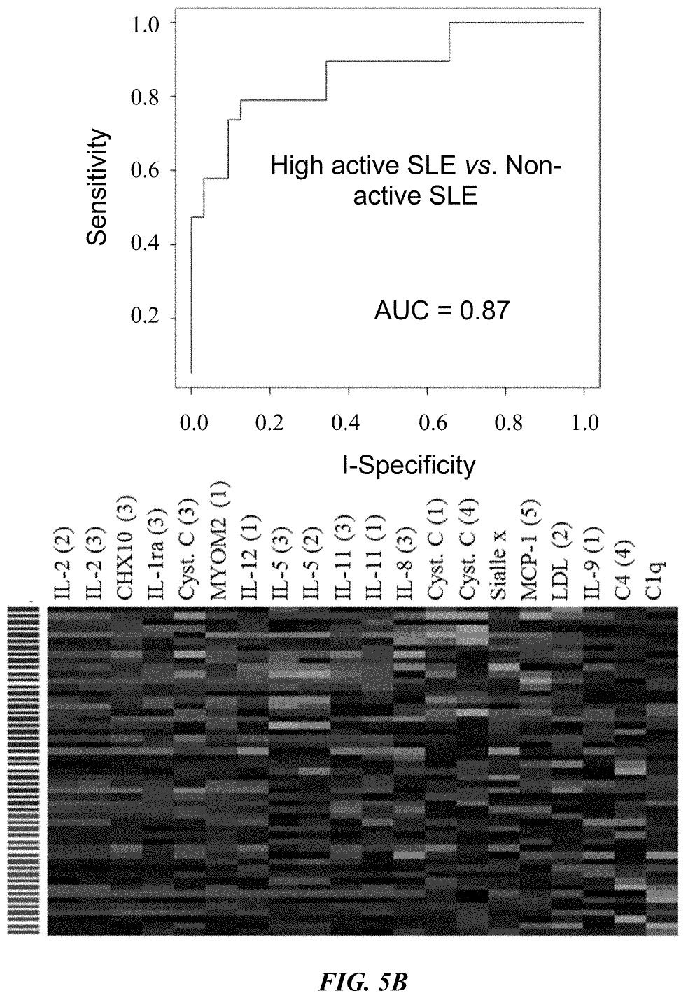

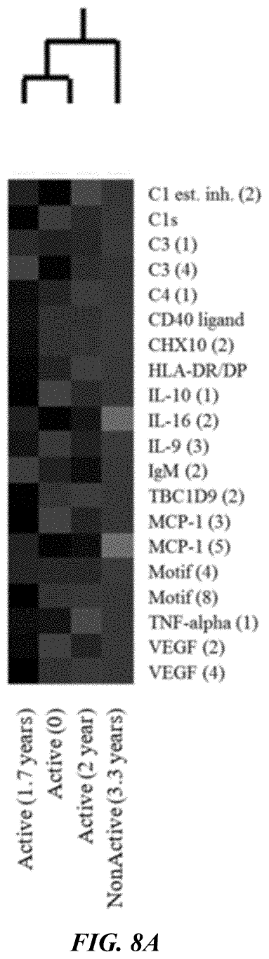

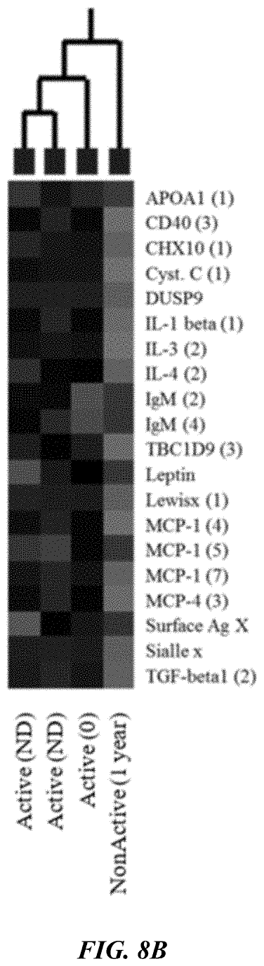

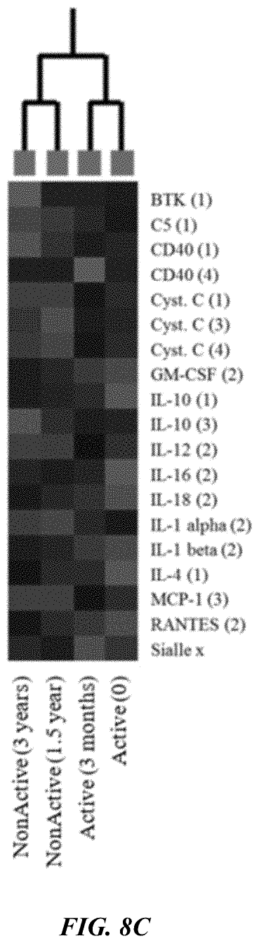

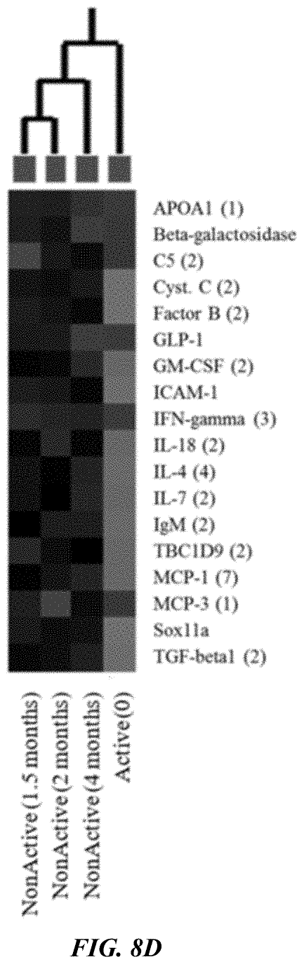

Alternatively or additionally step (b) comprises or consists of measuring the presence and/or amount in the test sample of one or more of the biomarkers listed in FIG. 1(E), FIG. 2(D), FIG. 2(H), FIG. 3(D), FIG. 3(E), FIG. 3(F), FIG. 4(A), FIG. 5(A), FIG. 5(B), FIG. 8(A), FIG. 8(B), FIG. 8(C) and/or FIG. 8(D).

In an alternative or additional embodiment the method comprises recording the diagnosis, prognosis or characterisation on a physical or electronic data carrier (i.e., physical or electronic file).

In an alternative or additional embodiment the method comprises the step of:
(g) determining an/the systemic lupus erythematosus-associated disease state in the subject based on the presence and/or amount in the test sample of the one or more biomarker(s) selected from the group defined in Table A.

In an alternative or additional embodiment in the event that the individual is diagnosed with SLE, the method comprises the step of:
(h) providing the individual with appropriate SLE therapy.

In the event that the individual is not diagnosed with SLE, they may be subjected to further monitoring for SLE (for example, using the method of the present invention).

In an alternative or additional embodiment in the event that the individual is characterised or prognosed as having a flare in SLE (i.e., active or highly active SLE), the method comprises the step of:
(h) providing the individual with appropriate SLE flare therapy.

Treatment may be withdrawn, reduced or otherwise modified in individuals being treated for SLE flare where it is found that the individual is not or is no longer experiencing flare. Hence, the patient is provided treatment appropriate to their SLE-associated disease state. In an alternative or additional embodiment, a more aggressive treatment may be provided for more aggressive SLE types (e.g., SLE3) or during an SLE flare. Suitable therapeutic approaches can be determined by the skilled person according to the prevailing guidance at the time, for example, the American College of Rheumatology Guidelines for Screening, Treatment, and Management of Lupus Nephritis (Hahn et al., 2012, Arthritis Care & Research, 64(6):797-808) which is incorporated herein by reference.

As noted above, in the event that the individual is not diagnosed with SLE flare, they may be subjected to further monitoring for SLE flare (for example, using the method of the present invention).

The repeated monitoring may be repeated at least every 5 days, for example, at least every 10 days, at least every 15 days, at least every 20 days, at least every 25 days, at least every 30 days, at least every 2 months, at least every 3 months, at least every 4 months, at least every 5 months, at least every 6 months, at least every 7 months, at least every 8 months, at least every 9 months, at least every 10 months, at least every 11 months, at least every 12 months, at least every 18 months or at least every 24 months.

Monitoring may also continue in a repeated fashion regardless of whether or not the individual is found to have SLE or SLE flare.

In an alternative or additional embodiment the SLE therapy is selected from the group consisting of systemic inflammation directed treatment (Antimalarials (Hydroxychloroquine), Corticosteroids, Pulse (or mini-pulse) cyclophosphamide (CTX) (with or without corticosteroid co-administration), Mycophenolate mofetil (MMF), Azathioprine (AZA), Methotrexate (MTX)), immune cell targeted therapies (Anti-CD20 antibodies (rituximab, atumumab, ocrelizumab and veltuzumumab), anti-CD22 (Epratuzumab), abetimus (LJP-394), belimumab, atacicept), co-stimulatory signalling pathway targeting (anti-ICOS (inducible costimulator) antibody, anti-ICOS-L (inducible costimulator ligand) antibody, anti-B7RP1 antibody (AMG557)), anti-cytokine therapy (anti-TNF therapy, anti-IL-10, anti-IL-1, anti-IL-18, anti-IL-6, anti-IL-15, memantine, anti-interferon-alpha (IFN-α), plasmapheresis (or plasma exchange), intravenous immunoglobulin (IVIG), DNA vaccination, statins, antioxidants (N-acetylcysteine (NAC), Cysteamine (CYST)), anti-IgE antibodies and anti-FcεRIa antibodies, Syk (spleen tyrosine kinase) inhibition, and Jak (Janus kinase) inhibition), kidney excision, kidney transplant.

Accordingly, the present invention comprises an anti-SLE agent for use in treating SLE wherein the dosage regime is determined based on the results of the method of the first aspect of the invention.

The present invention comprises the use of an anti-SLE agent in treating SLE wherein the dosage regime is determined based on the results of the method of the first aspect of the invention.

A second aspect of the invention provides an array for determining a systemic lupus erythematosus-associated disease state in an individual comprising one or more binding agent as defined in the first aspect of the invention.

Alternatively or additionally the array is for use in a method according to the first aspect of the invention. Alternatively or additionally the array is an array as defined in the first aspect of the invention. Alternatively or additionally the array is capable of binding to all of the proteins defined in Table A.

A third aspect of the invention provides the use of one or more biomarkers selected from the group defined in Table A as a biomarker for determining a systemic lupus erythematosus-associated disease state in an individual. Alternatively or additionally all of the biomarkers defined in Table A are used as a biomarker for determining a systemic lupus erythematosus-associated disease state in an individual.

A fourth aspect of the invention provides the use of one or more biomarkers selected from the group defined in Table A in the manufacture of a medicament (e.g. a diagnostic agent) for determining a systemic lupus erythematosus-associated disease state in an individual.

A fifth aspect of the invention provides one or more biomarkers selected from the group defined in Table A for determining a systemic lupus erythematosus-associated disease state in an individual.

A sixth aspect of the invention provides use of one or more binding agent as defined in the first aspect of the invention for determining a systemic lupus erythematosus-associated disease state in an individual. Alternatively or additionally all of the biomarkers defined in Table A are used for determining a systemic lupus erythematosus-associated disease state in an individual. In one embodiment, the binding agent(s) is/are antibodies or antigen-binding fragments thereof.

A seventh aspect of the invention provides use of one or more binding agent as defined in the first aspect of the invention for the manufacture of a medicament (e.g. a diagnostic agent) for determining a systemic lupus erythematosus-associated disease state in an individual. In one embodiment, the binding agent(s) is/are antibodies or antigen-binding fragments thereof.

An eighth aspect of the invention provides one or more binding agent as defined in the first aspect of the invention for determining a systemic lupus erythematosus-associated disease state in an individual. In one embodiment, the binding agent(s) is/are antibodies or antigen-binding fragments thereof.

A ninth aspect of the invention provides a kit for determining a systemic lupus erythematosus-associated disease state in an individual comprising:
i) one or more binding agent or as defined in the first aspect of the invention or an array as defined in the first or second aspects of the invention, and
ii) (optionally) instructions for performing the method as defined in the first aspect of the invention.

A tenth aspect of the invention provides a method of treating systemic lupus erythematosus in an individual comprising the steps of:
(a) determining a systemic lupus erythematosus-associated disease state in an individual according to the method defined in the first aspect of the invention; and
(b) providing the individual with systemic lupus erythematosus therapy.

By “Systemic lupus erythematosus therapy” we include treatment of the symptoms of systemic lupus erythematosus (SLE), most notably fatigue, joint pain/swelling and/or skin rashes.

Other symptoms of SLE can include:
a fever (high temperature)
swollen lymph glands (small glands found throughout your body, including in your neck, armpits and groin)
recurring mouth ulcers
hair loss (alopecia)
high blood pressure (hypertension)
headaches and migraines
stomach (abdominal) pain
chest pain
depression
dry eyes
memory loss
seizures (fits)
problems thinking clearly and difficulty telling the difference between reality and imagination (psychosis)
shortness of breath
Raynaud's phenomenon—a condition that limits the blood supply to your hands and feet when it is cold
ankle swelling and fluid retention (oedema)

Typically, treatment for SLE may include one or more of the following (see also above):
(a) Limiting exposure to the sun;
(b) Vitamin D supplements;
(c) Non-steroidal anti-inflammatory drugs (NSAIDs), such as ibuprofen;
(d) Antimalarial agents, such as hydroxychloroquine;
(e) Corticosteroids;
(f) Immunosuppressants;
(g) Rituximab; and
(h) Belimumab.

An eleventh aspect of the invention provides a computer program for operating the methods the invention, for example, for interpreting the expression data of step (c) (and subsequent expression measurement steps) and thereby diagnosing or determining a pancreatic cancer-associated disease state. The computer program may be a programmed SVM. The computer program may be recorded on a suitable computer-readable carrier known to persons skilled in the art. Suitable computer-readable-carriers may include compact discs (including CD-ROMs, DVDs, Blue Rays and the like), floppy discs, flash memory drives, ROM or hard disc drives. The computer program may be installed on a computer suitable for executing the computer program.

Preferred, non-limiting examples which embody certain aspects of the invention will now be described with reference to the following tables and above-mentioned figures:

### Examples

Introduction

Objective. To define a multiplex serum biomarker panel reflecting disease activity in systemic lupus erythematosus (SLE), taking the next steps towards serum-based detection of flares.

Methods. Affinity proteomics, represented by 195-plex recombinant antibody microarrays, targeting mainly immunoregulatory proteins, was used to perform protein expression profiling of non-fractionated, biotinylated serum samples. State-of-the-art bioinformatics was used to define biomarkers and condensed multiplex signatures mirroring disease activity in SLE.

Results. The results showed that a single drop of blood contained significant amount of biological information, in the form of immunoregulatory proteins (e.g. C1q, C3, C4, Factor B, MCP-1, CD40L, IL-1ra, IL-5, IL-12, IL-16 and IFN-γ) reflecting SLE flares that could be harvested using affinity proteomics. The first condensed (n≤25) multiplexed serum biomarker panels detecting (classifying) active SLE with high discriminatory power were deciphered. Further, the potential of the approach for serological monitoring of flares over time was indicated.

Conclusion. Our study demonstrated that the immune system could be used as a unique sensor for SLE flares. High-performing serum biomarker panels associated with SLE disease activity were identified, allowing and monitoring and forecasting of disease outbreaks.

Materials and Methods

Clinical Samples

In total, 197 serum samples were collected at the Department of Rheumatology, Skåne University Hospital (Lund, Sweden), including SLE patients (n=86) and normal controls (N) (n=50) (Table I). The SLE patients had clinical SLE diagnosis (22) and displayed four or more of American College of Rheumatology classification criteria (23, 24). The SLE samples were collected over time during follow-up and the patients were presented with either flare or remission, i.e. for some patients up to four samples were collected at different time-points. The SLE patients (samples) were marked according to disease phenotype (25); 1) skin and musculoskeletal involvement (SLE1, n=30); 2) serositis, systemic vasculitis but not kidney involvement (SLE2, n=30); 3) presence of SLE glomerulonephritis (SLE3, n=87).

The clinical disease activity was defined as SLE disease activity index 2000 (SLEDAI-2K) score (5). The SLE samples were grouped in three groups, according to SLEDAI-2K scores; <5=NonActive (n=63), >5=Active (n=83), >16=HighActive (n=28). All samples were aliquoted and stored at −80° C. until analysis. This retrospective study was approved by the regional ethics review board in Lund, Sweden. The serum levels of C1q and C4 were determined using rocket immunoelectrophoresis (C1q) and tubidometry (C4). The same samples have been used in a parallel, but separate study, aiming to define serum biomarkers for SLE diagnosis (Delfani et al, 2016, supra).

Labelling of Serum Samples

The serum samples were labelled with EZ-link Sulfo-NHS-LC-Biotin (Pierce, Rockford, Ill., USA) using a previously optimized labelling protocol for serum proteomes (26-28). Briefly, the samples were diluted 1:45 in PBS (about 2 mg protein/ml), and biotinylated at a molar ratio of biotin:protein of 15:1. Unreacted biotin was removed by extensive dialysis against PBS (pH 7.4) for 72 h at 4° C. The samples were aliquoted and stored at −20° C. until further use.

Production and Purification of Antibodies

In total, 195 human recombinant single-chain fragment variable (scFv) antibodies, including 180 antibodies targeting 73 mainly immunoregulatory analytes, anticipated to reflect the events taking place in SLE, and 15 scFv antibodies targeting 15 short amino acid motifs (4 to 6 amino acids long) (29) were selected from a large phage display library (Supplementary Table I) (30) (Säll et al, submitted). The specificity, affinity, and on-chip functionality of the scFv antibodies have been previously validated (see Supplementary Appendix 1 for details).

All scFv antibodies were produced in E. coli and purified from expression supernatants using affinity chromatography on Ni²⁺-NTA agarose (Qiagen, Hilden, Germany) validated (see Supplementary Appendix 1 for details).

Production and Analysis of Antibody Microarrays

The scFv microarrays were produced and handled using a previously optimized and validated set-up (19) (Delfani et al, 2016, supra) (see Supplementary Appendix 1 for details). Briefly, 14 identical 25×28 subarrays were printed on each black polymer MaxiSorp microarray slide (NUNC A/S, Roskilde, Denmark) using a non-contact printer (SciFlexarrayer S11, Scienion, Berlin, Germany). Biotinylated samples were added and any bound protein antigens were visualized using Alexa 647-labelled streptavidin (SA647) (Invitrogen). Finally, the slides were scanned with a confocal microarray scanner (ScanArray Express, PerkinElmer Life & Analytical Sciences).

Data Pre-Processing

The ScanArray Express software v4.0 (PerkinElmer Life & Analytical Sciences) was used to quantify spot signal intensities. Signal intensities with local background subtraction were used for data analysis. Each data point represents the mean value of all three technical replicate spots, unless any replicate CV exceeded 15%, in which case the worst performing replicate was eliminated and the average value of the two remaining replicates was used instead. Log¹⁰ values of signal intensities were used for subsequent analysis. The microarray data was normalized in a two-step procedure using a semi-global normalization method (19, 31, 32) and the “subtract by group mean” approach (see Supplementary Appendix 1 for details).

Data Analysis

Where applicable, the sample cohort was randomly divided into a training set (⅔ of the samples) and a test set (⅓ of the samples), making sure that the distribution of SLE vs. controls and/or samples with active vs. inactive disease was similar between the two sets. It should be noted that for those SLE patients where more than one sample was at hand, the sample was randomly selected for each comparison, and only one sample per patient was included in each subset comparison in order to avoid bias (i.e. over-representation of certain patients).

The support vector machine (SVM) is a supervised learning method in R (33-35) that we used to classify the samples (see Supplementary Appendix 1 for details). For classification of HighActive SLE vs. N, HighActive SLE vs. NonActive SLE, the SVM was trained using a leave-one-out cross-validation procedure (31), and the prediction performance of the classifier was evaluated by constructing a receiver operating characteristics (ROC) curve and calculating the area under the curve (AUC).

In the case of Active SLE vs. N, NonActive SLE vs. N, and Active SLE vs. NonActive SLE, the samples were randomly divided into a training set and a test set, and a backward elimination algorithm (36) combined with a leave-one-out cross-validation procedure was applied on the training set to determine a condensed panel of antibodies displaying the highest combined discriminatory power. A single SVM model was then calibrated on the training set using the condensed antibody panel, where after the classifier was frozen and evaluated on the test set. This process was iterated nine additional times, in nine different, randomly generated pairs of training sets and test sets, with subsequent generation of ROC AUC curves. In the end, a median AUC value was calculated based on all ten runs, and used as a measure of the accuracy of the biomarker signatures in the tests.

To investigate whether phenotype was a confounding factor for classification of Active SLE vs. N and NonActive SLE vs. N, the SLE samples were also grouped according to phenotype (SLE1, SLE2, and SLE3) and the above analysis were re-run.

Significantly differentially expressed analytes (p<0.05) were identified based on t-tests when performing two-group comparisons. Longitudinal analysis of SLE samples were conducted using multi-group comparison. Heat maps and visualization of the samples by principal component analysis (PCA) were carried using Qlucore Omics Explorer 2.2. (Qlucore AB, Lund, Sweden).

Supplementary Materials and Methods

Production and Purification of Antibodies

In total, 195 human recombinant scFv antibodies, including 180 antibodies targeting 73 mainly immunoregulatory analytes, anticipated to reflect the events taking place in SLE, and 15 scFv antibodies targeting 15 short amino acid motifs (4 to 6 amino acids long) (1) were selected from a large phage display library (Supplementary Table I) (2) (Säll et al, unpublished data). The specificity, affinity (normally in the nM range), and on-chip functionality of these phage display derived scFv antibodies was ensured by using i) stringent phage-display selection and screening protocols (2), ii) multiple clones (1-9) per target, and iii) a molecular design, adapted for microarray applications (3). In addition, the specificity of several of the antibodies have previously also been validated using well-characterized, standardized serum samples (with known analytes of the targeted analytes), and orthogonal methods, such as mass spectrometry (affinity pull-down experiments), ELISA, MesoScaleDiscovery (MSD) assay, cytometric bead assay, and MS, as well as using spiking and blocking (Supplementary Table I) (4-12). Notably, the reactivity of some antibodies might be lost since the label (biotin) used to label the sample to enable detection could block the affinity binding to the antibodies (epitope masking). However, we addressed this potential problem by frequently including more than one antibody clone against the same protein, but directed against different epitopes (3).

All scFv antibodies were produced in 100 ml E. coli and purified from expression supernatants using affinity chromatography on Ni²⁺-NTA agarose (Qiagen, Hilden, Germany). ScFvs were eluted using 250 mM imidazole, extensively dialyzed against PBS (pH 7.4), and stored at 4° C. until use. The protein concentration was determined by measuring the absorbance at 280 nm (average 340 μg/ml, range 30-1500 μg/ml). The degree of purity and integrity of the scFv antibodies was evaluated by 10% SDS-PAGE (Invitrogen, Carlsbad, Calif., USA).

Production and Analysis of Antibody Microarrays

The scFv microarrays were produced using a previously optimized and validated set-up (9) (Delfani et al, unpublished data). Briefly, the antibodies were printed on black polymer MaxiSorp microarray slides (NUNC A/S, Roskilde, Denmark), by spotting one drop (˜330 pL) at each position, using a non-contact printer (SciFlexarrayer S11, Scienion, Berlin, Germany). Each microarray, composed of 195 scFvs antibodies, one negative control (PBS) and one positive control (biotinylated BSA, b-BSA), was split into 14 sub-arrays of 25×28 spots. Furthermore, each sub-array was divided in three segments where a row of b-BSA consisting of 25 replicate spots was printed at the beginning and the end of each segment. Each scFv antibody was dispensed in three replicates, one in each segment, to assure adequate reproducibility.

For handling the arrays, we used a recently optimized protocol (Delfani et al, unpublished data). Briefly, the printed microarrays were allowed to dry for 2 h at RT and were then mounted in a multi-well incubation chambers (NEXTERION® IC-16) (Schott, Jena, Germany). Next, the slides were blocked with 1% (v/v) Tween-20 (Merck Millipore) and 1% (w/v) fat-free milk powder (Semper, Sundbyberg, Sweden) in PBS (MT-PBS solution) for 2 h at RT. Subsequently, the slides were washed for four times with 150 μl 0.05% (v/v) Tween-20 in PBS (T-PBS solution), and then incubated with 100 μl biotinylated serum sample, diluted 1:10 in MT-PBS solution (corresponding to a total serum dilution of 1:450), for 2 h at RT under gentle agitation using an orbital shaker. After another washing, the slides were incubated with 100 μl 1 μg/ml Alexa 647-labelled streptavidin (SA647) (Invitrogen) in MT-PBS for 1 h at RT under agitation. Finally, the slides were washed in T-PBS, and dried under a stream of nitrogen gas, and immediately scanned with a confocal microarray scanner (ScanArray Express, PerkinElmer Life & Analytical Sciences) at 10 μm resolution, using fixed scanner settings of 60% PMT gain and 90% laser power.

Data Pre-Processing

The ScanArray Express software v4.0 (PerkinElmer Life & Analytical Sciences) was used to quantify spot signal intensities, using the fixed circle method. Signal intensities with local background subtraction were used for data analysis. Each data point represents the mean value of all three replicate spots, unless any replicate CV exceeded 15%, in which case the worst performing replicate was eliminated and the average value of the two remaining replicates was used instead. Log¹⁰ values of signal intensities were used for subsequent analysis.

For evaluation of normalization strategies and initial analysis on variance, the data was visualized using principal component analysis (PCA) and hierarchical clustering In Qluecore Omics Explorer (Qlucore AB, Lund, Sweden). Subsequently, the data normalization procedure was carried out in two steps. First, the microarray data was normalized for array-to-array variations using a semi-global normalization method, where 20% of the analytes displaying the lowest CV-values over all samples were identified and used to calculate a scaling factor, as previously described (9, 13, 14). Second, the data was normalized for day-to-day variation using the “subtract by group mean” approach. In this approach, the mean value (x̅) of each analyte (i) within each day of analysis was calculated (=x̅ᵢ), and subtracted from the respective individual values (xᵢ), thus zero centering the data (=xᵢ−x̅ᵢ). Finally, the global mean signal for each antibody was calculated and added to each respective data point in order to avoid negative values in the data set.

Data Analysis

The support vector machine (SVM) is a supervised learning method in R (15-17) that was used to classify the samples. The supervised classification was conducted using a linear kernel, and the cost of constraints was set to 1, which is the default value in the R function SVM, and no attempt was performed to tune it. This absence of parameter tuning was chosen to avoid over fitting. No filtration on the data was done before training the SVM, i.e. all antibodies used on the microarray were included in the analysis. Further, a receiver operating characteristics (ROC) curve, as constructed using the SVM decision values and the area under the curve (AUC), was calculated.

The samples were first randomly divided into a training set (⅔ of the data) and a test set (⅓ of the data) while maintaining the same ratios of samples from each group. It should be noted that for those SLE patients where more than one sample was at hand, the sample was randomly selected for each comparison, and only one sample per patient was included in each subset comparison in order to avoid bias. A backward elimination algorithm (18) combined with a leave-one-out cross-validation procedure was then applied to the training set to create a condensed panel of antibodies displaying the highest combined discriminatory power. The condensed panel of antibodies was then employed to train a single SVM model on the training set. The trained SVM model was then frozen and applied to the test set, and a ROC AUC was calculated and used to evaluate the performance of the SVM classifier. In order to demonstrate the robustness of the data set, 9 additional training and test sets were generated and the above data analysis process was repeated. Finally, the frequency at which each antibody was included in all 10 different defined antibody panels was assessed.

The SVM was trained using the leave-one-out cross-validation procedure as previously described (13). By iterating all samples, a ROC curve was constructed using the decision values and the corresponding AUC value was determined, and used for evaluating the prediction performance of the classifier.

Significantly differentially expressed analytes (p<0.05) were identified based on t-tests. Heat maps and visualization of the samples by principal component analysis (PCA) were carried using Qlucore Omics Explorer.

Results

In this study, we have used recombinant scFv antibody microarrays for pin-pointing serum biomarker panels reflecting disease activity in SLE. A total of 197 biotinylated serum samples (SLE n=147, normal controls n=50) representing 136 patients (86 SLE and 50 controls) (Table I) were profiled using 195-plex antibody microarrays, targeting mainly immunoregulatory analytes. The generated microarray images were transformed into protein expression profiles, or protein maps, and SLE-associated serum biomarkers were deciphered.

Profiling of Active SLE

To decode serum biomarkers reflecting active SLE, we first investigated whether SLE patients with active disease (denoted Active SLE) vs. normal controls could be discriminated. To this end, the data set was randomly divided into a training set (⅔ of all samples) and a test set (⅓ of all samples). A stepwise backward elimination procedure was then applied to the training set in order to identify the smallest set of antibodies, i.e. biomarkers, required for differentiating Active SLE vs. normal controls. The results showed that a combination of 10 antibodies, evaluated in terms of the smallest error, provided the best classification (FIG. 1A). But in order to allow some flexibility in the signature, the top 25 antibodies were selected to represent a condensed biomarker panel (FIG. 1A).

In order to evaluate the classification power of this 25-plex biomarker signature, the panel was first used to train a single SVM model, denoted frozen SVM, on the training set. Next, the frozen SVM model was applied to the independent test set. The results showed that a ROC AUC value of 0.96 was obtained (FIG. 1B), demonstrating that Active SLE vs. normal controls could be differentiated with a high discriminatory power. Visualizing the data using a principle component analysis (PCA) based approach, a similar distinct discrimination was observed (FIGS. 1C and 1D). A heat map for the test set, based on the 25-plex signature, is shown in FIG. 1E. The biomarker panel was found to be composed of both up- (e.g. IL-6, IL-8, MCP-1, and TNF-α) and down-regulated proteins (e.g. C3), although the former dominated. It should be noted that we did not differentiate whether the observed up- and down-regulated levels of a protein was due to an in-/decreased production or in-/decreased consumption. Hence, the results showed that a multiplexed, discriminatory biomarker panel reflecting active SLE could be deciphered from crude serum.

Profiling of NonActive SLE

Next, we focused on SLE patients at remission (denoted NonActive SLE). As above, the samples were randomly divided into a training set and test set, whereafter a condensed 25-plex biomarker signatures discriminating NonActive SLE vs. normal controls was defined and used to train a frozen SVM model (training set). Subsequently, the model was evaluated using the independent test set. The results showed that a ROC AUC value of 0.89 was obtained (FIG. 2A), demonstrating that NonActive SLE vs. normal controls could be differentiated. PCA-based analysis showed that a similar distinct discrimination was obtained (FIGS. 2B and 2C). A heat map for the test set, based on the 25-plex signature, is shown in FIG. 2D. The panel was found to be composed of both up- (e.g. IL-6, IL-18, and TNF-α) and down-regulated proteins (e.g. C3 and C4), but the former dominated. Thus, the data demonstrated that a multiplexed, discriminatory panel of biomarkers reflecting also NonActive SLE could be defined from crude serum.

Profiling of NonActive SLE and Active SLE

Next, we compared the serum protein expression profiles of NonActive SLE (SLEDAI-2K: mean 2, range 0-5) and Active SLE(SLEDAI-2K: mean 13, range 6-32). Using the same stringent bioinformatics approach as above, a condensed 25-plex serum biomarker signature discriminating NonActive SLE vs. Active SLE with a ROC AUC value of 0.83 was deciphered (FIG. 2E). A similar distinct discrimination was observed using PCA-based analysis (FIGS. 2F and 2G). The panel, illustrated as a heat map in FIG. 2H, was found to be composed of cytokines (e.g. IL-16 and IFN-γ), complement proteins (e.g. C4 and Factor B), soluble surface proteins (e.g. CD40 and CD40L) as well as other proteins (e.g. IgM). Taken together, the results thus showed that a multiplexed panel of serum biomarkers discriminating NonActive SLE vs. Active SLE, i.e. reflecting disease activity, could be delineated.

Robustness of the Classifications

To test the robustness of the data set with respect to the above classifications, we randomly divided the entire data set in 9 additional pairs of training and test sets, and re-ran all three of the above comparisons. The results showed that all 10 comparisons resulted in a median ROC AUC value of 0.94 (range 0.83-0.98) for Active SLE vs. controls (FIG. 3A), 0.77 (0.65-0.98) for NonActive SLE vs. controls (FIG. 3B), and 0.72 (0.59-0.88) for Active SLE vs. NonActive SLE (FIG. 3C). Thus, the data indicated that the power and robustness of the classification varied, and decreased in the order of Active SLE vs. N (high)>NonActive SLE vs. N (medium to high), and Active SLE vs. NonActive SLE (low to medium). Apart from illustrating the robustness of the data, it also outlined the importance of how the samples were divided on the subsequent data analysis.

Furthermore, the frequency at which each biomarker occurred in these ten 25-plex signatures is shown in FIGS. 3D to 3F for all markers present six or more times. The data showed that the identity of the top markers varied, as could be expected, but that a core of 8 biomarkers were constant (present in at least 8 of 10 signatures) and highly overlapping (Cystatin C, Sialle x, C3, CD40, TGF-β1, and MCP-1) between Active SLE vs. Normal and NonActive SLE vs. Normal. In contrast, only three core biomarkers (Factor B, Cystatin C, and C1q) were pinpointed for Active SLE vs. NonActive SLE. Of note, the latter classification also resulted in the lowest median ROC AUC value (cfs. FIGS. 3A to 3C). This could indicate a more pronounced impact of biological heterogeneity among, in particular the active SLE patients, on the process of defining serum biomarkers reflecting disease activity. In more detail, how the samples were divided between the training and test sets will more likely play a key role in this particular biomarker identification process, since SLE patients can display similar disease activity in terms of SLEDAI-2K, but based on very different biological (clinical) features.

Biomarkers Reflecting the Biology of Disease Activity

Since the biology of disease activity will not only be reflected by biomarkers identified as being best suited for classification based on backward elimination (see above), we also addressed biomarkers identified as being significantly differentially expressed (p<0.05) based on signal intensities and/or fold changes. To this end, the combined non-redundant top 31 differentially expressed biomarker list for Active SLE vs. Normal, NonActive SLE vs. Normal, and Active SLE vs. NonActive SLE is shown in FIG. 4A. Interestingly, a variety of biomarkers were found to be de-regulated, such as soluble cytokine receptors (e.g. IL-1ra), cytokines (IL-16, and IFN-γ), soluble surface proteins (e.g. CD40), complement proteins (e.g. C1q, C3, and C4), and several other proteins (e.g. Cystatin C and IgM). The de-regulated patterns of C1q, C4, and cystatin C is highlighted (FIG. 4B). Thus, the results showed that a multiplexed panel of deregulated biomarkers reflecting SLE disease activity could be deciphered.

Next, C1q was selected in an attempt to validate the array findings using an orthogonal method. To this end, the levels of C1q, as determined using our recombinant antibody arrays, were compared to those obtained using a clinically implement method (rocket immunoelectrophoresis) (FIG. 4C). The results showed that a similar pattern of de-regulated levels of C1q was observed for Active SLE vs. NonActive SLE. Hence, the observed array data for C1 q was validated using an orthogonal method.

Refined Biomarkers Reflecting High Disease Activity

To find better biomarkers reflecting disease activity, SLE patients displaying high activity (in terms of SLEDAI-2K) were selected (denoted HighActive SLE) (SLEDAI-2k ≥16) and their serum protein profiles were re-compared to those of normal controls and NonActive SLE. The classification was performed adopting a leave-one-out cross-validation, the most stringent approach that can be employed when the sample cohorts were too small to justify the samples to be split into training and test sets.

The results showed that a ROC AUC value of 0.98 was obtained (FIG. 5A), demonstrating that HighActive SLE vs. normal controls could be differentiated with a high discriminatory power. The top 20 significantly differentially expressed (p<0.05) proteins are shown as a heat map (FIG. 4A). The biomarker list contained variety of de-regulated proteins, such as soluble cytokine receptors (e.g. IL-1ra), cytokines (IL-2, IL-8, IL-18, and MCP-1), complement proteins (e.g. C1 esterase inhibitory), and several other proteins (e.g. Cystatin C, Sialle x, and IgM). Of note, several antibodies directed against the same protein, but targeting different epitopes, gave similar results, further supporting the observations.

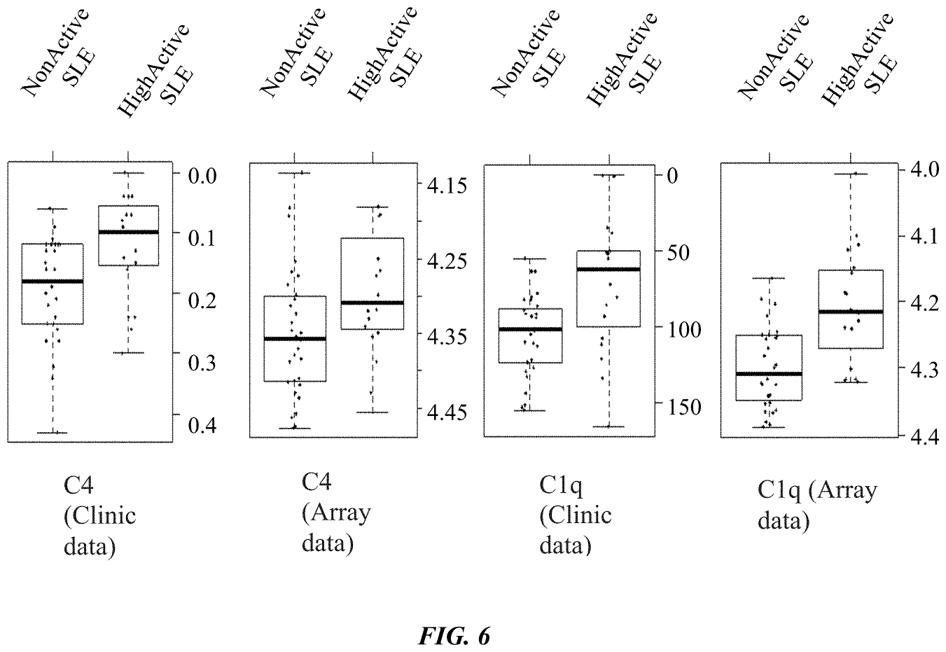

In comparison, HighActive SLE vs. NonActive SLE displayed a ROC AUC value of 0.87, also indicating a high discriminatory power (FIG. 5B). Compared to HighActive SLE vs. normal controls, the top 20 significantly differentially expressed (p<0.05) proteins were, as could be expected, found to display a less distinct heat map (cfs. FIGS. 5A and 5B). Among the de-regulated biomarkers, a range of proteins were observed, such as soluble cytokine receptors (e.g. IL-1ra), cytokines (IL-2, IL-5, IL-12, and MCP-1), complement proteins (e.g. C4 and C1q), and several other proteins (e.g. Cystatin C and Sialle x). Again, the observations were supported by the fact that several antibodies directed against the same protein gave similar profiles. To further support the data, two proteins (C4 and C1q), were selected, and their expression profiles were compared to those determined using orthogonal methods (rocket immunoelectrophoresis (C1q) and tubidometry (C4)). The data showed that the protein expression profiles, obtained using antibody microarrays, could be validated in both cases (FIG. 6).

Importance of Phenotype

Disease phenotype could be one, of several, potential confounding factors, in defining serum biomarkers reflecting SLE disease activity. In an attempt to address this, the samples were also grouped according to phenotype (SLE1, SLE2, and SLE3) and parts of the classifications were re-run (for those groups where sufficient number of samples were still obtained). The classifications were performed adopting a leave-one-out cross-validation.

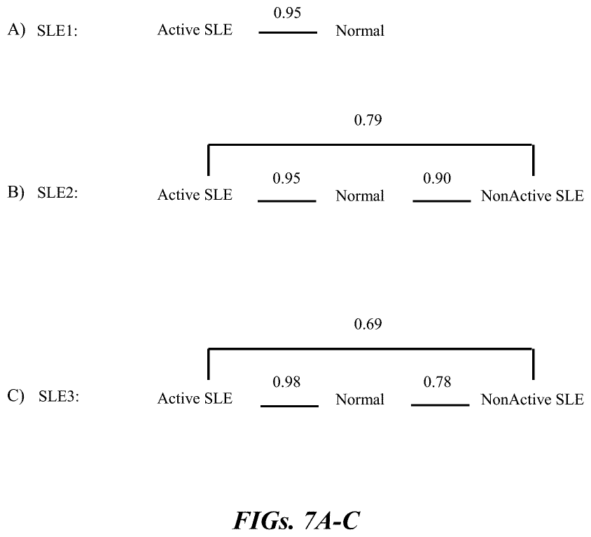

The results showed Active SLE vs. normal controls could be discriminated with a high ROC AUC value independent of phenotype (SLE1—0.95; SLE2—0.95; SLE3—0.98) (FIG. 7). Similarly, NonActive SLE vs. normal controls could also be discriminated irrespective of phenotype (SLE2—0.090; SLE3—0.78) (FIGS. 7B and 7C). Finally, Active SLE vs. NonActive SLE could also be classified irrespective of disease phenotype, albeit with lower ROC AUC values (SLE2—0.79; SLE3—0.69). Hence, the data indicated that phenotype was not a key confounding factor for pinpointing multiplexed serum biomarkers mirroring disease activity.

Monitoring of Disease Activity Over Time

Finally, in a first attempt to explore whether SLE disease activity could be monitored over time, a limited set of longitudinal samples were profiled. To this end, 4 patients were selected as showcase, and 4 serum samples per patient were collected over time (within S 3.3. years) at flares and remissions (3 Active vs. 1 NonActive, 2 Active vs. 2 NonActive, or 1 Active vs. 3 NonActive). For each patient, the top ≤20 de-regulated proteins were identified using multi-group comparisons, and the samples were visualized using supervised hierarchical clustering in combination with heat-maps (FIG. 8). A variety of de-regulated proteins were indicated, including cytokines (e.g. IL-10, IL-12, IL-18, IFN-γ, and MCP-1), complement proteins (e.g. C3, C4, and C5), soluble surface proteins (e.g. CD40), as well as other proteins (e.g. IgM and Sialle x). As before, these pilot observations were supported by that several antibodies directed against the same protein, but targeting different epitopes, gave similar profiles. For all 4 patients, the samples clustered as two groups, Active vs. NonActive, meaning that the longitudinal samples collected ate flares were more similar to each other than to those collected at remission, and vice versa. This further supports the notion that crude serum contains information (biomarkers) reflecting disease activity that could be harvested using affinity proteomics, indicating the potential for monitoring disease activity over time.

Discussion

Biomarkers that could be used to detect, monitor, and/or even forecast SLE flares would be a very valuable clinical tool (9, 11). Despite major efforts, it is clear that the quest for such high-performing markers, preferentially based on a blood test, is still at an early stage (37). Since we can only manage what we can measure, additional and/or refined methodologies for protein expression profiling of crude clinical samples will be essential. Here, we have expanded previous efforts (19, 20) (Nordström et al, submitted; Delfani et al, 2016, supra), and further showed that a single drop of blood harboured significant amount of SLE related information, in the format of relevant biological biomarkers, that could be harvested, using recombinant antibody microarrays.

25-plex panels of biomarkers reflecting SLE disease activity (and SLE) were defined, including group of proteins such as complement proteins (e.g. C1q, C1 esterase inhibitor, C3, C4, C5, and Factor B), cytokines (e.g. IL-1ra, IL-2, IL-5, IL-6, IL-8, IL-12, IL-16, IL-18, IFN-γ, MCP-1, TGF-β1, and TNF-α), cytokine receptors (cytokines (e.g. IL1ra), soluble surface proteins (e.g. CD40 and CD40L), and other proteins (e.g. Cystatin C, Sialle x, and IgM). These biomarker panels could be used to classify active SLE, although the power of classification varied from high (ROC AUC of 0.98) to low (AUC of 0.69) depending the precise comparison at hand. In agreement, some of these markers have in previous work also been found to be associated with SLE flares (and/or SLE per see), see e.g. (4, 7-11, 38, 39). But the markers were then mainly explored as single biomarkers and/or low-plex panels, displaying varying (low) performance.

When comparing the core of the 8 most robust (frequently occurring) markers for Active SLE vs. N with NonActive SLE vs. N, 6 of 8 biomarkers were found to overlap (Cystatin C, Sialle x, CD40, TGF-β1, C3, and MCP-1). Serum Cystatin C is a biomarker that has been found to be associated with renal impairment in SLE, and thus deregulated in SLE (40, 41). Imbalance of T-helper subsets (TH1, TH2, and TH17) and regulatory T-cells has been suggested to contribute to the pathogenesis of SLE (42). In such context, sialle x, or sialyl lewis x, has been shown to identify highly differentiated and most suppressive FXP3^{high} regulatory T cells involved in flares (43). Deregulated levels of CD40 has been observed in SLE, and autoreactive B cells and its abnormal CD40 signalling play important roles in the pathogenesis of SLE (44). Of note, de-regulated levels of CD40L, which binds to CD40, has also been frequently observed and correlated with SLE disease activity (45). In fact, CD40L was differentially expressed when comparing Active SLE vs. NonActive SLE. Further, deregulated serum levels of TGF-β1 has been shown to be associated with renal damage in SLE, in particular for patients with high disease activity (46). Altered levels of several complement proteins, including C3, has often been used as a marker for disease activity (47). MCP-1 is a leukocyte chemotactic factor that has been associated with renal injury, poor prognosis, and disease activity (48). Hence, the biomarkers of this core overlap were found to be biologically relevant markers.

Instead, focusing on the serum signature discriminating Active SLE vs. NonActive SLE, the core of 3 most robust (frequently occurring) included Factor B, C1q, and Cystatin C. Factor B is often used as an indicator for alternative pathway activation of the complement system. Notably, previous work has shown Factor B activation products in SLE to be marker(s) of severe disease activity (49). Furthermore, the levels of classical complement pathway components, such as C1q, have often been found to be altered in patients with severe disease and high disease activity (47). When reviewing the remaining seven most robust (frequently occurring) biomarkers reflecting disease activity, at least five (MCP-1, IL-9, IL-5, IL-1β, and CD40) have been shown to be associated with SLE and reflect disease activity (44, 48, 50-52). While RANTES have been shown to be associated with SLE, no correlation with disease activity has yet been confirmed (53). In our study, RANTES was indicated in the core signature reflecting flares 7 of 10 times. Several serum biomarkers reflecting flares were detected, outlining the potential of the approach. Notably, additional biomarkers (e.g. IL1-ra, IL-2, and IL-12), e priori known to be associated with SLE disease activity (11, 39, 54), were delineated when the group of SLE patients with active disease were reduced to those with high activity only. Although the precise role of IL-11 in SLE is yet unknown, considering its similarities with IL-6, it could lead to a lupus flare.

While this study showed that flares could be detected, it is limited by the fact that only the endpoints, remission vs. “full flare”, were studied. As a limited showcase, we analyzed 4 patients with 4 samples each collected over time at flares and/or remissions. While the sample set is too small to draw definite conclusions, it indicated the potential of using the approach for monitoring flares over time. It will, however, be required to profile larger independent sample cohorts, composed of many well-characterized patients, each with numerous samples collected over time at frequent time-points at flares and/or remissions to demonstrate and establish serological-based tests for monitoring and potentially even forecasting of flares in a stringent manner. The clinical impact of such potential tests are, however, significant.

Taken together, in this study we have shown that condensed (n≤25), multiplexed panels of serum biomarkers detecting, and monitoring, SLE flares could be delineated.

### REFERENCES

1. Tsokos G C. systemic lupus erythematosus. N Engl J Med 2011; 365:2110-21.
2. Rahman A, Isenberg D A. systemic lupus erythematosus. N Engl J Med 2008; 358:929-39.
3. Eisenberg R. Why can't we find a new treatment for SLE? J Autoimmun 2009; 32:223-30.
4. Ahearn J M, Liu C C, Kao A H, Manzi S. Biomarkers for systemic lupus erythematosus. Transl Res 2012; 159:326-42.
5. Gladman D D, Ibañez D, Urowitz M B. systemic lupus erythematosus disease activity index 2000. J Rheumatol 2002; 29:288-91.
6. Rovin B H, Birmingham D J, Nagaraja H N, Yu C Y, Hebert L A. Biomarker discovery in human SLE nephritis. Bull NYU Hosp Jt Dis 2007; 65:187-93.
7. Mok C C. Biomarkers for lupus nephritis: a critical appraisal. J Biomed Biotechnol 2010; 2010:638413.
8. Liu C C, Manzi S, Ahearn J M. Biomarkers for systemic lupus erythematosus: a review and perspective. Curr Opin Rheumatol 2005; 17:543-9.
9. Rovin B H, Zhang X. Biomarkers for lupus nephritis: the quest continues. Clin J Am Soc Nephrol 2009; 4:1858-65.
10. Gigante A, Gasperini M L, Afeltra A, et al. Cytokines expression in SLE nephritis. Eur Rev Med Pharmacol Sci 2011; 15:15-24.
11. Liu C C, Ahearn J M. The search for lupus biomarkers. Best Pract Res Clin Rheumatol 2009; 23:507-23.
12. Putterman C, Wu A, Reiner-Benaim A, et al. SLE-key((R)) rule-out serologic test for excluding the diagnosis of systemic lupus erythematosus: Developing the ImmunArray iCHIP((R)). J Immunol Methods 2016; 429:1-6.
13. Li Q Z, Zhou J, Wandstrat A E, et al. Protein array autoantibody profiles for insights into systemic lupus erythematosus and incomplete lupus syndromes. Clin Exp Immunol 2007; 147:60-70.
14. Fattal I, Shental N, Mevorach D, et al. An antibody profile of systemic lupus erythematosus detected by antigen microarray. Immunology 2010; 130:337-43.
15. Huang W, Hu C, Zeng H, et al. Novel systemic lupus erythematosus autoantigens identified by human protein microarray technology. Biochem Biophys Res Commun 2012; 418:241-6.
16. Bauer J W, Petri M, Batliwalla F M, et al. Interferon-regulated chemokines as biomarkers of systemic lupus erythematosus disease activity: a validation study. Arthritis Rheum 2009; 60:3098-107.
17. Bauer J W, Baechler E C, Petri M, et al. Elevated serum levels of interferon-regulated chemokines are biomarkers for active human systemic lupus erythematosus. PLoS Med 2006; 3:e491.
18. Li Q Z, Xie C, Wu T F, et al. Identification of autoantibody clusters that best predict lupus disease activity using glomerular proteome arrays (vol 115, pg 3248, 2005). J Clin Invest 2006; 116:548-.
19. Carlsson A, Wuttge D M, Ingvarsson J, et al. Serum protein profiling of systemic lupus erythematosus and systemic sclerosis using recombinant antibody microarrays. Mol Cell Proteomics 2011; M110.005033.
20. Petersson L, Dexlin-Mellby L, Bengtsson A A, Sturfelt G, Borrebaeck C A K, Wingren C. Multiplexing of miniaturized planar antibody arrays for serum protein profiling—a biomarker discovery in SLE nephritis. Lab on a Chip 2014; 14:1931-42.
21. Tjalsma H, Schaeps R M, Swinkels D W. Immunoproteomics: From biomarker discovery to diagnostic applications. Proteomics Clin Appl 2008; 2:167-80.
22. Fries J F, Holman H R. systemic lupus erythematosus: a clinical analysis. Major Probl Intern Med 1975; 6:v-199.
23. MC. H. Updating the American College of Rheumatology revised criteria for the classification of systemic lupus erythematosus. Arthritis Rheum 1997; 40:1725.
24. Tan E M, Cohen A S, Fries J F, et al. The 1982 revised criteria for the classification of systemic lupus erythematosus. Arthritis Rheum 1982; 25:1271-7.
25. Sturfelt G, Sjöholm A G. Complement components, complement activation, and acute phase response in systemic lupus erythematosus. Int Arch Allergy Appl Immunol 1984; 75:75-83.
26. Ingvarsson J, Larsson A, Sjöholm A G, et al. Design of recombinant antibody microarrays for serum protein profiling: targeting of complement proteins. J Proteome Res 2007; 6:3527-36.
27. Wingren C, Ingvarsson J, Dexlin L, Szul D, Borrebaeck C A K. Design of recombinant antibody microarrays for complex proteome analysis: choice of sample labeling-tag and solid support. Proteomics 2007; 7:3055-65.
28. Wingren C, Borrebaeck C A. Antibody microarray analysis of directly labelled complex proteomes. Curr Opin Biotechnol 2008 19:55-61.
29. Olsson N, Wallin S, James P, Borrebaeck C A K, Wingren C. Epitope-specificity of recombinant antibodies reveals promiscuous peptide-binding properties. Protein Science 2012; 21:1897-910.
30. Söderlind E, Strandberg L, Jirholt P, et al. Recombining germline-derived CDR sequences for creating diverse single-framework antibody libraries. Nat Biotechnol 2000; 18:852-6.
31. Carlsson A, Wingren C, Ingvarsson J, et al. Serum proteome profiling of metastatic breast cancer using recombinant antibody microarrays. Eur J Cancer 2008; 44:472-80.
32. Carlsson A, Persson O, Ingvarsson J, et al. Plasma proteome profiling reveals biomarker patterns associated with prognosis and therapy selection in glioblastoma multiforme patients. Proteomics Clin Appl 2010; 4:591-602.
33. Chih-chung C, Chih-Jen L. LIBSVM: a library for support vector machines. http/::wwwcsientuedutw/cjlin/libsvm 2007.
34. Cristianini N, Shawe-Taylor J. An introduction to support vector machines and other kernel-based learning methods. Cambridge University Press 2000.
35. Ihaka R, Gentleman R. A language for data analysis and graphics. J Comp Graph Stat 1996; 5:299-314.
36. Carlsson A, Wingren C, Kristensson M, et al. Molecular serum portraits in patients with primary breast cancer predict the development of distant metastases. Proc Natl Acad Sci USA 2011; 108:14252-7.
37. Misra R, Gupta R. Biomarkers in lupus nephritis. Int J Rheum Dis 2015; 18:219-32.
38. Su D L, Lu Z M, Shen M N, Li X, Sun LY. Roles of pro- and anti-inflammatory cytokines in the pathogenesis of SLE. J Biomed Biotechnol 2012; 2012:347141.
39. Sedighi S, Aghaei M, Musavi S, Nomali M. Relationship between Serum Level of Interleukin-2 in Patients with systemic lupus erythematosus and Disease Activity in Comparison with Control Group. J Clin Diagn Res 2014; 8:MC16-8.
40. Chew C, Pemberton P W, Husain A A, Haque S, Bruce I N. Serum cystatin C is independently associated with renal impairment and high sensitivity C-reactive protein in systemic lupus erythematosus. Clin Exp Rheumatol 2013; 31:251-5.
41. Lertnawapan R, Bian A, Rho Y H, et al. Cystatin C is associated with inflammation but not atherosclerosis in systemic lupus erythematosus. Lupus 2012; 21:279-87.
42. Talaat R M, Mohamed S F, Bassyouni I H, Raouf A A. Th1/Th2/Th17/Treg cytokine imbalance in systemic lupus erythematosus (SLE) patients: Correlation with disease activity. Cytokine 2015; 72:146-53.
43. Miyara M, Chader D, Sage E, et al. Sialyl Lewis x (CD15s) identifies highly differentiated and most suppressive FOXP3high regulatory T cells in humans. Proc Natl Acad Sci USA 2015; 112:7225-30.
44. Zhang W, Shi Q, Xu X, et al. Aberrant CD40-induced NF-kappaB activation in human lupus B lymphocytes. PLoS One 2012; 7:e41644.
45. de Sanctis J B, Garmendia J V, Chaurio R, Zabaleta M, Rivas L. Total and biologically active CD154 in patients with SLE. Autoimmunity 2009; 42:263-5.
46. Jin T, Almehed K, Carlsten H, Forsblad-d'Elia H. Decreased serum levels of TGF-beta1 are associated with renal damage in female patients with systemic lupus erythematosus. Lupus 2012; 21:310-8.
47. Pickering M C, Walport M J. Links between complement abnormalities and systemic lupus erythematosus. Rheumatology (Oxford) 2000; 39:133-41.
48. Choe J Y, Kim S K. Serum TWEAK as a biomarker for disease activity of systemic lupus erythematosus. Inflamm Res 2016; 65:479-88.
49. Kerr L D, Adelsberg B R, Schulman P, Spiera H. Factor B activation products in patients with systemic lupus erythematosus. A marker of severe disease activity. Arthritis Rheum 1989; 32:1406-13.
50. Ouyang H, Shi Y B, Su N, Li L Y. [Abnormality and significance of interleukin-9 and CD4(+)interleukin-9(+) T-cells in peripheral blood of patients with systemic lupus erythematosus]. Zhonghua Yi Xue Za Zhi 2013; 93:99-103.
51. Brito T N, Vilar M J, Almeida J B, et al. Measuring eosinophiluria, urinary eosinophil cationic protein and urinary interleukin-5 in patients with Lupus Nephritis. Allergy Asthma Clin Immunol 2014; 10:61.
52. Umare V, Pradhan V, Nadkar M, et al. Effect of proinflammatory cytokines (IL-6, TNF-alpha, and IL-1beta) on clinical manifestations in Indian SLE patients. Mediators Inflamm 2014; 2014:385297.
53. Lu M M, Wang J, Pan H F, et al. Increased serum RANTES in patients with systemic lupus erythematosus. Rheumatol Int 2012; 32:1231-3.
54. Capper E R, Maskill J K, Gordon C, Blakemore A I. Interleukin (IL)-10, IL-1ra and IL-12 profiles in active and quiescent systemic lupus erythematosus: could longitudinal studies reveal patient subgroups of differing pathology? Clin Exp Immunol 2004; 138:348-56.

### SUPPLEMENTARY REFERENCES

1. Olsson N, Wallin S, James P, Borrebaeck C A K, Wingren C. Epitope-specificity of recombinant antibodies reveals promiscuous peptide-binding properties. Protein Science 2012; 21:1897-910.
2. Söderlind E, Strandberg L, Jirholt P, et al. Recombining germline-derived CDR sequences for creating diverse single-framework antibody libraries. Nat Biotechnol 2000 18:852-6.
3. Borrebaeck C A K, Wingren C. Recombinant Antibodies for the Generation of Antibody Arrays. In: Korf U, editor. Protein Microarrays: Humana Press; 2011. p. 247-62.
4. Ingvarsson J, Larsson A, Sjöholm A G, et al. Design of recombinant antibody microarrays for serum protein profiling: targeting of complement proteins. J Proteome Res 2007 6:3527-36.
5. Kristensson M, Olsson K, Carlson J, et al. Design of recombinant antibody microarrays for urinary proteomics. Proteomics Clin Appl 2012 6:291-6.
6. Wingren C, Ingvarsson J, Dexlin L, Szul D, Borrebaeck C A. Design of recombinant antibody microarrays for complex proteome analysis: choice of sample labeling-tag and solid support. Proteomics 2007 7:3055-65.
7. Persson J, Bäckström M, Johansson H, Jirström K, Hansson G C, Ohlin M. Molecular Evolution of Specific Human Antibody against MUC1 Mucin Results in Improved Recognition of the Antigen on Tumor Cells. Tumor Biology 2009; 30:221-31.
8. Gustavsson E, Ek S, Steen J, et al. Surrogate antigens as targets for proteome-wide binder selection. New Biotechnology 2011; 28:302-11.
9. Carlsson A, Wuttge D M, Ingvarsson J, et al. Serum protein profiling of systemic lupus erythematosus and systemic sclerosis using recombinant antibody microarrays. Mol Cell Proteomics 2011 M110.005033.
10. Dexlin-Mellby L, Sandström A, Centlow M, et al. Tissue proteome profiling of preeclamptic placenta using recombinant antibody microarrays. Proteomics Clin Appl 2010 4:794-807.
11. Ingvarsson J, Wingren C, Carlsson A, et al. Detection of pancreatic cancer using antibody microarray-based serum protein profiling. Proteomics 2008 8:2211-9.
12. Pauly F, Dexlin-Mellby L, Ek S, et al. Protein Expression Profiling of Formalin-Fixed Paraffin-Embedded Tissue Using Recombinant Antibody Microarrays. Journal of Proteome Research 2013; 12:5943-53.
13. Carlsson A, Wingren C, Ingvarsson J, et al. Serum proteome profiling of metastatic breast cancer using recombinant antibody microarrays. Eur J Cancer 2008 44:472-80.
14. Carlsson A, Persson O, Ingvarsson J, et al. Plasma proteome profiling reveals biomarker patterns associated with prognosis and therapy selection in glioblastoma multiforme patients. Proteomics Clin Appl 2010 4:591-602.
15. Chih-chung C, Chih-Jen L. LIBSVM: a library for support vector machines. http/::wwwcsientuedutw/cjlin/libsvm 2007.
16. Cristianini N, Shawe-Taylor J. An introduction to support vector machines and other kernel-based learning methods. Cambridge University Press 2000.
17. Ihaka R, Gentleman R. A language for data analysis and graphics. J Comp Graph Stat 1996; 5:299-314.
18. Carlsson A, Wingren C, Kristensson M, et al. Molecular serum portraits in patients with primary breast cancer predict the development of distant metastases. Proc Natl Acad Sci USA 2011 108:14252-7.

Tables

TABLE A
Core, preferred and optional biomarkers for determining a
systemic lupus erythematosus-associated disease state

| | |
|---|---|
| Biomarker | Exemplary sequence(s) |

I-core

| | | |
|---|---|---|
| 1 | CHX10 (3) | P58304 |
| 2 | LUM | P51884 |
| 3 | Cyst. C | P01034 |

II-preferred

| | | |
|---|---|---|
| 4 | ATP5B (2) | P06576 |
| 5 | Beta-galactosidase | P16278 |
| 6 | DUSP9 | Q99956 |
| 7 | MYOM2 (1) | P54296 |
| 8 | PSA | P07288 |
| 9 | Sox11a | P35716 |
| 10 | Surface Ag X | NA |
| 11 | TBC1D9 (2) | Q6ZT07 |
| 12 | IL-1 alpha | P01583 |
| 13 | IL-1 beta | P01584 |
| 14 | Motif (13) | SGSG-QEASFK(-COOH) |
| | | [SEQ ID NO: 1] |
| 15 | Motif (14) | SGSG-EDFR(-COOH) |
| | | [SEQ ID NO: 2] |
| 16 | Motif (3) | SGSG-DFAEDK(-COOH) |
| | | [SEQ ID NO: 3] |
| 17 | Motif (4) | SGSG-TEEQLK(-COOH) |
| | | [SEQ ID NO: 4] |
| 18 | Motif (5) | SGSG-LSADHR(-COOH) |
| | | [SEQ ID NO: 5] |
| 19 | Motif (7) | SGSG-TEEQLK(-COOH) |
| | | [SEQ ID NO: 4] |
| 20 | Motif (8) | SGSG-TEEQLK(-COOH) |
| | | [SEQ ID NO: 4] |

III-optional

| | | |
|---|---|---|
| 21 | Angiomotin (1) | Q4VCS5 |
| 22 | APOA1 (1) | P02647 |
| 23 | BTK(1) | Q06187 |
| 24 | C1 est. inh. (3) | P05155 |
| 25 | C1q | P02745/6/7 |
| 26 | C1s | P09871 |
| 27 | C3 | P01024 |
| 28 | C4 | P0COL4/5 |
| 29 | C5(1) | P01031 |
| 30 | CD40 | Q6P2H9 |
| 31 | CD40 ligand | P29965 |
| 32 | Eotaxin (3) | P51671 |
| 33 | Factor B | P00751 |
| 34 | GLP-1 | P01275 |
| 35 | GM-CSF | P04141 |
| 36 | HLA-DR/DP | P01903/P01911/P79483/ |
| | | P13762/Q30154/P20036/P04440 |
| 37 | ICAM-1 | P05362 |
| 38 | IFN-gamma (1) | P01579 |
| 39 | igM | e.g. P01871 (not complete |
| | | protein); isotype-specific for |
| | | IgM on Ramos B cells) |
| 40 | IL-10 (1) | P22301 |
| 41 | IL-11 (1) | P20809 |
| 42 | IL-12 (1) | P29459/60 |
| 43 | IL-13 (1) | P35225 |
| 44 | IL-16 (1) | Q14005 |
| 45 | IL-18 | Q14116 |
| 46 | IL-1ra | P18510 |
| 47 | IL-2 (2) | P60568 |
| 48 | IL-3 | P08700 |
| 49 | IL-4 | P05112 |
| 50 | IL-5 | P05113 |
| 51 | IL-6 | P05231 |
| 52 | IL-7 | P13232 |
| 53 | IL-8 | P10145 |
| 54 | IL-9 | P15248 |
| 55 | Integrin alpha-10 | 075578 |
| 56 | JAK3 | P52333 |
| 57 | LDL (1) | P04114 |
| 58 | Leptin | P41159 |
| 59 | Lewis x (1) | NA |
| 60 | MCP-1 | P13500 |
| 61 | MCP-3 (1) | P80098 |
| 62 | MCP-4 (2) | Q99616 |
| 63 | Procathepsin W | P56202 |
| 64 | RANTES | P13501 |
| 65 | Sialle x | NA |
| 66 | TGF-beta1 | P01137 |
| 67 | TNF-alpha(l) | PO1375 |
| 68 | TNF-beta | P01374 |
| 69 | VEGF (1) | P15692 |

TABLE B
Biomarkers for determining a systemic lupus
erythematosus-associated disease state

| | | | | | |
|---|---|---|---|---|---|
| Biomarker | AvN | NAvN | AvNA | HAvN | HAvNA |

I

| | | | | | | |
|---|---|---|---|---|---|---|
| 1 | Cyst. C | x | x | x | x | x |
| 2 | MCP-1 | x | x | x | x | x |
| 3 | Sialle x | x | x | x | x | x |

II

| | | | | | |
|---|---|---|---|---|---|
| 4 | C1 est. inh. (3) | x | x | x | x |
| 5 | IgM | x | x | x | x |
| 6 | TNF-beta | x | x | x | x |

III

| | | | | | |
|---|---|---|---|---|---|
| 7 | C1q | x | x | x | x |
| 8 | C4 | x | x | x | x |

IV

| | | | | | |
|---|---|---|---|---|---|
| 9 | IL-11 (1) | x | x | x | x |
| 10 | IL-1ra | x | x | x | x |

V

| | | | | |
|---|---|---|---|---|
| 11 | IL-1 beta | x | x | x |
| 12 | CD40 | x | x | x |
| 13 | Factor B | x | x | x |
| 14 | Integrin | x | x | x |
| | alpha-10 | | | |

VI1

| | | | | |
|---|---|---|---|---|
| 15 | CHX10 (3) | x | x | x |
| 16 | IL-8 | x | x | x |

VII

| | | | | |
|---|---|---|---|---|
| 17 | IL-5 | x | x | x |

VIII

| | | | | |
|---|---|---|---|---|
| 18 | IL-9 | x | x | x |

IX

| | | | | |
|---|---|---|---|---|
| 19 | LDL (1) | x | x | x |

X

| | | | |
|---|---|---|---|
| 20 | C3 | x | x |
| 21 | IL-6 | x | x |
| 22 | GLP-1 | x | x |
| 23 | TGF-beta1 | x | x |
| 24 | TNF-alpha(1) | x | x |
| 25 | APOA1 (1) | x | x |
| 26 | VEGF (1) | x | x |

XI

| | | | |
|---|---|---|---|
| 27 | LUM | x | x |
| 28 | Motif (4) | x | x |
| 29 | PSA | x | x |
| 30 | GM-CSF | x | x |
| 31 | IFN-gamma | x | x |
| | (1) | | |
| 32 | IL-13 (1) | x | x |
| 33 | IL-16 (1) | x | x |
| 34 | RANTES | x | x |

XII

| | | | | |
|---|---|---|---|---|
| 35 | ATP5B (2) | x | x | |
| 36 | IL-1 alpha | x | x | |
| 37 | IL-18 | x | x | |
| 38 | IL-12 (1) | | x | x |
| 39 | IL-2 (2) | | x | x |

XIV

| | | | |
|---|---|---|---|
| 40 | Motif (14) | x | x |

XV

| | | | |
|---|---|---|---|
| 41 | Motif (3) | x | x |

XVI

| | | |
|---|---|---|
| 42 | Motif (5) | x |
| 43 | Motif (7) | x |
| 44 | TBC1D9 (2) | x |
| 45 | Eotaxin (3) | x |
| 46 | IL-3 | x |
| 47 | IL-4 | x |
| 48 | MCP-4 (2) | x |
| 49 | Procathepsin | x |
| | W | |

XVII

| | | |
|---|---|---|
| 50 | DUSP9 | x |
| 51 | Angiomotin (1) | x |
| 52 | CD40 ligand | x |
| 53 | JAK3 | x |
| 54 | Beta- | x |
| | galactosidase | |
| 55 | Motif (8) | x |
| 56 | Sox11a | x |
| 57 | Surface Ag X | x |
| 58 | BTK (1) | x |
| 59 | C1s | x |
| 60 | C5 (1) | x |
| 61 | HLA-DR/DP | x |
| 62 | ICAM-1 | x |
| 63 | IL-10 (1) | x |
| 64 | IL-7 | x |
| 65 | Leptin | x |
| 66 | Lewis x (1) | x |
| 67 | MCP-3 (1) | x |

XVIII

| | | |
|---|---|---|
| 68 | Motif (13) | x |

XIX

| | | |
|---|---|---|
| 69 | MYOM2 (1) | x |

TABLE 1
Demographic data of SLE patients and
normal controls included in the study.

| | | | | Normal |
|---|---|---|---|---|
| Parameter | | | SLE | controls |
| No. of patients | | | 86 | 50 |
| No. of serum samples | | | 147* | 50 |
| Gender (female:male ratio) | | | (76:10) | (48:2) |
| Mean age (range) | | | 39 (18-72) | 48 (19-68) |
| SLEDAI-2K, mean (range), | | - All | 8 (0-32) | n.a |
| | - All | - Active | 13 (6-32) | |
| | - Active | - NonActive | 2 (0-5) | |

| | SLE1 | SLE2 | SLE3** |
|---|---|---|---|
| No. of SLE samples/phenotype | 30 | 30 | 87 |
| No. of NonActive (SLEDAI ≤ 5) | 15 | 18 | 30 |
| No. of Active (SLEDAI > 5) | 15 | 12 | 56 |
| No. of HighActive (SLEDAI ≥ 16) | 1 | 3 | 24 |

*The samples were collected over time during follow-up and the patients were presented with either flare or remission, i.e. for some patients up to four samples were collected at different time-points.
**One SLE3 sample lacked clinical information on disease activity status.

TABLE 2
30 most frequent markers for the 3 main comparisons

| Active SLE vs | | NonActive SLE vs | | Active SLE vs | |
|---|---|---|---|---|---|
| Normal | | Normal | | NonActive SLE | |
| | Itera- | | Itera- | | Itera- |
| | tions | | tions | | tions |
| Analytes | (%) | Analytes | (%) | Analytes | (%) |
| Cyst. C | 100 | C3 | 100 | Factor B | 100 |
| IL-1ra | 100 | C4 | 100 | C1q | 80 |
| C3 | 90 | Cyst. C | 100 | Cyst. C | 80 |
| CD40 | 90 | MCP-1 | 100 | RANTES | 70 |
| Sialle x | 90 | Sialle x | 90 | CD40 | 60 |
| IL-3 | 80 | CD40 | 80 | GM-CSF | 60 |
| MCP-1 | 80 | IgM | 80 | IL-1β | 60 |
| TGF-β1 | 80 | TGF-β1 | 80 | IL-5 | 60 |
| C4 | 70 | C1 est. inh. | 60 | IL-9 | 60 |
| Motif (4) | 70 | Factor B | 60 | MCP-1 | 60 |
| IL-4 | 70 | IL-18 | 60 | BTK | 50 |
| IL-6 | 70 | IL-1α | 60 | C3 | 50 |
| IL-8 | 70 | Angiomotin | 50 | IL-18 | 50 |
| LUM | 70 | IL-6 | 50 | IL-1ra | 50 |
| TNF-α | 70 | S Ag X | 50 | IL-3 | 50 |
| IgM | 60 | IL-12 | 40 | IL-8 | 50 |
| Procathepsin | 60 | IL-13 | 40 | LUM | 50 |
| W | | | | | |
| RANTES | 60 | IL-1β | 40 | MCP-4 | 50 |
| C1q | 50 | TNF-β | 40 | Sialle x | 50 |
| Factor B | 50 | APOA1 | 30 | TNF-β | 50 |
| Motif (5) | 50 | ATP5B | 30 | APOA4 | 40 |
| LDL | 50 | C1q | 30 | C1 est. inh. | 40 |
| APOA1 | 40 | Motif (14) | 30 | C4 | 40 |
| GLP-1 | 40 | GLP-1 | 30 | C5 | 40 |
| TNF-β | 40 | IL-2 | 30 | CD40 | 40 |
| | | | | ligand | |
| APOA4 | 30 | IL-3 | 30 | Motif (3) | 40 |
| Motif (7) | 30 | IL-5 | 30 | IL-16 | 40 |
| GM-CSF | 30 | IL-9 | 30 | IL-4 | 40 |
| IL-13 | 30 | Integrin α- | 30 | JAK3 | 40 |
| | | 10 | | | |
| IL-18 | 30 | OSBPL3 | 30 | Lewis^{y} | 40 |

Supplementary Table 1.
Antigens targeted on the antibody microarray^{a)}

| | | No of |
|---|---|---|
| | | antibody |
| Protein | Full name | clones |
| Angiomotin | Angiomotin | 2 |
| APOA1 | Apolipoprotein A1 | 3 |
| APOA4 | Apolipoprotein A4 | 3 |
| ATP5B | ATP synthase subunit beta, | 3 |
| | mitochondrial | |
| Beta- | Beta-galactosidase | 1 |
| galactosidase | | |
| BTK | Tyrosine-protein kinase BTK | 1 |
| C1 est. inh. | Plasma protease C1 inhibitor | 4 |
| C1q* | Complement C1q | 1 |
| C1s | Complement C1s | 1 |
| C3* | Complement C3 | 6 |
| C4* | Complement C4 | 4 |
| C5* | Complement C5 | 3 |
| CD40 | CD40 protein | 4 |
| CD40 ligand | CD40 ligand | 1 |
| CHX10 | Visual system homeobox 2 | 3 |
| CT | Cholera toxin subunit B (Control) | 1 |
| Cyst. C | Cystatin-C | 4 |
| Digoxin | Digoxin | 1 |
| DUSP9 | Dual specificity protein phosphatase 9 | 1 |
| Eotaxin | Eotaxin | 3 |
| Factor B* | Complement factor B | 4 |
| GLP-1 | Glucagon-like peptide-1 | 1 |
| GLP-1 R | Glucagon-like peptide 1 receptor | 1 |
| GM-CSF | Granulocyte-macrophage colony- | 3 |
| | stimulating factor | |
| HLA-DR/DP | HLA-DR/DP | 1 |
| ICAM-1 | Intercellular adhesion molecule 1 | 1 |
| IFN-gamma | Interferon gamma | 3 |
| IgM | Immunoglobulin M | 5 |
| IL-1 alpha* | lnterleukin-1 alpha | 3 |
| IL-1 beta | lnterleukin-1 beta | 3 |
| IL-10* | Interleukin-10 | 3 |
| IL-11 | Interleukin-11 | 3 |
| IL-12* | Interleukin-12 | 4 |
| IL-13* | Interleukin-13 | 3 |
| IL-16 | Interleukin-16 | 3 |
| IL-18 | Interleukin-18 | 3 |
| IL-1ra | Interleukin-1 receptor antagonist protein | 3 |
| IL-2 | Interleukin-2 | 3 |
| IL-3 | Interleukin-3 | 3 |
| IL-4* | Interleukin-4 | 4 |
| IL-5* | Interleukin-5 | 3 |
| IL-6* | Interleukin-6 | 4 |
| IL-7 | Interleukin-7 | 2 |
| IL-8* | Interleukin-8 | 3 |
| IL-9 | Interleukin-9 | 3 |
| Integrin alpha-10 | Integrin alpha-10 | 1 |
| Integrin alpha-11 | Integrin alpha-11 | 1 |
| JAK3 | Tyrosine-protein kinase JAK3 | 1 |
| LDL | Apolipoprotein B-100 | 2 |
| Leptin | Leptin | 1 |
| Lewisx | Lewis x | 2 |
| Lewisy | Lewis y | 1 |
| LUM | Lumican | 1 |
| MCP-1* | C-C motif chemokine 2 | 9 |
| MCP-3 | C-C motif chemokine 7 | 3 |
| MCP-4 | C-C motif chemokine 13 | 3 |
| Motif | Peptide motifs | 15 |
| MYOM2 | Myomesin-2 | 2 |
| ORP-3 | Oxysterol-binding protein-related | 2 |
| | protein 3 | |
| Procathepsin W | Procathepsin W | 1 |
| Properdin* | Properdin | 1 |
| PSA | Prostate-specific antigen | 1 |
| RANTES | C-C motif chemokine 5 | 3 |
| Sialle x | Sialyl Lewis x | 1 |
| Sox11a | Transcription factor SOX-11 | 1 |
| Surface Ag X | Surface Ag X | 1 |
| TBC1D9 | TBC1 domain family member 9 | 3 |
| VEGF* | Vascular endothelial growth factor | 4 |

^{a)}The specificity, affinity (normally in the nM range), and on-chip functionality of all of these phage display derived scFv antibodies were ensured by using i) stringent phage-display selection and screening protocols (using different sample formats, ranging from pure proteins and mixtures of pure proteins to crude samples) (16), ii) multiple clones (1 to 9) per protein, and iii) a molecular design, adapted for microarray applications (1-3) (Säll et al unpublished observations). In addition, the specificity of several selected antibodies (marked with an *) have been further validated using pure proteins, mixtures of pure proteins, as well as well-characterized, standardized serum samples (with known levels of the targeted analytes, spiked with known level of specific protein(s) and/or specific protein(s) depleted), and/or orthogonal methods, such as mass spectrometry (affinity pull-down experiments), ELISA, MesoScaleDiscovery assay, and cytometric bead assay, as well as using blocking experiments (4-12).

TABLE C
Amino acid sequences of the scFv antibodies used in the Examples

| Ab | Full protein Sequence (VH-linker-VL-tag) |
|---|---|
| IL-1a (1) | EVQLLESGGGLVQPGGSLRLSCAASGFTFSSYAMHWVRQAPGKGLEWVSGVSWNGSRTHYADSVKGRFTISRDNSKNTLYLQMNSLRAEDTAVYYCARSSGYYSWAFDIW |
| | GQGTLVTVSSGGGGSGGGGSGGGGSQSVLTQPPSASGTPGQRVTISCSGSSSNIGRNTVNWYQQLPGTAPKLLIYGNSNRPSXVPDRFSGSKSGTSASLAISGLRSEDEADYY |
| | CAAWDDSLNGWAFGGXTKLTVLGEQKLISXXXLSGSAA [SEQ ID NO: 6] |
| IL-1a (2) | EVQLLESGGGLVQPGGSLRLSCAASGFTFSSYSMNWVRQAPGKGLEWVALISYDGSQKYYADSMKGRFTISRDNSKNTLYLQMNSLRAEDTAVYYCAKGHTSGTKAYYFDS |
| | WGQGTLVTVSSGGGGSGGGGSGGGGSQSVLTQPPSASGTPGQRVTISCTGTSSNIGAGYSVHWYQQLPGTAPKLLIYGNSNRPSGVPDRFSGSKSGTSASLAISGLRSEDXA |
| | DYYCQSYDSSLSGWVFGGXTKLTVLGEQKLISEEDLSGSAA [SEQ ID NO: 7] |
| IL-2 (1) | EVQLLESGGGLVQPGGSLRLSCAASGFTFGDYAMSWVRQAPGKGLEWVSSISSRGSYIYFADSVKGRFTISRDNSKNTLYLQMNSLRAEDTAVYYCAKKKTGYYGLDAWGQG |
| | TLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCSS |
| | YAGSNNLVFGGXTKLTXLGEQKLISXXDLSGSAA [SEQ ID NO: 8] |
| IL-2 (2) | EVXXLESGGGLVQPGGSLRLSCAASGFTFGDYAMSWVRQAPGKGLEWVSSISSRGSYIYFADSVKGRFTISRDNSKNTLYLQMNSLRAEDTAVYYCAKKKTGYYGLDAWGQG |
| | TLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCSS |
| | YAGSNNLVFGGXXKLTVLGEQKLISEXXLSGSAA [SEQ ID NO: 9] |
| IL-2 (3) | EVQLLESGGGLVQPGGSLRLSCAASGFTFGDYAMSWVRQAPGKGLEWVSSISSRGSYIYFADSVKGRFTISRDNSKNTLYLQMNSLRAEDTAVYYCAKKKTGYYGLDAWGQG |
| | TLVTVSSGGGGSGGGGSGGGGSQSVLTQPPSASGTPGQRVTISCTGSSSXIGAGYDVHWYQQLPGTAPKLLIYGNSNRP [SEQ ID NO: 10] |
| IL-3 (1) | EVQLLESGGGLVQPGGSLRLSCAASGFTFGRYTMHWVRQAPGKGLEWVSSISSSSSYIYYADSVKGRFTISRDNSKNTLYLQMNSLRAEDTAVYYCARHFFESSGGYFDYWGQ |
| | GTLVTVSSGGGGSGGGGSGGGGSQSVLTQPPSASGTPGQRVTISCSGSSSNIGSNTVNWYQQLPGTAPKLLIYRNNQRPSGVPDRFSGSKSGTSASLAISGLRSEDXADYYCA |
| | AWDDSLNGWVFGGXXKLTVLGEQKLISXXXLSGXAA [SEQ ID NO: 11] |
| IL-3 (2) | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGGARYDYWGQGTL |
| | VTVSSGGGGSGGGGSGGGGSQSVLTQPPSASGTPGQRVTISCSGSSSNIGSNTVNWYQQLPGTAPKLLIYDNNKRPSGVPDRFSGSKSGTSASLAISGLRSEDEADYYCQSYD |
| | NILRGVVFGGGTKLTVLXEQKLISEXDLSGSAA [SEQ ID NO: 12] |
| IL-3 (3) | EVXXXESGGGLVQPGGSLRLSCAASGFTFSSYAMSWVRQAPGKGLEWVSAISGRGEYTYYAGSVKGRFTISRDNSKNTLYLQMNSLRAEDTAVYYCATGATRFGYWGQGTL |
| | VTVSSGGGGSGGGGSGGGGSQSVLTQPPSASGTPGQRVTISCTGSSSNIGAGYGVQWYQQLPGTAPKLLIYRNNQRPSGVPDRFSGSKSGTSXSLAISGLRSEDEADYYCQSY |
| | DSSLSYSVFGGXTKLTVLGEQKLISXXDLSGSAA [SEQ ID NO: 13] |
| IL-4 (1) | EVQLLESGGGLVQPGGSLRLSCAASGFTFSNAWMSWVRQAPGKGLEWVSSLHGGGDTFYTDSVKGRFTISRDNSKNTLYLQMNSLRAEDTAVYYCASLYGSGSYYYYYYGM |
| | DVWGQGTLVTVSSGGGGSGGGGSGGGGSQSVLTQPPSASGTPGQRVTISCSGNNSNTGNNAVNWYQQLPGTAPKLLIYDNNKRPSGVPDRFSGSKSGTSASLAISGLRSE |
| | DEADYYCCSYAGSYIWVFGGXTKLTVLGEQKLISXEXLSGSAA [SEQ ID NO: 14] |
| IL-4 (2) | EVQLLESGGGLVQPGGSLRLSCAASGFTFSDYGMHWVRQAPGKGLEWVSGISWNGGKTHYVDSVKGQFTISRDNSKNTLYLQMNSLRAEDTAVYYCARDRGYCSNGVCYT |
| | ILDYWGQGTLVTVSSGGGGSGGGGSGGGGSQSVLTQPPSASGTPGQRVTISCSGSSSNIGSNTINWYQQLPGTAPKLLIYGNSNRPSGVPDRFSGSKSGTSASLAISGLRSEDE |
| | ADYYCQSYDSSLSGWVFGGXTKLXVLXEQKLISXXDLSGSAA [SEQ ID NO: 15] |
| IL-5 (1) | EVQLLESGGGLVQPGGSLRLSCAASGFTFSSYAMSWVRQAPGKGLEWVSSISSRSNYIYYSDSVKGRFTISRDNSKNTLYLQMNSLRAEDTAVYYCARNFRFFDKWGQGTLVT |
| | VSSGGGGSGGGGSGGGGSQSVLTQPPSASGTPGQRVTISCSGGSSXIGANPVSWYQQLPGTAPKLLIYGNSNRP [SEQ ID NO: 16] |
| IL-5 (2) | EVQLLESGGGLVQPGGSLRLSCAASGFTFSSYAMSWVRQAPGKGLEWVSSISSRSNYIYYSDSVKGRFTISRDNSKNTLYLQMNSLRAEDTAVYYCARNFRFFDKWGQGTLVT |
| | VSSGGGGSGGGGSGGGGSQSVLTQPPSASGTPGQRVTISCSGGSSNIGANPVSWYQQLPGTAPKLLIYGNSNRPSGVPDRFSGSKSGTSASLAISGLRSEDEADYYCQSYDSS |
| | LSGSVFGGXTKLTVLXEQKLISEXXLSGSAA [SEQ ID NO: 17] |
| IL-5 (3) | EVQLLESGGGLVQPGGSLRLSCAASGFTFSSYAMSWVRQAPGKGLEWVSSISSRSNYIYYSDSVKGRFTISRDNSKNTLYLQMNSLRAEDTAVYYCARNFRFFDKWGQGTLVT |
| | VSSGGGGSGGGGSGGGGSQSVLTQPPSASGTPGQRVTISCSGGSSNIGANPVSWYQQLPGTAPKLLIYGNSNRPSGVPDRFSGSKSGTSASLAISGLRSEDEADYYCQSYDSS |
| | LSGSVFGGXTKLTVLGEQKLISXEDLSGSAA [SEQ ID NO: 18] |
| IL-6 (1) | EVQLLESGGGLVQPGGSLRLSCAASGFTFSSYGMHWVRQAPGKGLEWVSGINWNGGSTGYADSVKGRFTISRDNSKNTLYLQMNSLRAEDTAVYYCARNRGSSLYYGMDV |
| | WGQGTLVTVSSGGGGSGGGGSGGGGSQSVLTQPPSASGTPGQRVTISCAGSSSNIGSKSVHWYQQLPGTAPKLLIYRNNRRPSGVPDRFSGSXSGTSXSLAIXGLRSXDXAD |
| | YYCXXWDDRVNXXXFGGXTXLTVLXXQKLISXXXLSGSXXXPSSSXXLIXXGXXXXLX-XXLXFTGRXFXTX-LXXX [SEQ ID NO: 19] |
| IL-6 (2) | EVQLLESGGGLVQPGGSLRLSCAASGFTFSNYGMHWVRQAPGKGLEWVSSITSSGDGTYFADSVKGRFTISRDNSKNTLYLQMNSLRAEDTAVYYCARAGGIAAAYAFDIW |
| | GQGTLVTVSSGGGGSGGGGSGGGGSQSVLTQPPSASGTPGQRVTISCSGSSSNVGSNYVYWYQQLPGTAPKLLIYDNNKRPSGVPDRFSGSKSGTSASLAISGLRSEDEADYY |
| | CQSYDSSRWVFGGXTKLTVLGEQXLISEEXLSGSAA [SEQ ID NO: 20] |
| IL-7 (1) | EVQLLESGGGLVQPGGSLRLSCAASGFTFSSYGMHWVRQAPGKGLEWVSGITWNSGSIGYVDSVKGRFTISRDNSKNTLYLQMNSLRAEDTAVYYCARGPSVAARRIGRHW |
| | YNWFDPWGQGTLVTVSSGGGGSGGGGSGGGGSQSVLTQPPSASGTPGQRVTISCSGSSSNIGSNSVYWYQQLPGTAPKLLIYDNNKRPSGVPDRFSGSKSGTSASLAISGLR |
| | SEXXADYYCQSYDSSLSGSVFGGXXKLXVLGEQKLISEXXLSGSAA [SEQ ID NO: 21] |
| IL-7 (2) | EVQLLESGGGLVQPGGSLRLSCAASGFTFSDYNIHWVRQPPGKGLEWVSGVSWNGSRTHYADSVKGQFTISRDNSKNTLYLQMNSLRAEDTAVYYCARDPAMVRGVVLPN |
| | YYGLDVWGQGTLVTVSSGGGGSGGGGSGGGGSQSVLTQPPSASGTPGQRVTISCTGSSSNIGAGYDVHWYQQLPGTAPKLLIYGHSNRPSGVPDRFSGSKSGTSASLAISGL |
| | RSEXXADYYCQSYDSSLSYPVFGGXTKLTVLGEQ [SEQ ID NO: 22] |
| IL-8 (1) | EVQLLESGGGLVQPGGSLRLSCAASGFTFDDYGMSWVRQAPGKGLEWVSLISWDGGSTYYADSVKGRFTISRDNSKNTLYLQMNSLRAEDTAVYYCARDDLYGMDVWGQ |
| | GTLVTVSSGGGGSGGGGSGGGGSQSVLTQPPSASGTPGQRVTISCTGSSSNIGAGYDVHWYQQLPGTAPKLLIYDNNKRPSXVPDRFSGSKSGTSASLAISGLRSEDEADYYC |
| | AAWDDSLSGWVFGGXTKLTVLXEQKLISEXXLSGSAA [SEQ ID NO: 23] |
| IL-8 (2) | EVQLLESGGGLVQPGGSLRLSCAASGFTFSSYEMNWVRQAPGKGLEWVSSISSSSSYIFYADSMKGRFTISRDNSKNTLYLQMNSLRAEDTAVYYCARNESVDPLGGQYFQH |
| | WGQGTLVTVSSGGGGSGGGGSGGGGSQSVLTQPPSASGTPGQRVTISCTGSSSNIGAGYDVHWYQQLPGTAPKLLIYDNNKRPSGVPDRFSGSKSGTSASLAISGLRSEDEA |
| | DYYCSAWDDNLDGPVFGGXTKLTVLXEQKLISXXXLSGSAA [SEQ ID NO: 24] |
| IL-9 (1) | EVQLLESGGGLVQPGGSLRLSCAASGFTFSSYAMSWVRQAPGKGLEWVSSISSSSSYIYYADSVKGRFTISRDNSKNTLYLQMNSLRAEDTAVYYCTTFGHWGQGTLVTVSSG |
| | GGGSGGGGSGGGGSQSVLTQPPSASGTPGQRVTISCSGSGSNIGDNSVNWYQQLPGTAPKLLIYGNNNRPSGVPDRFSGSKSGTSASLAISGLRSEDEADYYCSSYTSSSVVF |
| | GGXTKLTVLXEQKLISEXDLSGSAA [SEQ ID NO: 25] |
| IL-9 (2) | EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCAKSPGGSPYYFDYWG |
| | QGTLVTVSSGGGGSGGGGSGGGGSQSVLTQPPSASGTPGQRVTISCSGSVSNIGSNVVSWYQQLPGTAPKLLIYDNNKRPS [SEQ ID NO: 26] |
| IL-9 (3) | EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCAKSPGGSPYYFDYWG |
| | QGTLVTVSSGGGGSGGGGSGGGGSQSVLTQPPSASGTPGQRVTISCSGSVSNIGSNVVSWYQQLPGTAPKLLIYDNNKRPSGVPDRFSGSKSGTSASLAISGLRSEDEADYYC |
| | QSYDSSLGGWVFGGXTKLTVLGEQKLISEXDLSGSAA [SEQ ID NO: 27] |
| IL-10 (1) | EVQLLESGGGLVQPGGSLRLSCAASGFTFRSYVMSWVRQAPGKGLEWVSAISGSGGSTYYADSVKGRFTISRDNSKNTLYLQMNSLRAEDTAVYYCARGKGRWAFDIWGQ |
| | GTLVTVSSGGGGSGGGGSGGGGSQSVLTQPPSASGTPGQRVTISCTGSSSNVGAGYDVHWYQQLPGTAPKLLIYRNNQRPSGVPDRFSGSKSGTSASLAISGLRSXDXADYY |
| | CAAWDDSLSAHVVFGGXTKLTVLGEQKLISXXDLSGSAA [SEQ ID NO: 28] |
| IL-10 (2) | EVQLLESGGGLVQPGGSLRLSCAASGFTFRSYVMSWVRQAPGKGLEWVSAISGSGGSTYYADSVKGRFTISRDNSKNTLYLQMNSLRAEDTAVYYCARGKGRWAFDIWGQ |
| | GTLVTVSSGGGGSGGGGSGGGGSQSVLTQPPSASGTPGQRVTISCTGSSSNVGAGYDVHWYQQLPGTAPKLLIYRNNQRPSGVPDRFSGSKSGTSASLAISGLRSEDEADYY |
| | CAAWDDSLSAHVVFGGXTKLTVLGEQKLISEXDLSGSAA [SEQ ID NO: 29] |
| IL-10 (3) | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKGRWAFDIWGQG |
| | TLVTVSSGGGGSGGGGSGGGGSQSVLTQPPSASGTPGQRVTISCTGSSSNIGAGYGVHWYQQLPGTAPKLLIYGNSNRPSGVPDRFSGSKSGTSASLAISGLRSEDXADYYCA |
| | AWDDSLSGLVFGGXTKLTVLXEQKLISEXXLSGSAA [SEQ ID NO: 30] |
| IL-11 (1) | EVQLLESGGGLVQPGGSLRLSCAASGFTFSNFGMHWVRQAPGKGLEWVAFIRYDGSNKYYADSVKGRFTISRDNSKNTLYLQMNSLRAEDTAVYYCARHYYYSETSGHPGG |
| | FDPWGQGTLVTVSSGGGGSGGGGSGGGGSQSVLTQPPSASGTPGQRVTISCSGSSSNIGSYPVNWYQQLPGTAPKLLIYGNSNRPSGVPDRFSGSKSGTSASLAISGLRSEDE |
| | ADYYCQXWGTGVFGGXTKLTVLGEQKLISXEXLSGSAA [SEQ ID NO: 31] |
| IL-11 (2) | EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARHYYDVSYRGQQDA |
| | FDIWGQGTLVTVSSGGGGSGGGGSGGGGSQSVLTQPPSASGTPGQRVTISCTGSSSNLGSPYDVHWYQQLPGTAPKLLIYRNDQRASGVPDRFSGSXSGTSASLAISGLRSE |
| | DEADYYCAAWDDSLNAWVFGGXTKLTVLGEQKLISEXXLSGSAA [SEQ ID NO: 32] |
| IL-11 (3) | EVQLLESGGGLVQPGGSLRLSCAASGFTFSDYYMSWIRQAPGKGLEWVAYISGISGYTNYADSVRGRFTISRDNSKNTLYLQMNSLRAEDTAVYYCAKSKDWVNGGEMDVW |
| | GQGTLVTVSSGGGGSGGGGSGGGGSQSVLTQPPSASGTPGQRVTISCTGSSSNIGAGYVVHWYQQLPGTAPKLLIYSNNQRPSGVPDRFSGSKSGTSASLAISGLRSEDEADY |
| | YCAAWDDSLRGWVFGGXTKLTVLGEQKLISEEDLSGSAA [SEQ ID NO: 33] |
| IL-12 (1) | EVQLLESGGGLVQPGGSLRLSCAASGFTFSSYSMNWVRQAPGKGLEWVSAIGTGGGTYYADSVKGRFTISRDNSKNTLYLQMNSLRAEDTAVYYCARAFRAFDIWGQGTLV |
| | TVSSGGGGSGGGGSGGGGSQSVLTQPPSASGTPGQRVTISCSGSRSNIGNNFVSWYQQLPGTAPKLLIYGNSNRPSGVPDRFSGSKSGTSASLAISGLRSEDEADYYCAAWD |
| | DSLSGPVFGGXTKLTVLGEQKLISEXDLSGSAA [SEQ ID NO: 34] |
| IL-12 (2) | EVQLLESGGGLVQPGGSLRLSCAASGFTFSDYYMSWVRQAPGKGLEWVSGVSWNGSRTHYADSVKGQFTISRDNSKNTLYLQMNSLRAEDTAVYYCARGSRSSPDAFDIW |
| | GQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADY |
| | YCAAWDDRVNGRVFGGGTKLTVLGEQKLISEXXLSGSAA [SEQ ID NO: 35] |
| IL-13 (1) | EVQLLESGGGLVQPGGSLRLSCAASGFTFSSYSMNWVRQAPGKGLEWVSSISSGSSYIYYADSVKGRFTISRDNSKNTLYLQMNSLRAEDTAVYYCARSQGWWTYYYGMDV |
| | WGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEAD |
| | YYCETWGQ [SEQ ID NO: 36] |
| IL-13 (2) | EVQLLESGGGLVQPGGSLRLSCAASGFTFSSYSMNWVRQAPGKGLEWVSSISSGSSYIYYADSVKGRFTISRDNSKNTLYLQMNSLRAEDTAVYYCARSQGWWTYYYGMDV |
| | WGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDXAD |
| | YYCETXDSNTQIFGGXTKLTVLGEQKLISEEXLSGSAXAHHHHHH-SXRXPIXXIVSXITIHXXSFXNVVTGKXXALPXXXALQHIPXXXAXXXX [SEQ ID NO: 37] |
| IL-13 (3) | EVQLLESGGGLVQPGGSLRLSCAASGFTFSSYSMNWVRQAPGKGLEWVSSISSGSSYIYYADSVKGRFTISRDNSKNTLYLQMNSLRAEDTAVYYCARSQGWWTYYYGMDV |
| | WGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEAD |
| | YYCETWDSNTQIFGGXTKLTVLGEQKLISEXDLSGSAA [SEQ ID NO: 38] |
| VEGF (1) | EVQLLESGGGLVQPGGSLRLSCAASGFTFSSNEMSWIRQAPGKGLEWVSSISGSGGFTYYADSVKGRYTISRDNSKNTLYLQMNSLRAEDTAVYYCARETTVRGNAFDIWGQ |
| | GTLVTVSSGGGGSGGGGSGGGGSQSVLTQPPSASGTPGQRVTISCTGGSSNIGAGYDVHWYQQLPGTAPKLLIYRNNQRPSGVPDRFSGSXSGTSASLAISGLRSEDEADYYC |
| | AAWDDSLSVPMFGGXTKLTVLGEQKLISEXDLSGSAA [SEQ ID NO: 39] |
| VEGF (2) | EVQLLESGGGLVQPGGSLRLSCAASGFTFSSYSMNWVRQAPGKGLEWVSGINWNGGSTGYADSVKGRFTISRDNSKNTLYLQMNSLRAEDTAVYYCASSVGGWYEGDNW |
| | FDPWGQGTLVTVSSGGGGSGGGGSGGGGSQSVLTQPPSASGTPGQRVTISCTGSSSNIGAGYDVHWYQQLPGTAPKLLIYGNSNRPSXVPDRFSGSXSGTSASLAISGLRSE |
| | XEADYYCQSYDGSLSGSVFGGXTKLTVLGEXKLISEXXLSGSAA [SEQ ID NO: 40] |
| TGF-β1 (1) | EVQLLESGGGLVQPGGSLRLSCAASGFTFSTYAMSWVRQAPGKGLEWVAVVSIDGGTTYYGDPVKGRFTISRDNSKNTLYLQMNSLRAEDTAVYYCTRGPTLTYYFDYWGQ |
| | GTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEADYYCQ |
| | SYDSSLSGWVFGGXTKLXVLGEQKLISEEDLSGSAA [SEQ ID NO: 41] |
| TGF-β1 (2) | EVQLLESGGGLVQPGGSLRLSCAASGFTFGDYAMSWFRQAPGKGLEWVSGVSWNGSRTHYADSVKGRFTISRDNSKNTLYLQMNSLRAEDTAVYYCARDGNRPLDYWGQ |
| | GTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYC |
| | AAWDDRLNGWVFGGGTKLXVLGEQKLISEXDLSGSAA [SEQ ID NO: 42] |
| TGF-β1 (3) | EVQLLESGGGLVQPGGSLRLSCAASGFTFSDYYIGWIRQAPGKGLEWVSGINWNGGSTGYADSVKGRFTISRDNSKNTLYLQMNSLRAEDTAVYYCARRSTPSSSWALPDFF |
| | DYWGQGTLVTVSSGGGGSGGGGSGGGGSQSVLTQPPSASGTPGQRVTISCTGSSSNIGANYDVHWYQQLPGTAPKLLIYSNNQRPSGVPDRFSGSKSGTSASLAISGLRSED |
| | XADYYCQSYDSSLSGWVFGGXTKLTVLGEQKLISXXXLSGSAA [SEQ ID NO: 43] |
| TNF-α (1) | EVQLLESGGGLVQPGGSLRLSCAASGFTFDDYGMSWVRQAPGKGLEWVAVISYDGSNKYYADSVKGRFTISRDNSKNTLYLQMNSLRAEDTAVYYCTRHLGSAMGYWGQG |
| | TLVTVSSGGGGSGGGGSGGGGSQSVLTQPPSASGTPGQRVTISCTGSSSNIGAGYDVHWYQQLPGTAPKLLIYGNSNRPSXVPDRFSGSXSGTSASLAISGLRSEDEADYYCQ |
| | SYDSSLSGWVFGGXTKLTVLXEQKLISXXDLSGSAA [SEQ ID NO: 44] |
| TNF-α (2) | EVQLLESGGGLVQPGGSLRLSCAASGFTFSSYGMHWVRQAPGKGLEWVSAISGSGGSTYYADSVKGRFTISRDNSKNTLYLQMNSLRAEDTAVYYCARGGWGPRSAFDIWG |
| | QGTLVTVSSGGGGSGGGGSGGGGSQSVLTQPPSASGTPGQRVTISCSGSSSNIGSNTVTWYQQLPGTAPKLLIYGNTNRLSGVPDRFSGSKSGTSASLAISGLRSEDEADYYCE |
| | AWDDKLFGPVFGGXTXLTVLXEQKLISEXXLSGSAA [SEQ ID NO: 45] |
| TNF-α (3) | EVQLLESGGGLVQPGGSLRLSCAASGFTFSSYWMSWVRQAPGKGLEWVSGVNWNGSRTHYADSVKGRFTISRDNSKNTLYLQMNSLRAEDTAVYYCASIRANYYYGMDV |
| | WGQGTLVTVSSGGGGSGGGGSGGGGSQSVLTQPPSASGTPGQRVTISCSGGSSNIGSHPVNWYQQLPGTAPKLLIYGNSNRPSGVPDRFSGSKSGTSASLAISGLRSEDEAD |
| | YYCAAWDASLSGWVFGGGXKLTVLXEXKLISXXXLSGSAA [SEQ ID NO: 46] |
| GM-CSF (1) | EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARVGGMSAPVDYWG |
| | QGTLVTVSSGGGGSGGGGSGGGGSQSVLTQPPSASGTPGQRVTISCSGSSSNIGSNTVNWYQQLPGTAPKLLIYDNNKRPSGVPDRXSGSKSGTSASLAISGLRSEDEADYYC |
| | AAWDDSLIGLVVFGGXTKLTVLGEQKLISEXXLSGSAA [SEQ ID NO: 47] |
| GM-CSF (2) | EVQLLESGGGLVQPGGSLRLSCAASGFTFSSYAMSWVRQAPGKGLEWVAVISYDGSNEDSADSVKGRFTISRDNSKNTLYLQMNSLRAEDTAVYYCARGPSLRGVSDYWGQ |
| | GTLVTVSSGGGGSGGGGSGGGGSQSVLTQPPSASGTPGQRVTISCTGSSSNIGAGYDVHWYQQLPGTAPKLLIYNDNQRPSXVPDRFSGSKSGTSASLAISGLRSEDEADYYC |
| | QTWGTGINVIFGGXTKLXVLGEQKLISXEDLSGSAA [SEQ ID NO: 48] |
| GM-CSF (3) | EVQLLESGGGLVQPGGSLRLSCAASGFTFSSYAMSWVRQAPGKGLEWVAVISYDGSNEDSADSVKGRFTISRDNSKNTLYLQMNSLRAEDTAVYYCARGPSLRGVSDYWGQ |
| | GTLVTVSSGGGGSGGGGSGGGGSQSVLTQPPSASGTPGQRVTISCTGSSSNIGAGYDVHWYQQLPGTAPKLLIYNDNQRPSGVPDRFSGSKSGTSASLAISGLRSEDXADYYC |
| | QTWGTGINVIFGGXTKLTVLGEXKLISEXXLSGSAA [SEQ ID NO: 49] |
| TNF-β (1) | EVQLLESGGGLVQPGGSLRLSCAASGFTFSSFAMHWVRQAPGKGLEWVSAISGSGGSTYYADSVKGRFTISRDNSKNTLYLQMNSLRAEDTAVYYCASRSTLYYYYGMDVW |
| | GQGTLVTVSSGGGGSGGGGSGGGGSQSVLTQPPSASGTPGQRVTISCSGSTSNIGNSHVYWYQQLPGTAPKLLIYGNSNRPSXVPDRFSGSXSGTSASLAISGLRSEDEADYY |
| | CSSXAGSNNLVFGGXTKLTVLGEQKLISXXDLSGSAA [SEQ ID NO: 50] |
| TNF-β (2) | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARSPYYGMDVWGQGT |
| | LVTVSSGGGGSGGGGSGGGGSQSVLTQPPSASGTPGQRVTISCTGSSSNIGAGYDVHWYQQLPGTAPKLLIYRNDQRPSGVPDRFSGSKSGTSASLAISGLRSEDEADYYCSS |
| | YGGRDNVVFGGXTKLTVLXEQKLISXXXLSGSAA [SEQ ID NO: 51] |
| IL-1ra (1) | EVQLLESGGGLVQPGGSLRLSCAASGFTFDTHWMSWVRQAPGKGLEWVSAISGSGGSTYYADSVKGRFTISRDNSKNTLYLQMNSLRAEDTAVYYCARHDYGDYRAFDIW |
| | GQGTLVTVSSGGGGSGGGGSGGGGSQSVLTQPPSASGTPGQRVTISCTGSSSNIGAGYVVHWYQQLPGTAPKLLIYGNNNRPSGVPDRFSGSKSGTSASLAISGLRSEDEAD |
| | YYCQSYDSSLSGVVFGGXTKLXVLXEQKLISXEDLSGSAA [SEQ ID NO: 52] |
| IL-1ra (2) | EVQLLESGGGLVQPGGSLRLSCAASGFTFSKYAMTWVRQAPGKGLEWVSAISGSGGNTYYADSVKGRFTISRDNSKNTLYLQMNSLRAEDTAVYYCARLVRGLYYGMDVW |
| | GQGTLVTVSSGGGGSGGGGSGGGGSQSVLTQPPSASGTPGQRVTISCSGSSSNIGNNAVNWYQQLPGTAPKLLIYGNSNRPSGVPDRFSGSKSGTSASLAISGLRSXDEADYY |
| | CQTXGTGPVVFGGXTKLTVLGEQKLISXXXXSGSAA [SEQ ID NO: 53] |
| IL-1ra (3) | EVQLLESGGGLVQPGGSLRLSCAVSGFTFSSYSMNWVRQAPGKGLEWVAGIGGRGATTYYVDSVKGRFTISRDNSKNTLYLQMNSLRAEDTAVYYCARLRVVPAARFDYWG |
| | QGTLVTVSSGGGGSGGGGSGGGGSQSVLTQPPSASGTPGQRVTISCSGSSSNIGSNTVNWYQQLPGTAPKLLIYGNSNRPSGVPDRFSGSKSGTSASLAISGLRSEDEADYYC |
| | QSYDSSLSGPPWVFGGXXKLXVLXEQKLISEEDLSGSAA [SEQ ID NO: 54] |
| IL-16 (1) | EVQLLESGGGLVQPGGSLRLSCAASGFTFSNHAMSWVRQAPGKGLEWVSGVSWNGSRTHYADSVKGRFTISRDNSKNTLYLQMNSLRAEDTAVYYCARAALVQGVKHAFE |
| | IWGQGTLVTVSSGGGGSGGGGSGGGGSQSVLTQPPSASGTPGQRVTISCSGSSSNIGSNTVNWYQQLPGTALKLLIYRNNQRPSGVPDRFSGSKSGTSASLAISGLRSEDEAD |
| | YYCASWDDRLSGLVFGGXTKLTVLGEQKLISEXDLSGSAA [SEQ ID NO: 55] |
| IL-16 (2) | EVQLLESGGGLVQPGGSLRLSCAASGFTFSNHAMSWVRQAPGKGLEWVSGVSWNGSRTHYADSVKGRFTISRDNSKNTLYLQMNSLRAEDTAVYYCARAALVQGVKHAFE |
| | IWGQGTLVTVSSGGGGSGGGGSGGGGSQSVLTQPPSASGTPGQRVTISCSGSSSNIGSNTVNWYQQLPGTALKLLIYRNNQRPSGVPDRFSGSXSGTSASLAISGLRSEXEAD |
| | YYCASWDDRLSGLVFGGXTKLTVLXEQKLISEEDLSGSAA [SEQ ID NO: 56] |
| IL-18 (1) | EVQLLESGGGLVQPGGSLRLSCAASGFTFSSYGMHWVRQAPGKGLEWVSGINWNGGSTGYADSVKGRFTISRDNSKNTLYLQMNSLRAEDTAVYYCARDLRGGRFDPWG |
| | QGTLVTVSSGGGGSGGGGSGGGGSQSVLTQPPSASGTPGQRVTISCTGSSSNIGAGYVVHWYQQLPGTAPKLLIYRNNQRPSGVPDRFSGSXSGTSASLAISGLRSEDEADYY |
| | CSSXAGSKNLIFGGXTKLTVLGEQKLISXXXLSGSAA [SEQ ID NO: 57] |
| IL-18 (2) | EVQLLESGRGLVQPGGSLRLSCAASGFTFSSYGMHWVRQAPGKGLEWVSAIGTGGDTYYADSVMGRFTISRDNSKNTLYLQMNSLRAEDTAVYYCARSPRRGATAGTFDY |
| | WGQGTLVTVSSGGGGSGGGGSGGGGSQSVLTQPPSASGTPGQRVTISCSGSSSNIGSNIVNWYQQLPGTAPKLLIYRNNQRPSGVPDRFSGSKSGTSASLAISGLRSEDEADY |
| | YCXSYDNSLSGWVFGGXXKLXVLGEXKLISEXDLSGSAA [SEQ ID NO: 58] |
| MCP-4 (1) | EVQLLESGGGLVQPGGSLRLSCAASGFTFSSYGMHWVRQAPGKGLEWVSGISWNGGKTHYVDSVKGQFTISRDNSKNTLYLQMNSLRAEDTAVYYCARGGYSSGWAFDY |
| | WGQGTLVTVSSGGGGSGGGGSGGGGSQSVLTQPPSASGTPGQRVTISCSGRSSNIESNTVNWYQQLPGTAPKLLIYGNSNRPSGVPDRFSGSKSGTSASLAISGLRSEDEAD |
| | YYCAAWDDRLNAVVFGGXTKLTVLGEQKLISEXDLSGSAA [SEQ ID NO: 59] |
| IFN-γ (1) | EVQLLESGGGLVQPGGSLRLSCAASGFTFSSYGMHWVRQAPGKGLEWVSGVSWNGSRTHYADSVKGRFTISRDNSKNTLYLQMNSLRAEDTAVYYCARGRTGHGWKYYF |
| | DLWGQGTLVTVSSGGGGSGGGGSGGGGSQSVLTQPPSASGTPGQRVTISCSGSSSNIGNNAVNWYQQLPGTAPKLLIYGNSNRPSXVPDRFSGSXSGTSASLAISGLRSEDE |
| | ADYYCQXWGTGLGVFGGXTKLTVLGEXKLISEEXLSGSAA [SEQ ID NO: 60] |
| IFN-γ (2) | EVQLLESGGGLVQPGGSLRLSCAASGFTFSRHGFHWVRQGPGKGLEWVSGVSWNGSRTHYADSVKGRFTISRDNSKNTLYLQMNSLRAEDTAVYYCARGNWYRAFDIWG |
| | QGTLVTVSSGGGGSGGGGSGGGGSQSVLTQPPSASGTPGQRVTISCSGGSSHIGRNFISWYQQLPGTAPKLLIYAGNSRP [SEQ ID NO: 61] |
| IL-1β (1) | EVQLLESGGGLVQPGGSLRLSCAASGFTFSSYAMSWVRQAPGKGLEWVSYISSSGSTIYYADSVKGRSTISRDNSKNTLYLQMNSLRAEDTAVYYCARVRQNSGSYAYWGQG |
| | TLVTVSSGGGGSGGGGSGGGGSQSVLTQPPSASGTPGQRVTISCTGTSSNIGAPYDVHWYQQLPGTAPKLLIYGNSNRPSGVPDRFSGSKSGTSASLAISGLRSEDXADYYCQ |
| | SYDSSLSAVVFGGXTKLTVLGEQKLISEXXLSGSAA [SEQ ID NO: 62] |
| IL-1β (2) | EVQLLESGGGLVQPGGSLRLSCAASGFTFSRYVMTWVRQAPGKGLEWVSLISGGGSATYYADSMKGRFTISRDNSKNTLYLQMNSLRAEDTAVYYCAKRVPYDSSGYYPDAF |
| | DIWGQGTLVTVSSGGGGSGGGGSGGGGSQSVLTQPPSASGTPGQRVTISCTGSSSNIGAGYDVHWYQQLPGTAPKLLIYGNSNRPSGVPDQFSGSKSGTSASLAISGLRSED |
| | EADYYCAAWDDSLNGPVFGGXTXLTXLXEQKLISEEXLSGSAA [SEQ ID NO: 63] |
| IL-1β (3) | EVQLLESGGGLVQPGGSLRLSCAASGFTFSSYWMSWVRQAPGKGLEWVAVVSYDGNNKYYADSRKGRFTISRDNSKNTLYLQMNSLRAEDTAMYYCASYWYTSGWYPYG |
| | MDVWGQGTLGTVSSGGGGSGGGGSGGGGSQSVLTQPPSASGTPGQRVTISCTGSSSNIGAGYDLHWYQQLPGTAPKLLIYRNNQRPSGVPDRFSGSKSGTSASLAISGLRS |
| | EDEADYYCSSYVDNNNLVFGGXTKLTVLXEQKLISEXXLSGSAA [SEQ ID NO: 64] |
| Eotaxin (1) | EVQLLESGGGLVQPGGSLRLSCAASGFTFSSYWMSWVRQAPGKGLEWVSGVSWNGSRTHYADSVKGRFTISRDNSKNTLYLQMNSLRAEDTAVYYCVKGKGTIAMPGRAR |
| | VGWWGQGTLVTVSSGGGGSGGGGSGGGGSQSVLTQPPSASGTPGQRVTISCSGSSSNIGNNAVNWYQQLPGTAPKLLIYANSNRPSGVPDRFSGSXSGTSASLAISGLRSE |
| | DEADYYCAAWDDSLSGPVFGGXTKLTVLGEQKLISXXDLSXSAA [SEQ ID NO: 65] |
| Eotaxin (2) | EVQLLESGGGLVQPGGSLRLSCAASGFTFSAYWMTWVRQAPGKGLEWVSVIYSGGSTYYADSVKGRFTISRDNSKNTLYLQMNSLRAEDTAVYYCARQTQQEYFDYWGQG |
| | TLVTVSSGGGGSGGGGSGGGGSQSVLTQPPSASGTPGQRVTISCFGSNSNIGSSTVNWYQQLPGTAPKLLIYDNDKRPSGVPDRFSGSXSGTSASLAISGLRSEDEADYYCAA |
| | WDDSLNGPVFGGXTKLTVLGEQKLISXXXLSGSXAAHHHHHH-SPRXPIRPIVSXXTIHWPSFYNVXTGKXXXLPNXIXXXHIPLSPAXXIXXXPXXXXX [SEQ ID NO: 66] |
| Eotaxin (3) | EVQLLESGGGLVQPGGSLRLSCAASGFTFRGYAMSWVRQAPGKGLEWVSGVSWNGSRTHYADSVKGRFTISRDNSKNTLYLQMNSLRAEDTAVYYCARAPAVAGWFDPW |
| | GQGTLVTVSSGGGGSGGGGSGGGGSQSVLTQPPSASGTPGQRVTISCSGSSSNIGSHTVNWYQQLPGTAPKLLIYRNNQRPSGVPDRFSGSKSGTSASLAISGLRSEDXADYY |
| | CAAWDDSLSGRVXGGGXKLTVLGEQKLISEEDLSGSAA [SEQ ID NO: 67] |
| RANTES (1) | EVQLLESGGGLVQPGGSLRLSCAASGFTFSSYGMHWVRQAPGKGLEWVAVISNDGTKKDYADSVKGRFTISRDNSKNTLYLQMNSLRAEDTAVYYCARDASGYDDYYFDY |
| | WGQGTLVTVSSGGGGSGGGGSGGGGSQSVLTQPPSASGTPGQRVTISCTGSSSNIGAGSDVHWYQQLPGTAPKLLIYRDDQRSSGVPDRFSGSKSGTSAFLAISGLRSEDEA |
| | DYYCQSYDNSLSGWVFGGXTKLTVLGEQKLISEXXLSGSAA [SEQ ID NO: 68] |
| RANTES (2) | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDNDYSSDTFDYWG |
| | QGTLVTVSSGGGGSGGGGSGGGGSQSVLTQPPSAFGTPGQRVTISCSGSSSNIGSDYVYWYQQLPGTAPKLLIYSDNQRP [SEQ ID NO: 69] |
| RANTES (3) | EVQLLESGGGLVQPGGSLRLSCAASGFTFSNYGMNWVRQAPGKGLEWVSGVSWNGSRTHYVDSVKRRFTISRDNSKNTLYLQMNSLRAEDTAVYYCARPRLRSHNYYGM |
| | DVWGQGTLVTVSSGGGGSGGGGSGGGGSQSVLTQPPSASGTPGQRVTISCSGSSFKSGKNYVSWYQQLPGTAPKLLIYRNNQRPSGVPDRFSGSKSGTSASLAISGLRSEDE |
| | ADYYCAAWDVRVKGVIFGGXTKLTVLGEQKLISEXDLSGSAA [SEQ ID NO: 70] |
| MCP-1 (1) | EVQLLESGGGLVQPGGSLRLSCAASGFTFSSYAMSWVRQAPGKGLEWVSGVSWNGSRTHYADSVKGRFTISRDNSKNTLYLQMNSLRAEDTAVYYCARGGHQQLGQWG |
| | QGTLVTVSSGGGGSGGGGSGGGGSQSVLTQPPSASGTPGQRVTISCSGSSSNIGNNYVSWYQQLPGTAPKLLIYRDSRRPSGVPDRFSGSKSGTSASLAISGLRSEXEADYYCA |
| | AWDDSLKGWLFGGXTKLTVLXEQKLISEXXLSGSAA [SEQ ID NO: 71] |
| MCP-1 (2) | EVQLLESGGGLVQPGGSLRLSCAASGFTFSSYAMSWVRQAPGKGLEWVSYISSSSSYTNYADSVKGRFTISRDNSKNTLYLQMNSLRAEDTAVYYCARFRYNSGKMFDYWGQ |
| | GTLVTVSSGGGGSGGGGSGGGGSQSVLTQPPSASGTPGQRVTISCSGSSSNIGRNTVNWYQQLPGTAPKLLIYGNSNRRSGVPDRFSGSKSGTSASLAISGLRSEDEADYYCA |
| | AWDDSLSGVVFGGXTKLTVLXEQKLISEXDLSGSAA [SEQ ID NO: 72] |
| MCP-1 (3) | EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCAKSHYYDTTSFDYWG |
| | QGTLVTVSSGGGGSGGGGSGGGGSQSVLTQPPSASGTPGQRVTISCSGSSSNIGTNPVNWYQQLPGTAPKLLIYDNNKRPSGVPDRFSGSKSGTSASLAISGLRSEDXADYYC |
| | AAWDDSLSGVVFGGXTKLTVLGEQKLISXEDLSGSAA [SEQ ID NO: 73] |
| MCP-3 (1) | EVQLLESGGGLVQPGGSLRLSCAASGFTFSTYGMHWVRQAPGKGLEWVSGVSWNGSRTHYVNSVKRRFTISRDNSKNTLYLQMNSLRAEDTAVYYCARVAPGSGKRLRAF |
| | DIWGQGTLVTVSSGGGGSGGGGSGGGGSQSVLTQPPSASGTPGQRVTISCSGSSSNIGNNAVNWYQQLPGTAPKLLIYEVSKRPPGVPDRFSGSKSGTSASLAISGLRSEDXA |
| | DYYCSSYAGSSKWVFGGXTKLTVLGEQKLISEEDLSGSAA [SEQ ID NO: 74] |
| MCP-3 (2) | EVQLLESGGGLVQPGGSLRLSCAASGFTLSSNYMSWVRQAPGKGLEWVSGISASGHSTHYADSGKARFTISRDNSKNTLYLQMNSLRAEDTAVYYCARGKSLAYWGQGTLV |
| | TVSSGGGGSGGGGSGGGGSQSVLTQPPSASGTPGQRVTISCSGSSSNIGNNAVNWYQQLPGTAPKLLIYRNNQRPSGVPDRFSGSKSGTSASLAISGLRSEDEADYYCAAWD |
| | DSLSVVVFGGXTKLTVLGEQKLISXXXLSGSAA [SEQ ID NO: 75] |
| MCP-3 (3) | EVQLLESGGGLVQPGGSLRLSCAASGFTFSIYWMSWVRQAPGKGLEWVAYIGGISNTVSYSDSVKGRFTISRDNSKNTLYLQMNSLRAEDTAVYYCAKAPGYSSGWGWFDP |
| | WGQGTLVTVSSGGGGSGGGGSGGGGSQSVLTQPPSASGTPGQRVTISCSGSSSNIGTNSVFWYQQLPGTAPKLLIYGNNNRPSGVPDRFSGSKSGTSASLAISGLRSEDXAD |
| | YYCMIWHSSASVFGXXTKLTVLGEQKLISEXXLSGSAA [SEQ ID NO: 76] |
| β-gal | EVQLLESGGGLVQPGGSLRLSCAASGFTFSSYAMHWVRQAPGKGLEWVAVIAYDGINEYYGDSVKGRFTISRDNSKNTLYLQMNSLRAEDTAVYYCARGGIYHGFDIWGQG |
| | TLVTVSSGGGGSGGGGSGGGGSQSVLTQPPSASGTPGQRVTISCSGSSSNIGSNYVYWYQQLPGTAPKLLIYDNHKRPSGVPDRFSGSXSGTSASLAISGLRSEDEADYYCAA |
| | WDDNSWVFGGXTKLTVLGXYKDDDDKAA [SEQ ID NO: 77] |
| Angiomotin (1) | EVQLLESGGGLVQPGGSLRLSCAASGFTFSDHYMDWVRQAPGKGLEWVSGVSWNGSRTHYADSVKGRFTISRDNSKNTLYLQMNSLRAEDTAVYYCARDTWAYGAFDIW |
| | GQGTLVTVSSGGGGSGGGGSGGGGSQSVLTQPPSASGTPGQRVTISCSGSNSNIGRNTVNWYQQLPGTAPKLLIYRDNQRPSGVPDRFSGSXSGTPASLAISGLRSEDXADY |
| | YCAAWDVSLNGWVFGGXTKLTVLGDYXDHDGDYKDHDIDXXDDDDXXAAHHHHHH-SPRWXIRPIVSRITIXWXXFYXVXXXKXX [SEQ ID NO: 78] |
| Angiomotin (2) | EVQLLESGGGLVQPGGSLRLSCAASGFTFNDYYMTWIRQAPGKGLEWVSYISSSGSTIYYADSVKGRFTISRDNSKNTLYLQMNSLRAEDTAVYYCARERLPDVFDVWGQGTL |
| | VTVSSGGGGSGGGGSGGGGSQSVLTQPPSASGTPGQRVTISCSGSGSNIGTNSVSWYQQLPGTAPKLLIYFDDLLPSGVPDRFSGSKSGTSASLAISGLRSEDEADYYCAAWD |
| | DSLSGVVFGGXTKLTVLGXYKDHDGDYKDHDIDYKDDDXKAXAHHHHHH-SPRXXXRXIVSXIXIHXXXFYNXXTGKTXXXXXXIXXAAXXXFXX [SEQ ID NO: 79] |
| Leptin | EVQLLESGGGLVQPGGSLRLSCAASGFTFGDFAMSWVRQAPGKGLEWVANIKQDGSVKYYVDSVKGRFTISRDNSKNTLYLQMNSLRAEDTAVYYCARFLAGFYYGMDVW |
| | GQGTLVTVSSGGGGSGGGGSGGGGSQSVLTQPPSASGTPGQRVTISCSGSDSNIGGNTVNWYQQLPGMAPKLLIYYDDLLPSGVPDRFSGSKSGTSASLAISGLRSEDEADY |
| | YCAAYDDTMNGWGFGGXTKLTVLGXYKDXDDKAA [SEQ ID NO: 80] |
| Integrin α-10 | EVQLLESGGGLVQPGGSLRLSCAASGFTFSTYNMNWVRQAPGKGLEWVSTISGSGGRTYYADSVKGRFTISRDNSKNTLYLQMNSLRAEDTAVYYCARDRVATLDAFDIWG |
| | QGTLVTVSSGGGGSGGGGSGGGGSQSVLTQPPSASGTPGQRVTISCSGSSSNIGSNSVSWYQQLPGTAPKLLIYSNNQRPSGVPDRFSGSKSGTSASLAISGLRSEDEADYYCA |
| | AWDDSLSGVVFGGXTKLTVLGEQKLISEXDLSGSAA [SEQ ID NO: 81] |
| Integrin α-11 | EVQLLESGGGLVQPGGSLRLSCAASGFTFRRDWMSWVRQVPGKGLEWVSVISGSDGSTYYADSVKGRFTISRDNSKNTLYLQMNSLRAEDTAVYYCASYSPLGNWFDSWG |
| | QGTLVTVSSGGGGSGGGGSGGGGSQSVLTQPPSASGTPGQRVTISCTGSSSNIGAGYDVHWYQQLPGTAPKLLIYSDTYRPSGVPDRFSGSXSGTSASLAISGLRSEDEADYYC |
| | QSYDSSLXGFVVFGGXTKLTVLXEQKLISEXXLSGSAA [SEQ ID NO: 82] |
| IgM (1) | EVQLLESGGGLVQPGGSLRLSCAASGFTFSDYYMSWIRQAPGKGLEWVSAIGSGPYYAHSVRDRFTISRDNSKNTLYLQMNSLRAEDTAVYYCARGGVEASFDYWGQGTLVT |
| | VSSGGGGSGGGGSGGGGSQSVLTQPPSASGTPGQRVTISCTGSSSNIGAGYDVHWYQQLPGTAPKLLIYGNTNRPSGVPNRFSGSKSGTSASLAISGLRSEDEADYYCQSYDN |
| | DLSGWVFGGXTKLXVLGEQKLISXXXLSGSAA [SEQ ID NO: 83] |
| LDL (1) | EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCARAARYSYYYYGMD |
| | VWGQGTLVTVSSGGGGSGGGGSGGGGSQSVLTQPPSASGTPGQRVTISCSGSSSNIGNNAVNWYQQLPGTAPKLLIYGNDRRPSGVPDRFSGSKSGTSASLAISGLRSEDEA |
| | DYYCQTWGTGRGVFGGGTKLTVLGEQKLISEXXLSGSAA [SEQ ID NO: 84] |
| LDL (2) | EVQLLESGGGLVQPGGSLRLSCAASGFTFSNAWMSWVRQAPGKGLEWVSSISTSSNYIYYADSVKGRFTISRDNSKNTLYLQMNSLRAEDTAVYYCARVKKYSSGWYSNYAF |
| | DIWGQGTLVTVSSGGGGSGGGGSGGGGSQSVLTQPPSASGTPGQRVTISCSGSSSSIGNNFVSWYQQLPGTAPKLLIYDNNKRPSXVPDRFSGSXSGTSASLAISGLRSEDXA |
| | DYYCAAWDDSLNGWVFGGXTKLTVLXXYKDHDGDYXDHDIDYKDXXDKAA [SEQ ID NO: 85] |
| PSA | EVQLLESGGGLVQPGGSLRLSCAASGFTFRSYEMNWVRQAPGKGLEWVAVIGGNGVDTDYADSVKGRFTISRDNSKNTLYLQMNSLRAEDTAVYYCVREEVDFWSGYYSY |
| | GMDVWGQGTLVTVSSGGGGSGGGGSGGGGSQSVLTQPPSASGTPGQRVTISCSGSSSNIGDNFVSWYQQLPGTAPKLLIYRTNGRPSGVPDRFSGSXSGTSASLAISGLRSE |
| | DEADYYCATWDDNLNGRVVFGGXTKLTVLGDYKDXXDKAA [SEQ ID NO: 86] |
| Lewis^{x} (1) | EVQLLESGGGLVQPGGSLRLSCAASGFTFSNYWMHWVRQAPGKGLEWVANIKEDGSEKYYVDSVKGRFTISRDNSKNTLYLQMNSLRAEDTAVYYCAREGETSFGLDVWG |
| | QGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEADYYC |
| | ASWDDSLSGWVFGGXTKLTVLGDYKDDDDKAA [SEQ ID NO: 87] |
| Lewis^{x} (2) | EVQLLESGGGLVQPGGSLRLSCAASGFTFSRYWMHWVRQAPGKGLEWVANIKPDGSEQYYADSVKGRFTISRDNSKNTLYLQMNSLRAEDTAVYYCAREGLSSGWSYGMD |
| | VWGQGTLVTVSSGGGGSGGGGSGGGGSQSVLTQPPSASGTPGQRVTISCSGSNSNIGSNTVNWYQQLPGTAPKLLIYTNINRPSGVPDRFSGSKSGTSASLAISGLRSXDEA |
| | DYYCATWDDSLSGWVFGGXTKLTVLGXYKDXXDKAA [SEQ ID NO: 88] |
| Lewis^{y} | EVQLLESGGGLVQSGGSLRLSCAASGFTFSSYTLHWVRQAPGKGLEYVSAISSNGGSTYYADSVKGRFTISRDNSKNTLYLQMNSLRAEDTAVYYCASDVYGDYPRGLDYWGQ |
| | GTLVTVSSGGGGSGGGGSGGGGSQSVLTQPPSASGTPGQRVTISCSGTTSNIGSNYVHWYQQLPGTAPKLLIYGNNNRPSGVPDRFSGSKSGTSASLAISGLRSEDXADYYCQ |
| | SYDRSLGGLRVFGGXTKLTVLXDYKXDDDKAA [SEQ ID NO: 89] |
| Sialle x | EVQLLESGGGLVQPGGSLRLSCAASGFTLSSYAMSWVRQAPGKGLEWVSSISSGNSYIYYADSVKGRFTISRDNSKNTLYLQMNSLRAEDTAVYYCARGRGRGGGFELWGQG |
| | TLVTVSSGGGGSGGGGSGGGGSQSVLTQPPSASGTPGQRVTISCSGSSSNIGTYTVNWYQQLPGTAPKLLIYSNNQRPSGVPDRFSGSKSGTSASLAISGLRSEXEADYYCSSN |
| | AGIDNILFGGXTKLTVLGEQKLISEXDLSGSXAAHHHHXXXXXXXXIXXXXXXXXXXXXXXXXXXLXX [SEQ ID NO: 90] |
| TM peptide | EVQLLESGGGLVQPGGSLRLSCAASGFTFSSYGFHWVRQAPGKGLEWVSLISWDGGSTYYADSVKGRFTISRDNSKNTLYLQMNSLRAEDTAVYYCARGTWFDPWGQGTLV |
| | TVSSGGGGSGGGGSGGGGSQSVLTQPPSASGTPGQRVTISCSGSSSXIGNNAVNWYQQLPGTAPKLLIYRNNQRPSXVPDRFSGXXSGTSASLAIXGLRSEDEADYYCAAWD |
| | DSLSWVFGGXTKLTVLGDXXTMXVIIKIMTSXXXMTMXRRP [SEQ ID NO: 91] |
| Procathepsin W | EVQLLESGGGLVQPGGSLRLSCAASGFTFSSYAMSWVRQAPGKGLEWVSSMSASGGSTYYADSVKGRFTISRDNSKNTLYLQMNSLRAEDTAVYYCARDRGSYGMDVWG |
| | QGTLVTVSSGGGGSGGGGSGGGGSQSVLTQPPSASGTPGQRVTISCSGSTSNIGSYAVNWYQQLPGTAPKLLIYGNNNRPSGVPDRFSGSXSGTSASLAISGPRSEDEADYYC |
| | AAWDDSLNGGVFGGXTKLTVLGXYKXDDDKAA [SEQ ID NO: 92] |
| BTK (1) | EVQLLESGGGLVQPGGSLRLSCAASGFTFSNYAMSWVRQAPGKGLEWVSGINWNGGSTGYADSVKGRFTISRDNSKNTLYLQMNSLRAEDTAVYYCAKHLKRYSGSSYLFD |
| | YWGQGTLVTVSSGGGGSGGGGSGGGGSQSVLTQPPSASGTPGQRVTISCSGSSSXIGSNYVYWYQQLPGTAPKLLIY [SEQ ID NO: 93] |
| Digoxin | EVQLLESGGGLVQPGGSLRLSCAASGFTFSSYAMSWVRQAPGKGLEWVAVIWHDGSSKYYADSVKGRFTISRDNSKNTLYLQMNSLRAEDTAVYYCARATGDGFDYWGQG |
| | TLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAA |
| | WDDSLNGVVFGGXTKLTVLGEQKLISXXXLSXSAA [SEQ ID NO: 94] |
| GLP-1 R | EVQLLESGGGLVQPGGSLRLSCAASGFTFRSYGMHWVRQAPGKGLEWVSGLSWNSAGTGYADSVKGRFTISRDNSKNTLYLQMNSLRAEDTAVYYCAKEMGNNWDHIDY |
| | WGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEA |
| | DYYCAAWDDGLSGPVFGGGTKLTXLGEQKLISEEDLSGSAA [SEQ ID NO: 95] |
| GLP-1 | EVQLLESGGGLVQPGGSLRLSCAASGFTFNSYGMHWVRQAPGKGLEWVSAISGSGGSTYYAESVKGRSTISRDNSKNTLYLQMNSLRAEDTAVYYCVTRNAVFGFDVWGQ |
| | GTLVTVSSGGGGSGGGGSGGGGSQSVLTQPPSASGTPGQRVTISCTGSSSNIGAGFDVHWYQQLPGTAPKLLIYDNNKRPSGVPDRFSGSKSGTSASLAISGLRSEDEADYYC |
| | QSFDSSLSGVVFGGXTKLTVLXEQKLISXEXLSGSAA [SEQ ID NO: 96] |
| C1q | EVQLLESGGGLVQPGGSLRLSCAASGFTFDDYGMSWVRQVPGKGLEWVSAISGSGATTFYAHSVQGRFTISRDNSKNTLYLQMNSLRAEDTAVYYCARGGRGYDWPSGAF |
| | DIWGQGTLVTVSSGGGGSGGGGSGGGGSQSVLTQPPSASGTPGQRVTISCTGSSSNIGAGYDVHWYQQLPGTAPKLLIYENNKRPSXVPDRFSGSKSGTSASLAISGLRSEDX |
| | ADYYCAAWDDSVNGYVVFGGXTKLTVLGEQKLISEXXLSGSAAXXHHHHH-SPRWPIRPIXSRXTIXXPSFYXXXXXXTXXLPXXIXXXHXPXXXXXX [SEQ ID NO: 97] |
| C1s | EVQLLESGGGLVQPGGSLRLSCAASGFTFSSYAMSWVRQAPGKGLEWVSGVSWNGSRTHYADSVKGRFTISRDNSKNTLYLQMNSLRAEDTAVYYCARHMKAAAYVFEIW |
| | GQGTLVTVSSGGGGSGGGGSGGGGSQSVLTQPPSASGTPGQRVTISCSGSSSNIGSTAVNWYQQLPGTAPKLLIYSNNKRPSGVPDRFSGSXSGTSASLAISGLRSEDEADYY |
| | CAAWDDRLNGNVLFGGXXKLTVLXEQXLISXXXLSGSAA [SEQ ID NO: 98] |
| C3 (1) | EVQLLESGGGLVQPGGSLRLSCAASGFTFSSYAMSWVRQAPGKGLEWVSSVTGSGGGTYYADSVEGRFTISRDNSKNTLYLQMNSLRAEDTAVYYCARYRWFGNDAFDIW |
| | GQGTLVTVSSGGGGSGGGGSGGGGSQSVLTQPPSASGTPGQRVTISCSGSASNLGMHFVSWYQQLPGTAPKLLIYGNSNRPSGVPDRFSGSKSGTSASLAISGLRSEDEADY |
| | YCAAWDDTLNIWVFGGXTKLTVLGEQKLISXXXLSGSAA [SEQ ID NO: 99] |
| C3 (2) | EVQLLESGGGLVQPGGSLRLSCAASGFTFSTYRMIWVRQAPGKGLEWVSSISGSNTYIHYADSVRGRFTISRDNSKNTLYLQMNSLRAEDTAVYYCARDRHPLLPSGMDVWG |
| | QGTLVTVSSGGGGSGGGGSGGGGSQSVLTQPPSASGTPGQRVTISCSGSSSNIGKHPVNWYQQLPGTAPKLLIYRNDQRPSGVPDRFSGSKSGTSASLAISGLRSEDXADYYC |
| | QSYDSSLSGSWVFGGXTKLTVLGXQKLISEEDLSGSAA [SEQ ID NO: 100] |
| C4 (1) | EVQLLESGGGLVQPGGSLRLSCAASGFTFSSYPMSWVRQAPGKGLEWVSTLYAGGWTSYADSVWGRFTISRDNSKNTLYLQMNSLRAEDTAVYYCARPKVESLSRYGMDV |
| | WGQGTLVTVSSGGGGSGGGGSGGGGSQSVLTQPPSASGTPGQRVTISCTGSSSNIGAGYVVHWYQQLPGTAPKLLIYDNSKRPSGVPDRFSGSKSGTSASLAISGLRSEDEA |
| | DYYCQSYDSSLSGVVFGGXTKLTVLXEQKLISEXXLSGSAA [SEQ ID NO: 101] |
| C5 (1) | EVQLLESGGGLVQPGGSLRLSCAASGFTFSSYRMNWVRQAPGKGLEWVSAISGSGGSTYYADSVKGRFTISRDNSKNTLYLQMNSLRAEDTAVYYCARGGGWFSGHYYFDY |
| | WGQGTLVTVSSGGGGSGGGGSGGGGSQSVLTQPPSASGTPGQRVTISCTGATSNIGAGYDVHWYQQLPGTAPKLLIYRNNQRPSXVPDRFSGSXSGTSASLAIXGLRSEDXA |
| | DYYCQSYDSSLRHWVFXGXXKLTVLXEQKLISEXXLSGSXA [SEQ ID NO: 102] |
| C5 (2) | EVQLLESGGGLVQPGGSLRLSCAASGFTFSAYSMNWVRQAPGKGLEWVSGVSWNGSRTHYADSVKGRFTISRDNSKNTLYLQMNSLRAEDTAVYYCARENSGFFDYWGQ |
| | GTLVTVSSGGGGSGGGGSGGGGSQSVLTQPPSASGTPGQRVTISCTGSSSNIGSNTVNWYQQLPGTAPKLLIYGNSNRPSGVPDRFSGSKSGTSASLTISGLRSEDXADYYCA |
| | AWDDSLSGWVFGGXTKLTVLXEQKLISEEXLSGSAA [SEQ ID NO: 103] |
| C1 inh (1) | EVQLLESGGGLVQPGGSLXLSCAASGFTFSDYYMSWIRQAPGKGLEWVSGISRGGEYTFYVDSVKGRFTISRDNSKNTLYLQMNSLRAEDTAVYYCARDPGGLDAFDIWGQG |
| | TLVTVSSGGGGSGGGGSGGGGSQSVLTQPPSASGTPGQRVTISCTGSSSNIGARYDVQWYQQLPGTAPKLLIYGNSNRPSGVPDRFSGSKSGTSASLAISGLRSEXXADYYCAS |
| | WDDSLSGPVFGGXTKLTVLXEQKLISEXXLSXSAA [SEQ ID NO: 104] |
| Factor B (1) | EVQLLESGGGLVQPGGSLRLSCAASGFTFSSYSMNWVRQAPGKGLEWVAVISYDGRFIYYSDSVKGRFTISRDNSKNTLYLQMNSLRAEDTAVYYCARSYGGNLAMDVWGQ |
| | GTLVTVSSGGGGSGGGGSGGGGSQSVLTQPPSASGTPGQRVTISCTGSSSNIGAGYDVHWYQQLPGTAPKLLIYDNNKRPSGVPDRFSGSNSGTSASLAISGLRSEDXADYYC |
| | AAWDDRLNGRVVFGGXTKLTVLGEQKLISEXDLSGSAA [SEQ ID NO: 105] |
| IL-12 (3) | EVQLLESGGGLVQPGGSLRLSCAASGFTFSRYGMHWVRQAPGKGLEWVASIRGNARGSFYADSVKGRFTISRDNSKNTLYLQMNSLRAEDTAVYYCAKGDSSGWYFFDYW |
| | GQGTLVTVSSGGGGSGGGGSGGGGSQSVLTQPPSASGTPGQRVTISCTGSDSXIGAGFDVHWYQQLPGTAPKLLIYGNNNRPSGVPDRFSGSKSGTSASLAISGLRSEDXAD |
| | YYCQSYDTSLSGVLFGGXXKLTVLGEQKLISEXDLSGSAA [SEQ ID NO: 106] |
| IL-12 (4) | EVQLLESGGGLVQPGGSLRLSCAASGFTFSDYGMHWVRQAPGKGLEWVSTVSGSGDNTYYADSVKGRFTISRDNSKNTLYLQMNSLRAEDTAVYYCTTTWRYWGQGTLVT |
| | VSSGGGGSGGGGSGGGGSQSVLTQPPSASGTPGQRVTISCSGSSSNIGSNTVNWYQQLPGTAPKLLIYGNSNRPSGVPDRFSGSKSGTSASLAISGLRSEDEADYYCQSYDSSL |
| | SGWVFGGXTKLTVLXEQKLISXEDLSGSAA [SEQ ID NO: 107] |
| IL-16 (3) | EVXLLESGGGLVQPGGSLRLSCAASGFTFSSYGMHWVRQAPGKGLEWVSGINWNGGSTGYADSVKGRFTISRDNSKNTLYLQMNSLRAEDTAVYYCARERGDAFDIWGQG |
| | TLVTVSSGGGGSGGGGSGGGGSQSVLTQPPSASGTPGQRVTISCSGSSSNIGSNTVNWYQQLPGTAPKLLIYSDNQRPSGVPDRFSGSKSGTSASLAISGLRSXXEADYYCAA |
| | WXDSLNGPWVFGGXTKLXVLGEQKLISEEDLSGSAA [SEQ ID NO: 108] |
| IL-18 (3) | EVQLLESGGGLVQPGGSLRLSCAASGFTFSRYGMHWVRQAPGKGLEWVAVISYDGSNKYYADSVKGRFTISRDNSKNTLYLQMNSLRAEDTAVYYCARHGYGDSRSAFDIW |
| | GQGTLVTVSSGGGGSGGGGSGGGGSQSVLTQPPSASGTPGQRVTISCTGSSSNIGAGYDVHWYQQLPGTAPKLLIYRNNQRPSGVPDRFSGSXSGTSASLAISGLRSEXXADY |
| | YCQSYDSSLSRWVFGGXTKLXVLGEQKLISXXXLSXSAA [SEQ ID NO: 109] |
| IL-1a (3) | EVQLLESGGGLVQPGGSLRLSCAASGFTFSSYSMNWVRQAPGKGLEWVSYISSSSSYTNYADSVKGRFTISRDNSKNTLYLQMNSLRAEDTAVYYCARSVTRRAGYYYYYSGM |
| | DVWGQGTLVTVSSGGGGSGGGGSGGGGSQSVLTQPPSASGTPGQRVTISCSGSSSXIGSNTVNWYQQLPGTAPKLLIYRNNQRPSXVPDRFSGSXSGTSASLAISGLRSEDE |
| | AXYYCSSXAGSNSXVFGGXTKLTVLGEQKLISXXXLSGSAA [SEQ ID NO: 110] |
| IL-6 (3) | EVQLLESGGGLVQPGGSLRLSCAASGFTFSNYGMHWVRQAPGKGLEWVSSITSSGDGTYFADSVKGRFTISRDNSKNTLYLQMNSLRAEDTAVYYCARAGGIAAAYAFDIW |
| | GQGTLVTVSSGGGGSGGGGSGGGGSQSVLTQPPSASGTPGQRVTISCSGSSSNVGSNYVYWYQQLPGTAPKLLIYDNNKRPSGVPDRFSGSKSGTSASLAISGLRSEXEADYY |
| | CQSYDSSRWVFGGXTKLTVLGEQKLISEXXLSGSAA [SEQ ID NO: 111] |
| IL-6 (4) | EVQLLESGGGLVQPGGSLRLSCAASGFTFSSNYMSWVRQAPGKGLEWVSSISSSSTIYYADSVKGRFTISRDNSKNTLYLQMNSLRAEDTAVYYCARQPASGTYDAFDIWGQG |
| | TLVTVSSGGGGSGGGGSGGGGSQSVLTQPPSXSGTPGQRVTISCTGSSSNIGAGYDVHWYQQLPGTAPKLLIYYDDLLPSGVPDRFSGSKSGTSASLAISXLRSEDEADYYCAV |
| | WDDSLSGWVFGGXTKLTVLXEQKLISXXDLSGSAXAHHHHHHXSPRXXIRPIVSXITIHXXVVLXRRDWEXPXXTQLNXXXAHXPFXXXXNX [SEQ ID NO: 112] |
| IL-8 (3) | EVQXLESGGGLVQPGGSLRLSCAASGFTFDDYGMSWVRQAPGKGLEWVSLISWDGGSTYYADSVKGRFTISRDNSKNTLYLQMNSLRAEDTAVYYCARDDLYGMDVWGQ |
| | GTLVTVSSGGGGSGGGGSGGGGSQSVLTQPPSASGTPGQRVTISCTGSSSNIGAGYDVHWYQQLPGTAPKLLIYDNNKRPSGVPDRFSGSKSGTSASLAISGLRSXDEADYYC |
| | AAWDDSLSGWVFGGXTKLTVLGEQKLISEXXLSGSAA [SEQ ID NO: 113] |
| MCP-4 (3) | EVQLLESGGGLVQPGGSLRLSCAASGFTFSSYGMHWVRQAPGKGLEWVSGISWNGGKTHYVDSVKGQFTISRDNSKNTLYLQMNSLRAEDTAVYYCARGGYSSGWAFDY |
| | WGQGTLVTVSSGGGGSGGGGSGGGGSQSVLTQPPSASGTPGQRVTISCSGRSSNIESNTVNWYQQLPGTAPKLLIYGNSNRPSGVPDRFSGSKSGTSASLAISGLRSEXEADY |
| | YCAAWDDRLNAVVFGGXTKLXVLXEQKLISEXXLSGSAA [SEQ ID NO: 114] |
| Properdin | EVQLLESGGGLVQPGGSLRLSCAASGFTFSSNYMSWVRQAPGKGLEWVSAISGSGGSTYYADSVKGRFTISRDNSKNTLYLQMNSLRAEDTAVYYCAKGGSGWYDYFDYWG |
| | QGTLVTVSSGGGGSGGGGSGGGGSQSVLTQPPSASGTPGQRVTISCTGSSSNIGAGYDVHWYQQLPGTAPKLLIYRNNQRPSGVPDRFSGSKSGTSASLAISGLRSEDEAXYY |
| | CAAXDDGLNSPVFGGGTKLXVLXEQKLISEEDLSGSAXAHHHHHH-SPRXXIRPIVSRITIHWXXFXXXXXGKTXXXPXLXXXXXXPPFX [SEQ ID NO: 115] |
| TNF-β (3) | EVQLLESGGGLVQPGGSLRLSCAASGFTFSDYYMSWIRQAPGKGLEWVSGLSGSAGRTHYADSVRGRFTISRDNSKNTLYLQMNSLRAEDTAMYYCASSLFDYWGQGTLVT |
| | VSSGGGGSGGGGSGGGGSQSVLTQPPSASGTPGQRVTISCSGSSSNIGSNYVYWYQQLPGTAPKLLIYSNNQRPSGVPDRFSGSKSGTSASLAISGLRSEDXADYYCAAWDDS |
| | LNAVVFGGXTKLTVLGEQKLISEXDLSGSAA [SEQ ID NO: 116] |
| TNF-β (4) | EVQLLESGGGLVQPGGSLRLSCAASGFTFSDYYMNWVRQAPGKGLEWVSGINWNSDDIDYVDSVKGRFTISRDNSKNTLYLQMNSLRAEDTAMYYCAIDSRYSSGWSFEY |
| | WGQGTLVTVSSGGGGSGGGGSGGGGSQSVLTQPPSASGTPGQRVTISCSGSTSNIGNSHVYWYQQLPGTAPKLLIYSNNQRPSGVPDRFSGSKSGTSASLAISGLRSEDEAD |
| | YYCQSYDSSLSGVVFGGXTKLTVLGEQKLISXXXLSGSAA [SEQ ID NO: 117] |
| VEGF (3) | EVQLLESGGGLVQPGGSLRLSCAASGFTFSSYEMNWVRQAPGKGLEWVSGISGSGGFTYYADSVKGRFTISRDNSKNTLYLQMNSLRAEDTAMYYCAREGYQDAFDIWGQ |
| | GTLVTVSSGGGGSGGGGSGGGGSQSVLTQPPSASGTPGQRVTISCTGSSSNIGAGYDVHWYQQLPGTAPKLLIYSNNQRPSXVPDRFSGSXSGTSASLAISGLRSEDXADYYC |
| | AAWDDSLSGPPWVFGGGXKLXVLXEQKLISXXXLSGSXAAHHHHHH-SPRXPIRPIVSXIXIHWPXFYNVXXXXTXXXPXLX [SEQ ID NO: 118] |
| VEGF (4) | EVQLLESGGGLVQPGGSLRLSCAASGFTFXXXYXSWVRQAPGKGLEWVSXISWXXGSIGYADSVKGRFTISRDNSKNTLYLQMNSLRAEDTAVYYCXXXXXXXXNYFDYWGQ |
| | GTLVTVSSGGGGSGGGGSGGGGSQSVLTQPPSASGTPGQRVTISCSGSNSNIGGNFVYWYQQLPGTAPKLLIYENSKRPSXVPDRFSGSXSGTSASLAISGLRSEDXADYYCAA |
| | WDDSLXXVVFGGXTKLTVLGEQKLISXXXLSGSAA [SEQ ID NO: 119] |
| IL-4 (3) | EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARAIAARPFDYWGQ |
| | GTLVTVSSGGGGSGGGGSGGGGSQSVLTQPPSASGTPGQRVTISCTGATSNIGAGYDIHWYQQLPGTAPKLLIYSTNNRPSGVPDRFSGSKSGTSASLAISGLRSEDXADYYCA |
| | AWDDSLNGPVFGGXXKLTVLGEQKLISEXDLSGSAA [SEQ ID NO: 120] |
| CD40 (2) | EVQLLESGGGLVQPGGSLRLSCAASGFTFSAYWMHWVRQAPGKGLEWVSGISGGGGSTYYADSVKGRFTISRDNSKNTLYLQMNSLRAEDTAVYYCARMTPWYYGMDV |
| | WGQGTLVTVSSGGGGSGGGGSGGGGSQSMLTQPPSASGTPGQRVTISCSGSTS [SEQ ID NO: 121] |
| CD40 (3) | EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQG |
| | TLVTVSSGGGGSGGGGSGGGGSQSVLTQPPXXSGTPGQRVTISC [SEQ ID NO: 122] |
| CD40 (4) | EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQG |
| | TLVTVSSGGGGSGGGGSGGGGSQSVLTQPPSASGTPGQRVTISCTGSSSNIGAGYDVYWYQQLPGTAPKLLIYGNINRPSGVPDRFSGSKSGTSASLAISGLRSEDXADYYCAA |
| | WDDSLXGLVFGGXXKLTVLXXYKDDDDKAA [SEQ ID NO: 123] |
| CT17 | EVQLLESGGGLVQPGGSLRLSCAASGFTFSSSAMHWVRQAPGKGLEWVSAISGSGGSTYYADSVKGRFTISRDNSKNTLYLQMNSLRAEDTAVYYCARVKGRVTIFGVVINSN |
| | YGMDVWGQGTLVTVSSGGGGSGGGGSGGGGSQSVLTQPPSASGTPGQRVTISCSGSISSIGSNAVSWYQQLPGTAPKLLIYGNSNRPSGVPDRFSGSKSGTSASLAISGLRSE |
| | DXADYYCAAWDDSLNGHDVVFGGXTKLTVLXDYKDXDXKAA [SEQ ID NO: 124] |
| IgM (3) | EVQLLESGGGLVQPGGSLRLSCAASGFTFSSYAMSWVRQAPGKGLEWVSGISWNSGSIGYADSVKGRFTISRDNSKNTLYLQMNSLRAEDTAVYYCARGDYSSSPGGYYYYM |
| | DVWGQGTLVTVSSGGGGSGGGGSGGGGSQSVLTQPPSASGTPGQRVTISCSGSSSXIGSNTVNWYQQLPGTAPKLLIYGNSNRPSXVPDRFSGXXSGTSASLAIXGLRSXDX |
| | ADYYCSSXXSTNTVIFGGXTKLTVLGEQKLISXXDLSGSAA [SEQ ID NO: 125] |
| IgM (4) | EVQLLESGGGLVQPGGSLRLSCAASGFTFSSNEMSWIRQAPGKGLEWVSAIYSGGGTYYADSVKGRFTISRDNSKNTLYLQMNSLRAEDTAVYYCARVNDYGDNVYFDHWG |
| | QGTLVTVSSGGGGSGGGGSGGGGSQSVLTQPPSASGTPGQRVTISCSGSSSNIGNNYVSWYQQLPGTAPKLLIYENNKRPSXVPDRFSGSXSGTSASLAISGLRSEDEADYYCA |
| | AWDDSLSVYVVFGGXTKLXVLGEQKLISXXDLSGSAA [SEQ ID NO: 126] |
| IgM (5) | EVQLLESGGGLVQPGGSLRLSCAASGFTFGSYEMNWVRQAPGKGLEWVSVIYSGGSTYYADSVEGRFTISRDNSKNTLYLQMNSLRAEDTAVYYCARDTNPYYYYGMDVW |
| | GQGTLVTVSSGGGGSGGGGSGGGGSQSVLTQPPSASGTPGQRVTISCSGSSSNIGNNAVNWYQQLPGTAPKLLIYRNNQRPSXVPDRFSGSXSGTSASLAISGLRSEDEADYY |
| | CQSYDSSLNGQVFGGXTKLTVLXEQKLISXEXLSGSAA [SEQ ID NO: 127] |
| HLA-DR/DP | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDGLLPLDYWGQGTL |
| | VTVSSGGGGSGGGGSGGGGSQSVLTQPPSASGTPGQRVTISCSGGSSNIGGNAVNWYQQLPGTAPKLLIYENNKRPSXVPDRFSGSXSGTSASLAISGLRSEDXADYYCSSYA |
| | VSNNFEVLFGGXTKLTVLXEQKLISXXDLSGSAA [SEQ ID NO: 128] |
| ICAM-1 | EVQLLESGGGLVQPGGSLRLSCAASGFTFSNAWMSWVRQAPGKGLEWVAFIWYDGSNKYYADSVKGRFTISRDNSKNTLYLQMNSLRAEDTAVYYCARYSGWYFDYWGQ |
| | GTLVTVSSGGGGSGGGGSGGGGSQSVLTQPPSASGTPGQRVTISCTGSSSXIGAGYDVHWYQQLPGTAPKLLIYDNNNRPSXVPDRFSGSXSGTSASLAISGLRSEDEADYYC |
| | QSYDSSLSAWLFGGXTKLTVLGEQKLISXXDLSGSXAAHHHHHH-SPRWPIRXIVSXXTIXXPXFYXVXXXKPXXTXLXRXXAHPXX [SEQ ID NO: 129] |
| IgM (2) | EVQLLESGGGLVQPGGSLRLSCAASGFTFSDYYMSWIRQAPGKGLEWVSAIGSGPYYAHSVRDRFTISRDNSKNTLYLQMNSLRAEDTAVYYCARGGVEASFDYWGQGTLVT |
| | VSSGGGGSGGGGSGGGGSQSVLTQPPSASGTPGQRVTISCTGSSSNIGAGYDVHWYQQLPGTAPKLLIYGNTNRPSXVPNRFSGSXSGTSASLAISGLRSEDEADYYCQSYDN |
| | DLSGWVFGGXTKLTVLGEQKLISEEXLSGSAA [SEQ ID NO: 130] |
| MCP-1 (4) | QSVLTQPPSASGTPGQRVTISCTGSSSNIGSDYGVQWYQQLPGTAPKLLIYSNNQRPSGVPDRFSGSKSGTSASLAISGLRSEDEADYYCAAWDDSLSGPVFGGGTKLTVLG |
| | [SEQ ID NO: 131] |
| MCP-1 (5) | QSVLTQPASASGTPGQRVTISCTGNSSNIGAGYDVHWYQQLPGTAPKLLIYRNNQRPSGVPDRFSGSKSGTSASLAISGLLSEDEADYYCAAWDYSLNGWVFGGGTKLTVLG |
| | [SEQ ID NO: 132] |
| MCP-1 (6) | QSVLTQPSSASGTPGQRVTISCTGNSSNIGAGYDVHWYQQLPGTAPNLLIYRNNQRPSGVPDRFSGSKSGTSASLAISGLRSEDEADYYCAAWDDSLNGWVFGGGTKLTVLG |
| | Q [SEQ ID NO: 133] |
| MCP-1 (7) | EVQLLESGGGLVQPGGSLRLSCAASGFTFSSYAMSWVRQAPGKGLEWVAYINRGSTYTNYADSMKGRFTISRDNSKNTLYLQMNSLRAEDTAVYYCARRGYGSGSYYAFDI |
| | WGQGTLVTVSSGGGGSGGGGSGGGGSQSVLTQPPSASGTPGQRVTISCTGSSSNIGSDYGVQWYQQLPGTAPKLLIYSNNQRPSXVPDRFSGSKSGTSASLAISGLRSEDXA |
| | DYYCAAWDDSLSGPVFGGX [SEQ ID NO: 134] |
| MCP-1 (8) | EVQLLESGGGLVQPGGSLRLSCAASGFTFGDYAMSWVRQAPGKGLEWVAVISYDGSNKYYADSVKGRFTISRDNSKNTLYLQMNSLRAEDTAVYYCARDPDPSGTDAFDFW |
| | GQGTLVTVSSGGGGSGGGGSGGGGSQSVLTQPPSASGTPGQRVTISCSGSSSNIGAGYDVHWYQQLPGTAPKLLIYGNSNRPSXVPDRFSGSXSGTSASLAISGLRSEDXADY |
| | YCAAWDDSLSAWAFGG [SEQ ID NO: 135] |
| MCP-1 (9) | EVQLLESGGGLVQPGGSLRLSCAASGFTFSSYEMNWVRQAPGKGLEWVSAISGPGGSTYYADSVKGRFTISRDNSKNTLYLQMNSLRAEDTAVYYCARDLSDYGDFDYWGQ |
| | GTLVTVSSGGGGSGGGGSGGGGSQSVLTQPPSASGTPGQRVTISCTGSSSNIGAGYDAHWYQQLPGTAPKLLIYDNNKRPXXVPDRFSGSXSGTSASLAISGLRSEDEADYYC |
| | ATWDDSLRGWVFG [SEQ ID NO: 136] |
| Cystatin C (1) | EVQLLESGGGLVQPGGSLRLSCAASGFTFSSYAMNWVRQAPGKGLEWVGLISYDGRTTYYADSVKGRSTISRDNSKNTLYLQMNSLRAEDTAVYYCATTTGTTLDYWGQGTL |
| | VTVSSGGGGSGGGGSGGGGSQSVLTQPPSASGTPGQRVTISCTGSSSNIGAGYDVHWYQQLPGTAPKLLIYGNTNRPSXVPDRFSGSXSGTSASLAISGLRSEDEADYYCAA |
| | WDDSLYGWVFGGXTKLTVLGDYXDHDGDYXDHDIDXXDDDDKAA [SEQ ID NO: 137] |
| Cystatin C (2) | EVQLLESGGGLVQPGGSLRLSCAASGFTFSSYSMNWVRQAPGKGLEWVAFISYDGSNKYYVDSVKGRFTISRDNSKNTLYLQMNSLRAEDTAVYYCARDGVPAVPFDYWGQ |
| | GTLVTVSSGGGGSGGGGSGGGGSQSVLTQPPSASGTPGQRVTISCSGSSSNIGAGYDVHWYQQLPGTAPKLLIYSNNQRPS [SEQ ID NO: 138] |
| Cystatin C (3) | EVQLLESGGGLVQPGGSLRLSCAASGFTFSNYAMTWVRQAPGKGLEWVADISHDGFHKYYADSVRGRFTISRDNSKNTLYLQMNSLRAEDTAVYYCARYGRVLPYYYYYGM |
| | DVWGQGTLVTVSSGGGGSGGGGSGGGGSQSVLTQPPSASGTPRQRVTISCTGSSSXIGAGYDVHWYQQLPGTAPKLLIYGNSNRP [SEQ ID NO: 139] |
| Cystatin C (4) | EVQLLESGGGLVQPGGSLRLSCAASGFTFSSYAMNWVRQAPGKGLEWVGLISYDGRTTYYADSVKGRSTISRDNSKNTLYLQMNSLRAEDTAVYYCATTTGTTLDYWGQGTL |
| | VTVSSGGGGSGGGGSGGGGSQSVLTQPPSASGTPGQRVTISCTGSSSNIGAGYDVHWYQQLPGTAPKLLIYGNTNRPSXVPDRFSGSXSGTSXSLAISGLRSXDEADYYCAA |
| | WDDSLYGWVFGG [SEQ ID NO: 140] |
| Apo-A1 (1) | EVQLLESGGGLVQPGGSLRLSCAASGFTFSNNGMHWVRQAPGKGLEWVSAISASGGSTYYADSVKGRFTISRDNSKNTLYLQMNSLRAEDTAVYYCATHGGSSYDAFDIWG |
| | QGTLVTVSSGGGGSGGGGSGGGGSQSVLTQPPSASGTPGQRVTISCTGSSSNIGAGYVVHWYQQLPGTAPKLLIYG [SEQ ID NO: 141] |
| Apo-A1 (2) | EVQLLESGGGLVQPGGSLRLSCAASGFTFRDYYMSWIRQAPGKGLEWVAVTSYDGSKKYYADSVKGRFTISRDNSKNTLYLQMNSLRAEDTAVYYCAKDYADDSIAAPAFDI |
| | WGQGTLVTVSSGGGGSGGGGSGGGGSQSVLTQPPSASGTPGQRVTISCTGSSSXIGAGYDVHWYQQLPGTAPKLLIYGNSNRPS [SEQ ID NO: 142] |
| Apo-A1 (3) | EVXXLESGGGLVQPGGSLRLSCAASGFTFRDYYMSWIRQAPGKGLEWVAVTSYDGSKKYYADSVKGRFTISRDNSKNTLYLQMNSLRAEDTAVYYCAKDYADDSIAAPAFDI |
| | WGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSXDEA |
| | XYYCQSYDSSLSVVFGGGTKLTVLXXYXDHDGDYKDHDIDYXDDXXXAXAHHHHHH-SPXXXIRXXXSXXTIHXXXXXXXXDWXXXXXXXXX [SEQ ID NO: 143] |
| Factor B (2) | EVQLLESGGGLVQPGGSLRLSCAASGFTFSSYSMNWVRQAPGKGLEWVAVISYDGRFIYYSDSVKGRFTISRDNSKNTLYLQMNSLRAEDTAVYYCARSYGGNLAMDVWGQ |
| | GTLVTVSSGGGGSGGGGSGGGGSQSVLTQPPSASGTPGQRVTISCTGSSSNIGAGYDVHWYQQLPGTAPKLLIYDNNKRPSGVPDRFSGSNSGTSASLAISGLRSEDEADYYC |
| | AAWDDRLNGRVVFGGXTKLTVLGDYXDHDGDYKDHDIDXKDDDXKAA [SEQ ID NO: 144] |
| Factor B (3) | EVQLLESGGGLVQPGGSLRLSCAASGFTFSSYSMNWVRQAPGKGLEWVAVISYDGSNQYYADSVRGRFTISKDNSKNTLYLQMNSLRAEDTAVYYCAREWHYSLDVWGQG |
| | TLVTVSSGGGGSGGGGSGGGGSQSVLTQPPSASGTPGQRVTISCSGSSSXIGSNTVNWYQQLPGTAPKLLIYRNNQRPSXVPDRFSGSXSGTSASLAIXGLRSEDXADYYCAA |
| | WDD [SEQ ID NO: 145] |
| Factor B (4) | EVQLLESGGGLVQPGGSLRLSCAASGFTFSKHSMNWVRQAPGKGLEWVATVSYDGNYKYYADSVKGRFTISRDNSKNTLYLQMNSLRAEDTAVYYCAREGYYYYGMDVWG |
| | QGTLVTVSSGGGGSGGGGSGGGGSQSVLTQPPSASGTPGQRVTISCTGSSSNIGNNAVNWYQQLPGTAPKLLIYNNNQRPXXVPDRFSGSXSGTSXSLAISGLRSEDEADYY |
| | CQPYXDXLSSVVFGGXTXLTVLXDXXDHDXXYKDHDIXYXDXDXXXXAHHHHHH-SPRWPIRPIVSXIXIXWXXVLXRXXXXNXXXXXXXXXXXXHXXXXXX [SEQ ID |
| | NO: 146] |
| C1 inh (2) | EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARNRGNWGTYYFDY |
| | WGQGTLVTVSSGGGGSGGGGSGGGGSQSVLTQPPSASGTPGQRVTISCSGSSSNIGSNYVSWYQQLPGTAPKLLIYGSSNRPSGVPDRFSGSXSGTSASLAISGLRSEDEADY |
| | YCQSYDSSLSDHVVFGGXTKLTVLXDYXDHDGDYKDHDIDXXDDDDXAA [SEQ ID NO: 147] |
| C1 inh (3) | EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARNRGNWGTYYFDY |
| | WGQGTLVTVSSGGGGSGGGGSGGGGSQSVLTQPPSASGTPGQRVTISCSGSSSXIGSNYVSWYQQLPGTAPKLLIYGSSNRPSXVPDRFSGSXSGTSASLAISGLRSEDXADYY |
| | CQSYDSSLSDHVVFGGXTKLTVLGDYXDHDGDYKDHDXDXXDDXXXAA [SEQ ID NO: 148] |
| C1 inh (4) | EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARNRGNWGTYYFDY |
| | WGQGTLVTVSSGGGGSGGGGSGGGGSQSVLTQPPSASGTPGQRVTISCSGSSSXIGSNYVSWYQQLPGTAPKLLIYGSSNRPSXVPDRFSGXXSGTSASLAISGLRSEDEADY |
| | YCQSYDSSLSXHVVFGGXTKLTVL [SEQ ID NO: 149] |
| C5 (3) | EVQLLESGGGLVQPGGSLRLSCAASGFTFSSYGMHWVRQAPGKGLEWVSYISSSGSTIYYADSVKGRFTISRDNSKNTLYLQMNSLRAEDTAVYYCLTLGGYWGQGTLVTVSS |
| | GGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSXDEADYYCQSYDSSLSG |
| | WVFGGXTKLTVLXDYKXHDGDYKDHDIDXKDDDXXAA [SEQ ID NO: 150] |
| C4 (2) | EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCAGYGSGSRATGYNW |
| | FAPWGQGTLVTVSSGGGGSGGGGSGGGGSQSVLTQPPSASGTPGQRVTISCTGSSSNIGAGYDVHWYQQLPGTAPKLLIYRNNQRPSGVPDRFSGSXSGTSXSLAISGLRSE |
| | DXADYYCQSYDSSLXGPYWVFXXXNQXDGPRXXXKTMTXXXXXXDIDYXXXXXQXRXAXXXHHH-SPXXP [SEQ ID NO: 151] |
| C4 (3) | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGWSTSSFDYWGQG |
| | TLVTVSSGGGGSGGGGSGGGGSQSVLTQPPSASGTPGQRVTISCSGSSSXIGNHYVSWYQQLPGTATKLLIYXDDLLPSXVPDRFSGSXSGTSASLAIXGLRSEDEADYYCAAW |
| | DDRSGQVLFGGXTKLTVLGDYXDHDGDYXDHDIDXXDDDXKAXAHHHHHH-XXRWPIRPXVSXXTIHXXXFXXXXXXKT [SEQ ID NO: 152] |
| C4 (4) | EVQLLESGGGLVQPGGSLRLSCAASGFTFSSYSMNWVRQAPGKGLEWVSGISGSGGSTYYADSVKGRFTISRDNSKNTLYLQMNSLRAEDTAVYYCAKHSGYGFDIWGQGTL |
| | VTVSSGGGGSGGGGSGGGGSQSVLTQPPSASGTPGQRVTISCSGGASXLGMHFVSWYQQLPGTAPKLLIYYDDLLPSGVPDRFSGXXSGTSASLAISGLRSEDEADYYCAAW |
| | DDSLNGWVFGGXTKLXVLGDYXDXXGDYKDHDIDXKDXXXXAXAHXHHHH-SPXWXXRPIVXXITXXXXVXLQRXDWXXPXVXXXXXXXXXXPX [SEQ ID NO: 153] |
| C3 (3) | EVQLLESGGGLVQPGGSLRLSCAASGFTFSSYAMNWVRQAPGKGLEWVANINQDGSTKFYVDSVKGRFTISRDNSKNTLYLQMNSLRAEDTAVYYCARDTGGNYLGGYYYY |
| | GMDVWGQGTLVTVSSGGGGSGGGGSGGGGSQSVLTQPPSASGTPGQRVTISCSGSSSNIGSNYVYWYQQLPGTAPKLLIYRNDQRPSXVPDRFSGSXSGTSASLAISGLRSX |
| | DXADYYCSSYAGNNNLVFGGXTKLTVLGDYXDHDGDYKDHDIDYXDXDXXAA [SEQ ID NO: 154] |
| C3 (4) | EVQLLESGGGLVQPGGSLRLSCAASGFTFSDHYMDWVRQAPGKGLEWVSGISGNGATIDYADSVKGRFTISRDNSKNTLYLQMNSLRAEDTAVYYCARPSITAAGSEDAFDL |
| | WGQGTLVTVSSGGGGSGGGGSGGGGSQSVLTQPPSASGTPGQRVTISCSGSSSNIGSNYVYWYQQLPGTAPKLLIYGNSNRPSGVPDRFSGSKSGTSASLAISGLRSXDGAD |
| | YYCQSYDSSLSGWVFGGXTKLTVLGXYXDHDGDYKDXDIDYKDDXXKAA [SEQ ID NO: 155] |
| C3 (5) | EVQLLESGGGLVQPGGSLRLSCAASGFTFSNYWMSWVRQAPGKGLEWVSGISGSGGTTYYADFVKGRFTISRDNSKNTLYLQMNSLRAEDTAVYYCARKYYYGSSGAFDIW |
| | GQGTLVTVSSGGGGSGGGGSGGGGSQSVLTQPPSASGTPGQRVTISCTGSSSNIGAGYDVHWYQQLPGTAPKLLIYRNNQRPSXVPDRFSGSXSGTSASLAIXGLRSEDXAD |
| | YYCAAWDDSLXGPVFXGXTKLTVL [SEQ ID NO: 156] |
| C3 (6) | EVQLLESGGGLVQPGGSLRLSCAASGFTFSSYAMNWVRQAPGKGLEWVANINQDGSTKFYVDSVKGRFTISRDNSKNTLYLQMNSLRAEDTAVYYCARDTGGNYLGGYYYY |
| | GMDVWGQGTLVTVSSGGGGSGGGGSGGGGSQSVLTQPPSASGTPGQRVTISCSGSSSNIGSNYVYWYQQLPGTAPKLLIYRNDQRPSGVPDRFSGSXSGTSASLAISGLRSE |
| | DXADYYCSSYAGNNNLVFGGXXKLTVLGXXXDHDGDYKDHDIDXXDXDXXAA [SEQ ID NO: 157] |
| MYOM2 (1) | EVQLLESGGGLVQPGGSLRLSCAASGFTFSSYAMSWVRQAPGKGLEWVSGISGSGGSTYYADSVKGRFTISRDNSKNTLYLQMNSLRAEDTAVYYCARGVVAGSWGQGTLV |
| | TVSSGGGGSGGGGSGGGGSQSVLTQPPSASGTPGQRVTISCSGSSSXIGNNAVNWYQQLPGTAPKLLIYDNNKRPSXVPDRFSGXXSGTSXSLAIXGLRSEDEADYYCA |
| | [SEQ ID NO: 158] |
| MYOM2 (2) | EVQLLESGGGLVQPGGSLRLSCAASGFTFSNEWMAWVRQAPGKGLEWVSSISSSSSYIYYADSVKGRFTISRDNSKNTLYLQMNSLRAEDTAVYYCAGTYHDFWSATYWGQ |
| | GTLVTVSSGGGGSGGGGSGGGGSQSVLTQPPSASGTPGQRVTISCSGSSSNIGSNTVNWYQQLPGTAPKLLIYGNSNRPSXVPDRFSGSXSGTSASLAISGLRSEDXADYYCAA |
| | WDDSLNGWVFGGXTKLTVLGD [SEQ ID NO: 159] |
| LUM | EVQLLESGGGLVQPGGSLRLSCAASGFTFSSNYMSWVRQAPGKGLEWVSAISASGTYTYYTDSVNGRFTISRDNSKNTLYLQMNSLRAEDTAVYYCARVNTVGLGTPFDNW |
| | GQGTLVTVSSGGGGSGGGGSGGGGSQSVLTQPPSASGTPGQRVTISCSGSSSNIGSNTVNWYQQLPGTAPKLLIYGNRNRPSGVPDRFSGSXSGTSASLAISGLRSEDEADYY |
| | CAAWDDSLSGWVFGGXTKLTVLXDYXDHDGDYKDHDIDXXXDDXXAA [SEQ ID NO: 160] |
| DUSP9 | EVQLLESGGGLVQPGGSLRLSCAASGFTFSSYGFHWVRQAPGKGLEWVAVISYDGSNKYYADSVKGRFTISRDNSKNTLYLQMNSLRAEDTAVYYCARGEFGVYWGQGTLV |
| | TVSSGGGGSGGGGSGGGGSQSVLTQPPSASGTPGQRVTISCSGSSSNIGSNYVYWYQQLPGTAPKLLIYGNRNRPSGVPDRFSGSXSGTSASLAISGLRSEDEADYYCSSYAGS |
| | NNFEVVFGGXTKLTVLGDYXDHDGDYKDHDIDYKDDDXKAA [SEQ ID NO: 161] |
| CHX10 (1) | EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARNSDYYGMDVWGQ |
| | GTLVTVSSGGGGSGGGGSGGGGSQSVLTQPPSASGTPGQRVTISCSGSSSNIGYSDVYWYQQLPGTAPKLLIYENNKRPSXVPDRFSGSXSGTSASLAISGLRSEDEADYYCST |
| | WDDSLNGHVIFGG [SEQ ID NO: 162] |
| CHX10 (3) | EVQLLESGGGLVQPGGSLRLSCAASGFTFSSYAMSWVRQAPGKGLEWVAVISYDGSNKYYADSVKGRFTISRDNSKNTLYLQMNSLRAEDTAVYYCARNYGDSINWFDPWG |
| | QGTLVTVSSGGGGSGGGGSGGGGSQSVLTQPPSASGTPGQRVTISCSGSSSXIRSNTVNWYQQLPGTAPKLLIYGNSNRPSXVPDRFSGXXSGTSXSLAISGLRSEDXADYYCA |
| | XWDDSLN [SEQ ID NO: 163] |
| ATP-5B (1) | EVQLLESGGGLVQPGGSLRLSCAASGFTFSSYAMHWVRQAPGKGLEWVAVISYDGSKTYHADSVEGRFTISRDNSKNTLYLQMNSLRAEDTAVYYCARHLRPYYFDYWGQG |
| | TLVTVSSGGGGSGGGGSGGGGSQSVLTQPPSASGTPGQRVTISCSGSSSXIGSNTVNWYQQLPGTAPKLLIYGNSNRPSXVPDRFSGXXSGTSASLAISGLRSEDXADYYCSA |
| | WDDRLRGRVFGG [SEQ ID NO: 164] |
| ATP-5B (2) | EVQLLESGGGLVQPGGSLRLSCAASGFTFSSYGMHWVRQAPGKGLEWVSLISSASSYIYHADSVKGRFTISRDNSKNTLYLQMNSLRAEDTAVYYCARAGRVCTNGVCHTTFD |
| | YWGQGTLVTVSSGGGGSGGGGSGGGGSQSVLTQPPSASGTPGQRVTISCSGDRSNIGSNTVNWYQQLPGTAPKLLIYGNSNRPSGVPXRFSGSXSGTSXSLAISGLRSEDEA |
| | DYYCQSYDSSLSAVVFGGXTKLTVLGDYXXHDXXYKDHDIDYXXDXDXAXAHXHHHH-SPRXXXXPIVSXXXXXXXXXXXXXXLXKXXXXPTXXXXXXXX [SEQ ID NO: 165] |
| ATP-5B (3) | EVQLLESGGGLVQPGGSLRLSCAASGFTFSTYAMSWVRQAPGKGLEWVSSISSTSTYIHYADSVKGRFTISRDNSKNTLYLQMNSLRAEDTAVYYCARVSSWYSAFDIWGQGT |
| | LVTVSSGGGGSGGGGSGGGGSQSVLTQPPSASGTPGQRVTISCTGSSSNIGNNAVNWYQQLPGTAPKLLIYSNNQRPSXVPDRFSGSXSGTSASLAISGLRSEDXADYYCQSY |
| | DSSLSGVIFGGXTKLXVLXDYXDHDGDYXDHDIDXXXDDDKAA [SEQ ID NO: 166] |
| Sox11a | EVQLLESGGGLVQPGGSLRLSCAASGFTFSDFWMSWVRQAPGKGLEWVSSISGGGGTAFYVDSVKGRFTISRDNSKNTLYLQMNSLRAEDTALYYCARMTDLESGDAFDIW |
| | GQGTLVTVSSGGGGSGGGGSGGGGSQSVLTQPPSASGTPGQRVTISCSGSSSNIGSNYVNWYQQLPGTAPKLLIYNDNVRPSGVPDRFSGSXSGTSASLAISGLRSEDXADYY |
| | CQXWGTGVFGGXTKLTVLXDYXDHDGDXXDHDIDXKDXDXKAA [SEQ ID NO: 167] |
| TBC1D9 (1) | EVQLLESGGGLVQPGGSLRLSCAASGFTFSSYSMSWVRQAPGKGLEWVAVISYDGSNKYYADSVKGRFTISRDNSKNTLYLQMNSLRAEDTAVYYCARDRTRGSTALDIWGQ |
| | GTLVTVSSGGGGSGGGGSGGGGSQSVLTQPPSASGTPGQRVTISCSGSSSYIGSNYVYWYQQLPGTAPKLLIYRNNQRPXXVPDRFSGXXSGTSASLAISGLRSEDEADYYCAA |
| | WDDSLSGWVFGGXTKLTVLGD [SEQ ID NO: 168] |
| UPF3B (1) | EVQLLESGGGLVQPGGSLRLSCAASGFTFSDYYMTWIRQAPGKGLEWVSDISWNGSRTHYADSVKGRFTISRDNSKNTLYLQMNSLRAEDTAVYYCSSHLVYWGQGTLVTV |
| | SSGGGGSGGGGSGGGGSQSVLTQPPSASGTPGQRVTISCTGSSSNIGAGYDVHWYQQLPGTAPKLLIYDNNKRPSXVPDRFSGSXSGTSASLAIXGLRSEXXADYYCQTYDSS |
| | LSGSVVFGGXTKLTVLGDYXDHDXDY [SEQ ID NO: 169] |
| UPF3B (2) | EVQLLESGGGLVQPGGSLRLSCAASGFTFSSYAMSWVRQAPGKGLEWVSYISSSSSYANYADSVKGRFTISRDNSKNTLYLQMNSLRAEDTAVYYCARLGVYSGTYLFAFDIW |
| | GQGTLVTVSSGGGGSGGGGSGGGGSQSVLTQPPSASGTPGQRVTISCTGSSSXIGAGYDVHWYQQLPGTAPKLLIYGNSNRPSXVPDRFSGSXSGTSASLAISGLRSXDEADY |
| | YCQSRDSSLSGWVFGGXTKLTVLGD [SEQ ID NO: 170] |
| Apo-A4 (1) | EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCARVAYDIDAFDMW |
| | GQGTLVTVSSGGGG [SEQ ID NO: 171] |
| Apo-A4 (2) | EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCARVAYDIDAFDMW |
| | GQGTLVTVSSGGGGSGGGGSGGGGSQSVLTQPPSASGTPGQRVTISCSGSFSNIGSNYVYWYQQLPGTAPKLLIYENNKRPSGVPDRFSGSXSGTSASLAISGLRSEDEADYYC |
| | AAWDDSLNGPMFGGXTKLTVLXDYKDHDGDYKDHDIDYKDDXXXXAAHHHHHH-SPRWXIRPXXSXXTIHXXXXLXXXD [SEQ ID NO: 172] |
| Apo-A4 (3) | EVQLLESGGGLVQPGGSLRLSCAASGFTFSSYSMNWVRQAPGKGLEWVSAITGSGNATFYADSVKGRFTISRDNSKNTLYLQMNSLRAEDTAVYYCTTGATTRWGQGTLVT |
| | VSSGGGGSGGGGSGGGGSQSVLTQPPSASGTPGQRVTISCSGSRSNIGSNHVFWYQQLPGTAPKLLIYENNKRPSGVPDRFSGSXSGTSASLAISGLRSEDXADYYCAAWDD |
| | SLSGWVFGG [SEQ ID NO: 173] |
| TBC1D9 (2) | EVQLLESGGGLVQPGGSLRLSCAASGFTFSNAWMSWVRQAPGKGLEWVSFISSSSSYIYYADSVKGRFTISRDNSKNTLYLQMNSLRAEDTAVYYCARVNLVGCTNGVCNGH |
| | DYWGQGTLVTVSSGGGGSGGGGSGGGGSQSVLTQPPSASGTPGQRVTISCSGSSSXIGSNTVNWYQQLPGTAPKLLIYDNNKRP [SEQ ID NO: 174] |
| TBC1D9 (3) | EVQLLESGGGLVQPGGSLRLSCAASGFTFGDYAMSWVRQAPGKGLEWVSAISGSGGSTYYADSVKGRFTISRDNSKNTLYLQMNSLRAEDTAVYYCAKGRTMASHWGQGT |
| | LVTVSSGGGGSGGGGSGGGGSQSVLTQPPSASGTPGQRVTISCSGSSSXIGNNHVSWYQQLPGTAPKLLIYGNSNRPSXVPDRFSGSXSGTSASLAISGLRSEDXADYYCAAW |
| | DNSLKVWMFGG [SEQ ID NO: 175] |
| ORP-3 (1) | EVQLLESGGGLVQPGGSLRLSCAASGFTFSSNYMSWVRQAPGKGLEWVSYISGNSGYTNYADSVKGRFTISRDNSKNTLYLQMNSLRAEDTAVYYCARHAGSYDMYGMDV |
| | WGQGTLVTVSSGGGGSGGGGSGGGGSQSVLTQPPSASGTPGQRVTISCSGSTSXIGSHYVYWYQQLPGTAPKLLIYGNSNRPXXVPDRFSGXXSGTSXSLAISGLRSEDXADY |
| | YCQSYDSRLSGWVFGG [SEQ ID NO: 176] |
| ORP-3 (2) | EVQLLESGGGLVQPGGSLRLSCAASGFTFSSYAMHWVRQAPGKGLEWVAVISYDGSNKYYADSVKGRFTISRDNSKNTLYLQMNSLRAEDTAVYYCARKSSLDVWGQGTLV |
| | TVSSGGGGSGGGGSGGGGSQSVLTQPPSASGTPGQRVTISCSGSSSXIGNNYVSWYQQLPGTAPKLLIYDDNKRPSGVPDRFSGSXSDTSASLAISGLRSEDEADYYCAAWDD |
| | SLXGRVFGGXTKLTVLG [SEQ ID NO: 177] |
| CIMS (5) | EVXLLESGGGLVQPGGSLRLSCAASGFTFSDHYMDWVRQAPGKGLEWVSGISGSGGSTYYGDSVKGRFTISRDNSKNTLYLQMNSLRAEDTAVYYCASRLYWGQGTLVTVSS |
| | GGGGSGGGGSGGGGSQSVLTQPPSASGTPGQRVTISCTGSSSNIGAGYVVHWYQQLPGTAPKLLIYDNDKRPSGVPDRFSGSKSGTSASLAISGLRSEDEADYYCAAWDDSL |
| | DAVLFGGXXKLTVLGEQKLISEXDLSGSAA [SEQ ID NO: 178] |
| CIMS (13) | EVQLLESGGGLVQPGGSLRLSCAASGFTFSSYAMSWVRQAPGKGLEWVSAISGSGGRTYYTDSVRDRFTISRDNSKNTLYLQMNSLRAEDTAVYYCARDLMPVCQYCYGMD |
| | VWGQGTLVTVSSGGGGSGGGGSGGGGSQSVLTQPPSASGTPGQRVTISCTGSSSNIGAGYDVHWYQQLPGTAPKLLIYSNNQRPSGVPDRFSGSXSGTSASLAISGLRSEDE |
| | ADYXCQSYDSSLNKDVVFGGXTKLTVLGEQKLISXXDLSGSAXAHHHHHH-SPRXPIRPIVSRXTIHWXXXLXXXDWENXXXTXLXXXAXXPPFXXXXX [SEQ ID NO: 179] |

*The structure of the scFv antibodies is described in Sbderlind et al., 2000, ′Recombining germline-derived CDR sequences for creating diverse single-framework antibody libraries′ Nature Biotechnol., 18(8): 852-6, which is incorporated herein by reference in its entirety.

### SEQUENCE LISTING

Submitted with this application is a Sequence Listing in the form of an ASCII text (.txt) file, which is hereby incorporated by reference into the specification of the application. The ASCII text file (448 KB) was created on Jun. 16, 2022 and has the file name 20220623_Sequence_Listing_147432_001142.txt.

1. A method for determining a systemic lupus erythematosus-associated disease state in a subject comprising the steps of:
a) providing a sample to be tested and an array comprising a plurality of binding agents that bind to a biomarker selected from the group defined in Table A; and
b) using said array, measuring the presence and/or amount in the test sample of one or more biomarker(s) selected from the group defined in Table A;
wherein the presence and/or amount in the test sample of the one or more biomarker(s) selected from the group defined in Table A is indicative of the systemic lupus erythematosus-associated disease state in the subject.

2. The method according to claim 1 further comprising the steps of:
c) providing a control sample from an individual with a different systemic lupus erythematosus-associated disease state to the test subject; and
d) using said array, measuring the presence and/or amount in the control sample of the one or more biomarkers measured in step (b);
wherein the systemic lupus erythematosus-associated disease state is identified in the event that the presence and/or amount in the test sample of the one or more biomarkers measured in step (b) is different from the presence and/or amount in the control sample.

3. The method according to claim 2 further comprising or consisting of the steps of:
e) providing a control sample from an individual with the same systemic lupus erythematosus-associated disease state to the test subject; and
f) using said array, measuring the presence and/or amount in the control sample of the one or more biomarkers measured in step (b);
wherein the systemic lupus erythematosus-associated disease state is identified in the event that the expression in the test sample of the one or more biomarkers measured in step (b) corresponds to the expression in the control sample of the one or more biomarkers measured in step (f).

4. The method according to claim 1 wherein step (b) comprises or consists of measuring the presence and/or amount in the test sample of two or more of the biomarkers defined in Table A

5-6. (canceled)

7. The method according to claim 1 wherein step (b) comprises or consists of:
a) measuring the presence and/or amount in the test sample of one or more of the biomarkers defined in Table B(I);
b) measuring the presence and/or amount in the test sample of one or more of the biomarkers defined in Table B(II);
c) measuring the presence and/or amount in the test sample of one or more of the biomarkers defined in Table B(III);
d) measuring the presence and/or amount in the test sample of one or more of the biomarkers defined in Table B(IV);
e) measuring the presence and/or amount in the test sample of one or more of the biomarkers defined in Table B(V);
f) measuring the presence and/or amount in the test sample of one or more of the biomarkers defined in Table B(VI);
g) measuring the presence and/or amount in the test sample the biomarker defined in Table B(VII);
h) measuring the presence and/or amount in the test sample the biomarker defined in Table B(VIII);
i) measuring the presence and/or amount in the test sample the biomarker defined in Table B(IX);
j) measuring the presence and/or amount in the test sample of one or more of the biomarkers defined in Table B(X);
k) measuring the presence and/or amount in the test sample of one or more of the biomarkers defined in Table B(XI);
l) measuring the presence and/or amount in the test sample of one or more of the biomarkers defined in Table B(XII);
m) measuring the presence and/or amount in the test sample of one or more of the biomarkers defined in Table B(XIII);
n) measuring the presence and/or amount in the test sample the biomarker defined in Table B(XIV);
o) measuring the presence and/or amount in the test sample the biomarker defined in Table B(XV);
p) measuring the presence and/or amount in the test sample of one or more of the biomarkers defined in Table B(XVI);
q) measuring the presence and/or amount in the test sample of one or more of the biomarkers defined in Table B(XVII);
r) measuring the presence and/or amount in the test sample the biomarker defined in Table B(XVIII); and/or
s) measuring the presence and/or amount in the test sample the biomarker defined in Table B(XIX).

8. The method according to claim 1 wherein the method comprises, consists of, or is for determining whether the SLE-associated-disease state is active SLE or non-SLE.

9. The method according to claim 8 wherein step (b) comprises or consists of measuring the presence and/or amount in the test sample of one or more of the biomarkers defined in Table B(I), (II), (III), (IV), (V), (VI), (VIII), (IX), (X), (XI), (XIV) and/or (XVI).

10. The method according to claim 1 wherein the method comprises, consists of, or is for determining whether the SLE-associated-disease state is non-active SLE or non-SLE.

11. The method according to claim 10 wherein step (b) comprises or consists of measuring the presence and/or amount in the test sample of one or more of the biomarkers defined in Table B(I), (II), (III), (V), (VII), (IX), (X), (XII) and/or (XV).

12. The method according to claim 1 wherein the method comprises, consists of, or is for determining whether the SLE-associated-disease state is highly active SLE or non-SLE.

13. The method according to claim 12 wherein step (b) comprises or consists of measuring the presence and/or amount in the test sample of one or more of the biomarkers defined in Table B(I), (II), (IV), (VI), (XII), (XIII), (XIV) and/or (XVIII).

14. The method according to claim 1 wherein the method comprises, consists of, or is for determining whether the SLE-associated-disease state is active SLE or non-active SLE.

15. The method according to claim 14 wherein step (b) comprises or consists of measuring the presence and/or amount in the test sample of one or more of the biomarkers defined in Table B(I), (II), (III), (IV), (V), (VII), (VIII), (XI), (XV) and/or (XVII).

16. The method according to claim 1 wherein the method comprises, consists of, or is for determining whether the SLE-associated-disease state is highly active SLE or non-active SLE.

17. The method according to claim 16 wherein step (b) comprises or consists of measuring the presence and/or amount in the test sample of one or more of the biomarkers defined in Table B(I), (II), (IV), (VI), (XII), (XIII), (XIV) and/or (XVIII).

18. The method according to claim 1 wherein the method comprises or consists of measuring all of the biomarkers listed in Table A and Table B.

19. The method according to claim 3 wherein the control sample of step (c) or step (e) is provided from:
a) a healthy individual (non-SLE);
b) an individual with non-active SLE (non-flaring SLE);
c) an individual with active SLE (flaring SLE); or
d) an individual with highly-active SLE (strongly flaring SLE).

20. The method according to claim 3 wherein the control sample of step (c) or step (e) is provided from:
a) an individual with SLE subtype 1 (SLE1);
b) an individual with SLE subtype 2 (SLE2); or
c) an individual with SLE subtype 3 (SLE3).

21. The method according to claim 1 wherein the physical symptoms of the SLE-associated disease state are present.

22. The method according to claim 1 wherein the SLE-associated disease state is determined before the appearance of the physical symptoms of the SLE-associated disease state.

23. The method according to claim 22 wherein the SLE-associated disease state is determined at least 1 day before the appearance of the physical symptoms of the SLE-associated disease state.

24. (canceled)

25. The method according to claim 1 wherein each of the plurality of binding agents is an antibody or a fragment thereof.

26-27. (canceled)

28. The method according to claim 24 wherein the one or more biomarker(s) in the test sample and/or one or more binding agent(s) are labelled with a detectable moiety.

29. The method according to claim 28 wherein the detectable moiety is selected from the group consisting of: a fluorescent moiety, a luminescent moiety, a chemiluminescent moiety, a radioactive moiety, and an enzymatic moiety.

30. (canceled)

31. The method according to claim 30 wherein the array is a bead-based array, a surface-based array, or a macroarray, microarray, or nanoarray.

32-33. (canceled)

34. The method according to claim 1 wherein step (b) is performed using ELISA (Enzyme Linked Immunosorbent Assay).

35. The method according to claim 1, wherein step (b) comprises or consists of measuring the presence and/or amount in the test sample of one or more of the biomarkers listed in FIG. 1(E), FIG. 2(D), FIG. 2(H), FIG. 3(D), FIG. 3(E), FIG. 3(F), FIG. 4(A), FIG. 5(A), FIG. 5(B), FIG. 8(A), FIG. 8(B), FIG. 8(C) and/or FIG. 8(D).

36. An array for determining a systemic lupus erythematosus-associated disease state in an individual comprising a plurality of binding agents each in the form of an antibody or fragment thereof that binds to one or more of the biomarkers selected from the group defined in Table A, Tables B(I)-B(XIX), FIG. 1(E), FIG. 2(D), FIG. 2(H), FIG. 3(D), FIG. 3(E), FIG. 3(F), FIG. 4(A), FIG. 5(A), FIG. 5(B), FIG. 8(A), FIG. 8(B), FIG. 8(C) and/or FIG. 8(D)

37. (canceled)

38. An array according to claim 36 wherein the array is a bead-based array, a surface-based array, or a macroarray, microarray, or nanoarray.

39. An array according to claim 36 wherein collectively the plurality of binding agents are capable of binding to all of the proteins defined in Table A.

40-43. (canceled)

44. A kit for determining a systemic lupus erythematosus-associated disease state in an individual comprising:
i) a plurality of binding agents each in the form or an antibody or fragment thereof that binds to one or more of the biomarkers selected from the group defined in Table A, Tables B(I)-B(XIX), FIG. 1(E), FIG. 2(D), FIG. 2(H), FIG. 3(D), FIG. 3(E), FIG. 3(F), FIG. 4(A), FIG. 5(A), FIG. 5(B), FIG. 8(A), FIG. 8(B), FIG. 8(C) and/or FIG. 8(D) or an array comprising said one or more binding agents.

45-46. (canceled)


SEQUENCE LISTING (179 sequences)

SEQ ID NO: 1 - protein, 10 aa, Artificial Sequence
  Motif (13)
SGSGQEASFK

SEQ ID NO: 2 - protein, 8 aa, Artificial Sequence
  Motif (14)
SGSGEDFR

SEQ ID NO: 3 - protein, 10 aa, Artificial Sequence
  Motif (3)
SGSGDFAEDK

SEQ ID NO: 4 - protein, 10 aa, Artificial Sequence
  Motif (4) or Motif (7) or Motif (8)
SGSGTEEQLK

SEQ ID NO: 5 - protein, 10 aa, Artificial Sequence
  Motif (5)
SGSGLSADHR

SEQ ID NO: 6 - protein, 261 aa, Artificial Sequence
  IL-1alpha (1) scFv antibody
  VARIANT (193)..(193): Any amino acid
  VARIANT (239)..(239): Any amino acid
  VARIANT (253)..(255): Any amino acids
EVQLLESGGGLVQPGGSLRLSCAASGFTFSSYAMHWVRQAPGKGLEWVSGVSWNGSRTHYADSVKGRFTISRDNSKNTLYLQMNSLRAEDTAVYYCARSSGYYSWAFDIWGQGTLVTVSSGGGGSGGGGSGGGGSQSVLTQPPSASGTPGQRVTISCSGSSSNIGRNTVNWYQQLPGTAPKLLIYGNSNRPSXVPDRFSGSKSGTSASLAISGLRSEDEADYYCAAWDDSLNGWAFGGXTKLTVLGEQKLISXXXLSGSAA

SEQ ID NO: 7 - protein, 264 aa, Artificial Sequence
  IL-1alpha (2) scFv antibody
  VARIANT (222)..(222): Any amino acid
  VARIANT (242)..(242): Any amino acid
EVQLLESGGGLVQPGGSLRLSCAASGFTFSSYSMNWVRQAPGKGLEWVALISYDGSQKYYADSMKGRFTISRDNSKNTLYLQMNSLRAEDTAVYYCAKGHTSGTKAYYFDSWGQGTLVTVSSGGGGSGGGGSGGGGSQSVLTQPPSASGTPGQRVTISCTGTSSNIGAGYSVHWYQQLPGTAPKLLIYGNSNRPSGVPDRFSGSKSGTSASLAISGLRSEDXADYYCQSYDSSLSGWVFGGXTKLTVLGEQKLISEEDLSGSAA

SEQ ID NO: 8 - protein, 260 aa, Artificial Sequence
  IL-2 (1) scFv antibody
  VARIANT (238)..(238): Any amino acid
  VARIANT (243)..(243): Any amino acid
  VARIANT (252)..(253): Any amino acids
EVQLLESGGGLVQPGGSLRLSCAASGFTFGDYAMSWVRQAPGKGLEWVSSISSRGSYIYFADSVKGRFTISRDNSKNTLYLQMNSLRAEDTAVYYCAKKKTGYYGLDAWGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCSSYAGSNNLVFGGXTKLTXLGEQKLISXXDLSGSAA

SEQ ID NO: 9 - protein, 260 aa, Artificial Sequence
  IL-2 (2) scFv antibody
  VARIANT (3)..(4): Any amino acids
  VARIANT (238)..(239): Any amino acids
  VARIANT (253)..(254): Any amino acids
EVXXLESGGGLVQPGGSLRLSCAASGFTFGDYAMSWVRQAPGKGLEWVSSISSRGSYIYFADSVKGRFTISRDNSKNTLYLQMNSLRAEDTAVYYCAKKKTGYYGLDAWGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCSSYAGSNNLVFGGXXKLTVLGEQKLISEXXLSGSAA

SEQ ID NO: 10 - protein, 191 aa, Artificial Sequence
  IL-2 (3) scFv antibody
  VARIANT (162)..(162): Any amino acid
EVQLLESGGGLVQPGGSLRLSCAASGFTFGDYAMSWVRQAPGKGLEWVSSISSRGSYIYFADSVKGRFTISRDNSKNTLYLQMNSLRAEDTAVYYCAKKKTGYYGLDAWGQGTLVTVSSGGGGSGGGGSGGGGSQSVLTQPPSASGTPGQRVTISCTGSSSXIGAGYDVHWYQQLPGTAPKLLIYGNSNRP

SEQ ID NO: 11 - protein, 262 aa, Artificial Sequence
  IL-3 (1) scFv antibody
  VARIANT (220)..(220): Any amino acid
  VARIANT (240)..(241): Any amino acids
  VARIANT (254)..(256): Any amino acids
  VARIANT (260)..(260): Any amino acid
EVQLLESGGGLVQPGGSLRLSCAASGFTFGRYTMHWVRQAPGKGLEWVSSISSSSSYIYYADSVKGRFTISRDNSKNTLYLQMNSLRAEDTAVYYCARHFFESSGGYFDYWGQGTLVTVSSGGGGSGGGGSGGGGSQSVLTQPPSASGTPGQRVTISCSGSSSNIGSNTVNWYQQLPGTAPKLLIYRNNQRPSGVPDRFSGSKSGTSASLAISGLRSEDXADYYCAAWDDSLNGWVFGGXXKLTVLGEQKLISXXXLSGXAA

SEQ ID NO: 12 - protein, 257 aa, Artificial Sequence
  IL-3 (2) scFv antibody
  VARIANT (242)..(242): Any amino acid
  VARIANT (250)..(250): Any amino acid
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGGARYDYWGQGTLVTVSSGGGGSGGGGSGGGGSQSVLTQPPSASGTPGQRVTISCSGSSSNIGSNTVNWYQQLPGTAPKLLIYDNNKRPSGVPDRFSGSKSGTSASLAISGLRSEDEADYYCQSYDNILRGVVFGGGTKLTVLXEQKLISEXDLSGSAA

SEQ ID NO: 13 - protein, 258 aa, Artificial Sequence
  IL-3 (3) scFv antibody
  VARIANT (3)..(5): Any amino acids
  VARIANT (204)..(204): Any amino acid
  VARIANT (236)..(236): Any amino acid
  VARIANT (250)..(251): Any amino acids
EVXXXESGGGLVQPGGSLRLSCAASGFTFSSYAMSWVRQAPGKGLEWVSAISGRGEYTYYAGSVKGRFTISRDNSKNTLYLQMNSLRAEDTAVYYCATGATRFGYWGQGTLVTVSSGGGGSGGGGSGGGGSQSVLTQPPSASGTPGQRVTISCTGSSSNIGAGYGVQWYQQLPGTAPKLLIYRNNQRPSGVPDRFSGSKSGTSXSLAISGLRSEDEADYYCQSYDSSLSYSVFGGXTKLTVLGEQKLISXXDLSGSAA

SEQ ID NO: 14 - protein, 264 aa, Artificial Sequence
  IL-4 (1) scFv antibody
  VARIANT (242)..(242): Any amino acid
  VARIANT (256)..(256): Any amino acid
  VARIANT (258)..(258): Any amino acid
EVQLLESGGGLVQPGGSLRLSCAASGFTFSNAWMSWVRQAPGKGLEWVSSLHGGGDTFYTDSVKGRFTISRDNSKNTLYLQMNSLRAEDTAVYYCASLYGSGSYYYYYYGMDVWGQGTLVTVSSGGGGSGGGGSGGGGSQSVLTQPPSASGTPGQRVTISCSGNNSNTGNNAVNWYQQLPGTAPKLLIYDNNKRPSGVPDRFSGSKSGTSASLAISGLRSEDEADYYCCSYAGSYIWVFGGXTKLTVLGEQKLISXEXLSGSAA

SEQ ID NO: 15 - protein, 266 aa, Artificial Sequence
  IL-4 (2) scFv antibody
  VARIANT (244)..(244): Any amino acid
  VARIANT (248)..(248): Any amino acid
  VARIANT (251)..(251): Any amino acid
  VARIANT (258)..(259): Any amino acids
EVQLLESGGGLVQPGGSLRLSCAASGFTFSDYGMHWVRQAPGKGLEWVSGISWNGGKTHYVDSVKGQFTISRDNSKNTLYLQMNSLRAEDTAVYYCARDRGYCSNGVCYTILDYWGQGTLVTVSSGGGGSGGGGSGGGGSQSVLTQPPSASGTPGQRVTISCSGSSSNIGSNTINWYQQLPGTAPKLLIYGNSNRPSGVPDRFSGSKSGTSASLAISGLRSEDEADYYCQSYDSSLSGWVFGGXTKLXVLXEQKLISXXDLSGSAA

SEQ ID NO: 16 - protein, 187 aa, Artificial Sequence
  IL-5 (1) scFv antibody
  VARIANT (159)..(159): Any amino acid
EVQLLESGGGLVQPGGSLRLSCAASGFTFSSYAMSWVRQAPGKGLEWVSSISSRSNYIYYSDSVKGRFTISRDNSKNTLYLQMNSLRAEDTAVYYCARNFRFFDKWGQGTLVTVSSGGGGSGGGGSGGGGSQSVLTQPPSASGTPGQRVTISCSGGSSXIGANPVSWYQQLPGTAPKLLIYGNSNRP

SEQ ID NO: 17 - protein, 257 aa, Artificial Sequence
  IL-5 (2) scFv antibody
  VARIANT (235)..(235): Any amino acid
  VARIANT (242)..(242): Any amino acid
  VARIANT (250)..(251): Any amino acids
EVQLLESGGGLVQPGGSLRLSCAASGFTFSSYAMSWVRQAPGKGLEWVSSISSRSNYIYYSDSVKGRFTISRDNSKNTLYLQMNSLRAEDTAVYYCARNFRFFDKWGQGTLVTVSSGGGGSGGGGSGGGGSQSVLTQPPSASGTPGQRVTISCSGGSSNIGANPVSWYQQLPGTAPKLLIYGNSNRPSGVPDRFSGSKSGTSASLAISGLRSEDEADYYCQSYDSSLSGSVFGGXTKLTVLXEQKLISEXXLSGSAA

SEQ ID NO: 18 - protein, 257 aa, Artificial Sequence
  IL-5 (3) scFv antibody
  VARIANT (235)..(235): Any amino acid
  VARIANT (249)..(249): Any amino acid
EVQLLESGGGLVQPGGSLRLSCAASGFTFSSYAMSWVRQAPGKGLEWVSSISSRSNYIYYSDSVKGRFTISRDNSKNTLYLQMNSLRAEDTAVYYCARNFRFFDKWGQGTLVTVSSGGGGSGGGGSGGGGSQSVLTQPPSASGTPGQRVTISCSGGSSNIGANPVSWYQQLPGTAPKLLIYGNSNRPSGVPDRFSGSKSGTSASLAISGLRSEDEADYYCQSYDSSLSGSVFGGXTKLTVLGEQKLISXEDLSGSAA

SEQ ID NO: 19 - protein, 297 aa, Artificial Sequence
  IL-6 (1) scFv antibody
  VARIANT (203)..(203): Any amino acid
  VARIANT (208)..(208): Any amino acid
  VARIANT (213)..(213): Any amino acid
  VARIANT (218)..(218): Any amino acid
  VARIANT (220)..(220): Any amino acid
  VARIANT (226)..(227): Any amino acids
  VARIANT (234)..(236): Any amino acids
  VARIANT (240)..(240): Any amino acid
  VARIANT (242)..(242): Any amino acid
  VARIANT (247)..(248): Any amino acids
  VARIANT (254)..(256): Any amino acids
  VARIANT (261)..(263): Any amino acids
  VARIANT (268)..(269): Any amino acids
  VARIANT (272)..(273): Any amino acids
  VARIANT (275)..(278): Any amino acids
  VARIANT (280)..(282): Any amino acids
  VARIANT (284)..(284): Any amino acid
  VARIANT (289)..(289): Any amino acid
  VARIANT (291)..(291): Any amino acid
  VARIANT (293)..(293): Any amino acid
  VARIANT (295)..(297): Any amino acids
EVQLLESGGGLVQPGGSLRLSCAASGFTFSSYGMHWVRQAPGKGLEWVSGINWNGGSTGYADSVKGRFTISRDNSKNTLYLQMNSLRAEDTAVYYCARNRGSSLYYGMDVWGQGTLVTVSSGGGGSGGGGSGGGGSQSVLTQPPSASGTPGQRVTISCAGSSSNIGSKSVHWYQQLPGTAPKLLIYRNNRRPSGVPDRFSGSXSGTSXSLAIXGLRSXDXADYYCXXWDDRVNXXXFGGXTXLTVLXXQKLISXXXLSGSXXXPSSSXXLIXXGXXXXLXXXLXFTGRXFXTXLXXX

SEQ ID NO: 20 - protein, 260 aa, Artificial Sequence
  IL-6 (2) scFv antibody
  VARIANT (238)..(238): Any amino acid
  VARIANT (248)..(248): Any amino acid
  VARIANT (254)..(254): Any amino acid
EVQLLESGGGLVQPGGSLRLSCAASGFTFSNYGMHWVRQAPGKGLEWVSSITSSGDGTYFADSVKGRFTISRDNSKNTLYLQMNSLRAEDTAVYYCARAGGIAAAYAFDIWGQGTLVTVSSGGGGSGGGGSGGGGSQSVLTQPPSASGTPGQRVTISCSGSSSNVGSNYVYWYQQLPGTAPKLLIYDNNKRPSGVPDRFSGSKSGTSASLAISGLRSEDEADYYCQSYDSSRWVFGGXTKLTVLGEQXLISEEXLSGSAA

SEQ ID NO: 21 - protein, 269 aa, Artificial Sequence
  IL-7 (1) scFv antibody
  VARIANT (226)..(227): Any amino acids
  VARIANT (247)..(248): Any amino acids
  VARIANT (251)..(251): Any amino acid
  VARIANT (262)..(263): Any amino acids
EVQLLESGGGLVQPGGSLRLSCAASGFTFSSYGMHWVRQAPGKGLEWVSGITWNSGSIGYVDSVKGRFTISRDNSKNTLYLQMNSLRAEDTAVYYCARGPSVAARRIGRHWYNWFDPWGQGTLVTVSSGGGGSGGGGSGGGGSQSVLTQPPSASGTPGQRVTISCSGSSSNIGSNSVYWYQQLPGTAPKLLIYDNNKRPSGVPDRFSGSKSGTSASLAISGLRSEXXADYYCQSYDSSLSGSVFGGXXKLXVLGEQKLISEXXLSGSAA

SEQ ID NO: 22 - protein, 256 aa, Artificial Sequence
  IL-7 (2) scFv antibody
  VARIANT (226)..(227): Any amino acid
  VARIANT (247)..(247): Any amino acid
EVQLLESGGGLVQPGGSLRLSCAASGFTFSDYNIHWVRQPPGKGLEWVSGVSWNGSRTHYADSVKGQFTISRDNSKNTLYLQMNSLRAEDTAVYYCARDPAMVRGVVLPNYYGLDVWGQGTLVTVSSGGGGSGGGGSGGGGSQSVLTQPPSASGTPGQRVTISCTGSSSNIGAGYDVHWYQQLPGTAPKLLIYGHSNRPSGVPDRFSGSKSGTSASLAISGLRSEXXADYYCQSYDSSLSYPVFGGXTKLTVLGEQ

SEQ ID NO: 23 - protein, 259 aa, Artificial Sequence
  IL-8 (1) scFv antibody
  VARIANT (191)..(191): Any amino acid
  VARIANT (237)..(237): Any amino acid
  VARIANT (244)..(244): Any amino acid
  VARIANT (252)..(253): Any amino acids
EVQLLESGGGLVQPGGSLRLSCAASGFTFDDYGMSWVRQAPGKGLEWVSLISWDGGSTYYADSVKGRFTISRDNSKNTLYLQMNSLRAEDTAVYYCARDDLYGMDVWGQGTLVTVSSGGGGSGGGGSGGGGSQSVLTQPPSASGTPGQRVTISCTGSSSNIGAGYDVHWYQQLPGTAPKLLIYDNNKRPSXVPDRFSGSKSGTSASLAISGLRSEDEADYYCAAWDDSLSGWVFGGXTKLTVLXEQKLISEXXLSGSAA

SEQ ID NO: 24 - protein, 265 aa, Artificial Sequence
  IL-8 (2) scFv antibody
  VARIANT (243)..(243): Any amino acid
  VARIANT (250)..(250): Any amino acid
  VARIANT (257)..(259): Any amino acids
EVQLLESGGGLVQPGGSLRLSCAASGFTFSSYEMNWVRQAPGKGLEWVSSISSSSSYIFYADSMKGRFTISRDNSKNTLYLQMNSLRAEDTAVYYCARNESVDPLGGQYFQHWGQGTLVTVSSGGGGSGGGGSGGGGSQSVLTQPPSASGTPGQRVTISCTGSSSNIGAGYDVHWYQQLPGTAPKLLIYDNNKRPSGVPDRFSGSKSGTSASLAISGLRSEDEADYYCSAWDDNLDGPVFGGXTKLTVLXEQKLISXXXLSGSAA

SEQ ID NO: 25 - protein, 251 aa, Artificial Sequence
  IL-9 (1) scFv antibody
  VARIANT (229)..(229): Any amino acid
  VARIANT (236)..(236): Any amino acid
  VARIANT (244)..(244): Any amino acid
EVQLLESGGGLVQPGGSLRLSCAASGFTFSSYAMSWVRQAPGKGLEWVSSISSSSSYIYYADSVKGRFTISRDNSKNTLYLQMNSLRAEDTAVYYCTTFGHWGQGTLVTVSSGGGGSGGGGSGGGGSQSVLTQPPSASGTPGQRVTISCSGSGSNIGDNSVNWYQQLPGTAPKLLIYGNNNRPSGVPDRFSGSKSGTSASLAISGLRSEDEADYYCSSYTSSSVVFGGXTKLTVLXEQKLISEXDLSGSAA

SEQ ID NO: 26 - protein, 192 aa, Artificial Sequence
  IL-9 (2) scFv antibody
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCAKSPGGSPYYFDYWGQGTLVTVSSGGGGSGGGGSGGGGSQSVLTQPPSASGTPGQRVTISCSGSVSNIGSNVVSWYQQLPGTAPKLLIYDNNKRPS

SEQ ID NO: 27 - protein, 261 aa, Artificial Sequence
  IL-9 (3) scFv antibody
  VARIANT (239)..(239): Any amino acid
  VARIANT (254)..(254): Any amino acid
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCAKSPGGSPYYFDYWGQGTLVTVSSGGGGSGGGGSGGGGSQSVLTQPPSASGTPGQRVTISCSGSVSNIGSNVVSWYQQLPGTAPKLLIYDNNKRPSGVPDRFSGSKSGTSASLAISGLRSEDEADYYCQSYDSSLGGWVFGGXTKLTVLGEQKLISEXDLSGSAA

SEQ ID NO: 28 - protein, 261 aa, Artificial Sequence
  IL-10 (1) scFv antibody
  VARIANT (216)..(216): Any amino acid
  VARIANT (218)..(218): Any amino acid
  VARIANT (239)..(239): Any amino acid
  VARIANT (253)..(254): Any amino acids
EVQLLESGGGLVQPGGSLRLSCAASGFTFRSYVMSWVRQAPGKGLEWVSAISGSGGSTYYADSVKGRFTISRDNSKNTLYLQMNSLRAEDTAVYYCARGKGRWAFDIWGQGTLVTVSSGGGGSGGGGSGGGGSQSVLTQPPSASGTPGQRVTISCTGSSSNVGAGYDVHWYQQLPGTAPKLLIYRNNQRPSGVPDRFSGSKSGTSASLAISGLRSXDXADYYCAAWDDSLSAHVVFGGXTKLTVLGEQKLISXXDLSGSAA

SEQ ID NO: 29 - protein, 261 aa, Artificial Sequence
  IL-10 (2) scFv antibody
  VARIANT (239)..(239): Any amino acid
  VARIANT (254)..(254): Any amino acid
EVQLLESGGGLVQPGGSLRLSCAASGFTFRSYVMSWVRQAPGKGLEWVSAISGSGGSTYYADSVKGRFTISRDNSKNTLYLQMNSLRAEDTAVYYCARGKGRWAFDIWGQGTLVTVSSGGGGSGGGGSGGGGSQSVLTQPPSASGTPGQRVTISCTGSSSNVGAGYDVHWYQQLPGTAPKLLIYRNNQRPSGVPDRFSGSKSGTSASLAISGLRSEDEADYYCAAWDDSLSAHVVFGGXTKLTVLGEQKLISEXDLSGSAA

SEQ ID NO: 30 - protein, 260 aa, Artificial Sequence
  IL-10 (3) scFv antibody
  VARIANT (218)..(218): Any amino acid
  VARIANT (238)..(238): Any amino acid
  VARIANT (245)..(245): Any amino acid
  VARIANT (253)..(254): Any amino acids
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKGRWAFDIWGQGTLVTVSSGGGGSGGGGSGGGGSQSVLTQPPSASGTPGQRVTISCTGSSSNIGAGYGVHWYQQLPGTAPKLLIYGNSNRPSGVPDRFSGSKSGTSASLAISGLRSEDXADYYCAAWDDSLSGLVFGGXTKLTVLXEQKLISEXXLSGSAA

SEQ ID NO: 31 - protein, 262 aa, Artificial Sequence
  IL-11 (1) scFv antibody
  VARIANT (231)..(231): Any amino acid
  VARIANT (240)..(240): Any amino acid
  VARIANT (254)..(254): Any amino acid
  VARIANT (256)..(256): Any amino acid
EVQLLESGGGLVQPGGSLRLSCAASGFTFSNFGMHWVRQAPGKGLEWVAFIRYDGSNKYYADSVKGRFTISRDNSKNTLYLQMNSLRAEDTAVYYCARHYYYSETSGHPGGFDPWGQGTLVTVSSGGGGSGGGGSGGGGSQSVLTQPPSASGTPGQRVTISCSGSSSNIGSYPVNWYQQLPGTAPKLLIYGNSNRPSGVPDRFSGSKSGTSASLAISGLRSEDEADYYCQXWGTGVFGGXTKLTVLGEQKLISXEXLSGSAA

SEQ ID NO: 32 - protein, 267 aa, Artificial Sequence
  IL-11 (2) scFv antibody
  VARIANT (208)..(208): Any amino acid
  VARIANT (245)..(245): Any amino acid
  VARIANT (260)..(261): Any amino acids
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARHYYDVSYRGQQDAFDIWGQGTLVTVSSGGGGSGGGGSGGGGSQSVLTQPPSASGTPGQRVTISCTGSSSNLGSPYDVHWYQQLPGTAPKLLIYRNDQRASGVPDRFSGSXSGTSASLAISGLRSEDEADYYCAAWDDSLNAWVFGGXTKLTVLGEQKLISEXXLSGSAA

SEQ ID NO: 33 - protein, 263 aa, Artificial Sequence
  IL-11 (3) scFv antibody
  VARIANT (241)..(241): Any amino acid
EVQLLESGGGLVQPGGSLRLSCAASGFTFSDYYMSWIRQAPGKGLEWVAYISGISGYTNYADSVRGRFTISRDNSKNTLYLQMNSLRAEDTAVYYCAKSKDWVNGGEMDVWGQGTLVTVSSGGGGSGGGGSGGGGSQSVLTQPPSASGTPGQRVTISCTGSSSNIGAGYVVHWYQQLPGTAPKLLIYSNNQRPSGVPDRFSGSKSGTSASLAISGLRSEDEADYYCAAWDDSLRGWVFGGXTKLTVLGEQKLISEEDLSGSAA

SEQ ID NO: 34 - protein, 256 aa, Artificial Sequence
  IL-12 (1) scFv antibody
  VARIANT (234)..(234): Any amino acid
  VARIANT (249)..(249): Any amino acid
EVQLLESGGGLVQPGGSLRLSCAASGFTFSSYSMNWVRQAPGKGLEWVSAIGTGGGTYYADSVKGRFTISRDNSKNTLYLQMNSLRAEDTAVYYCARAFRAFDIWGQGTLVTVSSGGGGSGGGGSGGGGSQSVLTQPPSASGTPGQRVTISCSGSRSNIGNNFVSWYQQLPGTAPKLLIYGNSNRPSGVPDRFSGSKSGTSASLAISGLRSEDEADYYCAAWDDSLSGPVFGGXTKLTVLGEQKLISEXDLSGSAA

SEQ ID NO: 35 - protein, 262 aa, Artificial Sequence
  IL-12 (2) scFv antibody
  VARIANT (255)..(256): Any amino acids
EVQLLESGGGLVQPGGSLRLSCAASGFTFSDYYMSWVRQAPGKGLEWVSGVSWNGSRTHYADSVKGQFTISRDNSKNTLYLQMNSLRAEDTAVYYCARGSRSSPDAFDIWGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCAAWDDRVNGRVFGGGTKLTVLGEQKLISEXXLSGSAA

SEQ ID NO: 36 - protein, 231 aa, Artificial Sequence
  IL-13 (1) scFv antibody
EVQLLESGGGLVQPGGSLRLSCAASGFTFSSYSMNWVRQAPGKGLEWVSSISSGSSYIYYADSVKGRFTISRDNSKNTLYLQMNSLRAEDTAVYYCARSQGWWTYYYGMDVWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEADYYCETWGQ

SEQ ID NO: 37 - protein, 317 aa, Artificial Sequence
  IL-13 (2) scFv antibody
  VARIANT (221)..(221): Any amino acid
  VARIANT (229)..(229): Any amino acid
  VARIANT (239)..(239): Any amino acid
  VARIANT (255)..(255): Any amino acid
  VARIANT (261)..(261): Any amino acid
  VARIANT (270)..(270): Any amino acid
  VARIANT (272)..(272): Any amino acid
  VARIANT (275)..(276): Any amino acids
  VARIANT (280)..(280): Any amino acid
  VARIANT (285)..(286): Any amino acids
  VARIANT (289)..(289): Any amino acid
  VARIANT (296)..(297): Any amino acids
  VARIANT (301)..(303): Any amino acids
  VARIANT (310)..(312): Any amino acids
  VARIANT (314)..(317): Any amino acids
EVQLLESGGGLVQPGGSLRLSCAASGFTFSSYSMNWVRQAPGKGLEWVSSISSGSSYIYYADSVKGRFTISRDNSKNTLYLQMNSLRAEDTAVYYCARSQGWWTYYYGMDVWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDXADYYCETXDSNTQIFGGXTKLTVLGEQKLISEEXLSGSAXAHHHHHHSXRXPIXXIVSXITIHXXSFXNVVTGKXXALPXXXALQHIPXXXAXXXX

SEQ ID NO: 38 - protein, 261 aa, Artificial Sequence
  IL-13 (3) scFv antibody
  VARIANT (239)..(239): Any amino acid
  VARIANT (254)..(254): Any amino acid
EVQLLESGGGLVQPGGSLRLSCAASGFTFSSYSMNWVRQAPGKGLEWVSSISSGSSYIYYADSVKGRFTISRDNSKNTLYLQMNSLRAEDTAVYYCARSQGWWTYYYGMDVWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEADYYCETWDSNTQIFGGXTKLTVLGEQKLISEXDLSGSAA

SEQ ID NO: 39 - protein, 262 aa, Artificial Sequence
  VEGF (1) scFv antibody
  VARIANT (203)..(203): Any amino acid
  VARIANT (240)..(240): Any amino acid
  VARIANT (255)..(255): Any amino acid
EVQLLESGGGLVQPGGSLRLSCAASGFTFSSNEMSWIRQAPGKGLEWVSSISGSGGFTYYADSVKGRYTISRDNSKNTLYLQMNSLRAEDTAVYYCARETTVRGNAFDIWGQGTLVTVSSGGGGSGGGGSGGGGSQSVLTQPPSASGTPGQRVTISCTGGSSNIGAGYDVHWYQQLPGTAPKLLIYRNNQRPSGVPDRFSGSXSGTSASLAISGLRSEDEADYYCAAWDDSLSVPMFGGXTKLTVLGEQKLISEXDLSGSAA

SEQ ID NO: 40 - protein, 265 aa, Artificial Sequence
  VEGF (2) scFv antibody
  VARIANT (197)..(197): Any amino acid
  VARIANT (206)..(206): Any amino acid
  VARIANT (222)..(222): Any amino acid
  VARIANT (243)..(243): Any amino acid
  VARIANT (252)..(252): Any amino acid
  VARIANT (258)..(259): Any amino acids
EVQLLESGGGLVQPGGSLRLSCAASGFTFSSYSMNWVRQAPGKGLEWVSGINWNGGSTGYADSVKGRFTISRDNSKNTLYLQMNSLRAEDTAVYYCASSVGGWYEGDNWFDPWGQGTLVTVSSGGGGSGGGGSGGGGSQSVLTQPPSASGTPGQRVTISCTGSSSNIGAGYDVHWYQQLPGTAPKLLIYGNSNRPSXVPDRFSGSXSGTSASLAISGLRSEXEADYYCQSYDGSLSGSVFGGXTKLTVLGEXKLISEXXLSGSAA

SEQ ID NO: 41 - protein, 260 aa, Artificial Sequence
  TGF-beta1 (1) scFv antibody
  VARIANT (238)..(238): Any amino acid
  VARIANT (242)..(242): Any amino acid
EVQLLESGGGLVQPGGSLRLSCAASGFTFSTYAMSWVRQAPGKGLEWVAVVSIDGGTTYYGDPVKGRFTISRDNSKNTLYLQMNSLRAEDTAVYYCTRGPTLTYYFDYWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEADYYCQSYDSSLSGWVFGGXTKLXVLGEQKLISEEDLSGSAA

SEQ ID NO: 42 - protein, 259 aa, Artificial Sequence
  TGF-beta1 (2) scFv antibody
  VARIANT (241)..(241): Any amino acid
  VARIANT (252)..(252): Any amino acid
EVQLLESGGGLVQPGGSLRLSCAASGFTFGDYAMSWFRQAPGKGLEWVSGVSWNGSRTHYADSVKGRFTISRDNSKNTLYLQMNSLRAEDTAVYYCARDGNRPLDYWGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCAAWDDRLNGWVFGGGTKLXVLGEQKLISEXDLSGSAA

SEQ ID NO: 43 - protein, 267 aa, Artificial Sequence
  TGF-beta1 (3) scFv antibody
  VARIANT (225)..(225): Any amino acid
  VARIANT (245)..(245): Any amino acid
  VARIANT (259)..(261): Any amino acids
EVQLLESGGGLVQPGGSLRLSCAASGFTFSDYYIGWIRQAPGKGLEWVSGINWNGGSTGYADSVKGRFTISRDNSKNTLYLQMNSLRAEDTAVYYCARRSTPSSSWALPDFFDYWGQGTLVTVSSGGGGSGGGGSGGGGSQSVLTQPPSASGTPGQRVTISCTGSSSNIGANYDVHWYQQLPGTAPKLLIYSNNQRPSGVPDRFSGSKSGTSASLAISGLRSEDXADYYCQSYDSSLSGWVFGGXTKLTVLGEQKLISXXXLSGSAA

SEQ ID NO: 44 - protein, 259 aa, Artificial Sequence
  TNF-alpha (1) scFv antibody
  VARIANT (191)..(191): Any amino acid
  VARIANT (200)..(200): Any amino acid
  VARIANT (237)..(237): Any amino acid
  VARIANT (244)..(244): Any amino acid
  VARIANT (251)..(252): Any amino acids
EVQLLESGGGLVQPGGSLRLSCAASGFTFDDYGMSWVRQAPGKGLEWVAVISYDGSNKYYADSVKGRFTISRDNSKNTLYLQMNSLRAEDTAVYYCTRHLGSAMGYWGQGTLVTVSSGGGGSGGGGSGGGGSQSVLTQPPSASGTPGQRVTISCTGSSSNIGAGYDVHWYQQLPGTAPKLLIYGNSNRPSXVPDRFSGSXSGTSASLAISGLRSEDEADYYCQSYDSSLSGWVFGGXTKLTVLXEQKLISXXDLSGSAA

SEQ ID NO: 45 - protein, 261 aa, Artificial Sequence
  TNF-alpha (2) scFv antibody
  VARIANT (239)..(239): Any amino acid
  VARIANT (241)..(241): Any amino acid
  VARIANT (246)..(246): Any amino acid
  VARIANT (254)..(255): Any amino acids
EVQLLESGGGLVQPGGSLRLSCAASGFTFSSYGMHWVRQAPGKGLEWVSAISGSGGSTYYADSVKGRFTISRDNSKNTLYLQMNSLRAEDTAVYYCARGGWGPRSAFDIWGQGTLVTVSSGGGGSGGGGSGGGGSQSVLTQPPSASGTPGQRVTISCSGSSSNIGSNTVTWYQQLPGTAPKLLIYGNTNRLSGVPDRFSGSKSGTSASLAISGLRSEDEADYYCEAWDDKLFGPVFGGXTXLTVLXEQKLISEXXLSGSAA

SEQ ID NO: 46 - protein, 261 aa, Artificial Sequence
  TNF-alpha (3) scFv antibody
  VARIANT (240)..(240): Any amino acid
  VARIANT (246)..(246): Any amino acid
  VARIANT (248)..(248): Any amino acid
  VARIANT (253)..(255): Any amino acids
EVQLLESGGGLVQPGGSLRLSCAASGFTFSSYWMSWVRQAPGKGLEWVSGVNWNGSRTHYADSVKGRFTISRDNSKNTLYLQMNSLRAEDTAVYYCASIRANYYYGMDVWGQGTLVTVSSGGGGSGGGGSGGGGSQSVLTQPPSASGTPGQRVTISCSGGSSNIGSHPVNWYQQLPGTAPKLLIYGNSNRPSGVPDRFSGSKSGTSASLAISGLRSEDEADYYCAAWDASLSGWVFGGGXKLTVLXEXKLISXXXLSGSAA

SEQ ID NO: 47 - protein, 261 aa, Artificial Sequence
  GM-CSF (1) scFv antibody
  VARIANT (197)..(197): Any amino acid
  VARIANT (239)..(239): Any amino acid
  VARIANT (254)..(255): Any amino acids
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARVGGMSAPVDYWGQGTLVTVSSGGGGSGGGGSGGGGSQSVLTQPPSASGTPGQRVTISCSGSSSNIGSNTVNWYQQLPGTAPKLLIYDNNKRPSGVPDRXSGSKSGTSASLAISGLRSEDEADYYCAAWDDSLIGLVVFGGXTKLTVLGEQKLISEXXLSGSAA

SEQ ID NO: 48 - protein, 260 aa, Artificial Sequence
  GM-CSF (2) scFv antibody
  VARIANT (193)..(193): Any amino acid
  VARIANT (238)..(238): Any amino acid
  VARIANT (242)..(242): Any amino acid
  VARIANT (252)..(252): Any amino acid
EVQLLESGGGLVQPGGSLRLSCAASGFTFSSYAMSWVRQAPGKGLEWVAVISYDGSNEDSADSVKGRFTISRDNSKNTLYLQMNSLRAEDTAVYYCARGPSLRGVSDYWGQGTLVTVSSGGGGSGGGGSGGGGSQSVLTQPPSASGTPGQRVTISCTGSSSNIGAGYDVHWYQQLPGTAPKLLIYNDNQRPSXVPDRFSGSKSGTSASLAISGLRSEDEADYYCQTWGTGINVIFGGXTKLXVLGEQKLISXEDLSGSAA

SEQ ID NO: 49 - protein, 260 aa, Artificial Sequence
  GM-CSF (3) scFv antibody
  VARIANT (219)..(219): Any amino acid
  VARIANT (238)..(238): Any amino acid
  VARIANT (247)..(247): Any amino acid
  VARIANT (253)..(254): Any amino acids
EVQLLESGGGLVQPGGSLRLSCAASGFTFSSYAMSWVRQAPGKGLEWVAVISYDGSNEDSADSVKGRFTISRDNSKNTLYLQMNSLRAEDTAVYYCARGPSLRGVSDYWGQGTLVTVSSGGGGSGGGGSGGGGSQSVLTQPPSASGTPGQRVTISCTGSSSNIGAGYDVHWYQQLPGTAPKLLIYNDNQRPSGVPDRFSGSKSGTSASLAISGLRSEDXADYYCQTWGTGINVIFGGXTKLTVLGEXKLISEXXLSGSAA

SEQ ID NO: 50 - protein, 261 aa, Artificial Sequence
  TNF-beta (1) scFv antibody
  VARIANT (194)..(194): Any amino acid
  VARIANT (203)..(203): Any amino acid
  VARIANT (228)..(228): Any amino acid
  VARIANT (239)..(239): Any amino acid
  VARIANT (253)..(254): Any amino acids
EVQLLESGGGLVQPGGSLRLSCAASGFTFSSFAMHWVRQAPGKGLEWVSAISGSGGSTYYADSVKGRFTISRDNSKNTLYLQMNSLRAEDTAVYYCASRSTLYYYYGMDVWGQGTLVTVSSGGGGSGGGGSGGGGSQSVLTQPPSASGTPGQRVTISCSGSTSNIGNSHVYWYQQLPGTAPKLLIYGNSNRPSXVPDRFSGSXSGTSASLAISGLRSEDEADYYCSSXAGSNNLVFGGXTKLTVLGEQKLISXXDLSGSAA

SEQ ID NO: 51 - protein, 258 aa, Artificial Sequence
  TNF-beta (2) scFv antibody
  VARIANT (236)..(236): Any amino acid
  VARIANT (243)..(243): Any amino acid
  VARIANT (250)..(252): Any amino acids
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARSPYYGMDVWGQGTLVTVSSGGGGSGGGGSGGGGSQSVLTQPPSASGTPGQRVTISCTGSSSNIGAGYDVHWYQQLPGTAPKLLIYRNDQRPSGVPDRFSGSKSGTSASLAISGLRSEDEADYYCSSYGGRDNVVFGGXTKLTVLXEQKLISXXXLSGSAA

SEQ ID NO: 52 - protein, 262 aa, Artificial Sequence
  IL-1ra (1) scFv antibody
  VARIANT (240)..(240): Any amino acid
  VARIANT (244)..(244): Any amino acid
  VARIANT (247)..(247): Any amino acid
  VARIANT (254)..(254): Any amino acid
EVQLLESGGGLVQPGGSLRLSCAASGFTFDTHWMSWVRQAPGKGLEWVSAISGSGGSTYYADSVKGRFTISRDNSKNTLYLQMNSLRAEDTAVYYCARHDYGDYRAFDIWGQGTLVTVSSGGGGSGGGGSGGGGSQSVLTQPPSASGTPGQRVTISCTGSSSNIGAGYVVHWYQQLPGTAPKLLIYGNNNRPSGVPDRFSGSKSGTSASLAISGLRSEDEADYYCQSYDSSLSGVVFGGXTKLXVLXEQKLISXEDLSGSAA

SEQ ID NO: 53 - protein, 259 aa, Artificial Sequence
  IL-1ra (2) scFv antibody
  VARIANT (217)..(217): Any amino acid
  VARIANT (227)..(227): Any amino acid
  VARIANT (237)..(237): Any amino acid
  VARIANT (251)..(254): Any amino acids
EVQLLESGGGLVQPGGSLRLSCAASGFTFSKYAMTWVRQAPGKGLEWVSAISGSGGNTYYADSVKGRFTISRDNSKNTLYLQMNSLRAEDTAVYYCARLVRGLYYGMDVWGQGTLVTVSSGGGGSGGGGSGGGGSQSVLTQPPSASGTPGQRVTISCSGSSSNIGNNAVNWYQQLPGTAPKLLIYGNSNRPSGVPDRFSGSKSGTSASLAISGLRSXDEADYYCQTXGTGPVVFGGXTKLTVLGEQKLISXXXXSGSAA

SEQ ID NO: 54 - protein, 263 aa, Artificial Sequence
  IL-1ra (3) scFv antibody
  VARIANT (241)..(242): Any amino acids
  VARIANT (245)..(245): Any amino acid
  VARIANT (248)..(248): Any amino acid
EVQLLESGGGLVQPGGSLRLSCAVSGFTFSSYSMNWVRQAPGKGLEWVAGIGGRGATTYYVDSVKGRFTISRDNSKNTLYLQMNSLRAEDTAVYYCARLRVVPAARFDYWGQGTLVTVSSGGGGSGGGGSGGGGSQSVLTQPPSASGTPGQRVTISCSGSSSNIGSNTVNWYQQLPGTAPKLLIYGNSNRPSGVPDRFSGSKSGTSASLAISGLRSEDEADYYCQSYDSSLSGPPWVFGGXXKLXVLXEQKLISEEDLSGSAA

SEQ ID NO: 55 - protein, 263 aa, Artificial Sequence
  IL-16 (1) scFv antibody
  VARIANT (241)..(241): Any amino acid
  VARIANT (256)..(256): Any amino acid
EVQLLESGGGLVQPGGSLRLSCAASGFTFSNHAMSWVRQAPGKGLEWVSGVSWNGSRTHYADSVKGRFTISRDNSKNTLYLQMNSLRAEDTAVYYCARAALVQGVKHAFEIWGQGTLVTVSSGGGGSGGGGSGGGGSQSVLTQPPSASGTPGQRVTISCSGSSSNIGSNTVNWYQQLPGTALKLLIYRNNQRPSGVPDRFSGSKSGTSASLAISGLRSEDEADYYCASWDDRLSGLVFGGXTKLTVLGEQKLISEXDLSGSAA

SEQ ID NO: 56 - protein, 263 aa, Artificial Sequence
  IL-16 (2) scFv antibody
  VARIANT (204)..(204): Any amino acid
  VARIANT (220)..(220): Any amino acid
  VARIANT (241)..(241): Any amino acid
  VARIANT (248)..(248): Any amino acid
EVQLLESGGGLVQPGGSLRLSCAASGFTFSNHAMSWVRQAPGKGLEWVSGVSWNGSRTHYADSVKGRFTISRDNSKNTLYLQMNSLRAEDTAVYYCARAALVQGVKHAFEIWGQGTLVTVSSGGGGSGGGGSGGGGSQSVLTQPPSASGTPGQRVTISCSGSSSNIGSNTVNWYQQLPGTALKLLIYRNNQRPSGVPDRFSGSXSGTSASLAISGLRSEXEADYYCASWDDRLSGLVFGGXTKLTVLXEQKLISEEDLSGSAA

SEQ ID NO: 57 - protein, 259 aa, Artificial Sequence
  IL-18 (1) scFv antibody
  VARIANT (201)..(201): Any amino acid
  VARIANT (226)..(226): Any amino acid
  VARIANT (237)..(237): Any amino acid
  VARIANT (251)..(253): Any amino acids
EVQLLESGGGLVQPGGSLRLSCAASGFTFSSYGMHWVRQAPGKGLEWVSGINWNGGSTGYADSVKGRFTISRDNSKNTLYLQMNSLRAEDTAVYYCARDLRGGRFDPWGQGTLVTVSSGGGGSGGGGSGGGGSQSVLTQPPSASGTPGQRVTISCTGSSSNIGAGYVVHWYQQLPGTAPKLLIYRNNQRPSGVPDRFSGSXSGTSASLAISGLRSEDEADYYCSSXAGSKNLIFGGXTKLTVLGEQKLISXXXLSGSAA

SEQ ID NO: 58 - protein, 262 aa, Artificial Sequence
  IL-18 (2) scFv antibody
  VARIANT (226)..(226): Any amino acid
  VARIANT (240)..(241): Any amino acids
  VARIANT (244)..(244): Any amino acid
  VARIANT (249)..(249): Any amino acid
  VARIANT (255)..(255): Any amino acid
EVQLLESGRGLVQPGGSLRLSCAASGFTFSSYGMHWVRQAPGKGLEWVSAIGTGGDTYYADSVMGRFTISRDNSKNTLYLQMNSLRAEDTAVYYCARSPRRGATAGTFDYWGQGTLVTVSSGGGGSGGGGSGGGGSQSVLTQPPSASGTPGQRVTISCSGSSSNIGSNIVNWYQQLPGTAPKLLIYRNNQRPSGVPDRFSGSKSGTSASLAISGLRSEDEADYYCXSYDNSLSGWVFGGXXKLXVLGEXKLISEXDLSGSAA

SEQ ID NO: 59 - protein, 261 aa, Artificial Sequence
  MCP-4 (1) scFv antibody
  VARIANT (239)..(239): Any amino acid
  VARIANT (254)..(254): Any amino acid
EVQLLESGGGLVQPGGSLRLSCAASGFTFSSYGMHWVRQAPGKGLEWVSGISWNGGKTHYVDSVKGQFTISRDNSKNTLYLQMNSLRAEDTAVYYCARGGYSSGWAFDYWGQGTLVTVSSGGGGSGGGGSGGGGSQSVLTQPPSASGTPGQRVTISCSGRSSNIESNTVNWYQQLPGTAPKLLIYGNSNRPSGVPDRFSGSKSGTSASLAISGLRSEDEADYYCAAWDDRLNAVVFGGXTKLTVLGEQKLISEXDLSGSAA

SEQ ID NO: 60 - protein, 261 aa, Artificial Sequence
  IFN-gamma (1) scFv antibody
  VARIANT (195)..(195): Any amino acid
  VARIANT (204)..(204): Any amino acid
  VARIANT (228)..(228): Any amino acid
  VARIANT (239)..(239): Any amino acid
  VARIANT (248)..(248): Any amino acid
  VARIANT (255)..(255): Any amino acid
EVQLLESGGGLVQPGGSLRLSCAASGFTFSSYGMHWVRQAPGKGLEWVSGVSWNGSRTHYADSVKGRFTISRDNSKNTLYLQMNSLRAEDTAVYYCARGRTGHGWKYYFDLWGQGTLVTVSSGGGGSGGGGSGGGGSQSVLTQPPSASGTPGQRVTISCSGSSSNIGNNAVNWYQQLPGTAPKLLIYGNSNRPSXVPDRFSGSXSGTSASLAISGLRSEDEADYYCQXWGTGLGVFGGXTKLTVLGEXKLISEEXLSGSAA

SEQ ID NO: 61 - protein, 189 aa, Artificial Sequence
  IFN-gamma (2) scFv antibody
EVQLLESGGGLVQPGGSLRLSCAASGFTFSRHGFHWVRQGPGKGLEWVSGVSWNGSRTHYADSVKGRFTISRDNSKNTLYLQMNSLRAEDTAVYYCARGNWYRAFDIWGQGTLVTVSSGGGGSGGGGSGGGGSQSVLTQPPSASGTPGQRVTISCSGGSSHIGRNFISWYQQLPGTAPKLLIYAGNSRP

SEQ ID NO: 62 - protein, 261 aa, Artificial Sequence
  IL-1beta (1) scFv antibody
  VARIANT (219)..(219): Any amino acid
  VARIANT (239)..(239): Any amino acid
  VARIANT (254)..(255): Any amino acids
EVQLLESGGGLVQPGGSLRLSCAASGFTFSSYAMSWVRQAPGKGLEWVSYISSSGSTIYYADSVKGRSTISRDNSKNTLYLQMNSLRAEDTAVYYCARVRQNSGSYAYWGQGTLVTVSSGGGGSGGGGSGGGGSQSVLTQPPSASGTPGQRVTISCTGTSSNIGAPYDVHWYQQLPGTAPKLLIYGNSNRPSGVPDRFSGSKSGTSASLAISGLRSEDXADYYCQSYDSSLSAVVFGGXTKLTVLGEQKLISEXXLSGSAA

SEQ ID NO: 63 - protein, 267 aa, Artificial Sequence
  IL-1beta (2) scFv antibody
  VARIANT (245)..(245): Any amino acid
  VARIANT (247)..(247): Any amino acid
  VARIANT (250)..(250): Any amino acid
  VARIANT (252)..(252): Any amino acid
  VARIANT (261)..(261): Any amino acid
EVQLLESGGGLVQPGGSLRLSCAASGFTFSRYVMTWVRQAPGKGLEWVSLISGGGSATYYADSMKGRFTISRDNSKNTLYLQMNSLRAEDTAVYYCAKRVPYDSSGYYPDAFDIWGQGTLVTVSSGGGGSGGGGSGGGGSQSVLTQPPSASGTPGQRVTISCTGSSSNIGAGYDVHWYQQLPGTAPKLLIYGNSNRPSGVPDQFSGSKSGTSASLAISGLRSEDEADYYCAAWDDSLNGPVFGGXTXLTXLXEQKLISEEXLSGSAA

SEQ ID NO: 64 - protein, 264 aa, Artificial Sequence
  IL-1beta (3) scFv antibody
  VARIANT (242)..(242): Any amino acid
  VARIANT (249)..(249): Any amino acid
  VARIANT (257)..(258): Any amino acids
EVQLLESGGGLVQPGGSLRLSCAASGFTFSSYWMSWVRQAPGKGLEWVAVVSYDGNNKYYADSRKGRFTISRDNSKNTLYLQMNSLRAEDTAMYYCASYWYTSGWYPYGMDVWGQGTLGTVSSGGGGSGGGGSGGGGSQSVLTQPPSASGTPGQRVTISCTGSSSNIGAGYDLHWYQQLPGTAPKLLIYRNNQRPSGVPDRFSGSKSGTSASLAISGLRSEDEADYYCSSYVDNNNLVFGGXTKLTVLXEQKLISEXXLSGSAA

SEQ ID NO: 65 - protein, 265 aa, Artificial Sequence
  Eotaxin (1) scFv antibody
  VARIANT (206)..(206): Any amino acid
  VARIANT (243)..(243): Any amino acid
  VARIANT (257)..(258): Any amino acids
  VARIANT (262)..(262): Any amino acid
EVQLLESGGGLVQPGGSLRLSCAASGFTFSSYWMSWVRQAPGKGLEWVSGVSWNGSRTHYADSVKGRFTISRDNSKNTLYLQMNSLRAEDTAVYYCVKGKGTIAMPGRARVGWWGQGTLVTVSSGGGGSGGGGSGGGGSQSVLTQPPSASGTPGQRVTISCSGSSSNIGNNAVNWYQQLPGTAPKLLIYANSNRPSGVPDRFSGSXSGTSASLAISGLRSEDEADYYCAAWDDSLSGPVFGGXTKLTVLGEQKLISXXDLSXSAA

SEQ ID NO: 66 - protein, 322 aa, Artificial Sequence
  Eotaxin (2) scFv antibody
  VARIANT (199)..(199): Any amino acid
  VARIANT (236)..(236): Any amino acid
  VARIANT (250)..(252): Any amino acids
  VARIANT (257)..(257): Any amino acid
  VARIANT (269)..(269): Any amino acid
  VARIANT (277)..(278): Any amino acids
  VARIANT (289)..(289): Any amino acid
  VARIANT (293)..(295): Any amino acids
  VARIANT (299)..(299): Any amino acid
  VARIANT (301)..(303): Any amino acids
  VARIANT (311)..(312): Any amino acids
  VARIANT (314)..(316): Any amino acids
  VARIANT (318)..(322): Any amino acids
EVQLLESGGGLVQPGGSLRLSCAASGFTFSAYWMTWVRQAPGKGLEWVSVIYSGGSTYYADSVKGRFTISRDNSKNTLYLQMNSLRAEDTAVYYCARQTQQEYFDYWGQGTLVTVSSGGGGSGGGGSGGGGSQSVLTQPPSASGTPGQRVTISCFGSNSNIGSSTVNWYQQLPGTAPKLLIYDNDKRPSGVPDRFSGSXSGTSASLAISGLRSEDEADYYCAAWDDSLNGPVFGGXTKLTVLGEQKLISXXXLSGSXAAHHHHHHSPRXPIRPIVSXXTIHWPSFYNVXTGKXXXLPNXIXXXHIPLSPAXXIXXXPXXXXX

SEQ ID NO: 67 - protein, 260 aa, Artificial Sequence
  Eotaxin (3) scFv antibody
  VARIANT (218)..(218): Any amino acid
  VARIANT (235)..(235): Any amino acid
  VARIANT (239)..(239): Any amino acid
EVQLLESGGGLVQPGGSLRLSCAASGFTFRGYAMSWVRQAPGKGLEWVSGVSWNGSRTHYADSVKGRFTISRDNSKNTLYLQMNSLRAEDTAVYYCARAPAVAGWFDPWGQGTLVTVSSGGGGSGGGGSGGGGSQSVLTQPPSASGTPGQRVTISCSGSSSNIGSHTVNWYQQLPGTAPKLLIYRNNQRPSGVPDRFSGSKSGTSASLAISGLRSEDXADYYCAAWDDSLSGRVXGGGXKLTVLGEQKLISEEDLSGSAA

SEQ ID NO: 68 - protein, 263 aa, Artificial Sequence
  RANTES (1) scFv antibody
  VARIANT (241)..(241): Any amino acid
  VARIANT (256)..(257): Any amino acids
EVQLLESGGGLVQPGGSLRLSCAASGFTFSSYGMHWVRQAPGKGLEWVAVISNDGTKKDYADSVKGRFTISRDNSKNTLYLQMNSLRAEDTAVYYCARDASGYDDYYFDYWGQGTLVTVSSGGGGSGGGGSGGGGSQSVLTQPPSASGTPGQRVTISCTGSSSNIGAGSDVHWYQQLPGTAPKLLIYRDDQRSSGVPDRFSGSKSGTSAFLAISGLRSEDEADYYCQSYDNSLSGWVFGGXTKLTVLGEQKLISEXXLSGSAA

SEQ ID NO: 69 - protein, 191 aa, Artificial Sequence
  RANTES (2) scFv antibody
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDNDYSSDTFDYWGQGTLVTVSSGGGGSGGGGSGGGGSQSVLTQPPSAFGTPGQRVTISCSGSSSNIGSDYVYWYQQLPGTAPKLLIYSDNQRP

SEQ ID NO: 70 - protein, 263 aa, Artificial Sequence
  RANTES (3) scFv antibody
  VARIANT (241)..(241): Any amino acid
  VARIANT (256)..(256): Any amino acid
EVQLLESGGGLVQPGGSLRLSCAASGFTFSNYGMNWVRQAPGKGLEWVSGVSWNGSRTHYVDSVKRRFTISRDNSKNTLYLQMNSLRAEDTAVYYCARPRLRSHNYYGMDVWGQGTLVTVSSGGGGSGGGGSGGGGSQSVLTQPPSASGTPGQRVTISCSGSSFKSGKNYVSWYQQLPGTAPKLLIYRNNQRPSGVPDRFSGSKSGTSASLAISGLRSEDEADYYCAAWDVRVKGVIFGGXTKLTVLGEQKLISEXDLSGSAA

SEQ ID NO: 71 - protein, 258 aa, Artificial Sequence
  MCP-1 (1) scFv antibody
  VARIANT (215)..(215): Any amino acid
  VARIANT (236)..(236): Any amino acid
  VARIANT (243)..(243): Any amino acid
  VARIANT (251)..(252): Any amino acids
EVQLLESGGGLVQPGGSLRLSCAASGFTFSSYAMSWVRQAPGKGLEWVSGVSWNGSRTHYADSVKGRFTISRDNSKNTLYLQMNSLRAEDTAVYYCARGGHQQLGQWGQGTLVTVSSGGGGSGGGGSGGGGSQSVLTQPPSASGTPGQRVTISCSGSSSNIGNNYVSWYQQLPGTAPKLLIYRDSRRPSGVPDRFSGSKSGTSASLAISGLRSEXEADYYCAAWDDSLKGWLFGGXTKLTVLXEQKLISEXXLSGSAA

SEQ ID NO: 72 - protein, 261 aa, Artificial Sequence
  MCP-1 (2) scFv antibody
  VARIANT (239)..(239): Any amino acid
  VARIANT (246)..(246): Any amino acid
  VARIANT (254)..(254): Any amino acid
EVQLLESGGGLVQPGGSLRLSCAASGFTFSSYAMSWVRQAPGKGLEWVSYISSSSSYTNYADSVKGRFTISRDNSKNTLYLQMNSLRAEDTAVYYCARFRYNSGKMFDYWGQGTLVTVSSGGGGSGGGGSGGGGSQSVLTQPPSASGTPGQRVTISCSGSSSNIGRNTVNWYQQLPGTAPKLLIYGNSNRRSGVPDRFSGSKSGTSASLAISGLRSEDEADYYCAAWDDSLSGVVFGGXTKLTVLXEQKLISEXDLSGSAA

SEQ ID NO: 73 - protein, 261 aa, Artificial Sequence
  MCP-1 (3) scFv antibody
  VARIANT (219)..(219): Any amino acid
  VARIANT (239)..(239): Any amino acid
  VARIANT (253)..(253): Any amino acid
EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCAKSHYYDTTSFDYWGQGTLVTVSSGGGGSGGGGSGGGGSQSVLTQPPSASGTPGQRVTISCSGSSSNIGTNPVNWYQQLPGTAPKLLIYDNNKRPSGVPDRFSGSKSGTSASLAISGLRSEDXADYYCAAWDDSLSGVVFGGXTKLTVLGEQKLISXEDLSGSAA

SEQ ID NO: 74 - protein, 263 aa, Artificial Sequence
  MCP-3 (1) scFv antibody
  VARIANT (222)..(222): Any amino acid
  VARIANT (241)..(241): Any amino acid
EVQLLESGGGLVQPGGSLRLSCAASGFTFSTYGMHWVRQAPGKGLEWVSGVSWNGSRTHYVNSVKRRFTISRDNSKNTLYLQMNSLRAEDTAVYYCARVAPGSGKRLRAFDIWGQGTLVTVSSGGGGSGGGGSGGGGSQSVLTQPPSASGTPGQRVTISCSGSSSNIGNNAVNWYQQLPGTAPKLLIYEVSKRPPGVPDRFSGSKSGTSASLAISGLRSEDXADYYCSSYAGSSKWVFGGXTKLTVLGEQKLISEEDLSGSAA

SEQ ID NO: 75 - protein, 256 aa, Artificial Sequence
  MCP-3 (2) scFv antibody
  VARIANT (234)..(234): Any amino acid
  VARIANT (248)..(250): Any amino acids
EVQLLESGGGLVQPGGSLRLSCAASGFTLSSNYMSWVRQAPGKGLEWVSGISASGHSTHYADSGKARFTISRDNSKNTLYLQMNSLRAEDTAVYYCARGKSLAYWGQGTLVTVSSGGGGSGGGGSGGGGSQSVLTQPPSASGTPGQRVTISCSGSSSNIGNNAVNWYQQLPGTAPKLLIYRNNQRPSGVPDRFSGSKSGTSASLAISGLRSEDEADYYCAAWDDSLSVVVFGGXTKLTVLGEQKLISXXXLSGSAA

SEQ ID NO: 76 - protein, 261 aa, Artificial Sequence
  MCP-3 (3) scFv antibody
  VARIANT (221)..(221): Any amino acid
  VARIANT (238)..(239): Any amino acids
  VARIANT (254)..(255): Any amino acids
EVQLLESGGGLVQPGGSLRLSCAASGFTFSIYWMSWVRQAPGKGLEWVAYIGGISNTVSYSDSVKGRFTISRDNSKNTLYLQMNSLRAEDTAVYYCAKAPGYSSGWGWFDPWGQGTLVTVSSGGGGSGGGGSGGGGSQSVLTQPPSASGTPGQRVTISCSGSSSNIGTNSVFWYQQLPGTAPKLLIYGNNNRPSGVPDRFSGSKSGTSASLAISGLRSEDXADYYCMIWHSSASVFGXXTKLTVLGEQKLISEXXLSGSAA

SEQ ID NO: 77 - protein, 252 aa, Artificial Sequence
  beta-gal scFv antibody
  VARIANT (200)..(200): Any amino acid
  VARIANT (235)..(235): Any amino acid
  VARIANT (243)..(243): Any amino acid
EVQLLESGGGLVQPGGSLRLSCAASGFTFSSYAMHWVRQAPGKGLEWVAVIAYDGINEYYGDSVKGRFTISRDNSKNTLYLQMNSLRAEDTAVYYCARGGIYHGFDIWGQGTLVTVSSGGGGSGGGGSGGGGSQSVLTQPPSASGTPGQRVTISCSGSSSNIGSNYVYWYQQLPGTAPKLLIYDNHKRPSGVPDRFSGSXSGTSASLAISGLRSEDEADYYCAAWDDNSWVFGGXTKLTVLGXYKDDDDKAA

SEQ ID NO: 78 - protein, 305 aa, Artificial Sequence
  Angiomotin (1) scFv antibody
  VARIANT (201)..(201): Any amino acid
  VARIANT (218)..(218): Any amino acid
  VARIANT (238)..(238): Any amino acid
  VARIANT (248)..(248): Any amino acid
  VARIANT (261)..(262): Any amino acids
  VARIANT (267)..(268): Any amino acids
  VARIANT (281)..(281): Any amino acid
  VARIANT (292)..(292): Any amino acid
  VARIANT (294)..(295): Any amino acids
  VARIANT (298)..(298): Any amino acid
  VARIANT (300)..(302): Any amino acids
  VARIANT (304)..(305): Any amino acids
EVQLLESGGGLVQPGGSLRLSCAASGFTFSDHYMDWVRQAPGKGLEWVSGVSWNGSRTHYADSVKGRFTISRDNSKNTLYLQMNSLRAEDTAVYYCARDTWAYGAFDIWGQGTLVTVSSGGGGSGGGGSGGGGSQSVLTQPPSASGTPGQRVTISCSGSNSNIGRNTVNWYQQLPGTAPKLLIYRDNQRPSGVPDRFSGSXSGTPASLAISGLRSEDXADYYCAAWDVSLNGWVFGGXTKLTVLGDYXDHDGDYKDHDIDXXDDDDXXAAHHHHHHSPRWXIRPIVSRITIXWXXFYXVXXXKXX

SEQ ID NO: 79 - protein, 320 aa, Artificial Sequence
  Angiomotin (2) scFv antibody
  VARIANT (237)..(237): Any amino acid
  VARIANT (245)..(245): Any amino acid
  VARIANT (265)..(265): Any amino acid
  VARIANT (268)..(268): Any amino acid
  VARIANT (279)..(281): Any amino acids
  VARIANT (283)..(283): Any amino acid
  VARIANT (287)..(287): Any amino acid
  VARIANT (289)..(289): Any amino acid
  VARIANT (292)..(294): Any amino acids
  VARIANT (298)..(299): Any amino acids
  VARIANT (304)..(309): Any amino acids
  VARIANT (311)..(312): Any amino acids
  VARIANT (315)..(317): Any amino acids
  VARIANT (319)..(320): Any amino acids
EVQLLESGGGLVQPGGSLRLSCAASGFTFNDYYMTWIRQAPGKGLEWVSYISSSGSTIYYADSVKGRFTISRDNSKNTLYLQMNSLRAEDTAVYYCARERLPDVFDVWGQGTLVTVSSGGGGSGGGGSGGGGSQSVLTQPPSASGTPGQRVTISCSGSGSNIGTNSVSWYQQLPGTAPKLLIYFDDLLPSGVPDRFSGSKSGTSASLAISGLRSEDEADYYCAAWDDSLSGVVFGGXTKLTVLGXYKDHDGDYKDHDIDYKDDDXKAXAHHHHHHSPRXXXRXIVSXIXIHXXXFYNXXTGKTXXXXXXIXXAAXXXFXX

SEQ ID NO: 80 - protein, 256 aa, Artificial Sequence
  Leptin scFv antibody
  VARIANT (239)..(239): Any amino acid
  VARIANT (247)..(247): Any amino acid
  VARIANT (251)..(251): Any amino acid
EVQLLESGGGLVQPGGSLRLSCAASGFTFGDFAMSWVRQAPGKGLEWVANIKQDGSVKYYVDSVKGRFTISRDNSKNTLYLQMNSLRAEDTAVYYCARFLAGFYYGMDVWGQGTLVTVSSGGGGSGGGGSGGGGSQSVLTQPPSASGTPGQRVTISCSGSDSNIGGNTVNWYQQLPGMAPKLLIYYDDLLPSGVPDRFSGSKSGTSASLAISGLRSEDEADYYCAAYDDTMNGWGFGGXTKLTVLGXYKDXDDKAA

SEQ ID NO: 81 - protein, 261 aa, Artificial Sequence
  Integrin alpha-10 scFv antibody
  VARIANT (239)..(239): Any amino acid
  VARIANT (254)..(254): Any amino acid
EVQLLESGGGLVQPGGSLRLSCAASGFTFSTYNMNWVRQAPGKGLEWVSTISGSGGRTYYADSVKGRFTISRDNSKNTLYLQMNSLRAEDTAVYYCARDRVATLDAFDIWGQGTLVTVSSGGGGSGGGGSGGGGSQSVLTQPPSASGTPGQRVTISCSGSSSNIGSNSVSWYQQLPGTAPKLLIYSNNQRPSGVPDRFSGSKSGTSASLAISGLRSEDEADYYCAAWDDSLSGVVFGGXTKLTVLGEQKLISEXDLSGSAA

SEQ ID NO: 82 - protein, 262 aa, Artificial Sequence
  Integrin alpha-11 scFv antibody
  VARIANT (202)..(202): Any amino acid
  VARIANT (232)..(232): Any amino acid
  VARIANT (240)..(240): Any amino acid
  VARIANT (247)..(247): Any amino acid
  VARIANT (255)..(256): Any amino acids
EVQLLESGGGLVQPGGSLRLSCAASGFTFRRDWMSWVRQVPGKGLEWVSVISGSDGSTYYADSVKGRFTISRDNSKNTLYLQMNSLRAEDTAVYYCASYSPLGNWFDSWGQGTLVTVSSGGGGSGGGGSGGGGSQSVLTQPPSASGTPGQRVTISCTGSSSNIGAGYDVHWYQQLPGTAPKLLIYSDTYRPSGVPDRFSGSXSGTSASLAISGLRSEDEADYYCQSYDSSLXGFVVFGGXTKLTVLXEQKLISEXXLSGSAA

SEQ ID NO: 83 - protein, 257 aa, Artificial Sequence
  IgM (1) scFv antibody
  VARIANT (235)..(235): Any amino acid
  VARIANT (239)..(239): Any amino acid
  VARIANT (249)..(251): Any amino acids
EVQLLESGGGLVQPGGSLRLSCAASGFTFSDYYMSWIRQAPGKGLEWVSAIGSGPYYAHSVRDRFTISRDNSKNTLYLQMNSLRAEDTAVYYCARGGVEASFDYWGQGTLVTVSSGGGGSGGGGSGGGGSQSVLTQPPSASGTPGQRVTISCTGSSSNIGAGYDVHWYQQLPGTAPKLLIYGNTNRPSGVPNRFSGSKSGTSASLAISGLRSEDEADYYCQSYDNDLSGWVFGGXTKLXVLGEQKLISXXXLSGSAA

SEQ ID NO: 84 - protein, 261 aa, Artificial Sequence
  LDL (1) scFv antibody
  VARIANT (254)..(255): Any amino acids
EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCARAARYSYYYYGMDVWGQGTLVTVSSGGGGSGGGGSGGGGSQSVLTQPPSASGTPGQRVTISCSGSSSNIGNNAVNWYQQLPGTAPKLLIYGNDRRPSGVPDRFSGSKSGTSASLAISGLRSEDEADYYCQTWGTGRGVFGGGTKLTVLGEQKLISEXXLSGSAA

SEQ ID NO: 85 - protein, 275 aa, Artificial Sequence
  LDL (2) scFv antibody
  VARIANT (198)..(198): Any amino acid
  VARIANT (207)..(207): Any amino acid
  VARIANT (224)..(224): Any amino acid
  VARIANT (244)..(244): Any amino acid
  VARIANT (251)..(252): Any amino acids
  VARIANT (261)..(261): Any amino acid
  VARIANT (270)..(271): Any amino acids
EVQLLESGGGLVQPGGSLRLSCAASGFTFSNAWMSWVRQAPGKGLEWVSSISTSSNYIYYADSVKGRFTISRDNSKNTLYLQMNSLRAEDTAVYYCARVKKYSSGWYSNYAFDIWGQGTLVTVSSGGGGSGGGGSGGGGSQSVLTQPPSASGTPGQRVTISCSGSSSSIGNNFVSWYQQLPGTAPKLLIYDNNKRPSXVPDRFSGSXSGTSASLAISGLRSEDXADYYCAAWDDSLNGWVFGGXTKLTVLXXYKDHDGDYXDHDIDYKDXXDKAA

SEQ ID NO: 86 - protein, 262 aa, Artificial Sequence
  PSA scFv antibody
  VARIANT (207)..(207): Any amino acid
  VARIANT (245)..(245): Any amino acid
  VARIANT (257)..(258): Any amino acids
EVQLLESGGGLVQPGGSLRLSCAASGFTFRSYEMNWVRQAPGKGLEWVAVIGGNGVDTDYADSVKGRFTISRDNSKNTLYLQMNSLRAEDTAVYYCVREEVDFWSGYYSYGMDVWGQGTLVTVSSGGGGSGGGGSGGGGSQSVLTQPPSASGTPGQRVTISCSGSSSNIGDNFVSWYQQLPGTAPKLLIYRTNGRPSGVPDRFSGSXSGTSASLAISGLRSEDEADYYCATWDDNLNGRVVFGGXTKLTVLGDYKDXXDKAA

SEQ ID NO: 87 - protein, 255 aa, Artificial Sequence
  Lewis x (1) scFv antibody
  VARIANT (238)..(238): Any amino acid
EVQLLESGGGLVQPGGSLRLSCAASGFTFSNYWMHWVRQAPGKGLEWVANIKEDGSEKYYVDSVKGRFTISRDNSKNTLYLQMNSLRAEDTAVYYCAREGETSFGLDVWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEADYYCASWDDSLSGWVFGGXTKLTVLGDYKDDDDKAA

SEQ ID NO: 88 - protein, 258 aa, Artificial Sequence
  Lewis x (2) scFv antibody
  VARIANT (219)..(219): Any amino acid
  VARIANT (241)..(241): Any amino acid
  VARIANT (249)..(249): Any amino acid
  VARIANT (253)..(254): Any amino acids
EVQLLESGGGLVQPGGSLRLSCAASGFTFSRYWMHWVRQAPGKGLEWVANIKPDGSEQYYADSVKGRFTISRDNSKNTLYLQMNSLRAEDTAVYYCAREGLSSGWSYGMDVWGQGTLVTVSSGGGGSGGGGSGGGGSQSVLTQPPSASGTPGQRVTISCSGSNSNIGSNTVNWYQQLPGTAPKLLIYTNINRPSGVPDRFSGSKSGTSASLAISGLRSXDEADYYCATWDDSLSGWVFGGXTKLTVLGXYKDXXDKAA

SEQ ID NO: 89 - protein, 258 aa, Artificial Sequence
  Lewis y scFv antibody
  VARIANT (220)..(220): Any amino acid
  VARIANT (241)..(241): Any amino acid
  VARIANT (248)..(248): Any amino acid
  VARIANT (252)..(252): Any amino acid
EVQLLESGGGLVQSGGSLRLSCAASGFTFSSYTLHWVRQAPGKGLEYVSAISSNGGSTYYADSVKGRFTISRDNSKNTLYLQMNSLRAEDTAVYYCASDVYGDYPRGLDYWGQGTLVTVSSGGGGSGGGGSGGGGSQSVLTQPPSASGTPGQRVTISCSGTTSNIGSNYVHWYQQLPGTAPKLLIYGNNNRPSGVPDRFSGSKSGTSASLAISGLRSEDXADYYCQSYDRSLGGLRVFGGXTKLTVLXDYKXDDDKAA

SEQ ID NO: 90 - protein, 294 aa, Artificial Sequence
  Sialle x scFv antibody
  VARIANT (217)..(217): Any amino acid
  VARIANT (237)..(237): Any amino acid
  VARIANT (252)..(252): Any amino acid
  VARIANT (258)..(258): Any amino acid
  VARIANT (265)..(272): Any amino acids
  VARIANT (274)..(291): Any amino acids
  VARIANT (293)..(294): Any amino acids
EVQLLESGGGLVQPGGSLRLSCAASGFTLSSYAMSWVRQAPGKGLEWVSSISSGNSYIYYADSVKGRFTISRDNSKNTLYLQMNSLRAEDTAVYYCARGRGRGGGFELWGQGTLVTVSSGGGGSGGGGSGGGGSQSVLTQPPSASGTPGQRVTISCSGSSSNIGTYTVNWYQQLPGTAPKLLIYSNNQRPSGVPDRFSGSKSGTSASLAISGLRSEXEADYYCSSNAGIDNILFGGXTKLTVLGEQKLISEXDLSGSXAAHHHHXXXXXXXXIXXXXXXXXXXXXXXXXXXLXX

SEQ ID NO: 91 - protein, 264 aa, Artificial Sequence
  TM peptide scFv antibody
  VARIANT (158)..(158): Any amino acid
  VARIANT (188)..(188): Any amino acid
  VARIANT (196)..(197): Any amino acids
  VARIANT (207)..(207): Any amino acid
  VARIANT (233)..(233): Any amino acid
  VARIANT (242)..(243): Any amino acids
  VARIANT (246)..(246): Any amino acid
  VARIANT (255)..(257): Any amino acids
  VARIANT (261)..(261): Any amino acid
EVQLLESGGGLVQPGGSLRLSCAASGFTFSSYGFHWVRQAPGKGLEWVSLISWDGGSTYYADSVKGRFTISRDNSKNTLYLQMNSLRAEDTAVYYCARGTWFDPWGQGTLVTVSSGGGGSGGGGSGGGGSQSVLTQPPSASGTPGQRVTISCSGSSSXIGNNAVNWYQQLPGTAPKLLIYRNNQRPSXVPDRFSGXXSGTSASLAIXGLRSEDEADYYCAAWDDSLSWVFGGXTKLTVLGDXXTMXVIIKIMTSXXXMTMXRRP

SEQ ID NO: 92 - protein, 254 aa, Artificial Sequence
  Procathepsin W scFv antibody
  VARIANT (200)..(200): Any amino acid
  VARIANT (237)..(237): Any amino acid
  VARIANT (245)..(245): Any amino acid
  VARIANT (248)..(248): Any amino acid
EVQLLESGGGLVQPGGSLRLSCAASGFTFSSYAMSWVRQAPGKGLEWVSSMSASGGSTYYADSVKGRFTISRDNSKNTLYLQMNSLRAEDTAVYYCARDRGSYGMDVWGQGTLVTVSSGGGGSGGGGSGGGGSQSVLTQPPSASGTPGQRVTISCSGSTSNIGSYAVNWYQQLPGTAPKLLIYGNNNRPSGVPDRFSGSXSGTSASLAISGPRSEDEADYYCAAWDDSLNGGVFGGXTKLTVLGXYKXDDDKAA

SEQ ID NO: 93 - protein, 188 aa, Artificial Sequence
  BTK (1) scFv antibody
  VARIANT (166)..(166): Any amino acid
EVQLLESGGGLVQPGGSLRLSCAASGFTFSNYAMSWVRQAPGKGLEWVSGINWNGGSTGYADSVKGRFTISRDNSKNTLYLQMNSLRAEDTAVYYCAKHLKRYSGSSYLFDYWGQGTLVTVSSGGGGSGGGGSGGGGSQSVLTQPPSASGTPGQRVTISCSGSSSXIGSNYVYWYQQLPGTAPKLLIY

SEQ ID NO: 94 - protein, 258 aa, Artificial Sequence
  Digoxin scFv antibody
  VARIANT (236)..(236): Any amino acid
  VARIANT (250)..(252): Any amino acids
  VARIANT (255)..(255): Any amino acid
EVQLLESGGGLVQPGGSLRLSCAASGFTFSSYAMSWVRQAPGKGLEWVAVIWHDGSSKYYADSVKGRFTISRDNSKNTLYLQMNSLRAEDTAVYYCARATGDGFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLNGVVFGGXTKLTVLGEQKLISXXXLSXSAA

SEQ ID NO: 95 - protein, 262 aa, Artificial Sequence
  GLP-1 R scFv antibody
  VARIANT (245)..(245): Any amino acid
EVQLLESGGGLVQPGGSLRLSCAASGFTFRSYGMHWVRQAPGKGLEWVSGLSWNSAGTGYADSVKGRFTISRDNSKNTLYLQMNSLRAEDTAVYYCAKEMGNNWDHIDYWGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCAAWDDGLSGPVFGGGTKLTXLGEQKLISEEDLSGSAA

SEQ ID NO: 96 - protein, 260 aa, Artificial Sequence
  GLP-1 scFv antibody
  VARIANT (238)..(238): Any amino acid
  VARIANT (245)..(245): Any amino acid
  VARIANT (252)..(252): Any amino acid
  VARIANT (254)..(254): Any amino acid
EVQLLESGGGLVQPGGSLRLSCAASGFTFNSYGMHWVRQAPGKGLEWVSAISGSGGSTYYAESVKGRSTISRDNSKNTLYLQMNSLRAEDTAVYYCVTRNAVFGFDVWGQGTLVTVSSGGGGSGGGGSGGGGSQSVLTQPPSASGTPGQRVTISCTGSSSNIGAGFDVHWYQQLPGTAPKLLIYDNNKRPSGVPDRFSGSKSGTSASLAISGLRSEDEADYYCQSFDSSLSGVVFGGXTKLTVLXEQKLISXEXLSGSAA

SEQ ID NO: 97 - protein, 320 aa, Artificial Sequence
  C1q scFv antibody
  VARIANT (197)..(197): Any amino acid
  VARIANT (223)..(223): Any amino acid
  VARIANT (244)..(244): Any amino acid
  VARIANT (259)..(260): Any amino acids
  VARIANT (267)..(268): Any amino acids
  VARIANT (283)..(283): Any amino acid
  VARIANT (286)..(286): Any amino acid
  VARIANT (289)..(290): Any amino acids
  VARIANT (295)..(300): Any amino acids
  VARIANT (302)..(303): Any amino acids
  VARIANT (306)..(307): Any amino acids
  VARIANT (309)..(311): Any amino acids
  VARIANT (313)..(313): Any amino acid
  VARIANT (315)..(320): Any amino acids
EVQLLESGGGLVQPGGSLRLSCAASGFTFDDYGMSWVRQVPGKGLEWVSAISGSGATTFYAHSVQGRFTISRDNSKNTLYLQMNSLRAEDTAVYYCARGGRGYDWPSGAFDIWGQGTLVTVSSGGGGSGGGGSGGGGSQSVLTQPPSASGTPGQRVTISCTGSSSNIGAGYDVHWYQQLPGTAPKLLIYENNKRPSXVPDRFSGSKSGTSASLAISGLRSEDXADYYCAAWDDSVNGYVVFGGXTKLTVLGEQKLISEXXLSGSAAXXHHHHHSPRWPIRPIXSRXTIXXPSFYXXXXXXTXXLPXXIXXXHXPXXXXXX

SEQ ID NO: 98 - protein, 262 aa, Artificial Sequence
  C1s scFv antibody
  VARIANT (202)..(202): Any amino acid
  VARIANT (240)..(241): Any amino acids
  VARIANT (247)..(247): Any amino acid
  VARIANT (250)..(250): Any amino acid
  VARIANT (254)..(256): Any amino acids
EVQLLESGGGLVQPGGSLRLSCAASGFTFSSYAMSWVRQAPGKGLEWVSGVSWNGSRTHYADSVKGRFTISRDNSKNTLYLQMNSLRAEDTAVYYCARHMKAAAYVFEIWGQGTLVTVSSGGGGSGGGGSGGGGSQSVLTQPPSASGTPGQRVTISCSGSSSNIGSTAVNWYQQLPGTAPKLLIYSNNKRPSGVPDRFSGSXSGTSASLAISGLRSEDEADYYCAAWDDRLNGNVLFGGXXKLTVLXEQXLISXXXLSGSAA

SEQ ID NO: 99 - protein, 261 aa, Artificial Sequence
  C3 (1) scFv antibody
  VARIANT (239)..(239): Any amino acid
  VARIANT (253)..(255): Any amino acids
EVQLLESGGGLVQPGGSLRLSCAASGFTFSSYAMSWVRQAPGKGLEWVSSVTGSGGGTYYADSVEGRFTISRDNSKNTLYLQMNSLRAEDTAVYYCARYRWFGNDAFDIWGQGTLVTVSSGGGGSGGGGSGGGGSQSVLTQPPSASGTPGQRVTISCSGSASNLGMHFVSWYQQLPGTAPKLLIYGNSNRPSGVPDRFSGSKSGTSASLAISGLRSEDEADYYCAAWDDTLNIWVFGGXTKLTVLGEQKLISXXXLSGSAA

SEQ ID NO: 100 - protein, 263 aa, Artificial Sequence
  C3 (2) scFv antibody
  VARIANT (220)..(220): Any amino acid
  VARIANT (241)..(241): Any amino acid
  VARIANT (249)..(249): Any amino acid
EVQLLESGGGLVQPGGSLRLSCAASGFTFSTYRMIWVRQAPGKGLEWVSSISGSNTYIHYADSVRGRFTISRDNSKNTLYLQMNSLRAEDTAVYYCARDRHPLLPSGMDVWGQGTLVTVSSGGGGSGGGGSGGGGSQSVLTQPPSASGTPGQRVTISCSGSSSNIGKHPVNWYQQLPGTAPKLLIYRNDQRPSGVPDRFSGSKSGTSASLAISGLRSEDXADYYCQSYDSSLSGSWVFGGXTKLTVLGXQKLISEEDLSGSAA

SEQ ID NO: 101 - protein, 263 aa, Artificial Sequence
  C4 (1) scFv antibody
  VARIANT (241)..(241): Any amino acid
  VARIANT (248)..(248): Any amino acid
  VARIANT (256)..(257): Any amino acids
EVQLLESGGGLVQPGGSLRLSCAASGFTFSSYPMSWVRQAPGKGLEWVSTLYAGGWTSYADSVWGRFTISRDNSKNTLYLQMNSLRAEDTAVYYCARPKVESLSRYGMDVWGQGTLVTVSSGGGGSGGGGSGGGGSQSVLTQPPSASGTPGQRVTISCTGSSSNIGAGYVVHWYQQLPGTAPKLLIYDNSKRPSGVPDRFSGSKSGTSASLAISGLRSEDEADYYCQSYDSSLSGVVFGGXTKLTVLXEQKLISEXXLSGSAA

SEQ ID NO: 102 - protein, 264 aa, Artificial Sequence
  C5 (1) scFv antibody
  VARIANT (196)..(196): Any amino acid
  VARIANT (205)..(205): Any amino acid
  VARIANT (215)..(215): Any amino acid
  VARIANT (222)..(222): Any amino acid
  VARIANT (240)..(240): Any amino acid
  VARIANT (242)..(243): Any amino acids
  VARIANT (249)..(249): Any amino acid
  VARIANT (257)..(258): Any amino acids
  VARIANT (263)..(263): Any amino acid
EVQLLESGGGLVQPGGSLRLSCAASGFTFSSYRMNWVRQAPGKGLEWVSAISGSGGSTYYADSVKGRFTISRDNSKNTLYLQMNSLRAEDTAVYYCARGGGWFSGHYYFDYWGQGTLVTVSSGGGGSGGGGSGGGGSQSVLTQPPSASGTPGQRVTISCTGATSNIGAGYDVHWYQQLPGTAPKLLIYRNNQRPSXVPDRFSGSXSGTSASLAIXGLRSEDXADYYCQSYDSSLRHWVFXGXXKLTVLXEQKLISEXXLSGSXA

SEQ ID NO: 103 - protein, 258 aa, Artificial Sequence
  C5 (2) scFv antibody
  VARIANT (216)..(216): Any amino acid
  VARIANT (236)..(236): Any amino acid
  VARIANT (243)..(243): Any amino acid
  VARIANT (252)..(252): Any amino acid
EVQLLESGGGLVQPGGSLRLSCAASGFTFSAYSMNWVRQAPGKGLEWVSGVSWNGSRTHYADSVKGRFTISRDNSKNTLYLQMNSLRAEDTAVYYCARENSGFFDYWGQGTLVTVSSGGGGSGGGGSGGGGSQSVLTQPPSASGTPGQRVTISCTGSSSNIGSNTVNWYQQLPGTAPKLLIYGNSNRPSGVPDRFSGSKSGTSASLTISGLRSEDXADYYCAAWDDSLSGWVFGGXTKLTVLXEQKLISEEXLSGSAA

SEQ ID NO: 104 - protein, 261 aa, Artificial Sequence
  C1 inh. (1) scFv antibody
  VARIANT (19)..(19): Any amino acid
  VARIANT (218)..(219): Any amino acids
  VARIANT (239)..(239): Any amino acid
  VARIANT (246)..(246): Any amino acid
  VARIANT (254)..(255): Any amino acids
  VARIANT (258)..(258): Any amino acid
EVQLLESGGGLVQPGGSLXLSCAASGFTFSDYYMSWIRQAPGKGLEWVSGISRGGEYTFYVDSVKGRFTISRDNSKNTLYLQMNSLRAEDTAVYYCARDPGGLDAFDIWGQGTLVTVSSGGGGSGGGGSGGGGSQSVLTQPPSASGTPGQRVTISCTGSSSNIGARYDVQWYQQLPGTAPKLLIYGNSNRPSGVPDRFSGSKSGTSASLAISGLRSEXXADYYCASWDDSLSGPVFGGXTKLTVLXEQKLISEXXLSXSAA

SEQ ID NO: 105 - protein, 262 aa, Artificial Sequence
  Factor B (1) scFv antibody
  VARIANT (219)..(219): Any amino acid
  VARIANT (240)..(240): Any amino acid
  VARIANT (255)..(255): Any amino acid
EVQLLESGGGLVQPGGSLRLSCAASGFTFSSYSMNWVRQAPGKGLEWVAVISYDGRFIYYSDSVKGRFTISRDNSKNTLYLQMNSLRAEDTAVYYCARSYGGNLAMDVWGQGTLVTVSSGGGGSGGGGSGGGGSQSVLTQPPSASGTPGQRVTISCTGSSSNIGAGYDVHWYQQLPGTAPKLLIYDNNKRPSGVPDRFSGSNSGTSASLAISGLRSEDXADYYCAAWDDRLNGRVVFGGXTKLTVLGEQKLISEXDLSGSAA

SEQ ID NO: 106 - protein, 262 aa, Artificial Sequence
  IL-12 (3) scFv antibody
  VARIANT (163)..(163): Any amino acid
  VARIANT (220)..(220): Any amino acid
  VARIANT (240)..(241): Any amino acids
  VARIANT (255)..(255): Any amino acid
EVQLLESGGGLVQPGGSLRLSCAASGFTFSRYGMHWVRQAPGKGLEWVASIRGNARGSFYADSVKGRFTISRDNSKNTLYLQMNSLRAEDTAVYYCAKGDSSGWYFFDYWGQGTLVTVSSGGGGSGGGGSGGGGSQSVLTQPPSASGTPGQRVTISCTGSDSXIGAGFDVHWYQQLPGTAPKLLIYGNNNRPSGVPDRFSGSKSGTSASLAISGLRSEDXADYYCQSYDTSLSGVLFGGXXKLTVLGEQKLISEXDLSGSAA

SEQ ID NO: 107 - protein, 254 aa, Artificial Sequence
  IL-12 (4) scFv antibody
  VARIANT (232)..(232): Any amino acid
  VARIANT (239)..(239): Any amino acid
  VARIANT (246)..(246): Any amino acid
EVQLLESGGGLVQPGGSLRLSCAASGFTFSDYGMHWVRQAPGKGLEWVSTVSGSGDNTYYADSVKGRFTISRDNSKNTLYLQMNSLRAEDTAVYYCTTTWRYWGQGTLVTVSSGGGGSGGGGSGGGGSQSVLTQPPSASGTPGQRVTISCSGSSSNIGSNTVNWYQQLPGTAPKLLIYGNSNRPSGVPDRFSGSKSGTSASLAISGLRSEDEADYYCQSYDSSLSGWVFGGXTKLTVLXEQKLISXEDLSGSAA

SEQ ID NO: 108 - protein, 259 aa, Artificial Sequence
  IL-16 (3) scFv antibody
  VARIANT (3)..(3): Any amino acid
  VARIANT (214)..(215): Any amino acids
  VARIANT (225)..(225): Any amino acid
  VARIANT (237)..(237): Any amino acid
  VARIANT (241)..(241): Any amino acid
EVXLLESGGGLVQPGGSLRLSCAASGFTFSSYGMHWVRQAPGKGLEWVSGINWNGGSTGYADSVKGRFTISRDNSKNTLYLQMNSLRAEDTAVYYCARERGDAFDIWGQGTLVTVSSGGGGSGGGGSGGGGSQSVLTQPPSASGTPGQRVTISCSGSSSNIGSNTVNWYQQLPGTAPKLLIYSDNQRPSGVPDRFSGSKSGTSASLAISGLRSXXEADYYCAAWXDSLNGPWVFGGXTKLXVLGEQKLISEEDLSGSAA

SEQ ID NO: 109 - protein, 263 aa, Artificial Sequence
  IL-18 (3) scFv antibody
  VARIANT (204)..(204): Any amino acid
  VARIANT (220)..(221): Any amino acids
  VARIANT (241)..(241): Any amino acid
  VARIANT (245)..(245): Any amino acid
  VARIANT (255)..(257): Any amino acids
  VARIANT (260)..(260): Any amino acid
EVQLLESGGGLVQPGGSLRLSCAASGFTFSRYGMHWVRQAPGKGLEWVAVISYDGSNKYYADSVKGRFTISRDNSKNTLYLQMNSLRAEDTAVYYCARHGYGDSRSAFDIWGQGTLVTVSSGGGGSGGGGSGGGGSQSVLTQPPSASGTPGQRVTISCTGSSSNIGAGYDVHWYQQLPGTAPKLLIYRNNQRPSGVPDRFSGSXSGTSASLAISGLRSEXXADYYCQSYDSSLSRWVFGGXTKLXVLGEQKLISXXXLSXSAA

SEQ ID NO: 110 - protein, 266 aa, Artificial Sequence
  IL-1a (3) scFv antibody
  VARIANT (169)..(169): Any amino acid
  VARIANT (199)..(199): Any amino acid
  VARIANT (208)..(208): Any amino acid
  VARIANT (227)..(227): Any amino acid
  VARIANT (233)..(233): Any amino acid
  VARIANT (239)..(239): Any amino acid
  VARIANT (244)..(244): Any amino acid
  VARIANT (258)..(260): Any amino acids
EVQLLESGGGLVQPGGSLRLSCAASGFTFSSYSMNWVRQAPGKGLEWVSYISSSSSYTNYADSVKGRFTISRDNSKNTLYLQMNSLRAEDTAVYYCARSVTRRAGYYYYYSGMDVWGQGTLVTVSSGGGGSGGGGSGGGGSQSVLTQPPSASGTPGQRVTISCSGSSSXIGSNTVNWYQQLPGTAPKLLIYRNNQRPSXVPDRFSGSXSGTSASLAISGLRSEDEAXYYCSSXAGSNSXVFGGXTKLTVLGEQKLISXXXLSGSAA

SEQ ID NO: 111 - protein, 260 aa, Artificial Sequence
  IL-6 (3) scFv antibody
  VARIANT (219)..(219): Any amino acid
  VARIANT (238)..(238): Any amino acid
  VARIANT (253)..(254): Any amino acids
EVQLLESGGGLVQPGGSLRLSCAASGFTFSNYGMHWVRQAPGKGLEWVSSITSSGDGTYFADSVKGRFTISRDNSKNTLYLQMNSLRAEDTAVYYCARAGGIAAAYAFDIWGQGTLVTVSSGGGGSGGGGSGGGGSQSVLTQPPSASGTPGQRVTISCSGSSSNVGSNYVYWYQQLPGTAPKLLIYDNNKRPSGVPDRFSGSKSGTSASLAISGLRSEXEADYYCQSYDSSRWVFGGXTKLTVLGEQKLISEXXLSGSAA

SEQ ID NO: 112 - protein, 319 aa, Artificial Sequence
  IL-6 (4) scFv antibody
  VARIANT (145)..(145): Any amino acid
  VARIANT (214)..(214): Any amino acid
  VARIANT (240)..(240): Any amino acid
  VARIANT (247)..(247): Any amino acid
  VARIANT (254)..(255): Any amino acids
  VARIANT (262)..(262): Any amino acid
  VARIANT (270)..(270): Any amino acid
  VARIANT (274)..(275): Any amino acids
  VARIANT (282)..(282): Any amino acid
  VARIANT (287)..(288): Any amino acids
  VARIANT (292)..(292): Any amino acid
  VARIANT (298)..(298): Any amino acid
  VARIANT (300)..(301): Any amino acids
  VARIANT (306)..(308): Any amino acids
  VARIANT (311)..(311): Any amino acid
  VARIANT (314)..(317): Any amino acids
  VARIANT (319)..(319): Any amino acid
EVQLLESGGGLVQPGGSLRLSCAASGFTFSSNYMSWVRQAPGKGLEWVSSISSSSTIYYADSVKGRFTISRDNSKNTLYLQMNSLRAEDTAVYYCARQPASGTYDAFDIWGQGTLVTVSSGGGGSGGGGSGGGGSQSVLTQPPSXSGTPGQRVTISCTGSSSNIGAGYDVHWYQQLPGTAPKLLIYYDDLLPSGVPDRFSGSKSGTSASLAISXLRSEDEADYYCAVWDDSLSGWVFGGXTKLTVLXEQKLISXXDLSGSAXAHHHHHHXSPRXXIRPIVSXITIHXXVVLXRRDWEXPXXTQLNXXXAHXPFXXXXNX

SEQ ID NO: 113 - protein, 259 aa, Artificial Sequence
  IL-8 (3) scFv antibody
  VARIANT (4)..(4): Any amino acid
  VARIANT (215)..(215): Any amino acid
  VARIANT (237)..(237): Any amino acid
  VARIANT (252)..(253): Any amino acids
EVQXLESGGGLVQPGGSLRLSCAASGFTFDDYGMSWVRQAPGKGLEWVSLISWDGGSTYYADSVKGRFTISRDNSKNTLYLQMNSLRAEDTAVYYCARDDLYGMDVWGQGTLVTVSSGGGGSGGGGSGGGGSQSVLTQPPSASGTPGQRVTISCTGSSSNIGAGYDVHWYQQLPGTAPKLLIYDNNKRPSGVPDRFSGSKSGTSASLAISGLRSXDEADYYCAAWDDSLSGWVFGGXTKLTVLGEQKLISEXXLSGSAA

SEQ ID NO: 114 - protein, 261 aa, Artificial Sequence
  MCP-4 (3) scFv antibody
  VARIANT (218)..(218): Any amino acid
  VARIANT (239)..(239): Any amino acid
  VARIANT (243)..(243): Any amino acid
  VARIANT (246)..(246): Any amino acid
  VARIANT (254)..(255): Any amino acids
EVQLLESGGGLVQPGGSLRLSCAASGFTFSSYGMHWVRQAPGKGLEWVSGISWNGGKTHYVDSVKGQFTISRDNSKNTLYLQMNSLRAEDTAVYYCARGGYSSGWAFDYWGQGTLVTVSSGGGGSGGGGSGGGGSQSVLTQPPSASGTPGQRVTISCSGRSSNIESNTVNWYQQLPGTAPKLLIYGNSNRPSGVPDRFSGSKSGTSASLAISGLRSEXEADYYCAAWDDRLNAVVFGGXTKLXVLXEQKLISEXXLSGSAA

SEQ ID NO: 115 - protein, 313 aa, Artificial Sequence
  Properdin scFv antibody
  VARIANT (222)..(222): Any amino acid
  VARIANT (228)..(228): Any amino acid
  VARIANT (244)..(244): Any amino acid
  VARIANT (247)..(247): Any amino acid
  VARIANT (262)..(262): Any amino acid
  VARIANT (273)..(274): Any amino acids
  VARIANT (287)..(288): Any amino acids
  VARIANT (290)..(294): Any amino acids
  VARIANT (298)..(300): Any amino acids
  VARIANT (302)..(302): Any amino acid
  VARIANT (304)..(309): Any amino acids
  VARIANT (313)..(313): Any amino acid
EVQLLESGGGLVQPGGSLRLSCAASGFTFSSNYMSWVRQAPGKGLEWVSAISGSGGSTYYADSVKGRFTISRDNSKNTLYLQMNSLRAEDTAVYYCAKGGSGWYDYFDYWGQGTLVTVSSGGGGSGGGGSGGGGSQSVLTQPPSASGTPGQRVTISCTGSSSNIGAGYDVHWYQQLPGTAPKLLIYRNNQRPSGVPDRFSGSKSGTSASLAISGLRSEDEAXYYCAAXDDGLNSPVFGGGTKLXVLXEQKLISEEDLSGSAXAHHHHHHSPRXXIRPIVSRITIHWXXFXXXXXGKTXXXPXLXXXXXXPPFX

SEQ ID NO: 116 - protein, 255 aa, Artificial Sequence
  TNF-beta (3) scFv antibody
  VARIANT (213)..(213): Any amino acid
  VARIANT (233)..(233): Any amino acid
  VARIANT (248)..(248): Any amino acid
EVQLLESGGGLVQPGGSLRLSCAASGFTFSDYYMSWIRQAPGKGLEWVSGLSGSAGRTHYADSVRGRFTISRDNSKNTLYLQMNSLRAEDTAMYYCASSLFDYWGQGTLVTVSSGGGGSGGGGSGGGGSQSVLTQPPSASGTPGQRVTISCSGSSSNIGSNYVYWYQQLPGTAPKLLIYSNNQRPSGVPDRFSGSKSGTSASLAISGLRSEDXADYYCAAWDDSLNAVVFGGXTKLTVLGEQKLISEXDLSGSAA

SEQ ID NO: 117 - protein, 262 aa, Artificial Sequence
  TNF-beta (4) scFv antibody
  VARIANT (240)..(240): Any amino acid
  VARIANT (254)..(256): Any amino acids
EVQLLESGGGLVQPGGSLRLSCAASGFTFSDYYMNWVRQAPGKGLEWVSGINWNSDDIDYVDSVKGRFTISRDNSKNTLYLQMNSLRAEDTAMYYCAIDSRYSSGWSFEYWGQGTLVTVSSGGGGSGGGGSGGGGSQSVLTQPPSASGTPGQRVTISCSGSTSNIGNSHVYWYQQLPGTAPKLLIYSNNQRPSGVPDRFSGSKSGTSASLAISGLRSEDEADYYCQSYDSSLSGVVFGGXTKLTVLGEQKLISXXXLSGSAA

SEQ ID NO: 118 - protein, 304 aa, Artificial Sequence
  VEGF (3) scFv antibody
  VARIANT (192)..(192): Any amino acid
  VARIANT (201)..(201): Any amino acid
  VARIANT (218)..(218): Any amino acid
  VARIANT (241)..(241): Any amino acid
  VARIANT (244)..(244): Any amino acid
  VARIANT (247)..(247): Any amino acid
  VARIANT (254)..(256): Any amino acids
  VARIANT (261)..(261): Any amino acid
  VARIANT (273)..(273): Any amino acid
  VARIANT (281)..(281): Any amino acid
  VARIANT (283)..(283): Any amino acid
  VARIANT (288)..(288): Any amino acid
  VARIANT (293)..(296): Any amino acids
  VARIANT (298)..(300): Any amino acids
  VARIANT (302)..(302): Any amino acid
  VARIANT (304)..(304): Any amino acid
EVQLLESGGGLVQPGGSLRLSCAASGFTFSSYEMNWVRQAPGKGLEWVSGISGSGGFTYYADSVKGRFTISRDNSKNTLYLQMNSLRAEDTAMYYCAREGYQDAFDIWGQGTLVTVSSGGGGSGGGGSGGGGSQSVLTQPPSASGTPGQRVTISCTGSSSNIGAGYDVHWYQQLPGTAPKLLIYSNNQRPSXVPDRFSGSXSGTSASLAISGLRSEDXADYYCAAWDDSLSGPPWVFGGGXKLXVLXEQKLISXXXLSGSXAAHHHHHHSPRXPIRPIVSXIXIHWPXFYNVXXXXTXXXPXLX

SEQ ID NO: 119 - protein, 261 aa, Artificial Sequence
  VEGF (4) scFv antibody
  VARIANT (30)..(32): Any amino acids
  VARIANT (34)..(34): Any amino acid
  VARIANT (50)..(50): Any amino acid
  VARIANT (54)..(55): Any amino acids
  VARIANT (97)..(104): Any amino acids
  VARIANT (193)..(193): Any amino acid
  VARIANT (202)..(202): Any amino acid
  VARIANT (219)..(219): Any amino acid
  VARIANT (232)..(233): Any amino acids
  VARIANT (239)..(239): Any amino acid
  VARIANT (253)..(255): Any amino acids
EVQLLESGGGLVQPGGSLRLSCAASGFTFXXXYXSWVRQAPGKGLEWVSXISWXXGSIGYADSVKGRFTISRDNSKNTLYLQMNSLRAEDTAVYYCXXXXXXXXNYFDYWGQGTLVTVSSGGGGSGGGGSGGGGSQSVLTQPPSASGTPGQRVTISCSGSNSNIGGNFVYWYQQLPGTAPKLLIYENSKRPSXVPDRFSGSXSGTSASLAISGLRSEDXADYYCAAWDDSLXXVVFGGXTKLTVLGEQKLISXXXLSGSAA

SEQ ID NO: 120 - protein, 260 aa, Artificial Sequence
  IL-4 (3) scFv antibody
  VARIANT (218)..(218): Any amino acid
  VARIANT (238)..(239): Any amino acids
  VARIANT (253)..(253): Any amino acid
EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARAIAARPFDYWGQGTLVTVSSGGGGSGGGGSGGGGSQSVLTQPPSASGTPGQRVTISCTGATSNIGAGYDIHWYQQLPGTAPKLLIYSTNNRPSGVPDRFSGSKSGTSASLAISGLRSEDXADYYCAAWDDSLNGPVFGGXXKLTVLGEQKLISEXDLSGSAA

SEQ ID NO: 121 - protein, 161 aa, Artificial Sequence
  CD40 (2) scFv antibody
EVQLLESGGGLVQPGGSLRLSCAASGFTFSAYWMHWVRQAPGKGLEWVSGISGGGGSTYYADSVKGRFTISRDNSKNTLYLQMNSLRAEDTAVYYCARMTPWYYGMDVWGQGTLVTVSSGGGGSGGGGSGGGGSQSMLTQPPSASGTPGQRVTISCSGSTS

SEQ ID NO: 122 - protein, 156 aa, Artificial Sequence
  CD40 (3) scFv antibody
  VARIANT (143)..(144): Any amino acids
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSGGGGSGGGGSGGGGSQSVLTQPPXXSGTPGQRVTISC

SEQ ID NO: 123 - protein, 256 aa, Artificial Sequence
  CD40 (4) scFv antibody
  VARIANT (219)..(219): Any amino acid
  VARIANT (232)..(232): Any amino acid
  VARIANT (239)..(240): Any amino acids
  VARIANT (246)..(247): Any amino acids
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSGGGGSGGGGSGGGGSQSVLTQPPSASGTPGQRVTISCTGSSSNIGAGYDVYWYQQLPGTAPKLLIYGNINRPSGVPDRFSGSKSGTSASLAISGLRSEDXADYYCAAWDDSLXGLVFGGXXKLTVLXXYKDDDDKAA

SEQ ID NO: 124 - protein, 267 aa, Artificial Sequence
  CT17 scFv antibody
  VARIANT (228)..(228): Any amino acid
  VARIANT (250)..(250): Any amino acid
  VARIANT (257)..(257): Any amino acid
  VARIANT (262)..(262): Any amino acid
  VARIANT (264)..(264): Any amino acid
EVQLLESGGGLVQPGGSLRLSCAASGFTFSSSAMHWVRQAPGKGLEWVSAISGSGGSTYYADSVKGRFTISRDNSKNTLYLQMNSLRAEDTAVYYCARVKGRVTIFGVVINSNYGMDVWGQGTLVTVSSGGGGSGGGGSGGGGSQSVLTQPPSASGTPGQRVTISCSGSISSIGSNAVSWYQQLPGTAPKLLIYGNSNRPSGVPDRFSGSKSGTSASLAISGLRSEDXADYYCAAWDDSLNGHDVVFGGXTKLTVLXDYKDXDXKAA

SEQ ID NO: 125 - protein, 265 aa, Artificial Sequence
  IgM (3) scFv antibody
  VARIANT (168)..(168): Any amino acid
  VARIANT (198)..(198): Any amino acid
  VARIANT (206)..(207): Any amino acids
  VARIANT (217)..(217): Any amino acid
  VARIANT (222)..(222): Any amino acid
  VARIANT (224)..(224): Any amino acid
  VARIANT (232)..(233): Any amino acids
  VARIANT (243)..(243): Any amino acid
  VARIANT (257)..(258): Any amino acids
EVQLLESGGGLVQPGGSLRLSCAASGFTFSSYAMSWVRQAPGKGLEWVSGISWNSGSIGYADSVKGRFTISRDNSKNTLYLQMNSLRAEDTAVYYCARGDYSSSPGGYYYYMDVWGQGTLVTVSSGGGGSGGGGSGGGGSQSVLTQPPSASGTPGQRVTISCSGSSSXIGSNTVNWYQQLPGTAPKLLIYGNSNRPSXVPDRFSGXXSGTSASLAIXGLRSXDXADYYCSSXXSTNTVIFGGXTKLTVLGEQKLISXXDLSGSAA

SEQ ID NO: 126 - protein, 262 aa, Artificial Sequence
  IgM (4) scFv antibody
  VARIANT (193)..(193): Any amino acid
  VARIANT (202)..(202): Any amino acid
  VARIANT (240)..(240): Any amino acid
  VARIANT (244)..(244): Any amino acid
  VARIANT (254)..(255): Any amino acids
EVQLLESGGGLVQPGGSLRLSCAASGFTFSSNEMSWIRQAPGKGLEWVSAIYSGGGTYYADSVKGRFTISRDNSKNTLYLQMNSLRAEDTAVYYCARVNDYGDNVYFDHWGQGTLVTVSSGGGGSGGGGSGGGGSQSVLTQPPSASGTPGQRVTISCSGSSSNIGNNYVSWYQQLPGTAPKLLIYENNKRPSXVPDRFSGSXSGTSASLAISGLRSEDEADYYCAAWDDSLSVYVVFGGXTKLXVLGEQKLISXXDLSGSAA

SEQ ID NO: 127 - protein, 261 aa, Artificial Sequence
  IgM (5) scFv antibody
  VARIANT (193)..(193): Any amino acid
  VARIANT (202)..(202): Any amino acid
  VARIANT (239)..(239): Any amino acid
  VARIANT (246)..(246): Any amino acid
  VARIANT (253)..(253): Any amino acid
  VARIANT (255)..(255): Any amino acid
EVQLLESGGGLVQPGGSLRLSCAASGFTFGSYEMNWVRQAPGKGLEWVSVIYSGGSTYYADSVEGRFTISRDNSKNTLYLQMNSLRAEDTAVYYCARDTNPYYYYGMDVWGQGTLVTVSSGGGGSGGGGSGGGGSQSVLTQPPSASGTPGQRVTISCSGSSSNIGNNAVNWYQQLPGTAPKLLIYRNNQRPSXVPDRFSGSXSGTSASLAISGLRSEDEADYYCQSYDSSLNGQVFGGXTKLTVLXEQKLISXEXLSGSAA

SEQ ID NO: 128 - protein, 259 aa, Artificial Sequence
  HLA-DR/DP scFv antibody
  VARIANT (190)..(190): Any amino acid
  VARIANT (199)..(199): Any amino acid
  VARIANT (216)..(216): Any amino acid
  VARIANT (237)..(237): Any amino acid
  VARIANT (244)..(244): Any amino acid
  VARIANT (251)..(252): Any amino acids
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDGLLPLDYWGQGTLVTVSSGGGGSGGGGSGGGGSQSVLTQPPSASGTPGQRVTISCSGGSSNIGGNAVNWYQQLPGTAPKLLIYENNKRPSXVPDRFSGSXSGTSASLAISGLRSEDXADYYCSSYAVSNNFEVLFGGXTKLTVLXEQKLISXXDLSGSAA

SEQ ID NO: 129 - protein, 308 aa, Artificial Sequence
  ICAM-1 scFv antibody
  VARIANT (160)..(160): Any amino acid
  VARIANT (191)..(191): Any amino acid
  VARIANT (200)..(200): Any amino acid
  VARIANT (237)..(237): Any amino acid
  VARIANT (251)..(252): Any amino acids
  VARIANT (258)..(258): Any amino acid
  VARIANT (274)..(274): Any amino acid
  VARIANT (278)..(279): Any amino acids
  VARIANT (282)..(283): Any amino acids
  VARIANT (285)..(285): Any amino acid
  VARIANT (288)..(288): Any amino acid
  VARIANT (290)..(292): Any amino acids
  VARIANT (295)..(296): Any amino acids
  VARIANT (298)..(298): Any amino acid
  VARIANT (300)..(300): Any amino acid
  VARIANT (302)..(303): Any amino acids
  VARIANT (307)..(308): Any amino acids
EVQLLESGGGLVQPGGSLRLSCAASGFTFSNAWMSWVRQAPGKGLEWVAFIWYDGSNKYYADSVKGRFTISRDNSKNTLYLQMNSLRAEDTAVYYCARYSGWYFDYWGQGTLVTVSSGGGGSGGGGSGGGGSQSVLTQPPSASGTPGQRVTISCTGSSSXIGAGYDVHWYQQLPGTAPKLLIYDNNNRPSXVPDRFSGSXSGTSASLAISGLRSEDEADYYCQSYDSSLSAWLFGGXTKLTVLGEQKLISXXDLSGSXAAHHHHHHSPRWPIRXIVSXXTIXXPXFYXVXXXKPXXTXLXRXXAHPXX

SEQ ID NO: 130 - protein, 257 aa, Artificial Sequence
  IgM (2) scFv antibody
  VARIANT (189)..(189): Any amino acid
  VARIANT (198)..(198): Any amino acid
  VARIANT (235)..(235): Any amino acid
  VARIANT (251)..(251): Any amino acid
EVQLLESGGGLVQPGGSLRLSCAASGFTFSDYYMSWIRQAPGKGLEWVSAIGSGPYYAHSVRDRFTISRDNSKNTLYLQMNSLRAEDTAVYYCARGGVEASFDYWGQGTLVTVSSGGGGSGGGGSGGGGSQSVLTQPPSASGTPGQRVTISCTGSSSNIGAGYDVHWYQQLPGTAPKLLIYGNTNRPSXVPNRFSGSXSGTSASLAISGLRSEDEADYYCQSYDNDLSGWVFGGXTKLTVLGEQKLISEEXLSGSAA

SEQ ID NO: 131 - protein, 112 aa, Artificial Sequence
  MCP-1 (4) scFv antibody
QSVLTQPPSASGTPGQRVTISCTGSSSNIGSDYGVQWYQQLPGTAPKLLIYSNNQRPSGVPDRFSGSKSGTSASLAISGLRSEDEADYYCAAWDDSLSGPVFGGGTKLTVLG

SEQ ID NO: 132 - protein, 112 aa, Artificial Sequence
  MCP-1 (5) scFv antibody
QSVLTQPASASGTPGQRVTISCTGNSSNIGAGYDVHWYQQLPGTAPKLLIYRNNQRPSGVPDRFSGSKSGTSASLAISGLLSEDEADYYCAAWDYSLNGWVFGGGTKLTVLG

SEQ ID NO: 133 - protein, 113 aa, Artificial Sequence
  MCP-1 (6) scFv antibody
QSVLTQPSSASGTPGQRVTISCTGNSSNIGAGYDVHWYQQLPGTAPNLLIYRNNQRPSGVPDRFSGSKSGTSASLAISGLRSEDEADYYCAAWDDSLNGWVFGGGTKLTVLGQ

SEQ ID NO: 134 - protein, 242 aa, Artificial Sequence
  MCP-1 (7) scFv antibody
  VARIANT (196)..(196): Any amino acid
  VARIANT (222)..(222): Any amino acid
  VARIANT (242)..(242): Any amino acid
EVQLLESGGGLVQPGGSLRLSCAASGFTFSSYAMSWVRQAPGKGLEWVAYINRGSTYTNYADSMKGRFTISRDNSKNTLYLQMNSLRAEDTAVYYCARRGYGSGSYYAFDIWGQGTLVTVSSGGGGSGGGGSGGGGSQSVLTQPPSASGTPGQRVTISCTGSSSNIGSDYGVQWYQQLPGTAPKLLIYSNNQRPSXVPDRFSGSKSGTSASLAISGLRSEDXADYYCAAWDDSLSGPVFGGX

SEQ ID NO: 135 - protein, 240 aa, Artificial Sequence
  MCP-1 (8) scFv antibody
  VARIANT (195)..(195): Any amino acid
  VARIANT (204)..(204): Any amino acid
  VARIANT (221)..(221): Any amino acid
EVQLLESGGGLVQPGGSLRLSCAASGFTFGDYAMSWVRQAPGKGLEWVAVISYDGSNKYYADSVKGRFTISRDNSKNTLYLQMNSLRAEDTAVYYCARDPDPSGTDAFDFWGQGTLVTVSSGGGGSGGGGSGGGGSQSVLTQPPSASGTPGQRVTISCSGSSSNIGAGYDVHWYQQLPGTAPKLLIYGNSNRPSXVPDRFSGSXSGTSASLAISGLRSEDXADYYCAAWDDSLSAWAFGG

SEQ ID NO: 136 - protein, 237 aa, Artificial Sequence
  MCP-1 (9) scFv antibody
  VARIANT (192)..(193): Any amino acids
  VARIANT (202)..(202): Any amino acid
EVQLLESGGGLVQPGGSLRLSCAASGFTFSSYEMNWVRQAPGKGLEWVSAISGPGGSTYYADSVKGRFTISRDNSKNTLYLQMNSLRAEDTAVYYCARDLSDYGDFDYWGQGTLVTVSSGGGGSGGGGSGGGGSQSVLTQPPSASGTPGQRVTISCTGSSSNIGAGYDAHWYQQLPGTAPKLLIYDNNKRPXXVPDRFSGSXSGTSASLAISGLRSEDEADYYCATWDDSLRGWVFG

SEQ ID NO: 137 - protein, 268 aa, Artificial Sequence
  Cystatin C (1) scFv antibody
  VARIANT (191)..(191): Any amino acid
  VARIANT (200)..(200): Any amino acid
  VARIANT (237)..(237): Any amino acid
  VARIANT (247)..(247): Any amino acid
  VARIANT (254)..(254): Any amino acid
  VARIANT (260)..(261): Any amino acids
EVQLLESGGGLVQPGGSLRLSCAASGFTFSSYAMNWVRQAPGKGLEWVGLISYDGRTTYYADSVKGRSTISRDNSKNTLYLQMNSLRAEDTAVYYCATTTGTTLDYWGQGTLVTVSSGGGGSGGGGSGGGGSQSVLTQPPSASGTPGQRVTISCTGSSSNIGAGYDVHWYQQLPGTAPKLLIYGNTNRPSXVPDRFSGSXSGTSASLAISGLRSEDEADYYCAAWDDSLYGWVFGGXTKLTVLGDYXDHDGDYXDHDIDXXDDDDKAA

SEQ ID NO: 138 - protein, 192 aa, Artificial Sequence
  Cystatin C (2) scFv antibody
EVQLLESGGGLVQPGGSLRLSCAASGFTFSSYSMNWVRQAPGKGLEWVAFISYDGSNKYYVDSVKGRFTISRDNSKNTLYLQMNSLRAEDTAVYYCARDGVPAVPFDYWGQGTLVTVSSGGGGSGGGGSGGGGSQSVLTQPPSASGTPGQRVTISCSGSSSNIGAGYDVHWYQQLPGTAPKLLIYSNNQRPS

SEQ ID NO: 139 - protein, 196 aa, Artificial Sequence
  Cystatin C (3) scFv antibody
  VARIANT (167)..(167): Any amino acid
EVQLLESGGGLVQPGGSLRLSCAASGFTFSNYAMTWVRQAPGKGLEWVADISHDGFHKYYADSVRGRFTISRDNSKNTLYLQMNSLRAEDTAVYYCARYGRVLPYYYYYGMDVWGQGTLVTVSSGGGGSGGGGSGGGGSQSVLTQPPSASGTPRQRVTISCTGSSSXIGAGYDVHWYQQLPGTAPKLLIYGNSNRP

SEQ ID NO: 140 - protein, 236 aa, Artificial Sequence
  Cystatin C (4) scFv antibody
  VARIANT (191)..(191): Any amino acid
  VARIANT (200)..(200): Any amino acid
  VARIANT (205)..(205): Any amino acid
  VARIANT (215)..(215): Any amino acid
EVQLLESGGGLVQPGGSLRLSCAASGFTFSSYAMNWVRQAPGKGLEWVGLISYDGRTTYYADSVKGRSTISRDNSKNTLYLQMNSLRAEDTAVYYCATTTGTTLDYWGQGTLVTVSSGGGGSGGGGSGGGGSQSVLTQPPSASGTPGQRVTISCTGSSSNIGAGYDVHWYQQLPGTAPKLLIYGNTNRPSXVPDRFSGSXSGTSXSLAISGLRSXDEADYYCAAWDDSLYGWVFGG

SEQ ID NO: 141 - protein, 187 aa, Artificial Sequence
  Apo-A1 (1) scFv antibody
EVQLLESGGGLVQPGGSLRLSCAASGFTFSNNGMHWVRQAPGKGLEWVSAISASGGSTYYADSVKGRFTISRDNSKNTLYLQMNSLRAEDTAVYYCATHGGSSYDAFDIWGQGTLVTVSSGGGGSGGGGSGGGGSQSVLTQPPSASGTPGQRVTISCTGSSSNIGAGYVVHWYQQLPGTAPKLLIYG

SEQ ID NO: 142 - protein, 196 aa, Artificial Sequence
  Apo-A1 (2) scFv antibody
  VARIANT (166)..(166): Any amino acid
EVQLLESGGGLVQPGGSLRLSCAASGFTFRDYYMSWIRQAPGKGLEWVAVTSYDGSKKYYADSVKGRFTISRDNSKNTLYLQMNSLRAEDTAVYYCAKDYADDSIAAPAFDIWGQGTLVTVSSGGGGSGGGGSGGGGSQSVLTQPPSASGTPGQRVTISCTGSSSXIGAGYDVHWYQQLPGTAPKLLIYGNSNRPS

SEQ ID NO: 143 - protein, 315 aa, Artificial Sequence
  Apo-A1 (3) scFv antibody
  VARIANT (3)..(4): Any amino acids
  VARIANT (221)..(221): Any amino acid
  VARIANT (225)..(225): Any amino acid
  VARIANT (249)..(250): Any amino acids
  VARIANT (252)..(252): Any amino acid
  VARIANT (266)..(266): Any amino acid
  VARIANT (269)..(271): Any amino acids
  VARIANT (273)..(273): Any amino acid
  VARIANT (283)..(285): Any amino acids
  VARIANT (288)..(290): Any amino acids
  VARIANT (292)..(293): Any amino acids
  VARIANT (297)..(304): Any amino acids
  VARIANT (307)..(315): Any amino acids
EVXXLESGGGLVQPGGSLRLSCAASGFTFRDYYMSWIRQAPGKGLEWVAVTSYDGSKKYYADSVKGRFTISRDNSKNTLYLQMNSLRAEDTAVYYCAKDYADDSIAAPAFDIWGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSXDEAXYYCQSYDSSLSVVFGGGTKLTVLXXYXDHDGDYKDHDIDYXDDXXXAXAHHHHHHSPXXXIRXXXSXXTIHXXXXXXXXDWXXXXXXXXX

SEQ ID NO: 144 - protein, 271 aa, Artificial Sequence
  Factor B (2) scFv antibody
  VARIANT (240)..(240): Any amino acid
  VARIANT (250)..(250): Any amino acid
  VARIANT (263)..(263): Any amino acid
  VARIANT (268)..(268): Any amino acid
EVQLLESGGGLVQPGGSLRLSCAASGFTFSSYSMNWVRQAPGKGLEWVAVISYDGRFIYYSDSVKGRFTISRDNSKNTLYLQMNSLRAEDTAVYYCARSYGGNLAMDVWGQGTLVTVSSGGGGSGGGGSGGGGSQSVLTQPPSASGTPGQRVTISCTGSSSNIGAGYDVHWYQQLPGTAPKLLIYDNNKRPSGVPDRFSGSNSGTSASLAISGLRSEDEADYYCAAWDDRLNGRVVFGGXTKLTVLGDYXDHDGDYKDHDIDXKDDDXKAA

SEQ ID NO: 145 - protein, 226 aa, Artificial Sequence
  Factor B (3) scFv antibody
  VARIANT (160)..(160): Any amino acid
  VARIANT (190)..(190): Any amino acid
  VARIANT (199)..(199): Any amino acid
  VARIANT (209)..(209): Any amino acid
  VARIANT (216)..(216): Any amino acid
EVQLLESGGGLVQPGGSLRLSCAASGFTFSSYSMNWVRQAPGKGLEWVAVISYDGSNQYYADSVRGRFTISKDNSKNTLYLQMNSLRAEDTAVYYCAREWHYSLDVWGQGTLVTVSSGGGGSGGGGSGGGGSQSVLTQPPSASGTPGQRVTISCSGSSSXIGSNTVNWYQQLPGTAPKLLIYRNNQRPSXVPDRFSGSXSGTSASLAIXGLRSEDXADYYCAAWDD

SEQ ID NO: 146 - protein, 323 aa, Artificial Sequence
  Factor B (4) scFv antibody
  VARIANT (191)..(192): Any amino acids
  VARIANT (201)..(201): Any amino acid
  VARIANT (206)..(206): Any amino acid
  VARIANT (227)..(227): Any amino acid
  VARIANT (229)..(229): Any amino acid
  VARIANT (238)..(238): Any amino acid
  VARIANT (240)..(240): Any amino acid
  VARIANT (245)..(245): Any amino acid
  VARIANT (247)..(248): Any amino acids
  VARIANT (252)..(253): Any amino acids
  VARIANT (260)..(260): Any amino acid
  VARIANT (262)..(262): Any amino acid
  VARIANT (264)..(264): Any amino acid
  VARIANT (266)..(269): Any amino acids
  VARIANT (288)..(288): Any amino acid
  VARIANT (290)..(290): Any amino acid
  VARIANT (292)..(292): Any amino acid
  VARIANT (294)..(295): Any amino acids
  VARIANT (298)..(298): Any amino acid
  VARIANT (300)..(303): Any amino acids
  VARIANT (305)..(316): Any amino acids
  VARIANT (318)..(323): Any amino acids
EVQLLESGGGLVQPGGSLRLSCAASGFTFSKHSMNWVRQAPGKGLEWVATVSYDGNYKYYADSVKGRFTISRDNSKNTLYLQMNSLRAEDTAVYYCAREGYYYYGMDVWGQGTLVTVSSGGGGSGGGGSGGGGSQSVLTQPPSASGTPGQRVTISCTGSSSNIGNNAVNWYQQLPGTAPKLLIYNNNQRPXXVPDRFSGSXSGTSXSLAISGLRSEDEADYYCQPYXDXLSSVVFGGXTXLTVLXDXXDHDXXYKDHDIXYXDXDXXXXAHHHHHHSPRWPIRPIVSXIXIXWXXVLXRXXXXNXXXXXXXXXXXXHXXXXXX

SEQ ID NO: 147 - protein, 272 aa, Artificial Sequence
  C1 inh. (2) scFv antibody
  VARIANT (203)..(203): Any amino acid
  VARIANT (241)..(241): Any amino acid
  VARIANT (248)..(248): Any amino acid
  VARIANT (251)..(251): Any amino acid
  VARIANT (264)..(265): Any amino acids
  VARIANT (270)..(270): Any amino acid
EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARNRGNWGTYYFDYWGQGTLVTVSSGGGGSGGGGSGGGGSQSVLTQPPSASGTPGQRVTISCSGSSSNIGSNYVSWYQQLPGTAPKLLIYGSSNRPSGVPDRFSGSXSGTSASLAISGLRSEDEADYYCQSYDSSLSDHVVFGGXTKLTVLXDYXDHDGDYKDHDIDXXDDDDXAA

SEQ ID NO: 148 - protein, 272 aa, Artificial Sequence
  C1 inh. (3) scFv antibody
  VARIANT (164)..(164): Any amino acid
  VARIANT (194)..(194): Any amino acid
  VARIANT (203)..(203): Any amino acid
  VARIANT (220)..(220): Any amino acid
  VARIANT (241)..(241): Any amino acid
  VARIANT (251)..(251): Any amino acid
  VARIANT (262)..(262): Any amino acid
  VARIANT (264)..(265): Any amino acids
  VARIANT (268)..(270): Any amino acids
EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARNRGNWGTYYFDYWGQGTLVTVSSGGGGSGGGGSGGGGSQSVLTQPPSASGTPGQRVTISCSGSSSXIGSNYVSWYQQLPGTAPKLLIYGSSNRPSXVPDRFSGSXSGTSASLAISGLRSEDXADYYCQSYDSSLSDHVVFGGXTKLTVLGDYXDHDGDYKDHDXDXXDDXXXAA

SEQ ID NO: 149 - protein, 247 aa, Artificial Sequence
  C1 inh. (4) scFv antibody
  VARIANT (164)..(164): Any amino acid
  VARIANT (194)..(194): Any amino acid
  VARIANT (202)..(203): Any amino acids
  VARIANT (234)..(234): Any amino acid
  VARIANT (241)..(241): Any amino acid
EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARNRGNWGTYYFDYWGQGTLVTVSSGGGGSGGGGSGGGGSQSVLTQPPSASGTPGQRVTISCSGSSSXIGSNYVSWYQQLPGTAPKLLIYGSSNRPSXVPDRFSGXXSGTSASLAISGLRSEDEADYYCQSYDSSLSXHVVFGGXTKLTVL

SEQ ID NO: 150 - protein, 263 aa, Artificial Sequence
  C5 (3) scFv antibody
  VARIANT (210)..(210): Any amino acid
  VARIANT (232)..(232): Any amino acid
  VARIANT (239)..(239): Any amino acid
  VARIANT (243)..(243): Any amino acid
  VARIANT (255)..(255): Any amino acid
  VARIANT (260)..(261): Any amino acids
EVQLLESGGGLVQPGGSLRLSCAASGFTFSSYGMHWVRQAPGKGLEWVSYISSSGSTIYYADSVKGRFTISRDNSKNTLYLQMNSLRAEDTAVYYCLTLGGYWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSXDEADYYCQSYDSSLSGWVFGGXTKLTVLXDYKXHDGDYKDHDIDXKDDDXXAA

SEQ ID NO: 151 - protein, 291 aa, Artificial Sequence
  C4 (2) scFv antibody
  VARIANT (207)..(207): Any amino acid
  VARIANT (212)..(212): Any amino acid
  VARIANT (224)..(224): Any amino acid
  VARIANT (237)..(237): Any amino acid
  VARIANT (244)..(246): Any amino acids
  VARIANT (249)..(249): Any amino acid
  VARIANT (254)..(256): Any amino acids
  VARIANT (261)..(266): Any amino acids
  VARIANT (271)..(275): Any amino acids
  VARIANT (277)..(277): Any amino acid
  VARIANT (279)..(279): Any amino acid
  VARIANT (281)..(283): Any amino acids
  VARIANT (289)..(290): Any amino acids
EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCAGYGSGSRATGYNWFAPWGQGTLVTVSSGGGGSGGGGSGGGGSQSVLTQPPSASGTPGQRVTISCTGSSSNIGAGYDVHWYQQLPGTAPKLLIYRNNQRPSGVPDRFSGSXSGTSXSLAISGLRSEDXADYYCQSYDSSLXGPYWVFXXXNQXDGPRXXXKTMTXXXXXXDIDYXXXXXQXRXAXXXHHHSPXXP

SEQ ID NO: 152 - protein, 303 aa, Artificial Sequence
  C4 (3) scFv antibody
  VARIANT (161)..(161): Any amino acid
  VARIANT (184)..(184): Any amino acid
  VARIANT (191)..(191): Any amino acid
  VARIANT (200)..(200): Any amino acid
  VARIANT (210)..(210): Any amino acid
  VARIANT (237)..(237): Any amino acid
  VARIANT (247)..(247): Any amino acid
  VARIANT (254)..(254): Any amino acid
  VARIANT (260)..(261): Any amino acids
  VARIANT (265)..(265): Any amino acid
  VARIANT (268)..(268): Any amino acid
  VARIANT (276)..(277): Any amino acids
  VARIANT (284)..(284): Any amino acid
  VARIANT (287)..(288): Any amino acids
  VARIANT (292)..(294): Any amino acids
  VARIANT (296)..(301): Any amino acids
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGWSTSSFDYWGQGTLVTVSSGGGGSGGGGSGGGGSQSVLTQPPSASGTPGQRVTISCSGSSSXIGNHYVSWYQQLPGTATKLLIYXDDLLPSXVPDRFSGSXSGTSASLAIXGLRSEDEADYYCAAWDDRSGQVLFGGXTKLTVLGDYXDHDGDYXDHDIDXXDDDXKAXAHHHHHHXXRWPIRPXVSXXTIHXXXFXXXXXXKT

SEQ ID NO: 153 - protein, 317 aa, Artificial Sequence
  C4 (4) scFv antibody
  VARIANT (160)..(160): Any amino acid
  VARIANT (198)..(199): Any amino acids
  VARIANT (236)..(236): Any amino acid
  VARIANT (240)..(240): Any amino acid
  VARIANT (246)..(246): Any amino acid
  VARIANT (248)..(249): Any amino acids
  VARIANT (259)..(259): Any amino acid
  VARIANT (262)..(265): Any amino acids
  VARIANT (267)..(267): Any amino acid
  VARIANT (270)..(270): Any amino acid
  VARIANT (277)..(277): Any amino acid
  VARIANT (279)..(280): Any amino acids
  VARIANT (285)..(286): Any amino acids
  VARIANT (289)..(292): Any amino acids
  VARIANT (294)..(294): Any amino acid
  VARIANT (298)..(298): Any amino acid
  VARIANT (301)..(302): Any amino acids
  VARIANT (304)..(304): Any amino acid
  VARIANT (306)..(315): Any amino acids
  VARIANT (317)..(317): Any amino acid
EVQLLESGGGLVQPGGSLRLSCAASGFTFSSYSMNWVRQAPGKGLEWVSGISGSGGSTYYADSVKGRFTISRDNSKNTLYLQMNSLRAEDTAVYYCAKHSGYGFDIWGQGTLVTVSSGGGGSGGGGSGGGGSQSVLTQPPSASGTPGQRVTISCSGGASXLGMHFVSWYQQLPGTAPKLLIYYDDLLPSGVPDRFSGXXSGTSASLAISGLRSEDEADYYCAAWDDSLNGWVFGGXTKLXVLGDYXDXXGDYKDHDIDXKDXXXXAXAHXHHHHSPXWXXRPIVXXITXXXXVXLQRXDWXXPXVXXXXXXXXXXPX

SEQ ID NO: 154 - protein, 275 aa, Artificial Sequence
  C3 (3) scFv antibody
  VARIANT (199)..(199): Any amino acid
  VARIANT (208)..(208): Any amino acid
  VARIANT (223)..(223): Any amino acid
  VARIANT (225)..(225): Any amino acid
  VARIANT (244)..(244): Any amino acid
  VARIANT (254)..(254): Any amino acid
  VARIANT (268)..(268): Any amino acid
  VARIANT (270)..(270): Any amino acid
  VARIANT (272)..(273): Any amino acids
EVQLLESGGGLVQPGGSLRLSCAASGFTFSSYAMNWVRQAPGKGLEWVANINQDGSTKFYVDSVKGRFTISRDNSKNTLYLQMNSLRAEDTAVYYCARDTGGNYLGGYYYYGMDVWGQGTLVTVSSGGGGSGGGGSGGGGSQSVLTQPPSASGTPGQRVTISCSGSSSNIGSNYVYWYQQLPGTAPKLLIYRNDQRPSXVPDRFSGSXSGTSASLAISGLRSXDXADYYCSSYAGNNNLVFGGXTKLTVLGDYXDHDGDYKDHDIDYXDXDXXAA

SEQ ID NO: 155 - protein, 273 aa, Artificial Sequence
  C3 (4) scFv antibody
  VARIANT (220)..(220): Any amino acid
  VARIANT (242)..(242): Any amino acid
  VARIANT (250)..(250): Any amino acid
  VARIANT (252)..(252): Any amino acid
  VARIANT (261)..(261): Any amino acid
  VARIANT (269)..(270): Any amino acids
EVQLLESGGGLVQPGGSLRLSCAASGFTFSDHYMDWVRQAPGKGLEWVSGISGNGATIDYADSVKGRFTISRDNSKNTLYLQMNSLRAEDTAVYYCARPSITAAGSEDAFDLWGQGTLVTVSSGGGGSGGGGSGGGGSQSVLTQPPSASGTPGQRVTISCSGSSSNIGSNYVYWYQQLPGTAPKLLIYGNSNRPSGVPDRFSGSKSGTSASLAISGLRSXDGADYYCQSYDSSLSGWVFGGXTKLTVLGXYXDHDGDYKDXDIDYKDDXXKAA

SEQ ID NO: 156 - protein, 247 aa, Artificial Sequence
  C3 (5) scFv antibody
  VARIANT (195)..(195): Any amino acid
  VARIANT (204)..(204): Any amino acid
  VARIANT (214)..(214): Any amino acid
  VARIANT (221)..(221): Any amino acid
  VARIANT (234)..(234): Any amino acid
  VARIANT (239)..(239): Any amino acid
  VARIANT (241)..(241): Any amino acid
EVQLLESGGGLVQPGGSLRLSCAASGFTFSNYWMSWVRQAPGKGLEWVSGISGSGGTTYYADFVKGRFTISRDNSKNTLYLQMNSLRAEDTAVYYCARKYYYGSSGAFDIWGQGTLVTVSSGGGGSGGGGSGGGGSQSVLTQPPSASGTPGQRVTISCTGSSSNIGAGYDVHWYQQLPGTAPKLLIYRNNQRPSXVPDRFSGSXSGTSASLAIXGLRSEDXADYYCAAWDDSLXGPVFXGXTKLTVL

SEQ ID NO: 157 - protein, 275 aa, Artificial Sequence
  C3 (6) scFv antibody
  VARIANT (208)..(208): Any amino acid
  VARIANT (225)..(225): Any amino acid
  VARIANT (244)..(245): Any amino acids
  VARIANT (252)..(254): Any amino acids
  VARIANT (267)..(268): Any amino acids
  VARIANT (270)..(270): Any amino acid
  VARIANT (272)..(273): Any amino acids
EVQLLESGGGLVQPGGSLRLSCAASGFTFSSYAMNWVRQAPGKGLEWVANINQDGSTKFYVDSVKGRFTISRDNSKNTLYLQMNSLRAEDTAVYYCARDTGGNYLGGYYYYGMDVWGQGTLVTVSSGGGGSGGGGSGGGGSQSVLTQPPSASGTPGQRVTISCSGSSSNIGSNYVYWYQQLPGTAPKLLIYRNDQRPSGVPDRFSGSXSGTSASLAISGLRSEDXADYYCSSYAGNNNLVFGGXXKLTVLGXXXDHDGDYKDHDIDXXDXDXXAA

SEQ ID NO: 158 - protein, 220 aa, Artificial Sequence
  MYOM2 (1) scFv antibody
  VARIANT (158)..(158): Any amino acid
  VARIANT (188)..(188): Any amino acid
  VARIANT (196)..(197): Any amino acids
  VARIANT (202)..(202): Any amino acid
  VARIANT (207)..(207): Any amino acid
EVQLLESGGGLVQPGGSLRLSCAASGFTFSSYAMSWVRQAPGKGLEWVSGISGSGGSTYYADSVKGRFTISRDNSKNTLYLQMNSLRAEDTAVYYCARGVVAGSWGQGTLVTVSSGGGGSGGGGSGGGGSQSVLTQPPSASGTPGQRVTISCSGSSSXIGNNAVNWYQQLPGTAPKLLIYDNNKRPSXVPDRFSGXXSGTSXSLAIXGLRSEDEADYYCA

SEQ ID NO: 159 - protein, 246 aa, Artificial Sequence
  MYOM2 (2) scFv antibody
  VARIANT (192)..(192): Any amino acid
  VARIANT (201)..(201): Any amino acid
  VARIANT (218)..(218): Any amino acid
  VARIANT (238)..(238): Any amino acid
EVQLLESGGGLVQPGGSLRLSCAASGFTFSNEWMAWVRQAPGKGLEWVSSISSSSSYIYYADSVKGRFTISRDNSKNTLYLQMNSLRAEDTAVYYCAGTYHDFWSATYWGQGTLVTVSSGGGGSGGGGSGGGGSQSVLTQPPSASGTPGQRVTISCSGSSSNIGSNTVNWYQQLPGTAPKLLIYGNSNRPSXVPDRFSGSXSGTSASLAISGLRSEDXADYYCAAWDDSLNGWVFGGXTKLTVLGD

SEQ ID NO: 160 - protein, 271 aa, Artificial Sequence
  LUM scFv antibody
  VARIANT (203)..(203): Any amino acid
  VARIANT (240)..(240): Any amino acid
  VARIANT (247)..(247): Any amino acid
  VARIANT (250)..(250): Any amino acid
  VARIANT (263)..(265): Any amino acids
  VARIANT (268)..(269): Any amino acids
EVQLLESGGGLVQPGGSLRLSCAASGFTFSSNYMSWVRQAPGKGLEWVSAISASGTYTYYTDSVNGRFTISRDNSKNTLYLQMNSLRAEDTAVYYCARVNTVGLGTPFDNWGQGTLVTVSSGGGGSGGGGSGGGGSQSVLTQPPSASGTPGQRVTISCSGSSSNIGSNTVNWYQQLPGTAPKLLIYGNRNRPSGVPDRFSGSXSGTSASLAISGLRSEDEADYYCAAWDDSLSGWVFGGXTKLTVLXDYXDHDGDYKDHDIDXXXDDXXAA

SEQ ID NO: 161 - protein, 266 aa, Artificial Sequence
  DUSP9 scFv antibody
  VARIANT (197)..(197): Any amino acid
  VARIANT (235)..(235): Any amino acid
  VARIANT (245)..(245): Any amino acid
  VARIANT (263)..(263): Any amino acid
EVQLLESGGGLVQPGGSLRLSCAASGFTFSSYGFHWVRQAPGKGLEWVAVISYDGSNKYYADSVKGRFTISRDNSKNTLYLQMNSLRAEDTAVYYCARGEFGVYWGQGTLVTVSSGGGGSGGGGSGGGGSQSVLTQPPSASGTPGQRVTISCSGSSSNIGSNYVYWYQQLPGTAPKLLIYGNRNRPSGVPDRFSGSXSGTSASLAISGLRSEDEADYYCSSYAGSNNFEVVFGGXTKLTVLGDYXDHDGDYKDHDIDYKDDDXKAA

SEQ ID NO: 162 - protein, 237 aa, Artificial Sequence
  CHX10 (1) scFv antibody
  VARIANT (191)..(191): Any amino acid
  VARIANT (200)..(200): Any amino acid
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARNSDYYGMDVWGQGTLVTVSSGGGGSGGGGSGGGGSQSVLTQPPSASGTPGQRVTISCSGSSSNIGYSDVYWYQQLPGTAPKLLIYENNKRPSXVPDRFSGSXSGTSASLAISGLRSEDEADYYCSTWDDSLNGHVIFGG

SEQ ID NO: 163 - protein, 232 aa, Artificial Sequence
  CHX10 (3) scFv antibody
  VARIANT (163)..(163): Any amino acid
  VARIANT (193)..(193): Any amino acid
  VARIANT (201)..(202): Any amino acids
  VARIANT (207)..(207): Any amino acid
  VARIANT (219)..(219): Any amino acid
  VARIANT (226)..(226): Any amino acid
EVQLLESGGGLVQPGGSLRLSCAASGFTFSSYAMSWVRQAPGKGLEWVAVISYDGSNKYYADSVKGRFTISRDNSKNTLYLQMNSLRAEDTAVYYCARNYGDSINWFDPWGQGTLVTVSSGGGGSGGGGSGGGGSQSVLTQPPSASGTPGQRVTISCSGSSSXIRSNTVNWYQQLPGTAPKLLIYGNSNRPSXVPDRFSGXXSGTSXSLAISGLRSEDXADYYCAXWDDSLN

SEQ ID NO: 164 - protein, 236 aa, Artificial Sequence
  ATP-5B (1) scFv antibody
  VARIANT (161)..(161): Any amino acid
  VARIANT (191)..(191): Any amino acid
  VARIANT (199)..(200): Any amino acids
  VARIANT (217)..(217): Any amino acid
EVQLLESGGGLVQPGGSLRLSCAASGFTFSSYAMHWVRQAPGKGLEWVAVISYDGSKTYHADSVEGRFTISRDNSKNTLYLQMNSLRAEDTAVYYCARHLRPYYFDYWGQGTLVTVSSGGGGSGGGGSGGGGSQSVLTQPPSASGTPGQRVTISCSGSSSXIGSNTVNWYQQLPGTAPKLLIYGNSNRPSXVPDRFSGXXSGTSASLAISGLRSEDXADYYCSAWDDRLRGRVFGG

SEQ ID NO: 165 - protein, 324 aa, Artificial Sequence
  ATP-5B (2) scFv antibody
  VARIANT (201)..(201): Any amino acid
  VARIANT (207)..(207): Any amino acid
  VARIANT (212)..(212): Any amino acid
  VARIANT (244)..(244): Any amino acid
  VARIANT (254)..(255): Any amino acids
  VARIANT (258)..(259): Any amino acids
  VARIANT (268)..(269): Any amino acids
  VARIANT (271)..(271): Any amino acid
  VARIANT (273)..(273): Any amino acid
  VARIANT (275)..(275): Any amino acid
  VARIANT (278)..(278): Any amino acid
  VARIANT (286)..(289): Any amino acids
  VARIANT (294)..(307): Any amino acids
  VARIANT (309)..(309): Any amino acid
  VARIANT (311)..(314): Any amino acids
  VARIANT (317)..(324): Any amino acids
EVQLLESGGGLVQPGGSLRLSCAASGFTFSSYGMHWVRQAPGKGLEWVSLISSASSYIYHADSVKGRFTISRDNSKNTLYLQMNSLRAEDTAVYYCARAGRVCTNGVCHTTFDYWGQGTLVTVSSGGGGSGGGGSGGGGSQSVLTQPPSASGTPGQRVTISCSGDRSNIGSNTVNWYQQLPGTAPKLLIYGNSNRPSGVPXRFSGSXSGTSXSLAISGLRSEDEADYYCQSYDSSLSAVVFGGXTKLTVLGDYXXHDXXYKDHDIDYXXDXDXAXAHXHHHHSPRXXXXPIVSXXXXXXXXXXXXXXLXKXXXXPTXXXXXXXX

SEQ ID NO: 166 - protein, 269 aa, Artificial Sequence
  ATP-5B (3) scFv antibody
  VARIANT (192)..(192): Any amino acid
  VARIANT (201)..(201): Any amino acid
  VARIANT (218)..(218): Any amino acid
  VARIANT (238)..(238): Any amino acid
  VARIANT (242)..(242): Any amino acid
  VARIANT (245)..(245): Any amino acid
  VARIANT (248)..(248): Any amino acid
  VARIANT (255)..(255): Any amino acid
  VARIANT (261)..(263): Any amino acids
EVQLLESGGGLVQPGGSLRLSCAASGFTFSTYAMSWVRQAPGKGLEWVSSISSTSTYIHYADSVKGRFTISRDNSKNTLYLQMNSLRAEDTAVYYCARVSSWYSAFDIWGQGTLVTVSSGGGGSGGGGSGGGGSQSVLTQPPSASGTPGQRVTISCTGSSSNIGNNAVNWYQQLPGTAPKLLIYSNNQRPSXVPDRFSGSXSGTSASLAISGLRSEDXADYYCQSYDSSLSGVIFGGXTKLXVLXDYXDHDGDYXDHDIDXXXDDDKAA

SEQ ID NO: 167 - protein, 267 aa, Artificial Sequence
  Sox11a scFv antibody
  VARIANT (203)..(203): Any amino acid
  VARIANT (220)..(220): Any amino acid
  VARIANT (227)..(227): Any amino acid
  VARIANT (236)..(236): Any amino acid
  VARIANT (243)..(243): Any amino acid
  VARIANT (246)..(246): Any amino acid
  VARIANT (252)..(253): Any amino acids
  VARIANT (259)..(259): Any amino acid
  VARIANT (262)..(262): Any amino acid
  VARIANT (264)..(264): Any amino acid
EVQLLESGGGLVQPGGSLRLSCAASGFTFSDFWMSWVRQAPGKGLEWVSSISGGGGTAFYVDSVKGRFTISRDNSKNTLYLQMNSLRAEDTALYYCARMTDLESGDAFDIWGQGTLVTVSSGGGGSGGGGSGGGGSQSVLTQPPSASGTPGQRVTISCSGSSSNIGSNYVNWYQQLPGTAPKLLIYNDNVRPSGVPDRFSGSXSGTSASLAISGLRSEDXADYYCQXWGTGVFGGXTKLTVLXDYXDHDGDXXDHDIDXKDXDXKAA

SEQ ID NO: 168 - protein, 247 aa, Artificial Sequence
  TBC1D9 (1) scFv antibody
  VARIANT (192)..(193): Any amino acids
  VARIANT (201)..(202): Any amino acids
  VARIANT (239)..(239): Any amino acid
EVQLLESGGGLVQPGGSLRLSCAASGFTFSSYSMSWVRQAPGKGLEWVAVISYDGSNKYYADSVKGRFTISRDNSKNTLYLQMNSLRAEDTAVYYCARDRTRGSTALDIWGQGTLVTVSSGGGGSGGGGSGGGGSQSVLTQPPSASGTPGQRVTISCSGSSSYIGSNYVYWYQQLPGTAPKLLIYRNNQRPXXVPDRFSGXXSGTSASLAISGLRSEDEADYYCAAWDDSLSGWVFGGXTKLTVLGD

SEQ ID NO: 169 - protein, 250 aa, Artificial Sequence
  UPF3B (1) scFv antibody
  VARIANT (187)..(187): Any amino acid
  VARIANT (196)..(196): Any amino acid
  VARIANT (206)..(206): Any amino acid
  VARIANT (212)..(213): Any amino acids
  VARIANT (234)..(234): Any amino acid
  VARIANT (244)..(244): Any amino acid
  VARIANT (248)..(248): Any amino acid
EVQLLESGGGLVQPGGSLRLSCAASGFTFSDYYMTWIRQAPGKGLEWVSDISWNGSRTHYADSVKGRFTISRDNSKNTLYLQMNSLRAEDTAVYYCSSHLVYWGQGTLVTVSSGGGGSGGGGSGGGGSQSVLTQPPSASGTPGQRVTISCTGSSSNIGAGYDVHWYQQLPGTAPKLLIYDNNKRPSXVPDRFSGSXSGTSASLAIXGLRSEXXADYYCQTYDSSLSGSVVFGGXTKLTVLGDYXDHDXDY

SEQ ID NO: 170 - protein, 251 aa, Artificial Sequence
  UPF3B (2) scFv antibody
  VARIANT (166)..(166): Any amino acid
  VARIANT (197)..(197): Any amino acid
  VARIANT (206)..(206): Any amino acid
  VARIANT (221)..(221): Any amino acid
  VARIANT (243)..(243): Any amino acid
EVQLLESGGGLVQPGGSLRLSCAASGFTFSSYAMSWVRQAPGKGLEWVSYISSSSSYANYADSVKGRFTISRDNSKNTLYLQMNSLRAEDTAVYYCARLGVYSGTYLFAFDIWGQGTLVTVSSGGGGSGGGGSGGGGSQSVLTQPPSASGTPGQRVTISCTGSSSXIGAGYDVHWYQQLPGTAPKLLIYGNSNRPSXVPDRFSGSXSGTSASLAISGLRSXDEADYYCQSRDSSLSGWVFGGXTKLTVLGD

SEQ ID NO: 171 - protein, 123 aa, Artificial Sequence
  Apo-A4 (1) scFv antibody
EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCARVAYDIDAFDMWGQGTLVTVSSGGGG

SEQ ID NO: 172 - protein, 301 aa, Artificial Sequence
  Apo-A4 (2) scFv antibody
  VARIANT (201)..(201): Any amino acid
  VARIANT (238)..(238): Any amino acid
  VARIANT (245)..(245): Any amino acid
  VARIANT (265)..(268): Any amino acids
  VARIANT (281)..(281): Any amino acid
  VARIANT (285)..(286): Any amino acids
  VARIANT (288)..(289): Any amino acids
  VARIANT (293)..(296): Any amino acids
  VARIANT (298)..(300): Any amino acids
EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCARVAYDIDAFDMWGQGTLVTVSSGGGGSGGGGSGGGGSQSVLTQPPSASGTPGQRVTISCSGSFSNIGSNYVYWYQQLPGTAPKLLIYENNKRPSGVPDRFSGSXSGTSASLAISGLRSEDEADYYCAAWDDSLNGPMFGGXTKLTVLXDYKDHDGDYKDHDIDYKDDXXXXAAHHHHHHSPRWXIRPXXSXXTIHXXXXLXXXD

SEQ ID NO: 173 - protein, 232 aa, Artificial Sequence
  Apo-A4 (3) scFv antibody
  VARIANT (196)..(196): Any amino acid
  VARIANT (213)..(213): Any amino acid
EVQLLESGGGLVQPGGSLRLSCAASGFTFSSYSMNWVRQAPGKGLEWVSAITGSGNATFYADSVKGRFTISRDNSKNTLYLQMNSLRAEDTAVYYCTTGATTRWGQGTLVTVSSGGGGSGGGGSGGGGSQSVLTQPPSASGTPGQRVTISCSGSRSNIGSNHVFWYQQLPGTAPKLLIYENNKRPSGVPDRFSGSXSGTSASLAISGLRSEDXADYYCAAWDDSLSGWVFGG

SEQ ID NO: 174 - protein, 196 aa, Artificial Sequence
  TBC1D9 (2) scFv antibody
  VARIANT (168)..(168): Any amino acid
EVQLLESGGGLVQPGGSLRLSCAASGFTFSNAWMSWVRQAPGKGLEWVSFISSSSSYIYYADSVKGRFTISRDNSKNTLYLQMNSLRAEDTAVYYCARVNLVGCTNGVCNGHDYWGQGTLVTVSSGGGGSGGGGSGGGGSQSVLTQPPSASGTPGQRVTISCSGSSSXIGSNTVNWYQQLPGTAPKLLIYDNNKRP

SEQ ID NO: 175 - protein, 234 aa, Artificial Sequence
  TBC1D9 (3) scFv antibody
  VARIANT (159)..(159): Any amino acid
  VARIANT (189)..(189): Any amino acid
  VARIANT (198)..(198): Any amino acid
  VARIANT (215)..(215): Any amino acid
EVQLLESGGGLVQPGGSLRLSCAASGFTFGDYAMSWVRQAPGKGLEWVSAISGSGGSTYYADSVKGRFTISRDNSKNTLYLQMNSLRAEDTAVYYCAKGRTMASHWGQGTLVTVSSGGGGSGGGGSGGGGSQSVLTQPPSASGTPGQRVTISCSGSSSXIGNNHVSWYQQLPGTAPKLLIYGNSNRPSXVPDRFSGSXSGTSASLAISGLRSEDXADYYCAAWDNSLKVWMFGG

SEQ ID NO: 176 - protein, 239 aa, Artificial Sequence
  ORP-3 (1) scFv antibody
  VARIANT (164)..(164): Any amino acid
  VARIANT (193)..(194): Any amino acid
  VARIANT (202)..(203): Any amino acids
  VARIANT (208)..(208): Any amino acid
  VARIANT (220)..(220): Any amino acid
EVQLLESGGGLVQPGGSLRLSCAASGFTFSSNYMSWVRQAPGKGLEWVSYISGNSGYTNYADSVKGRFTISRDNSKNTLYLQMNSLRAEDTAVYYCARHAGSYDMYGMDVWGQGTLVTVSSGGGGSGGGGSGGGGSQSVLTQPPSASGTPGQRVTISCSGSTSXIGSHYVYWYQQLPGTAPKLLIYGNSNRPXXVPDRFSGXXSGTSXSLAISGLRSEDXADYYCQSYDSRLSGWVFGG

SEQ ID NO: 177 - protein, 241 aa, Artificial Sequence
  ORP-3 (2) scFv antibody
  VARIANT (158)..(158): Any amino acid
  VARIANT (197)..(197): Any amino acid
  VARIANT (227)..(227): Any amino acid
  VARIANT (234)..(234): Any amino acid
EVQLLESGGGLVQPGGSLRLSCAASGFTFSSYAMHWVRQAPGKGLEWVAVISYDGSNKYYADSVKGRFTISRDNSKNTLYLQMNSLRAEDTAVYYCARKSSLDVWGQGTLVTVSSGGGGSGGGGSGGGGSQSVLTQPPSASGTPGQRVTISCSGSSSXIGNNYVSWYQQLPGTAPKLLIYDDNKRPSGVPDRFSGSXSDTSASLAISGLRSEDEADYYCAAWDDSLXGRVFGGXTKLTVLG

SEQ ID NO: 178 - protein, 254 aa, Artificial Sequence
  CIMS (5) scFv antibody
  VARIANT (3)..(3): Any amino acid
  VARIANT (232)..(233): Any amino acids
  VARIANT (247)..(247): Any amino acid
EVXLLESGGGLVQPGGSLRLSCAASGFTFSDHYMDWVRQAPGKGLEWVSGISGSGGSTYYGDSVKGRFTISRDNSKNTLYLQMNSLRAEDTAVYYCASRLYWGQGTLVTVSSGGGGSGGGGSGGGGSQSVLTQPPSASGTPGQRVTISCTGSSSNIGAGYVVHWYQQLPGTAPKLLIYDNDKRPSGVPDRFSGSKSGTSASLAISGLRSEDEADYYCAAWDDSLDAVLFGGXXKLTVLGEQKLISEXDLSGSAA

SEQ ID NO: 179 - protein, 321 aa, Artificial Sequence
  CIMS (13) scFv antibody
  VARIANT (206)..(206): Any amino acid
  VARIANT (227)..(227): Any amino acid
  VARIANT (244)..(244): Any amino acid
  VARIANT (258)..(259): Any amino acids
  VARIANT (266)..(266): Any amino acid
  VARIANT (277)..(277): Any amino acid
  VARIANT (286)..(286): Any amino acid
  VARIANT (291)..(293): Any amino acids
  VARIANT (295)..(297): Any amino acids
  VARIANT (302)..(304): Any amino acids
  VARIANT (306)..(306): Any amino acid
  VARIANT (308)..(310): Any amino acids
  VARIANT (312)..(313): Any amino acids
  VARIANT (317)..(321): Any amino acids
EVQLLESGGGLVQPGGSLRLSCAASGFTFSSYAMSWVRQAPGKGLEWVSAISGSGGRTYYTDSVRDRFTISRDNSKNTLYLQMNSLRAEDTAVYYCARDLMPVCQYCYGMDVWGQGTLVTVSSGGGGSGGGGSGGGGSQSVLTQPPSASGTPGQRVTISCTGSSSNIGAGYDVHWYQQLPGTAPKLLIYSNNQRPSGVPDRFSGSXSGTSASLAISGLRSEDEADYXCQSYDSSLNKDVVFGGXTKLTVLGEQKLISXXDLSGSAXAHHHHHHSPRXPIRPIVSRXTIHWXXXLXXXDWENXXXTXLXXXAXXPPFXXXXX